(19) United States
(12) Patent Application Publication
(10) Pub. No.: US 2026/0201370 A1
(43) Pub. Date: Jul. 16, 2026

(54) COMPOSITIONS AND METHODS FOR TARGETED EPIGENETIC MODIFICATION

(71) Applicant: Mammoth Biosciences, Inc., Brisbane, CA (US)

(72) Inventors: Yuchen GAO, South San Francisco, CA (US); Alex Tianjiun WEI, San Francisco, CA (US); Kelsey Nicole DOCOUTO, San Francisco, CA (US); Aaron DELOUGHERY, San Francisco, CA (US); Wiputra Jaya HARTONO, San Francisco, CA (US); Benjamin Julius RAUCH, San Francisco, CA (US); Stepan TYMOSHENKO, Sacramento, CA (US); William Douglass WRIGHT, Fairfield, CA (US)

(73) Assignee: Mammoth Biosciences, Inc., Brisbane, CA (US)

(21) Appl. No.: 19/385,214

(22) Filed: Nov. 11, 2025

Related U.S. Application Data
(63) Continuation of application No. PCT/US2024/028769, filed on May 10, 2024.
(60) Provisional application No. 63/501,872, filed on May 12, 2023.
(60) Provisional application No. 63/510,678, filed on Jun. 28, 2023.
(60) Provisional application No. 63/586,712, filed on Sep. 29, 2023.
(60) Provisional application No. 63/597,841, filed on Nov. 10, 2023.
(60) Provisional application No. 63/624,457, filed on Jan. 24, 2024.

(51) Int. Cl. C12N 15/11; C12N 9/10; C12N 9/22; C12N 15/86; C12N 15/88

(52) U.S. Cl. CPC C12N 15/111; C12N 9/1007; C12N 9/226; C12N 15/86; C12N 15/88; C07K 2319/81; C12N 2310/20; C12N 2750/14143; C12Y 201/01037

(57) ABSTRACT

Provided herein are compositions, systems, and methods comprising effector proteins, and uses thereof. These effector proteins may be characterized as CRISPR-associated (Cas) proteins. Various compositions, systems, and methods of the present disclosure may leverage the activities of these effector proteins for the epigenetic modification of nucleic acids.

### CROSS-REFERENCE TO RELATED APPLICATIONS

The present application is a continuation of International PCT Application No. PCT/US2024/028769, filed May 10, 2024, which claims priority to U.S. Provisional Application 63/501,872, filed May 12, 2023; U.S. Provisional Application 63/510,678, filed Jun. 28, 2023; U.S. Provisional Application 63/586,712, filed Sep. 29, 2023; U.S. Provisional Application 63/597,841, filed Nov. 10, 2023; and U.S. Provisional Application 63/624,457, filed Jan. 24, 2024, each of which is herein incorporated by reference in its entirety.

### SEQUENCE LISTING

The contents of the electronic sequence listing (MABI_038_05US_SeqList_ST26.xml; Size: 2,811,137 bytes; and Date of Creation: Aug. 14, 2025) are herein incorporated by reference in its entirety.

### FIELD

The present disclosure relates generally to polypeptides, such as effector proteins, compositions of such polypeptides and guide nucleic acids, systems, and methods of using such polypeptides and compositions, including modifying target nucleic acids.

### BACKGROUND

Clustered Regularly Interspaced Short Palindromic Repeats (CRISPR) and associated proteins (Cas proteins), sometimes referred to as CRISPR/Cas systems, were first identified in certain bacterial species and are now understood to form part of a prokaryotic acquired immune system. CRISPR/Cas systems provide immunity in bacteria and archaea against viruses and plasmids by targeting the nucleic acids of the viruses and plasmids in a sequence-specific manner. While CRISPR/Cas proteins are involved in the acquisition, targeting and cleavage of foreign DNA or RNA, the systems may also contain a CRISPR array, which includes direct repeats flanking short spacer sequences that, in part, guide Cas proteins to their targets.

### SUMMARY

The present disclosure provides compositions, systems and methods for the epigenetic modification of target nucleic acids. In some embodiments, compositions, systems, and methods comprise or employ an effector protein (e.g., a dCas protein), an effector partner (e.g., a methyltransferase), and a guide nucleic acid. By way of non-limiting example, epigenetic modifications include methylation of target DNA. Methylation of target DNA may result in reducing or silencing target DNA expression. In some embodiments, the reducing or silencing of target DNA expression is not transient, but rather permanent or heritable. In some embodiments, compositions, systems and methods comprise or employ a catalytically inactive effector protein that does not create double stranded DNA breaks. In some instances, compositions, systems and methods are useful for the treatment of a disease or disorder.

In some aspects, disclosed herein are compositions comprising a fusion protein or a nucleic acid encoding the same, wherein the fusion protein comprises: (a) an effector protein comprising an amino acid sequence that is at least 90%, at least 95%, at least 97%, at least 98%, or at least 99% identical to a sequence selected from TABLE 1; (b) a methyltransferase; and (c) a repressor domain. In some embodiments, the effector protein comprises at least one amino acid substitution relative to the sequence selected from TABLE 1, wherein the amino acid substitution reduces a nuclease activity of the effector protein relative to the sequence selected from TABLE 1. In some embodiments, the amino acid sequence is at least 90%, at least 95%, at least 97%, at least 98%, or at least 99% identical to SEQ ID NO: 1 and wherein the effector protein comprises at least one amino acid substitution relative to SEQ ID NO: 1, wherein the amino acid substitution(s) is selected from: L26R, D369A, D369N, D658A, D658N, E567A, E567Q, A121Q, L26K, I471T, K189P, S638K, Q54R, E258K, Q79R, Y220S, N406K, E119S, S223P, K92E, K435Q, N568D, E109R, H208R, K184R, K38R, L182R, Q183R, S108R, S198R, T114R, and V521T, and a combination thereof. In some embodiments, the effector protein comprises the amino acid substitutions: L26R and E567Q. In some embodiments, the effector protein comprises an amino acid sequence that is 100% identical to SEQ ID NO: 2 with the exception of 1 to 10, 1 to 20, or 1 to 30 conservative amino acid substitutions at positions other than residue 26 and 567. In some embodiments, the effector protein comprises an amino acid sequence of SEQ ID NO: 2. In some embodiments, the length of the effector protein is 400-800 amino acids. In some embodiments, the effector protein is fused to a nuclear localization signal (NLS). In some embodiments, at least one methyltransferase is selected from DNMT3A, DNMT3L, and a combination thereof. In some embodiments, the methyltransferase comprises an amino acid sequence that is at least 90%, at least 95%, at least 97%, at least 98%, at least 99%, or 100% identical to a sequence selected from SEQ ID NO: 10 and SEQ ID NO: 11. In some embodiments, the repressor domain is selected from KRAB (ZNF10), KRAB (ZIM3), YAF2, MPP8, RYBP, CBX3VD, and CBX5VD, and a combination thereof. In some embodiments, the repressor domain comprises an amino acid sequence that is at least 90%, at least 95%, at least 97%, at least 98%, at least 99%, or 100% identical to a sequence selected from SEQ ID NOs: 12 and 3005-3009. In some embodiments, the composition comprises a guide nucleic acid or a nucleic acid encoding the guide nucleic acid, wherein the guide nucleic acid comprises a protein binding sequence and a spacer sequence. In some embodiments, the spacer sequence is at least 90% complementary to a target sequence in a eukaryotic genome. In some embodiments, the eukaryotic genome is a human genome. In some embodiments, the effector protein comprises an amino acid sequence that is at least 90%, at least 95%, at least 97%, at least 98%, at least 99%, or 100% identical to SEQ ID NO: 1, and the protein binding sequence comprises a sequence that is at least 80%, 85%, 90% or 95% identical to a nucleotide sequence selected from SEQ ID NOs: 5-9. In some embodiments, the target sequence is located in a human apolipoprotein C3 (APOC3) gene, a human Proprotein convertase subtilisin/kexin type 9 (PCSK9) gene, or a human Angiopoietin-like 3 (ANGPTL3) gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 37-359. In some embodiments, the guide RNA comprises a nucleotide sequence that is at least 90%, at least 95%, at least 97%, at least 98%, at least 99%, or 100% identical to any one of the nucleotide sequences of SEQ ID NOs: 529-851. In some embodiments, the target sequence is adjacent to a protospacer adjacent motif of 5′-NTTN-3′. In some embodiments, the effector protein comprises an amino acid sequence that is at least 90%, at least 95%, at least 97%, at least 98%, at least 99%, or 100% identical to SEQ ID NO: 35, and the protein binding sequence comprises a sequence that is at least 80%, 85%, 90% or 95% identical to SEQ ID NO: 36. In some embodiments, the target sequence is located in a human apolipoprotein C3 (APOC3) gene, a human Proprotein convertase subtilisin/kexin type 9 (PCSK9) gene, or a human Angiopoietin-like 3 (ANGPTL3) gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 1021-1582. In some embodiments, the guide RNA comprises a nucleotide sequence that is at least 90%, at least 95%, at least 97%, at least 98%, at least 99%, or 100% identical to any one of the nucleotide sequences of SEQ ID NOs: 1976-2537. In some embodiments, the target sequence is adjacent to a protospacer adjacent motif of 5′-NNTN-3′. In some embodiments, amongst the effector protein, methyltransferase, and repressor domain, the effector protein is nearest to the N terminus, the repressor domain is located nearest to the C terminus, and the methyltransferase is located between the effector protein and the repressor domain. In some embodiments, the nucleic acid encoding the fusion protein is an adeno associated viral (AAV) vector. In some embodiments, the AAV vector comprises the nucleic acid encoding the guide nucleic acid or encodes a guide nucleic acid. In some embodiments, the composition further comprises a lipid nanoparticle (LNP). In some embodiments, the nucleic acid encoding the fusion protein is a messenger RNA. In some embodiments, the fusion protein comprises an amino acid sequence that is at least 90%, at least 95%, at least 97%, at least 98%, at least 99%, or 100% identical to a sequence selected from SEQ ID NOs: 16-21, 3004, 3013-3025.

In some aspects, disclosed herein are compositions comprising a fusion protein or a nucleic acid encoding the fusion protein, wherein the fusion protein comprises: a) an effector protein; and b) a methyltransferase, wherein the methyltransferase is DNMT3L, wherein the composition does not comprise DNMT3A. In some embodiments, the effector protein is a Cas protein. In some embodiments, the effector protein comprises a Type V Cas protein. In some embodiments, the Cas protein comprises a Cas12J protein. In some embodiments, the effector protein is not greater than 800 contiguous amino acids in length.

In some aspects, disclosed herein are compositions comprising an effector protein that is at least 90%, at least 95%, at least 97%, at least 98%, or at least 99% identical to SEQ ID NO: 1, wherein the effector protein comprises amino acid substitutions of L26R and E567Q relative to SEQ ID NO: 1. In some embodiments, the effector protein comprises 1 to 10, 1 to 20, or 1 to 30 conservative amino acid substitutions relative to SEQ ID NO: 1 at positions other than residue 26 and 567. In some embodiments, the effector protein comprises an amino acid sequence of SEQ ID NO: 2.

In some aspects, disclosed herein are compositions comprising: a) a fusion protein or a nucleic acid encoding the fusion protein, wherein the fusion protein comprises: i. an effector protein; and ii. an effector partner; and b) a guide RNA or a nucleic acid encoding the guide RNA, wherein the guide RNA comprises: i. a first region comprising a protein binding sequence; and ii. a second region comprising a spacer sequence that hybridizes to a target sequence of a target nucleic acid. In some embodiments, the effector partner comprises a KRAB domain. In some embodiments, the effector partner comprises at least one methyltransferase selected from DNMT3A, DNMT3L, and a combination thereof. In some embodiments, the methyltransferase is DNMT3L. In some embodiments, the composition does not comprise DNMT3A. In some embodiments, the effector protein comprises an amino acid sequence that is at least 90%, at least 95%, at least 97%, at least 98%, or at least 99% identical to SEQ ID NO: 1. In some embodiments, the effector protein comprises the amino acid sequence that is 100% identical to SEQ ID NO: 1. In some embodiments, the protein binding sequence comprises a nucleotide sequence that is at least 90%, at least 95%, at least 97%, at least 98%, at least 99%, or 100% identical to the nucleotide sequences of SEQ ID NO: 9. In some embodiments, the target sequence is adjacent to a protospacer adjacent motif of 5′-NTTN-3′. In some embodiments, the target nucleic acid comprises a human apolipoprotein C3 (APOC3) gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 37-109. In some embodiments, the guide RNA comprises a nucleotide sequence that is at least 90%, at least 95%, at least 97%, at least 98%, at least 99%, or 100% identical to any one of the nucleotide sequences of SEQ ID NOs: 529-601. In some embodiments, the target nucleic acid comprises a mouse APOC3 gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 360-456. In some embodiments, the guide RNA comprises a nucleotide sequence that is at least 90%, at least 95%, at least 97%, at least 98%, at least 99%, or 100% identical to any one of the nucleotide sequences of SEQ ID NOs: 852-948. In some embodiments, the target nucleic acid comprises a human Proprotein convertase subtilisin/kexin type 9 (PCSK9) gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 110-177. In some embodiments, the guide RNA comprises a nucleotide sequence that is at least 90%, at least 95%, at least 97%, at least 98%, at least 99%, or 100% identical to any one of the nucleotide sequences of SEQ ID NOs: 602-669. In some embodiments, the target nucleic acid comprises a mouse PCSK9 gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 457-528. In some embodiments, the guide RNA comprises a nucleotide sequence that is at least 90%, at least 95%, at least 97%, at least 98%, at least 99%, or 100% identical to any one of the nucleotide sequences of SEQ ID NOs: 949-1020. In some embodiments, the target nucleic acid comprises a human Angiopoietin-like 3 (ANGPTL3) gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 178-359. In some embodiments, the guide RNA comprises a nucleotide sequence that is at least 90%, at least 95%, at least 97%, at least 98%, at least 99%, or 100% identical to any one of the nucleotide sequences of SEQ ID NOs: 670-851. In some embodiments, the effector protein comprises an amino acid sequence that is at least 90%, at least 95%, at least 97%, at least 98%, or at least 99% identical to SEQ ID NO: 35. In some embodiments, the effector protein comprises the amino acid sequence that is 100% identical to SEQ ID NO: 35. In some embodiments, the protein binding sequence comprises a nucleotide sequence that is at least 90%, at least 95%, at least 97%, at least 98%, at least 99%, or 100% identical to the nucleotide sequences of SEQ ID NO: 36. In some embodiments, the target sequence is adjacent to a protospacer adjacent motif of 5′-NNTR-3′. In some embodiments, the target nucleic acid comprises a human APOC3 gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 1021-1202. In some embodiments, the guide RNA comprises a nucleotide sequence that is at least 90%, at least 95%, at least 97%, at least 98%, at least 99%, or 100% identical to any one of the nucleotide sequences of SEQ ID NOs: 1976-2157. In some embodiments, the target nucleic acid comprises a mouse APOC3 gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 1583-1800. In some embodiments, the guide RNA comprises a nucleotide sequence that is at least 90%, at least 95%, at least 97%, at least 98%, at least 99%, or 100% identical to any one of the nucleotide sequences of SEQ ID NOs: 2538-2755. In some embodiments, the target nucleic acid comprises a human PCSK9 gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 1203-1361. In some embodiments, the guide RNA comprises a nucleotide sequence that is at least 90%, at least 95%, at least 97%, at least 98%, at least 99%, or 100% identical to any one of the nucleotide sequences of SEQ ID NOs: 2158-2316. In some embodiments, the target nucleic acid comprises a mouse PCSK9 gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 1801-1975. In some embodiments, the guide RNA comprises a nucleotide sequence that is at least 90%, at least 95%, at least 97%, at least 98%, at least 99%, or 100% identical to any one of the nucleotide sequences of SEQ ID NOs: 2756-2930. In some embodiments, the target nucleic acid comprises a human ANGPTL3 gene. In some embodiments, the spacer sequence comprises a nucleotide sequence that is at least 90%, at least 95%, at least 97%, at least 98%, at least 99%, or 100% identical to any one of the nucleotide sequences of SEQ ID NOs: 1362-1582. In some embodiments, the guide RNA comprises a nucleotide sequence that is at least 90%, at least 95%, at least 97%, at least 98%, at least 99%, or 100% identical to any one of the nucleotide sequences of SEQ ID NOs: 2317-2537. In some embodiments, the fusion protein comprises one effector partner, wherein the effector partner is linked to the C-terminus or N-terminus of the effector protein. In some embodiments, the fusion protein comprises a first effector partner and a second effector partner. In some embodiments, the first effector partner and the second effector partner are selected from DNMT3L, DNMT3A, KRAB(ZNF10), and KRAB(ZIM3). In some embodiments, the first effector partner and the second effector partner are selected from DNMT3L and KRAB(ZNF10). In some embodiments, the first effector partner is linked to the C-terminus of the effector protein. In some embodiments, the second effector partner is linked to the N-terminus of the effector protein. In some embodiments, the second effector partner is linked to the C-terminus of the first effector partner. In some embodiments, the first effector partner is linked to the N-terminus of the effector protein. In some embodiments, the second effector partner is linked to the N-terminus of the first effector partner. In some embodiments, the fusion protein comprises a first effector partner, a second effector partner and a third effector partner. In some embodiments, the first effector partner, the second effector partner and the third effector partner are selected from DNMT3L, DNMT3A, KRAB(ZNF10), and KRAB(ZIM3). In some embodiments, the first effector partner, the second effector partner, and the third effector partner are selected from DNMT3L, DNMT3A and KRAB(ZNF10). In some embodiments, the first effector partner is linked to the C-terminus of the effector protein. In some embodiments, the second effector partner is linked to the N-terminus of the effector protein. In some embodiments, the third effector partner is linked to the C-terminus of the first effector partner. In some embodiments, the third effector partner is linked to the N-terminus of the second effector partner. In some embodiments, the second effector partner is linked to the C-terminus of the first effector partner, and wherein the third effector partner is linked to the C-terminus of the second effector partner. In some embodiments, the first effector partner is linked to the N-terminus of the effector protein, wherein the second effector partner is linked to the N-terminus of the first effector partner, and wherein the third effector partner is linked to the N-terminus of the second effector partner. In some embodiments, the effector protein comprises an amino acid sequence that is at least 90%, 91%, 92%, 93%, 94%, 95%, 96%, 97%, 98%, or 99% identical to an amino acid sequence of SEQ ID NO: 2. In some embodiments, the effector protein comprises the amino acid sequence of SEQ ID NO: 2. In some embodiments, the effector protein comprises an amino acid sequence that is at least 90%, 91%, 92%, 93%, 94%, 95%, 96%, 97%, 98%, or 99% identical to an amino acid sequence of SEQ ID NO: 2950. In some embodiments, the effector protein comprises the amino acid sequence of SEQ ID NO: 2950. In some embodiments, the fusion protein comprises an amino acid sequence that is at least 90%, at least 95%, at least 97%, at least 98%, or at least 99% identical to a sequence selected from SEQ ID NOs: 16-21. In some embodiments, the fusion protein comprises an amino acid sequence selected from SEQ ID Nos: 16-21. In some embodiments, the fusion protein comprises the effector protein comprising an amino acid sequence that is at least 90%, 91%, 92%, 93%, 94%, 95%, 96%, 97%, 98%, or 99% identical to an amino acid sequence of SEQ ID NO: 2, the first effector partner comprising an amino acid sequence that is at least 90%, 91%, 92%, 93%, 94%, 95%, 96%, 97%, 98%, or 99% identical to an amino acid sequence of SEQ ID NO: 11, and the second effector partner comprising an amino acid sequence that is at least 90%, 91%, 92%, 93%, 94%, 95%, 96%, 97%, 98%, or 99% identical to an amino acid sequence of any of SEQ ID NOs: 3005-3009. In some embodiments, the fusion protein comprises the effector protein comprising the amino acid sequence of SEQ ID NO: 2, the first effector partner comprising the amino acid sequence of SEQ ID NO: 11, and the second effector partner comprising an amino acid sequence of any of SEQ ID NOs: 3005-3009. In some embodiments, the fusion protein comprises the effector protein comprising a Cas protein, the first effector partner comprising DNMT3L, and the second effector partner comprising bipartite repressors KRAB(ZNF10)-YAF2. In some embodiments, the fusion protein comprises the effector protein comprising a Cas protein, the first effector partner comprising an amino acid sequence that is at least 90%, 91%, 92%, 93%, 94%, 95%, 96%, 97%, 98%, or 99% identical to an amino acid sequence of SEQ ID NO: 11, and the second effector partner comprising an amino acid sequence that is at least 90%, 91%, 92%, 93%, 94%, 95%, 96%, 97%, 98%, or 99% identical to an amino acid sequence of any of SEQ ID NO: 3005. In some embodiments, the fusion protein comprises the effector protein comprising a Cas protein, the first effector partner comprising the amino acid sequence of SEQ ID NO: 11, and the second effector partner comprising an amino acid sequence of any of SEQ ID NO: 3005. In some embodiments, the fusion protein comprises the effector protein comprising a Cas protein, the first effector partner comprising DNMT3L, and the second effector partner comprising CBX3VD. In some embodiments, the second effector partner further comprises KRAB(ZIM3). In some embodiments, the fusion protein comprises the effector protein comprising a Cas protein, the first effector partner comprising DNMT3L, and the second effector partner comprising bipartite repressors CBX3VD-KRAB(ZIM3). In some embodiments, the fusion protein comprises the effector protein comprising a Cas protein, the first effector partner comprising an amino acid sequence that is at least 90%, 91%, 92%, 93%, 94%, 95%, 96%, 97%, 98%, or 99% identical to an amino acid sequence of SEQ ID NO: 11, and the second effector partner comprising an amino acid sequence that is at least 90%, 91%, 92%, 93%, 94%, 95%, 96%, 97%, 98%, or 99% identical to an amino acid sequence of any of SEQ ID NO: 3008. In some embodiments, the fusion protein comprises the effector protein comprising a Cas protein, the first effector partner comprising the amino acid sequence of SEQ ID NO: 11, and the second effector partner comprising an amino acid sequence of any of SEQ ID NO: 3008. In some embodiments, the fusion protein comprises the effector protein comprising a Cas protein, the first effector partner comprising DNMT3L, and the second effector partner comprising CBX5VD. In some embodiments, the second effector partner further comprises KRAB(ZIM3). In some embodiments, the fusion protein comprises the effector protein comprising a Cas protein, the first effector partner comprising DNMT3L, and the second effector partner comprising bipartite repressors CBX5VD-KRAB(ZIM3). In some embodiments, the fusion protein comprises the effector protein comprising a Cas protein, the first effector partner comprising an amino acid sequence that is at least 90%, 91%, 92%, 93%, 94%, 95%, 96%, 97%, 98%, or 99% identical to an amino acid sequence of SEQ ID NO: 11, and the second effector partner comprising an amino acid sequence that is at least 90%, 91%, 92%, 93%, 94%, 95%, 96%, 97%, 98%, or 99% identical to an amino acid sequence of any of SEQ ID NO: 3009. In some embodiments, the fusion protein comprises the effector protein comprising a Cas protein, the first effector partner comprising the amino acid sequence of SEQ ID NO: 11, and the second effector partner comprising an amino acid sequence of any of SEQ ID NO: 3009. In some embodiments, the Cas protein comprises an amino acid sequence that is at least 90%, 91%, 92%, 93%, 94%, 95%, 96%, 97%, 98%, or 99% identical to an amino acid sequence of SEQ ID NO: 1. In some embodiments, the Cas protein comprises the amino acid sequence of SEQ ID NO: 1. In some embodiments, the Cas protein comprises an amino acid sequence that is at least 90%, 91%, 92%, 93%, 94%, 95%, 96%, 97%, 98%, or 99% identical to an amino acid sequence of SEQ ID NO: 2. In some embodiments, the Cas protein comprises the amino acid sequence of SEQ ID NO: 2. In some embodiments, the first effector partner is linked to the C-terminus of the effector protein. In some embodiments, the second effector partner is linked to the C-terminus of the first effector partner.

In some aspects, disclosed herein are kits comprising the composition of any of the above aspects or embodiments.

In some aspects, disclosed herein are methods of modifying a target nucleic acid, the method comprising contacting the target nucleic acid with the composition of any of the above aspects or embodiments or the kit of any of the above aspects or embodiments.

In some aspects, disclosed herein are cells modified by the composition of any of the above aspects or embodiments, the kit of any of the above aspects or embodiments, or the method of any of the above aspects or embodiments. In some aspects, disclosed herein are cells comprising the composition of any of the above aspects or embodiments.

In some aspects, disclosed herein are methods of reducing expression of a gene in a cell, the method comprising contacting the cell with the composition of any of the above aspects or embodiments or the kit of any of the above aspects or embodiments. In some embodiments, the cell is a mammalian cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 illustrates schematic representations of exemplary engineered fusion proteins for epigenetic modification of target nucleic acids.

FIG. 2 shows that the fusion proteins of FIG. 1 can repress gene expression.

FIG. 3 shows indel % and the percentage of cells in a cell population wherein a GFP reporter expression was repressed by dCasPhi.12 (L26R and E567Q)-DNMT3L-KRAB.

FIGS. 4A and 4B show percentages of cells in a cell population for which GFP reporter expression was repressed by a fusion protein comprising a mutated CasPhi.12 paired with a first exemplary guide RNA (FIG. 4A) or a second exemplary guide RNA (FIG. 4B). (DNMT3L is abbreviated as D3L).

FIGS. 5A and 5B show percentages of cells in a cell population for which GFP reporter expression was repressed by a fusion protein comprising CasPhi12(L26R_E567Q), DNMT3L and KRAB in different orientations when paired with a first exemplary guide RNA (FIG. 5A) or second exemplary guide RNA (FIG. 5B).

FIGS. 6A-6F show percentages of the cells in a cell population for which GFP reporter expression was repressed by a fusion protein comprising CasPhi12(L26R_E567Q)-DNMT3L-X (FIG. 6A), X-CasPhi12(L26R_E567Q)-DNMT3L-DNMT3A (FIG. 6B), DNMT3A-DNMT3L-CasPhi12(L26R_E567Q)-X (FIG. 6C), DNMT3L-X-CasPhi12(L26R_E567Q) (FIG. 6D), X-CasPhi12(L26R_E567Q)-DNMT3L (FIG. 6E), or X-CasPhi12(L26R_E567Q) (FIG. 6F) when paired with exemplary guide RNAs. X refers to KRAB(ZIM3) or KRAB(ZNF10).

FIGS. 7A and 7B show percentages of the cells in a cell population for which GFP reporter expression was repressed by a fusion protein comprising KRAB(ZNF10), KRAB(ZIM3) or a combination of the two, in addition to CasPhi12(L26R_E567Q) and DNMT3L in different orientations when paired with a first exemplary guide RNA (FIG. 7A) or a second exemplary guide RNA (FIG. 7B).

FIG. 8 shows percentages of the cells in a cell population for which GFP reporter expression was repressed by a fusion protein comprising mutated CasM.265466 (dCas466) and at least one effector partner selected from DNMT3L, DNMT3A and KRAB(ZNF10) in different orientations 21 days after transfection when paired with guide nucleic acids that are complementary to and/or hybridize to a target sequence situated from about −400 nucleotides to +350 nucleotides relative to the transcription start site.

FIG. 9 shows percentages of cells in a cell population for which GFP reporter expression was repressed by a fusion protein comprising a mutated CasM.265466 (dCas466) and at least one effector partner selected from DNMT3L, DNMT3A and KRAB(ZNF10) in different orientations when paired with various guide nucleic acids.

FIG. 10 shows percentages of cells in a cell population for which GFP reporter expression was repressed by a fusion protein comprising KRAB(ZNF10)-dCas466-DNMT3L-DNMT3A when paired with various guide nucleic acids targeting a sequence at various positions relative to the reporter transcription start site.

FIG. 11 shows percentages of cells in a cell population for which GFP reporter expression was repressed by a fusion protein comprising mutated CasM.265466 (dCas466) and at least one effector partner selected from DNMT3L, DNMT3A and KRAB(ZNF10) in different orientations when paired with a guide targeting a sequence located 221 nucleotides 5′ of the reporter transcription start site (−221).

FIGS. 12A-12B show percentages of cells in a cell population for which GFP reporter expression was repressed by a fusion protein comprising a dCasM effector protein selected from dCasM.288094 (dCasM.094), dCasM.288886 (dCasM.886), and dCasM.2401960 (dCasM.960) and at least one effector partner selected from DNMT3L, DNMT3A and KRAB in different orientations. The measurements were taken on day 5 (FIG. 12A) and day 7 (FIG. 12B). Control fusion proteins include dCasM.886 with a NT guide (NT886), KRAB(ZNF10)-CasPhi.12 (KRABPhi), dCasPhi(L26R, E567Q)-DNMT3L-KRAB(ZNF10) with a NT guide (NTEpiCasPhi), dCasPhi(L26R, E567Q)-DNMT3L-KRAB(ZNF10) (EpiCasPhi), dCas9 (CRISPROff), and CRISPROff with a NT guide (NTCRISPROff).

FIG. 13 shows repressors fused to the C-terminus of dCasPhi(L26R, E567Q)-DNMT3L that provide persistent repression of a GFP reporter in mammalian cells 27 days after transfection of the reporter, fusion protein and guide RNA.

FIG. 14 shows fusion proteins comprising repressor(s) fused to the C-terminus of dCasPhi(L26R, E567Q)-DNMT3L that provided the greatest persistent repression of a GFP reporter in mammalian cells 27 days after transfection of the reporter, fusion protein and guide RNA.

FIGS. 15A-15B show that variants of CasPhi.12 fused to repressor domains achieve epigenetic silencing levels near that of Cas9 (CRISPR Off). FIG. 15A shows % GFP repression and FIG. 15B shows median levels of FITC positive cells.

FIGS. 16A-16B show that CasPhi^{L26K/E567Q}-mDnmt3l-KRAB^{ZNF10} can repress multiple genes in mammalian cells 7 days (FIG. 16A) and 30 days (FIG. 16B) post-transfection.

FIGS. 17A-17B show that pooling guide nucleic acids can have an additive effect on repressive activity.

FIGS. 18A-18B show various dCasPhi.12 repressor fusion proteins can repress GFP expression for at least 21 days with two different guide nucleic acids (gKD010 and gTR079).

### DETAILED DESCRIPTION OF THE INVENTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

The compositions, systems, and methods described herein are non-naturally occurring. In some embodiments, compositions, systems, and methods comprise an engineered guide nucleic acid (also referred to herein as a guide nucleic acid) or a use thereof. In some embodiments, compositions, systems, and methods comprise an engineered protein or a use thereof. In some embodiments, compositions, systems, and methods comprise an isolated polypeptide or a use thereof. In general, compositions, methods, and systems described herein are not found in nature. In some embodiments, compositions, methods, and systems described herein comprise at least one non-naturally occurring component. For example, disclosed compositions, methods, and systems may comprise a guide nucleic acid, wherein the sequence of the guide nucleic acid is different or modified from that of a naturally-occurring guide nucleic acid.

In some embodiments, compositions, systems, and methods comprise at least two components that do not naturally occur together. For example, disclosed compositions, systems, and methods may comprise a guide nucleic acid comprising a first region, at least a portion of which, interacts with a polypeptide, and a second region that is at least partially complementary to a target sequence in a target nucleic acid, wherein the first region and second region do not naturally occur together and/or are heterologous to each other. Also, by way of non-limiting example, disclosed compositions, systems, and methods may comprise a guide nucleic acid and an effector protein that do not naturally occur together. Likewise, by way of non-limiting example, disclosed compositions, systems, and methods may comprise a ribonucleotide-protein (RNP) complex comprising an effector protein and a guide nucleic acid that do not occur together in nature. Conversely, and for clarity, an effector protein or guide nucleic acid that is “natural,” “naturally-occurring,” or “found in nature” includes effector proteins and guide nucleic acids from cells or organisms that have not been genetically modified by a human or machine.

In some embodiments, the guide nucleic acid comprises a non-natural nucleotide sequence. In some embodiments, the non-natural nucleotide sequence is a nucleotide sequence that is not found in nature. The non-natural nucleotide sequence may comprise a portion of a naturally-occurring sequence, wherein the portion of the naturally-occurring sequence is not present in nature absent the remainder of the naturally-occurring sequence. In some embodiments, the guide nucleic acid comprises two naturally-occurring sequences arranged in an order or proximity that is not observed in nature. In some embodiments, compositions and systems comprise a ribonucleotide complex comprising an effector protein and a guide nucleic acid that do not occur together in nature. In some embodiments, compositions and systems comprise at least two components that do not occur together in nature, wherein the at least two components comprise at least one of an effector protein, an effector partner and a guide nucleic acid. Guide nucleic acids may comprise a first sequence and a second sequence that do not occur naturally together. For example, a guide nucleic acid may comprise a naturally-occurring repeat sequence and a spacer sequence that is complementary to a naturally-occurring eukaryotic sequence. The guide nucleic acid may comprise a repeat sequence that occurs naturally in an organism and a spacer sequence that does not occur naturally in that organism. A guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. In some embodiments, the guide nucleic acid comprises two heterologous sequences arranged in an order or proximity that is not observed in nature. Therefore, compositions and systems described herein are not naturally occurring.

In some embodiments, compositions, systems, and methods described herein comprise a polypeptide (e.g., an effector protein, an effector partner, a fusion protein, or a combination thereof) that is similar to a naturally occurring polypeptide. The polypeptide may lack a portion of the naturally occurring polypeptide. The polypeptide may comprise a mutation relative to the naturally-occurring polypeptide, wherein the mutation is not found in nature. The polypeptide may also comprise at least one additional amino acid relative to the naturally-occurring polypeptide. In some embodiments, the polypeptide may comprise a heterologous polypeptide. For example, the polypeptide may comprise an addition of a nuclear localization signal relative to the natural occurring polypeptide. In some embodiments, a nucleotide sequence encoding the polypeptide is codon optimized (e.g., for expression in a eukaryotic cell) relative to the naturally occurring sequence.

### Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, include plural references unless the context clearly dictates otherwise.

The terms, “or” and “and/or,” as used herein, include any and all combinations of one or more of the associated listed items.

The terms, “including,” “includes,” “included,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers +/−10% thereof, or 10% below the lower listed limit and 10% above the higher listed limit for the values listed for a range.

The terms, “% identical,” “% identity,” “percent identity,” and grammatical equivalents thereof, as used herein, in the context of an amino acid sequence or nucleotide sequence, refer to the percent of residues that are identical between respective positions of two sequences when the two sequences are aligned for maximum sequence identity. The % identity is calculated by dividing the total number of the aligned residues by the number of the residues that are identical between the respective positions of the at least two sequences and multiplying by 100. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

The terms, “% complementary”, “% complementarity”, “percent complementary”, “percent complementarity” and grammatical equivalents thereof, as used interchangeably herein, in the context of two or more nucleic acid molecules, refer to the percent of nucleotides in two nucleotide sequences in said nucleic acid molecules of equal length that can undergo cumulative base pairing at two or more individual corresponding positions in an antiparallel orientation. Accordingly, the terms include nucleic acid sequences that are not completely complementary over their entire length, which indicates that the two or more nucleic acid molecules include one or more mismatches. A “mismatch” is present at any position in the two opposed nucleotides that are not complementary. The % complementary is calculated by dividing the total number of the complementary residues by the total number of the nucleotides in one of the equal length sequences, and multiplying by 100. Complete or total complementarity describes nucleotide sequences in 100% of the residues of a nucleotide sequence are complementary to residues in a reference nucleotide sequence. “Partially complementarity” describes nucleotide sequences in which at least 20%, but less than 100%, of the residues of a nucleotide sequence are complementary to residues in a reference nucleotide sequence. In some instances, at least 50%, but less than 100%, of the residues of a nucleotide sequence are complementary to residues in a reference nucleotide sequence. In some instances, at least 70%, 80%, 90% or 95%, but less than 100%, of the residues of a nucleotide sequence are complementary to residues in a reference nucleotide sequence. “Noncomplementary” describes nucleotide sequences in which less than 20% of the residues of a nucleotide sequence are complementary to residues in a reference nucleotide sequence.

The term, “percent similarity,” or “% similarity,” as used herein, in the context of an amino acid sequence, refers to a value that is calculated by dividing a similarity score by the length of the alignment. The similarity of two amino acid sequences can be calculated by using a BLOSUM62 similarity matrix (Henikoff and Henikoff, Proc. Natl. Acad. Sci. USA., 89:10915-10919 (1992)) that is transformed so that any value≥1 is replaced with +1 and any value≤0 is replaced with 0. For example, an Ile (I) to Leu (L) substitution is scored at +2.0 by the BLOSUM62 similarity matrix, which in the transformed matrix is scored at +1. This transformation allows the calculation of percent similarity, rather than a similarity score. Alternately, when comparing two full protein sequences, the proteins can be aligned using pairwise MUSCLE alignment. Then, the % similarity can be scored at each residue and divided by the length of the alignment. For determining % similarity over a protein domain or motif, a multilevel consensus sequence (or PROSITE motif sequence) can be used to identify how strongly each domain or motif is conserved. In calculating the similarity of a domain or motif, the second and third levels of the multilevel sequence are treated as equivalent to the top level. Additionally, if a substitution could be treated as conservative with any of the amino acids in that position of the multilevel consensus sequence, +1 point is assigned. For example, given the multilevel consensus sequence: RLG and YCK, the test sequence QIQ would receive three points. This is because in the transformed BLOSUM62 matrix, each combination is scored as: Q-R: +1; Q-Y: +0; I-L: +1; I-C: +0; Q-G: +0; Q-K: +1. For each position, the highest score is used when calculating similarity. The % similarity can also be calculated using commercially available programs, such as the Geneious Prime software given the parameters matrix=BLOSUM62 and threshold≥1.

The terms, “bind,” “binding,” “interact” and “interacting,” as used herein, refer to a non-covalent interaction between macromolecules (e.g., between two polypeptides, between a polypeptide and a nucleic acid; between a polypeptide/guide nucleic acid complex and a target nucleic acid; and the like). While in a state of noncovalent interaction, the macromolecules are said to be “associated” or “interacting” or “binding” (e.g., when a molecule X is said to interact with a molecule Y, it is meant the molecule X binds to molecule Y in a non-covalent manner). Non-limiting examples of non-covalent interactions are ionic bonds, hydrogen bonds, van der Waals and hydrophobic interactions. Not all components of a binding interaction need be sequence-specific (e.g., contacts with phosphate residues in a DNA backbone), but some portions of a binding interaction may be sequence-specific.

The term, “catalytically inactive effector protein,” as used herein, refers to an effector protein that is modified relative to a naturally-occurring effector protein to have a reduced or eliminated catalytic activity relative to that of the naturally-occurring effector protein, but retains its ability to interact with a guide nucleic acid. The catalytic activity that is reduced or eliminated is often a nuclease activity. The naturally-occurring effector protein may be a wildtype protein. In some instances, the catalytically inactive effector protein is referred to as a catalytically inactive variant of an effector protein.

The term, “codon optimized,” as used herein, refers to a mutation of a nucleotide sequence encoding a polypeptide, such as a nucleotide sequence encoding an effector protein, to mimic the codon preferences of the intended host organism or cell while encoding the same polypeptide. Thus, the codons can be changed, but the encoded polypeptide remains unchanged. For example, if the intended target cell was a human cell, a human codon-optimized nucleotide sequence encoding an effector protein could be used. As another non-limiting example, if the intended host cell were a mouse cell, then a mouse codon-optimized nucleotide sequence encoding an effector protein could be generated. As another non-limiting example, if the intended host cell were a eukaryotic cell, then a eukaryote codon-optimized nucleotide sequence encoding an effector protein could be generated. As another non-limiting example, if the intended host cell were a prokaryotic cell, then a prokaryote codon-optimized nucleotide sequence encoding an effector protein could be generated. Codon usage tables are readily available, for example, at the “Codon Usage Database” available at www.kazusa.or.j//codon.

The terms, “complementary” and “complementarity,” as used herein, in the context of a nucleic acid molecule or nucleotide sequence, refer to the characteristic of a polynucleotide having nucleotides that can undergo cumulative base pairing with their Watson-Crick counterparts (C with G; or A with T) in a reference nucleic acid in antiparallel orientation. For example, when every nucleotide in a polynucleotide or a specified portion thereof forms a base pair with every nucleotide in an equal length sequence of a reference nucleic acid, that polynucleotide is said to be 100% complementary to the sequence of the reference nucleic acid. In a double stranded DNA or RNA sequence, the upper (sense) strand sequence is, in general, understood as going in the direction from its 5′- to 3′-end, and the complementary sequence is thus understood as the sequence of the lower (antisense) strand in the same direction as the upper strand. Following the same logic, the reverse sequence is understood as the sequence of the upper strand in the direction from its 3′- to its 5′-end, while the “reverse complement” sequence or the “reverse complementary” sequence is understood as the sequence of the lower strand in the direction of its 5′- to its 3′-end. Each nucleotide in a double stranded DNA or RNA molecule that is paired with its Watson-Crick counterpart can be referred to as its complementary nucleotide. The complementarity of modified or artificial base pairs can be based on other types of hydrogen bonding and/or hydrophobicity of bases and/or shape complementarity between bases.

The term, “conservative substitution,” as used herein, refers to the replacement of one amino acid for another such that the replacement takes place within a family of amino acids that are related in their side chains. Conversely, the term “non-conservative substitution” as used herein refers to the replacement of one amino acid residue for another that does not have a related side chain. Genetically encoded amino acids can be divided into four families having related side chains: (1) acidic (negatively charged): Asp (D), Glu (E); (2) basic (positively charged): Lys (K), Arg (R), His (H); (3) non-polar (hydrophobic): Cys (C), Ala (A), Val (V), Leu (L), Ile (I), Pro (P), Phe (F), Met (M), Trp (W), Gly (G), Tyr (Y), with non-polar also being subdivided into: (i) strongly hydrophobic: Ala (A), Val (V), Leu (L), Ile (I), Met (M), Phe (F); and (ii) moderately hydrophobic: Gly (G), Pro (P), Cys (C), Tyr (Y), Trp (W); and (4) uncharged polar: Asn (N), Gln (Q), Ser (S), Thr (T). Amino acids may be related by aliphatic side chains: Gly (G), Ala (A), Val (V), Leu (L), Ile (I), Ser (S), Thr (T), with Ser (S) and Thr (T) optionally being grouped separately as aliphatic-hydroxyl; Amino acids may be related by aromatic side chains: Phe (F), Tyr (Y), Trp (W). Amino acids may be related by amide side chains: Asn (N), Gln (Q). Amino acids may be related by sulfur-containing side chains: Cys (C) and Met (M).

The terms, “CRISPR RNA” and “crRNA,” as used herein, refer to a type of guide nucleic acid that is RNA comprising a first sequence that is capable of hybridizing to a target sequence of a target nucleic acid and a second sequence that is capable of interacting with an effector protein either directly (by being bound by an effector protein) or indirectly (e.g., by hybridization with a second nucleic acid molecule that can be bound by an effector). The first sequence and the second sequence are directly connected to each other or by a linker.

The term “dual nucleic acid system” as used herein refers to a system that uses a transactivated or transactivating tracrRNA-crRNA duplex complexed with one or more polypeptides described herein, wherein the complex is capable of interacting with a target nucleic acid in a sequence selective manner.

The term, “effector protein,” as used herein, refers to a protein, polypeptide, or peptide that is capable of interacting with a nucleic acid, such as a guide nucleic acid, to form a complex (e.g., a RNP complex), wherein the complex interacts with a target nucleic acid.

The term, “effector partner,” as used herein, refers to a protein, polypeptide or peptide that can impart a function, in combination with an effector protein and guide nucleic acid, that can be used to effectuate modification(s) of a target nucleic acid described herein and/or change expression of the target nucleic acid or other nucleic acids associated with the target nucleic acid.

The term, “engineered modification,” as used herein, refers to a structural change of one or more nucleic acid residues of a nucleotide sequence or one or more amino acid residue of an amino acid sequence. The engineered modifications of a nucleotide sequence can include chemical modification of one or more nucleobases, or a chemical change to the phosphate backbone, a nucleotide, a nucleobase or a nucleoside. The engineered modifications can be made to an effector protein amino acid sequence or guide nucleic acid nucleotide sequence, or any sequence disclosed herein (e.g., a nucleic acid encoding an effector protein or a nucleic acid that encodes a guide nucleic acid). Methods of modifying a nucleic acid or amino acid sequence are known. One of ordinary skill in the art will appreciate that the engineered modification(s) may be located at any position(s) of a nucleic acid such that the function of the nucleic acid, protein, composition or system is not substantially decreased. Nucleic acids provided herein can be prepared according to any available technique including, but not limited to chemical synthesis, enzymatic synthesis, which is generally termed in vitro-transcription, cloning, enzymatic, or chemical cleavage, etc. In some instances, the nucleic acids provided herein are not uniformly modified along the entire length of the molecule. Different nucleotide modifications and/or backbone structures can exist at various positions within the nucleic acid.

The term, “functional domain,” as used herein, refers to a region of one or more amino acids in a protein that is required for an activity of the protein, or the full extent of that activity, as measured in an in vitro assay. Activities include, but are not limited to nucleic acid binding, nucleic acid modifying, protein binding. The absence of the functional domain, including mutations of the functional domain, would abolish or reduce activity.

The term, “fused,” as used herein, refers to at least two sequences that are connected together, such as by a covalent bond (e.g., an amide bond or a phosphodiester bind) or by a linker. The covalent bond can be formed by a conjugation (e.g., chemical conjugation or enzymatic conjugation) reaction.

The term, “fusion protein,” as used herein, refers to a protein comprising at least two heterologous polypeptides. The fusion protein may comprise one or more effector protein and fusion partner. In some instances, an effector protein and fusion partner are not found connected to one another as a native protein or complex that occurs together in nature.

The term, “fusion partner,” as used herein, refers to an effector partner that is fused, or linked by a linker, to one or more effector protein. The fusion partner can impart some function to the fusion protein that is not provided by the effector protein.

The term, “genetic disease,” as used herein, refers to a disease, disorder, condition, or syndrome associated with or caused by one or more mutations in the DNA of an organism having the genetic disease.

The term, “guide nucleic acid,” as used herein, refers to a nucleic acid that, when in a complex with one or more polypeptides described herein (e.g., an RNP complex) can impart sequence selectivity to the complex when the complex interacts with a target nucleic acid. A guide nucleic acid may be referred to interchangeably as a guide RNA, however it is understood that guide nucleic acids may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The term, “handle sequence,” as used herein, refers to a sequence of nucleotides in a single guide RNA (sgRNA), that is: 1) capable of being non-covalently bound by an effector protein and 2) connects the portion of the sgRNA capable of being non-covalently bound by an effector protein to a nucleotide sequence that is hybridizable to a target nucleic acid. In general, the handle sequence comprises an intermediary sequence, that is capable of being non-covalently bound by an effector protein. In some instances, the handle sequence further comprises a repeat sequence. In such instances, the intermediary sequence or a combination of the intermediary sequence and the repeat sequence is capable of being non-covalently bound by an effector protein.

The term, “heterologous,” as used herein, refers to at least two different polypeptide sequences that are not found similarly connected to one another in a native nucleic acid or protein. A protein that is heterologous to the effector protein is a protein that is not covalently linked by an amide bond to the effector protein in nature. In some instances, a heterologous protein is not encoded by a species that encodes the effector protein. A guide nucleic acid may comprise “heterologous” sequences, which means that it includes a first sequence and a second sequence, wherein the first sequence and the second sequence are not found covalently linked by a phosphodiester bond in nature. Thus, the first sequence is considered to be heterologous with the second sequence, and the guide nucleic acid may be referred to as a heterologous guide nucleic acid. A heterologous system comprises at least one component that is not naturally occurring together with remaining components of the heterologous system.

The terms, “hybridize,” “hybridizable” and grammatical equivalents thereof, refer to a nucleotide sequence that is able to noncovalently interact, i.e. form Watson-Crick base pairs and/or G/U base pairs, or anneal, to another nucleotide sequence in a sequence-specific, antiparallel, manner (i.e., a nucleotide sequence specifically interacts to a complementary nucleotide sequence) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) for both DNA and RNA. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, a guanine (G) can be considered complementary to both an uracil (U) and to an adenine (A). Accordingly, when a G/U base-pair can be made at a given nucleotide position, the position is not considered to be non-complementary, but is instead considered to be complementary. While hybridization typically occurs between two nucleotide sequences that are complementary, mismatches between bases are possible. It is understood that two nucleotide sequences need not be 100% complementary to be specifically hybridizable, hybridizable, partially hybridizable, or for hybridization to occur. Moreover, a nucleotide sequence may hybridize over one or more segments such that intervening or adjacent segments are not involved in the hybridization event (e.g., a bulge, a loop structure or hairpin structure, etc.). The conditions appropriate for hybridization between two nucleotide sequences depend on the length of the sequence and the degree of complementarity, variables which are well known in the art. For hybridizations between nucleic acids with short stretches of complementarity (e.g. complementarity over 35 or less, 30 or less, 25 or less, 22 or less, 20 or less, or 18 or less nucleotides) the position of mismatches may become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). Any suitable in vitro assay may be utilized to assess whether two sequences “hybridize”. One such assay is a melting point analysis where the greater the degree of complementarity between two nucleotide sequences, the greater the value of the melting temperature (Tm) for hybrids of nucleic acids having those sequences. The conditions of temperature and ionic strength determine the “stringency” of the hybridization. Temperature, wash solution salt concentration, and other conditions may be adjusted as necessary according to factors such as length of the region of complementation and the degree of complementation. Hybridization and washing conditions are well known and exemplified in Sambrook, J. and Russell, W., Molecular Cloning: A Laboratory Manual, Third Edition, Cold Spring Harbor Laboratory Press, Cold Spring Harbor (2001); and in Green, M. and Sambrook, J., Molecular Cloning: A Laboratory Manual, Fourth Edition, Cold Spring Harbor Laboratory Press, Cold Spring Harbor (2012).

The terms, “intermediary RNA” and “intermediary sequence,” as used herein, in a context of a single nucleic acid system, refers to a nucleotide sequence in a handle sequence, wherein the nucleotide sequence is capable of, at least partially, being non-covalently bound to an effector protein to form a complex (e.g., an RNP complex). An intermediary sequence is not a transactivating nucleic acid in systems, methods, and compositions described herein.

The term, “in vitro,” as used herein, refers to describing something outside an organism. An in vitro system, composition or method may take place in a container for holding laboratory reagents such that it is separated from the biological source from which a material in the container is obtained. In vitro assays can encompass cell-based assays in which living or dead cells are employed. In vitro assays can also encompass a cell-free assay in which no intact cells are employed. The term “in vivo” is used to describe an event that takes place within an organism. The term “ex vivo” is used to describe an event that takes place in a cell that has been obtained from an organism. An ex vivo assay is not performed on a subject. Rather, it is performed upon a sample separate from a subject.

The terms, “length” and “linked” as used herein, refer to a nucleic acid (polynucleotide) or polypeptide, may be expressed as “kilobases” (kb) or “base pairs (bp),”. Thus, a length of 1 kb refers to a length of 1000 linked nucleotides, and a length of 500 bp refers to a length of 500 linked nucleotides. Similarly, a protein having a length of 500 linked amino acids may also be simply described as having a length of 500 amino acids.

The term, “linker,” as used herein, refers to a molecule that links a first polypeptide to a second polypeptide (e.g., by an amide bond) or a first nucleic acid to a second nucleic acid (e.g., by a phosphodiester bond).

The terms, “mutation associated with a disease” and “mutation associated with a genetic disorder,” as used herein, refer to the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation.

The terms, “non-naturally occurring” and “engineered,” as used herein, refer to indicate involvement of the hand of man. The terms, when referring to a nucleic acid, nucleotide, protein, polypeptide, peptide or amino acid, refer to a molecule, such as but not limited to, a nucleic acid, nucleotide, protein, polypeptide, peptide or amino acid refers to a modification of that molecule (e.g., chemical modification, nucleotide sequence, or amino acid sequence) that is not present in the naturally molecule. The terms, when referring to a composition or system described herein, refer to a composition or system having at least one component that is not naturally associated with the other components of the composition or system. By way of a non-limiting example, a composition may include an effector protein and a guide nucleic acid that do not naturally occur together. Conversely, and as a non-limiting further clarifying example, an effector protein or guide nucleic acid that is “natural,” “naturally-occurring,” or “found in nature” includes an effector protein and a guide nucleic acid from a cell or organism that have not been genetically modified by the hand of man.

The terms, “nuclease” and “endonuclease” as used herein, refer to an enzyme which possesses catalytic activity for nucleic acid cleavage.

The term, “nucleic acid,” as used herein, refers to a polymer of nucleotides. A nucleic acid may comprise ribonucleotides, deoxyribonucleotides, combinations thereof, and modified versions of the same. A nucleic acid may be single-stranded or double-stranded, unless specified. Non-limiting examples of nucleic acids are double stranded DNA (dsDNA), single stranded (ssDNA), messenger RNA, genomic DNA, cDNA, DNA-RNA hybrids, and a polymer comprising purine and pyrimidine bases or other natural, chemically or biochemically modified, non-natural, or derivatized nucleotide bases. Accordingly, nucleic acids as described herein may comprise one or more mutations, one or more engineered modifications, or both.

The term, “nucleic acid expression vector,” as used herein, refers to a plasmid that can be used to express a nucleic acid of interest.

The term, “nuclear localization signal (NLS),” as used herein, refers to an entity (e.g., peptide) that facilitates localization of a nucleic acid, protein, or small molecule to the nucleus, when present in a cell that contains a nuclear compartment.

The term, “pharmaceutically acceptable excipient, carrier or diluent,” as used herein, refers to any substance formulated alongside the active ingredient of a pharmaceutical composition that allows the active ingredient to retain biological activity and is non-reactive with the subject's immune system. Such a substance can be included for the purpose of long-term stabilization, bulking up solid formulations that contain potent active ingredients in small amounts, or to confer a therapeutic enhancement on the active ingredient in the final dosage form, such as facilitating absorption, reducing viscosity, or enhancing solubility. The selection of appropriate substance can depend upon the route of administration and the dosage form, as well as the active ingredient and other factors. Compositions having such substances can be formulated by suitable methods (see, e.g., Remington's Pharmaceutical Sciences, 23rd edition, A. Adejare, ed., Elsevier Inc., Philadelphia, Pa., 2020).

The terms, “polypeptide” and “protein,” as used herein, refer to a polymeric form of amino acids. A polypeptide may include coded and non-coded amino acids, chemically or biochemically modified or derivatized amino acids, and polypeptides having modified peptide backbones. Accordingly, polypeptides as described herein may comprise one or more mutations, one or more engineered modifications, or both. It is understood that when describing coding sequences of polypeptides described herein, said coding sequences do not necessarily require a codon encoding an N-terminal Methionine (M) or a Valine (V) as described for the effector proteins described herein. One skilled in the art would understand that a start codon could be replaced or substituted with a start codon that encodes for an amino acid residue sufficient for initiating translation in a host cell. In some instances, when a heterologous peptide, such as a fusion partner protein, protein tag or NLS, is located at the N terminus of the effector protein, a start codon for the heterologous peptide serves as a start codon for the effector protein as well. Thus, the natural start codon encoding an amino acid residue sufficient for initiating translation (e.g., Methionine (M) or a Valine (V)) of the effector protein may be removed or absent.

The terms, “promoter” and “promoter sequence,” as used herein, refer to a DNA regulatory region capable of binding RNA polymerase and initiating transcription of a downstream (3′ direction) coding or non-coding sequence. A transcription initiation site, as well as protein binding domains responsible for the binding of RNA polymerase, can also be found in a promoter region. Eukaryotic promoters will often, but not always, contain “TATA” boxes and “CAT” boxes. Various promoters, including inducible promoters, may be used to drive expression by the various vectors of the present disclosure.

The terms, “protospacer adjacent motif” and “PAM,” as used herein, refer to a nucleotide sequence found in a target nucleic acid that directs an effector protein to modify the target nucleic acid at a specific location. In some instances, a PAM is required for a complex of an effector protein and a guide nucleic acid (e.g., an RNP complex) to hybridize to and modify the target nucleic acid. In some instances, the complex does not require a PAM to modify the target nucleic acid.

The term, “recombinant,” as used herein, in the context of proteins, polypeptides, peptides and nucleic acids, refers to proteins, polypeptides, peptides and nucleic acids that are products of various combinations of cloning, restriction, and/or ligation steps resulting in a construct having a structural coding or non-coding sequence distinguishable from endogenous nucleic acids found in natural systems.

The term, “regulatory element,” used herein, refers to transcriptional and translational control sequences, such as promoters, enhancers, polyadenylation signals, terminators, protein degradation signals, and the like, that provide for and/or regulate transcription of a non-coding sequence (e.g., a guide nucleic acid) or a coding sequence (e.g., effector proteins, fusion proteins, and the like) and/or regulate translation of an encoded polypeptide.

The term, “repeat hybridization sequence,” as used herein, in the context of a dual nucleic acid system, refers to a sequence of nucleotides of a tracrRNA that is capable of hybridizing to a repeat sequence of a guide nucleic acid.

The term, “repeat sequence,” as used herein, refers to a sequence of nucleotides in a guide nucleic acid that is capable of, at least partially, interacting with an effector protein.

The terms, “ribonucleotide protein complex” and “RNP” as used herein, refer to a complex of one or more nucleic acids and one or more polypeptides described herein. While the term utilizes “ribonucleotides” it is understood that the one or more nucleic acid may comprise deoxyribonucleotides (DNA), ribonucleotides (RNA), a combination thereof (e.g., RNA with a thymine base), biochemically or chemically modified nucleobases (e.g., one or more engineered modifications described herein), or combinations thereof.

The terms, “RuvC” and “RuvC domain,” as used herein, refer to a region of an effector protein that is capable of cleaving a target nucleic acid, and in certain instances, of processing a pre-crRNA. In some instances, the RuvC domain is located near the C-terminus of the effector protein. A single RuvC domain may comprise RuvC subdomains, for example a RuvCI subdomain, a RuvCII subdomain and a RuvCIII subdomain. The term “RuvC” domain can also refer to a “RuvC-like” domain. Various RuvC-like domains are known in the art and are easily identified using online tools such as IntePro (https://www.ebi.ac.uk/interpro/). For example, a RuvC-like domain may be a domain which shares homology with a region of TnpB proteins of the IS605 and other related families of transposons.

The terms, “single guide nucleic acid”, “single guide RNA” and “sgRNA,” as used herein, in the context of a single nucleic acid system, refers to a guide nucleic acid, wherein the guide nucleic acid is a single polynucleotide chain having all the required sequence for a functional complex with an effector protein (e.g., being bound by an effector protein, including in some instances activating the effector protein, and hybridizing to a target nucleic acid, without the need for a second nucleic acid molecule). For example, an sgRNA can have two or more linked guide nucleic acid components (e.g., an intermediary sequence, a repeat sequence, a spacer sequence and optionally a linker, or a handle sequence and a spacer sequence).

The term, “single nucleic acid system,” as used herein, refers to a system that uses a guide nucleic acid complexed with one or more polypeptides described herein, wherein the complex is capable of interacting with a target nucleic acid in a sequence specific manner, and wherein the guide nucleic acid is capable of non-covalently interacting with the one or more polypeptides described herein, and wherein the guide nucleic acid is capable of hybridizing with a target sequence of the target nucleic acid. A single nucleic acid system lacks a duplex of a guide nucleic acid as hybridized to a second nucleic acid, wherein in such a duplex the second nucleic acid, and not the guide nucleic acid, is capable of interacting with the effector protein.

The term, “spacer sequence,” as used herein, refers to a nucleotide sequence in a guide nucleic acid that is capable of, at least partially, hybridizing to an equal length portion of a sequence (e.g., a target sequence) of a target nucleic acid.

The term, “subject,” as used herein, refers to an animal. The subject may be a mammal. The subject may be a human. The subject may be diagnosed or at risk for a disease.

The term, “sufficiently complementary,” as used herein, refers to a first nucleotide sequence that is partially complementarity to a second nucleotide sequence while still allowing the first nucleotide sequence to hybridize to the second nucleotide sequence with enough affinity to permit a biological activity to occur. Depending on the context, a biological activity may be the formation of a complex between two or more components described herein, such as an effector protein and a guide nucleic acid. A biological activity may also be bringing one or more components described herein into proximity of another component, such as bringing an effector protein-guide nucleic acid complex into proximity of a target nucleic acid. A biological activity may additionally be permitting a component described herein to act on another component described herein. In some instances, sequences are said to be sufficiently complementary when at least 85% of the residues of a nucleotide sequence are complementary to residues in a reference nucleotide sequence.

The term, “syndrome,” as used herein, refers to a group of symptoms which, taken together, characterize a condition.

The term, “target nucleic acid,” as used herein, refers to a nucleic acid that is selected as the nucleic acid for modification, binding, hybridization or any other activity of or interaction with a nucleic acid, protein, polypeptide, or peptide described herein. A target nucleic acid may comprise RNA, DNA, or a combination thereof. A target nucleic acid may be single-stranded (e.g., single-stranded RNA or single-stranded DNA) or double-stranded (e.g., double-stranded DNA).

The term, “target sequence,” as used herein, in the context of a target nucleic acid, refers to a nucleotide sequence found within a target nucleic acid. Such a nucleotide sequence can, for example, hybridize to a respective length portion of a guide nucleic acid.

The terms, “trans-activating RNA”, “transactivating RNA” and “tracrRNA,” refer to a transactivating or transactivated nucleic acid in a dual nucleic acid system that is capable of hybridizing, at least partially, to a crRNA to form a tracrRNA-crRNA duplex, and of interacting with an effector protein to form a complex (e.g., an RNP complex).

The terms, “transactivating”, “trans-activating”, “trans-activated”, “transactivated” and grammatical equivalents thereof, as used herein, in the context of a dual nucleic acid system refers to an outcome of the system, wherein a polypeptide is enabled to have a binding and/or nuclease activity on a target nucleic acid, by a tracrRNA or a tracrRNA-crRNA duplex.

The term, “transgene,” as used herein, refers to a nucleotide sequence that is inserted into a cell for expression of said nucleotide sequence in the cell. A transgene is meant to include (1) a nucleotide sequence that is not naturally found in the cell (e.g., a heterologous nucleotide sequence); (2) a nucleotide sequence that is a mutant form of a nucleotide sequence naturally found in the cell into which it has been introduced; (3) a nucleotide sequence that serves to add additional copies of the same (e.g., exogenous or homologous) or a similar nucleotide sequence naturally occurring in the cell into which it has been introduced; or (4) a silent naturally occurring or homologous nucleotide sequence whose expression is induced in the cell into which it has been introduced. The cell in which transgene expression occurs can be a target cell, such as a host cell.

The terms, “treatment” and “treating,” as used herein, refer to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term, “variant,” as used herein, refers to a form or version of a protein that differs from the wild-type protein. A variant may have a different function or activity relative to the wild-type protein.

The term, “viral vector,” as used herein, refers to a nucleic acid to be delivered into a host cell by a recombinantly produced virus or viral particle.

### I. Epigenetic Silencing Systems

Disclosed herein in some aspects are compositions, systems, and kits that comprise an effector protein, an effector partner protein (also referred to simply as an effector partner), and a guide nucleic acid, wherein the effector protein and the guide nucleic acid form a ribonucleoprotein (RNP) complex. In general, a portion of the guide nucleic acid is capable of hybridizing to a target sequence of a target nucleic acid. Thus, the RNP can interact with the target nucleic acid. An effector partner is either recruited to the effector protein and/or RNP or the effector partner is fused to the effector protein. Thus, the effector partner is brought into proximity of the target sequence of the target nucleic acid. The effector partner may comprise epigenetic activity that represses, inhibits, reduces or abolishes expression of the target nucleic acid. A non-limiting example of such an effector partner is a methyltransferase. Epigenetic modification of the target nucleic acid may permanently silence the gene. In some embodiments, the epigenetic modification is heritable.

In some embodiments, effector proteins, effector partner proteins, and guide nucleic acids are provided in the form of a single composition. In some embodiments, effector proteins, effector partner proteins, and guide nucleic acids are provided in a single solution. In some embodiments, effector proteins, effector partner proteins, and guide nucleic acids are provided in a single container. In some embodiments, effector proteins, effector partner proteins, and guide nucleic acids are provided as components of a system or kit, wherein a first component is provided in a first composition (e.g., solution, container), and a second component is provided in a second composition (e.g., solution, container), wherein the first composition and the second composition are not the same. In some embodiments, methods comprise administering the first composition and the second composition simultaneously. In some embodiments, methods comprise administering the first composition and the second composition sequentially.

In some embodiments, the effector partner is recruited to the effector protein, RNP, guide nucleic acid, or a combination thereof. In some embodiments, the guide nucleic acid comprises an aptamer and the effector partner comprises or is fused to an aptamer binding peptide/protein. In some embodiments, the effector protein is fused to an accessory protein or peptide that binds to the effector partner. The effector partner may be endogenous to a cell, and therefore not included in a pharmaceutical composition. In some embodiments, the effector partner is administered in a composition or otherwise exogenously added.

In some embodiments, the effector partner is a fusion partner. In some embodiments, compositions, systems, and methods comprise a fusion protein or uses thereof, wherein the fusion protein comprises an effector protein and an effector partner. In the context of a fusion protein, the effector partner may be referred to as a fusion partner. In some embodiments, the effector partner is fused to the N-terminus of the effector protein. In some embodiments, the effector partner is fused to the C-terminus of the effector protein. In some embodiments, the fusion protein complexes with a guide nucleic acid and the complex interacts with the target nucleic acid. In some embodiments, the interaction comprises one or more of: recognition of a protospacer adjacent motif (PAM) sequence within the target nucleic acid by the effector protein, hybridization of the guide nucleic acid to the target nucleic acid, modification of the target nucleic acid by the effector partner. In some embodiments, recognition of a PAM sequence within a target nucleic acid may direct the modification activity of a fusion protein.

### A. Effector Proteins

Provided herein are compositions, systems, and methods comprising an effector protein or a use thereof. An effector protein provided herein interacts with a guide nucleic acid to form a complex. In some embodiments, the complex interacts with a target nucleic acid. In some embodiments, an interaction between the complex and a target nucleic acid comprises one or more of: recognition of a protospacer adjacent motif (PAM) sequence within the target nucleic acid by the effector protein, hybridization of the guide nucleic acid to the target nucleic acid, modification of the target nucleic acid by the effector protein, or combinations thereof. In some embodiments, recognition of a PAM sequence within a target nucleic acid may direct the modification activity of an effector protein. In some embodiments, recognition of a PAM sequence adjacent to a target sequence of a target nucleic acid may direct the modification activity of an effector protein.

In some embodiments, an ability of an effector protein to modify a target nucleic acid may depend upon the effector protein being complexed with a guide nucleic acid, the guide nucleic acid being hybridized to a target sequence of the target nucleic acid, the distance between the target sequence and a PAM sequence, or a combination thereof.

The modification of the target nucleic acid generated by an effector protein may, as a non-limiting example, result in modulation of the expression of the target nucleic acid (e.g., decreasing expression of the nucleic acid). Accordingly, provided herein are methods of modulating expression of a target nucleic acid using an effector protein of the present disclosure, or compositions or systems thereof.

In some embodiments, the effector protein comprises one or more amino acid alterations relative to a naturally occurring effector protein. The one or more amino acid alterations may result in a change in activity of the effector protein relative to the naturally-occurring effector protein. In some embodiments, the one or more amino acid alterations results in a catalytically inactive effector protein variant. An effector protein that has decreased catalytic activity may be referred to as catalytically or enzymatically inactive, catalytically or enzymatically dead, as a dead protein or a dCas protein. In some embodiments, such a protein may comprise an enzymatically inactive domain (e.g. inactive nuclease domain). For example, a nuclease domain of an effector protein may be deleted or mutated relative to a wildtype counterpart so that it is no longer functional or comprises reduced nuclease activity. In some embodiments, a catalytically inactive effector protein may bind to a guide nucleic acid and/or a target nucleic acid but does not cleave the target nucleic acid. In some embodiments, a catalytically inactive effector protein may associate with a guide nucleic acid to activate or repress transcription of a target nucleic acid. In some embodiments, a catalytically inactive effector protein is fused to a fusion partner that confers an alternative activity to an effector protein activity. Such fusion proteins are described herein and throughout.

In some embodiments, an effector protein comprises a Type V Cas protein. In some embodiments, an effector protein comprises a RuvC domain. In some embodiments, an effector protein comprises an HNH domain. In some embodiments, an effector protein comprises an HNH domain. In some embodiments, an effector protein comprises a RuvC domain and does not comprise an HNH domain. In some embodiments, an effector protein comprises a Cas12J protein. Non-limiting examples of Cas12J proteins are described in WO2020181101, WO2021247924, WO2022159822, and WO2022055998. In some embodiments, effector proteins comprise less than 600, less than 700, less than 800, or less than 900 amino acids, but at least 300 amino acids. In some embodiments, the effector protein comprises about 300 to about 800 amino acids. In some embodiments, the effector protein comprises about 400 to about 800 amino acids.

In some embodiments, effector proteins comprise an amino acid sequence that is at least 80%, at least 85%, at least 90%, at least 95%, at least 97%, at least 98%, or at least 99% identical to SEQ ID NO: 1. In some embodiments, the effector protein comprises at least one amino acid substitution relative to SEQ ID NO: 1, wherein the at least one amino acid substitution is selected from: L26R, L26K, A121Q, D369A, D369N, D658A, D658N, E567A, and E567Q, and a combination thereof. In some embodiments, these amino acid substitutions reduce a catalytic activity of the effector protein relative to that of an effector protein that is 100% identical to SEQ ID NO: 1. In some embodiments, the catalytic activity is nuclease activity.

In some embodiments, effector proteins comprise an amino acid sequence that is at least 80%, at least 85%, at least 90%, at least 95%, at least 97%, at least 98%, or at least 99% identical to SEQ ID NO: 1, wherein the effector protein comprises at least one amino acid substitution relative to SEQ ID NO: 1, wherein the at least one amino acid substitution is selected from: L26R, D369A, D369N, D658A, D658N, E567A, E567Q, A121Q, L26K, I471T, K189P, S638K, Q54R, E258K, Q79R, Y220S, N406K, E119S, S223P, K92E, K435Q, N568D, E109R, H208R, K184R, K38R, L182R, Q183R, S108R, S198R, T114R, and V521T, and a combination thereof. In some embodiments, the amino acid substitution increases binding of the effector protein to a guide nucleic acid, target nucleic acid, or combination thereof.

In some embodiments, effector protein comprise at least two amino acid substitutions, wherein a first amino acid substitution reduces a catalytic activity of the effector protein relative to that of an effector protein that is 100% identical to SEQ ID NO: 1; and a second amino acid substitution increases binding of the effector protein to a guide nucleic acid, target nucleic acid, or combination thereof relative to that of an effector protein that is 100% identical to SEQ ID NO: 1.

In some embodiments, the effector protein comprise two amino acid substitutions relative to SEQ ID NO: 1, wherein the two amino acid substitutions are L26R and E567Q. In some embodiments, the effector protein comprise an amino acid sequence that is at least 80%, at least 85%, at least 90%, at least 95%, at least 97%, at least 98%, or at least 99% identical to SEQ ID NO: 2. In some embodiments, the effector protein comprise the amino acid sequence set forth in SEQ ID NO: 2.

In some embodiments, effector proteins comprise an amino acid sequence that is at least 80%, at least 85%, at least 90%, at least 95%, at least 97%, at least 98%, or at least 99% identical to SEQ ID NO: 35. In some embodiments, the effector protein comprises at least one amino acid substitution relative to SEQ ID NO: 35. In some embodiments, these amino acid substitutions reduce a catalytic activity of the effector protein relative to that of an effector protein that is 100% identical to SEQ ID NO: 35. In some embodiments, the catalytic activity is nuclease activity.

In some embodiments, effector proteins comprise an amino acid sequence that is at least 80%, at least 85%, at least 90%, at least 95%, at least 97%, at least 98%, or at least 99% identical to SEQ ID NO: 35, wherein the effector protein comprises at least one amino acid substitution relative to SEQ ID NO: 35, wherein the at least one amino acid substitution is selected from: D220R, E335Q, and a combination thereof.

In some embodiments, effector protein comprise at least two amino acid substitutions, wherein a first amino acid substitution reduces a catalytic activity of the effector protein relative to that of an effector protein that is 100% identical to SEQ ID NO: 35; and a second amino acid substitution increases binding of the effector protein to a guide nucleic acid, target nucleic acid, or combination thereof relative to that of an effector protein that is 100% identical to SEQ ID NO: 35.

In some embodiments, the effector protein comprise two amino acid substitutions relative to SEQ ID NO: 35, wherein the two amino acid substitutions are D220R and E335Q. In some embodiments, the effector protein comprise an amino acid sequence that is at least 80%, at least 85%, at least 90%, at least 95%, at least 97%, at least 98%, or at least 99% identical to SEQ ID NO: 2950. In some embodiments, the effector protein comprise the amino acid sequence set forth in SEQ ID NO: 2950. In some embodiments, the effector protein comprise an amino acid sequence that is at least 80%, at least 85%, at least 90%, at least 95%, at least 97%, at least 98%, or at least 99% identical to SEQ ID NO: 3010. In some embodiments, the effector protein comprise the amino acid sequence set forth in SEQ ID NO: 3010. In some embodiments, the effector protein comprise an amino acid sequence that is at least 80%, at least 85%, at least 90%, at least 95%, at least 97%, at least 98%, or at least 99% identical to SEQ ID NO: 3011. In some embodiments, the effector protein comprise the amino acid sequence set forth in SEQ ID NO: 3011. In some embodiments, the effector protein comprise an amino acid sequence that is at least 80%, at least 85%, at least 90%, at least 95%, at least 97%, at least 98%, or at least 99% identical to SEQ ID NO: 3012. In some embodiments, the effector protein comprise the amino acid sequence set forth in SEQ ID NO: 3012.

TABLE 1 provides illustrative amino acid sequences of effector proteins that are useful in the compositions, systems and methods described herein. In some embodiments, an effector protein, or a recombinant nucleic acid encoding an effector protein, comprises an amino acid sequence that is at least 85% identical to any one of the sequences set forth in TABLE 1. In some embodiments, the recombinant nucleic acid encoding the effector protein is operably linked to a promoter, wherein the promoter is functional in a eukaryotic cell or a prokaryotic cell. In some embodiments, the promoter is any one or more of: a constitutive promoter, an inducible promoter, a cell type-specific promoter, and a tissue-specific promoter. In some embodiments, the recombinant nucleic acid described herein wherein the promoter is functional in any one of: a plant cell, a fungal cell, an animal cell, cell of an invertebrate, a fly cell, a cell of a vertebrate, a mammalian cell, a primate cell, a non-human primate cell, and a human cell. In some embodiments, the recombinant nucleic acid is a nucleic acid expression vector as described herein.

In some embodiments, compositions, systems and methods described herein comprise an effector protein, or a nucleic acid encoding the effector protein, wherein the amino acid sequence of the effector protein comprises at least about 200 contiguous amino acids or more of any one of the sequences recited in TABLE 1. In some embodiments, the amino acid sequence of an effector protein provided herein comprises at least about 200, at least about 220, at least about 240, at least about 260, at least about 280, at least about 300, at least about 320, at least about 340, at least about 360, at least about 380, at least about 400 contiguous amino acids, at least about 420 contiguous amino acids, at least about 440 contiguous amino acids, at least about 460 contiguous amino acids, at least about 480 contiguous amino acids, at least about 500 contiguous amino acids, at least about 520 contiguous amino acids, at least about 540 contiguous amino acids, at least about 560 contiguous amino acids, at least about 580 contiguous amino acids, at least about 600 contiguous amino acids, at least about 620 contiguous amino acids, at least about 640 contiguous amino acids, at least about 660 contiguous amino acids, at least about 680 contiguous amino acids, at least about 700 contiguous amino acids of any one of the sequences of TABLE 1.

In some embodiments, compositions, systems and methods described herein comprise an effector protein or a nucleic acid encoding the effector protein, wherein the effector protein comprises a portion of any one of the sequences recited in TABLE 1. In some embodiments, the effector protein comprises a portion of any one of the sequences recited in TABLE 1, wherein the portion does not comprise at least the first 10 amino acids, at least the first 20 amino acids, at least the first 40 amino acids, at least the first 60 amino acids, at least the first 80 amino acids, at least the first 100 amino acids, at least the first 120 amino acids, at least the first 140 amino acids, at least the first 160 amino acids, at least the first 180 amino acids, or at least the first 200 amino acids of any one of the sequences recited in TABLE 1. In some embodiments, the effector protein comprises a portion of any one of the sequences recited in TABLE 1, wherein the portion does not comprise the last 10 amino acids, the last 20 amino acids, the last 40 amino acids, the last 60 amino acids, the last 80 amino acids, the last 100 amino acids, the last 120 amino acids, the last 140 amino acids, the last 160 amino acids, the last 180 amino acids, or the last 200 amino acids of any one of the sequences recited in TABLE 1.

In some embodiments, compositions, systems, and methods described herein comprise an effector protein, or a nucleic acid encoding the effector protein, wherein the effector protein comprises an amino acid sequence that is at least 65%, at least 70%, at least 75%, at least 80%, at least 85%, at least 90%, at least 95%, at least 97%, at least 98%, at least 99%, or 100% identical to any one of the sequences as set forth in TABLE 1. In some embodiments, an effector protein provided herein comprises an amino acid sequence that is at least 65% identical to any one of the sequences as set forth in TABLE 1. In some embodiments, an effector protein provided herein comprises an amino acid sequence that is at least 70% identical to any one of the sequences as set forth in TABLE 1. In some embodiments, an effector protein provided herein comprises an amino acid sequence that is at least 75% identical to any one of the sequences as set forth in TABLE 1. In some embodiments, an effector protein provided herein comprises an amino acid sequence that is at least 80% identical to any one of the sequences as set forth in TABLE 1. In some embodiments, an effector protein provided herein comprises an amino acid sequence that is at least 85% identical to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% identical to any one of the sequences as set forth in TABLE 1. In some embodiments, an effector protein provided herein comprises an amino acid sequence that is at least 97% identical to any one of the sequences as set forth in TABLE 1. In some embodiments, an effector protein provided herein comprises an amino acid sequence that is at least 98% identical to any one of the sequences as set forth in TABLE 1. In some embodiments, an effector protein provided herein comprises an amino acid sequence that is at least 99% identical to any one of the sequences as set forth in TABLE 1. In some embodiments, an effector protein provided herein comprises an amino acid sequence that is identical to any one of the sequences as set forth in TABLE 1.

In some embodiments, compositions, systems, and methods described herein comprise an effector protein, or a nucleic acid encoding the effector protein, wherein the effector protein comprises an amino acid sequence that is at least 80%, at least 85%, at least 90%, at least 95%, at least 97%, at least 98%, at least 99%, or 100% similar to any one of the sequences as set forth in TABLE 1. In some embodiments, an effector protein provided herein comprises an amino acid sequence that is at least 80% similar to any one of the sequences as set forth in TABLE 1. In some embodiments, an effector protein provided herein comprises an amino acid sequence that is at least 85% similar to any one of the sequences as set forth in TABLE 1. In some embodiments, an effector protein provided herein comprises an amino acid sequence that is at least 90% identical to any one of the sequences as set forth in TABLE 1. In some embodiments, an effector protein provided herein comprises an amino acid sequence that is at least 95% similar to any one of the sequences as set forth in TABLE 1. In some embodiments, an effector protein provided herein comprises an amino acid sequence that is at least 97% similar to any one of the sequences as set forth in TABLE 1. In some embodiments, an effector protein provided herein comprises an amino acid sequence that is at least 98% similar to any one of the sequences as set forth in TABLE 1. In some embodiments, an effector protein provided herein comprises an amino acid sequence that is at least 99% similar to any one of the sequences as set forth in TABLE 1. In some embodiments, an effector protein provided herein comprises an amino acid sequence that is 100% similar to any one of the sequences as set forth in TABLE 1.

In some embodiments, compositions, systems, and methods described herein comprise an effector protein, or a nucleic acid encoding the effector protein, wherein the effector protein comprises one or more amino acid alterations relative to any one of the sequences recited in TABLE 1. In some embodiments, the one or more alterations comprises at least one, at least two, at least three, at least four, at least five, at least six, at least seven, at least eight, at least nine, at least ten, at least twelve, at least sixteen, at least twenty, or more amino acid alterations relative to any one of the sequences recited in TABLE 1. In some embodiments, the one or more alterations comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty amino acid alterations relative to any one of the sequences recited in TABLE 1. In some embodiments, the one or more alterations comprises one, two, three, four, five, six, seven, eight, nine, ten, or more amino acid alterations relative to any one of the sequences recited in TABLE 1. In some embodiments, the effector protein comprising one or more amino acid alterations is a variant of an effector protein described herein. It is understood that any reference to an effector protein herein also refers to an effector protein variant as described herein. In some embodiments, the one or more amino acid alterations comprises conservative substitutions, non-conservative substitutions, deletions, or combinations thereof. In some embodiments, an effector protein or a nucleic acid encoding the effector protein comprises 1 amino acid alteration, 2 amino acid alterations, 3 amino acid alterations, 4 amino acid alterations, 5 amino acid alterations, 6 amino acid alterations, 7 amino acid alterations, 8 amino acid alterations, 9 amino acid alterations, 10 amino acid alterations or more relative to any one of the sequences recited in TABLE 1.

In some embodiments, compositions, systems, and methods described herein comprise an effector protein, or a nucleic acid encoding the effector protein, wherein the effector protein comprises one or more substitutions relative to any one of the sequences recited in TABLE 1. In some embodiments, the one or more substitutions comprises at least one, at least two, at least three, at least four, at least five, at least six, at least seven, at least eight, at least nine, at least ten, at least twelve, at least sixteen, at least twenty, or more substitutions relative to any one of the sequences recited in TABLE 1. In some embodiments, the one or more substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty substitutions relative to any one of the sequences recited in TABLE 1. In some embodiments, the one or more substitutions comprise one, two, three, four, five, six, seven, eight, nine, ten or more substitutions relative to any one of the sequences recited in TABLE 1. In some embodiments, the one or more substitutions comprise one or more conservative substitutions, one or more non-conservative substitutions, or combinations thereof.

In some embodiments, compositions, systems, and methods described herein comprise an effector protein, or a nucleic acid encoding the effector protein, wherein the effector protein comprises one or more conservative substitutions relative to any one of the sequences recited in TABLE 1. In some embodiments, the one or more conservative substitutions comprises at least one, at least two, at least three, at least four, at least five, at least six, at least seven, at least eight, at least nine, at least ten, at least twelve, at least sixteen, at least twenty, or more conservative substitutions relative to any one of the sequences recited in TABLE 1. In some embodiments, the one or more conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty conservative substitutions relative to any one of the sequences recited in TABLE 1. In some embodiments, the one or more conservative substitutions comprise one, two, three, four, five, six, seven, eight, nine, ten or more conservative substitutions relative to any one of the sequences recited in TABLE 1.

In some embodiments, compositions, systems, and methods described herein comprise an effector protein, or a nucleic acid encoding the effector protein, wherein the effector protein comprises one or more non-conservative substitutions relative to any one of the sequences recited in TABLE 1. In some embodiments, the one or more non-conservative substitutions comprises at least one, at least two, at least three, at least four, at least five, at least six, at least seven, at least eight, at least nine, at least ten, at least twelve, at least sixteen, at least twenty, or more non-conservative substitutions relative to any one of the sequences recited in TABLE 1. In some embodiments, the one or more non-conservative substitutions comprises one to twenty, one to sixteen, one to twelve, one to eight, one to four, four to twenty, four to sixteen, four to twelve, four to eight, eight to twenty, eight to sixteen, eight to twelve, twelve to twenty, twelve to sixteen, or sixteen to twenty non-conservative substitutions relative to any one of the sequences recited in TABLE 1. In some embodiments, the one or more non-conservative substitutions comprise one, two, three, four, five, six, seven, eight, nine, ten or more non-conservative substitutions relative to any one of the sequences recited in TABLE 1.

### Protospacer Adjacent Motif (PAM) Sequences

Effector proteins of the present disclosure may bind a target nucleic acid within or near a protospacer adjacent motif (PAM) sequence of the target nucleic acid. In some embodiments, the target nucleic acid is a double stranded nucleic acid comprising a target strand and a non-target strand. In some embodiments, binding occurs within 1, 2, 3, 4, 5, 6, 7, 8, 9, 10, 15, 20, 25, 30, 35, 40, 45, or 50 nucleotides of a 5′ or 3′ terminus of a PAM sequence. In some embodiments, effector proteins described herein recognize a PAM sequence. In some embodiments, recognizing a PAM sequence comprises interacting with a sequence adjacent to the PAM. In some embodiments, a target nucleic acid comprises a target sequence that is adjacent to a PAM sequence. In some embodiments, the effector protein does not require a PAM to bind a target nucleic acid.

In some embodiments, a target nucleic acid is a single stranded target nucleic acid comprising a target sequence. Accordingly, in some embodiments, the single stranded target nucleic acid comprises a PAM sequence described herein that is adjacent (e.g., within 1, 2, 3, 4, 5, 6, 7, 8, 9, 10, 15, 20, 25, 30, 35, 40, 45, or 50 nucleotides) or directly adjacent to the target sequence.

In some embodiments, a target nucleic acid is a double stranded nucleic acid comprising a target strand and a non-target strand, wherein the target strand comprises a target sequence. In some embodiments, the PAM sequence is located on the target strand. In some embodiments, the PAM sequence is located on the non-target strand. In some embodiments, the PAM sequence described herein is adjacent (e.g., within 1, 2, 3, 4, 5, 6, 7, 8, 9, 10, 15, 20, 25, 30, 35, 40, 45, or 50 nucleotides) to the target sequence on the target strand or the non-target strand. In some embodiments, the PAM sequence is located 5′ of the target sequence on the non-target strand. In some embodiments, such a PAM described herein is directly adjacent to the target sequence on the target strand or the non-target strand. In some embodiments, an RNP does not cleave the target strand or the non-target strand. In some embodiments, an RNP recognizes the PAM sequence, and hybridizes to a target sequence of the target nucleic acid.

In some embodiments, an effector protein described herein, or a multimeric complex thereof, recognizes a PAM on a target nucleic acid. In some embodiments, multiple effector proteins of the multimeric complex recognize a PAM on a target nucleic acid. In some embodiments, at least two of the multiple effector proteins recognize the same PAM sequence. In some embodiments, at least two of the multiple effector proteins recognize different PAM sequences. In some embodiments, only one effector protein of the multimeric complex recognizes a PAM on a target nucleic acid.

In some embodiments, a PAM sequence provided herein comprises any one of the nucleotide sequences recited in TABLE 3. In some embodiments, the PAM is 5′-NTTN-3′. In some embodiments, the PAM is 5′-NNTR-3′. In some embodiments, the effector protein set forth in SEQ ID NO: 1 or 2 recognizes the PAM sequence 5′-NTTN-3′. In some embodiments, the effector protein set forth in SEQ ID NO: 35, 2950, 3010, 3011, or 3012 recognizes the PAM sequence 5′-NNTR-3′. In some embodiments, the effector protein set forth in SEQ ID NO: 35 recognizes the PAM sequence 5′-NNTN-3′. PAMs used in compositions, systems, and methods herein are further described throughout the application.

### B. Effector Partners

Provided herein are compositions, systems, and methods comprising one or more effector partners or uses thereof. In some embodiments, the effector partner is a heterologous protein an effector protein described herein. In some embodiments, the effector partner is not an effector protein as described herein. In some embodiments, the effector partner is capable of imparting a function or activity that is not provided by an effector protein as described herein. In some embodiments, the effector partner comprises a second effector protein or a multimeric form thereof.

In some embodiments, an effector partner comprises a chromatin-modifying enzyme. In some embodiments, the effector partner chemically modifies a target nucleic acid, for example by methylating or acetylating the target nucleic acid in a sequence specific or non-specific manner. In some embodiments, an effector partner imparts a function or activity to a fusion protein comprising an effector protein that is not provided by the effector protein, including but not limited to methyltransferase activity.

In some embodiments, the effector partner is fused or linked to an effector protein described herein. In some embodiments, the amino terminus of the effector partner is linked to the carboxy terminus of the effector protein directly or by a linker. In some embodiments, the carboxy terminus of the effector partner is linked to the amino terminus of the effector protein directly or by a linker. In some embodiments, the effector partner may be functional when the effector protein is coupled to a guide nucleic acid. In some embodiments, the effector partner may be functional when the effector protein is coupled to a target nucleic acid. In some embodiments, the guide nucleic acid imparts sequence specific activity to the effector partner. By way of non-limiting example, the effector protein may comprise a catalytically inactive effector protein (e.g., a catalytically inactive variant of an effector protein described herein) when fused or linked to an effector partner.

In some embodiments, the effector partner modifies proteins associated with a target nucleic acid. In some embodiments, an effector partner may modulate transcription (e.g., inhibits transcription) of a target nucleic acid. In yet another example, an effector partner may directly or indirectly inhibit or reduce expression of a target nucleic acid. In some embodiments, compositions, systems and fusion proteins comprise two effector partners. In some embodiments, the two effector partners comprise two methyltransferases. In some embodiments, the two effector partners comprise a methyltransferase and a KRAB domain.

In some embodiments, the effector partner comprises a protein that provides methyltransferase activity. In some embodiments, the effector partner comprises a methyltransferase. In general, a methyltransferase is an enzyme that transfers a methyl group to a target nucleic acid. In some embodiments, the methyltransferase transfers a methyl group to a 5-carbon of the base cytosine. Commercial kits are available for assaying methyltransferase activity. Non-limiting examples of methyltransferases are HhaI DNA m5c-methyltransferase (M.HhaI), DNA methyltransferase 1 (DNMT1), DNA methyltransferase 3a (DNMT3A), DNA methyltransferase 3b (DNMT3B), and DNA methyltransferase 3 like (DNMT3L). In some embodiments, the effector partner is DNMT3A. In some embodiments, the effector partner is DNMT3L. In some embodiments, compositions comprise DNMT3L and DNMT3A. In some embodiments, fusion proteins comprise DNMT3L and DNMT3A.

In some embodiments, the effector partner comprises an H3K9 methylase. In general, an H3K9 methylase transfers a methyl group to Histone H3 lysine nine. Non-limiting examples of H3K9 methylases are KRAB domains, HPlalpha, KAP1, G9a, and SUV39H1. In some embodiments, the effector partner comprises a domain of a zinc finger protein. In some embodiments, the domain of the zinc finger protein is a KRAB domain. In some embodiments, the effector partner comprises a repressor comprising a KRAB domain or a variant thereof. In some embodiments, the KRAB domain is from zinc finger protein 10 (ZNF10). In some embodiments, the KRAB domain is from zinc finger imprinted 3 (ZIM3). In some embodiments, the KRAB domain is from zinc finger protein 28 (ZFP28). In some embodiments, the KRAB domain is from zinc finger protein 37A (ZNF37A). In some embodiments, the KRAB domain is from zinc finger protein 181 (ZNF181). In some embodiments, the KRAB domain is from zinc finger protein 208 (ZNF208). In some embodiments, the KRAB domain is from zinc finger protein 248 (ZNF248). In some embodiments, the KRAB domain is from zinc finger protein 334 (ZNF334). In some embodiments, the KRAB domain is from zinc finger protein 432 (ZNF432). In some embodiments, the KRAB domain is from zinc finger protein 510 (ZNF510). In some embodiments, the KRAB domain is from zinc finger protein 568 (ZNF568). In some embodiments, the KRAB domain is from zinc finger protein 862 (ZNF862). In some embodiments, the KRAB domain is from zinc finger protein 791 (ZNF791).In some embodiments, the KRAB domain is from zinc finger protein 398, (ZNF398). In some embodiments, the KRAB domain is from zinc finger protein 197 (ZNF197). In some embodiments, the KRAB domain is from zinc finger protein 382 (ZNF382). In some embodiments, the KRAB domain is from zinc finger protein 496 (ZNF496). In some embodiments, the KRAB domain is from zinc finger protein 534 (ZNF534). In some embodiments, the KRAB domain is from zinc finger protein 701 (ZNF701). In some embodiments, the KRAB domain is from zinc finger protein 141 (ZNF141). In some embodiments, the KRAB domain is from zinc finger protein 808 (ZNF808). In some embodiments, the KRAB domain is from zinc finger protein 773 (ZNF773). In some embodiments, the KRAB domain is from zinc finger protein 175 (ZNF175). In some embodiments, the KRAB domain is from zinc finger protein 331 (ZNF331). In some embodiments, the KRAB domain is from zinc finger protein 487 (ZNF487). In some embodiments, the KRAB domain is from zinc finger protein 233 (ZNF233). In some embodiments, the KRAB domain is from ZNF33A protein (ZNF33A). In some embodiments, the KRAB domain is from zinc finger protein 589 (ZNF589). In some embodiments, the KRAB domain is from zinc finger protein 431 (ZNF431). In some embodiments, the KRAB domain is from zinc finger protein 91 (ZNF91). In some embodiments, the KRAB domain is from putative zinc finger protein 730, (ZNF730). In some embodiments, the KRAB domain is from zinc finger protein 43 (ZNF43). In some embodiments, the KRAB domain is from zinc finger protein 136 (ZNF136). In some embodiments, the KRAB domain is from zinc finger protein 785 (ZNF785). In some embodiments, the KRAB domain is from zinc finger protein 764 (ZNF764). In some embodiments, the KRAB domain is from zinc finger protein 585A (ZNF585A). In some embodiments, the KRAB domain is from zinc finger protein 688 (ZNF688). In some embodiments, the KRAB domain is from zinc finger protein 182 (ZNF182). In some embodiments, the KRAB domain is from zinc finger protein 420 (ZNF420). In some embodiments, the KRAB domain is from zinc finger protein 675 (ZNF675). In some embodiments, the KRAB domain is from zinc finger protein 473 (ZNF473). In some embodiments, the KRAB domain is from zinc finger protein 30 homolog (ZFP30). In some embodiments, the KRAB domain is from zinc finger protein 658 (ZNF658). In some embodiments, the KRAB domain is from zinc finger protein 418 (ZNF418). In some embodiments, the KRAB domain is from zinc finger protein 846 (ZNF846). In some embodiments, the KRAB domain is from zinc finger protein 160 (ZNF160). In some embodiments, the KRAB domain is from zinc finger protein 468 (ZNF468). In some embodiments, the KRAB domain is from zinc finger protein 331 (ZNF331). In some embodiments, the KRAB domain is from zinc finger protein 425 (ZNF425). In some embodiments, the KRAB domain is from zinc finger protein 2 (ZNF2). In some embodiments, the KRAB domain is from zinc finger protein 212 (ZIN212). In some embodiments, the KRAB domain is from zinc finger protein 225 (ZNF225). In some embodiments, the KRAB domain is from zinc finger protein 568 (ZNF568). In some embodiments, the KRAB domain is from zinc finger protein 33A (ZNF33A). In some embodiments, the KRAB domain is from zinc finger protein 415 (ZNF415). In some embodiments, the KRAB domain is from zinc finger protein 548 (ZNF548). In some embodiments, the KRAB domain is from zinc finger protein 707 (ZNF707). In some embodiments, the KRAB domain is from zinc finger protein 721 (ZNF721). In some embodiments, the KRAB domain is from zinc finger protein 26 (ZNF26). In some embodiments, the KRAB domain is from zinc finger protein 461 (ZNG461). In some embodiments, the KRAB domain is from zinc finger protein 140 (ZNF140). In some embodiments, the KRAB domain is from zinc finger protein 707 (ZNF707). In some embodiments, the KRAB domain is from zinc finger protein 790 (ZNF790). In some embodiments, the KRAB domain is from zinc finger protein 717 (ZNF717). In some embodiments, the KRAB domain is from zinc finger protein 226 (ZNF226). In some embodiments, the KRAB domain is from zinc finger protein 19 (ZNF19). In some embodiments, the KRAB domain is from zinc finger protein 667 (ZNF667). In some embodiments, the KRAB domain is from zinc finger protein 789 (ZNF789). In some embodiments, the KRAB domain is from zinc finger protein 514 (ZNF514). In some embodiments, the KRAB domain is from zinc finger protein 677 (ZNF677). In some embodiments, the KRAB domain is from zinc finger protein 28 (ZNF28). In some embodiments, the KRAB domain is from zinc finger protein 83 (ZNF83). In some embodiments, the KRAB domain is from zinc finger protein 284 (ZNF284). In some embodiments, the KRAB domain is from zinc finger protein 324B, (ZNF324B). In some embodiments, the KRAB domain is from zinc finger protein 230 (ZNF230). In some embodiments, the KRAB domain is from zinc finger protein 115 (ZNF115). In some embodiments, the KRAB domain is from zinc finger protein 587B (ZNF587B). In some embodiments, the KRAB domain is from zinc finger protein 571 (ZNF571). In some embodiments, the KRAB domain is from zinc finger protein 233 (ZNF233). In some embodiments, the KRAB domain is from zinc finger protein 285 (ZNF285). In some embodiments, the KRAB domain is from zinc finger protein 320 (ZNF320). In some embodiments, the KRAB domain is from zinc finger protein 569 (ZNF569). In some embodiments, the KRAB domain is from zinc finger protein 84 (ZNF84). In some embodiments, the KRAB domain is from ZNF167 protein (ZNF167). In some embodiments, the KRAB domain is from zinc finger protein 234 (ZNF234). In some embodiments, the KRAB domain is from zinc finger protein 426 (ZNF426). In some embodiments, the KRAB domain is from zinc finger protein 264 (ZNF264). In some embodiments, the KRAB domain is from zinc finger protein 700 (ZNF700). In some embodiments, the KRAB variant comprises one or more substitutions relative to the wildtype KRAB. In some embodiments, the one or more substitutions are at the KRAB N terminus. In some embodiments, the repressor comprises a KRAB variant from ZNF10 comprising AW7EE substitutions (i.e., KRAB(ZNF10)_AW7EE). In some embodiments, the repressor comprises a KRAB variant from ZNF10 comprising WSR8EEE substitutions (i.e., KRAB(ZNF10)_WSR8EEE). In some embodiments, the repressor comprises a KRAB variant from ZNF791 comprising codopt1 substitutions (i.e., KRAB(ZNF791)_codopt1). In some embodiments, the repressor comprises a KRAB variant from ZNF791 comprising codopt2 substitutions (i.e., KRAB(ZNF791)_codopt2).

In some embodiments, the effector partner comprises a repressor comprising a chromodomain or a variant thereof. Non-limiting examples of chromodomains include CBX1, CBX2, CBX3, CBX4, CBX5, CBX6, CBX7, CBX8, and MPP8. In some embodiments, the chromodomain variant comprises Q9D/K33E substitutions (i.e., CBX1VD, CBX2VD, CBX3VD, CBX4VD, CBX5VD, CBX6VD, CBX7VD, and CBX8VD).

In some embodiments, the effector partner comprises a repressor selected from Ring1 and YY1 binding protein (RYBP), YY1 associated factor 2 (YAF2), ANM2, cyclic AMP response element modulator (CREM), GS homeobox 2 (GSX2), methyl CpG binding protein 2 (MECP2), small ubiquitin-like modifier 1(SUMO1), small ubiquitin-like modifier 3 (SUMO3), small ubiquitin-like modifier 5 (SUMOS), methyl-CpG-binding domain (MBD)-containing protein 2B (MBD2B), heterochromatin protein 1 (HPla), Spalt Like Transcription Factor 1 (SALL1), Sin3a corepressor complex component (SDS3), and Synovial sarcoma, X breakpoint 2 (SSX2).

In some embodiments, the effector partner comprises one or more repressors selected from a KRAB domain or a variant thereof described herein, a chromodomain or a variant thereof described herein, RYBP, YAF2, ANM2, CREM, GSX2, MECP2, SUMO1, SUMO3, SUMOS, HPla, SALL1, SDS3, SSX2, and any combination thereof. In some embodiments, the effector partner comprises one or more repressors selected from KRAB(ZIM3), KRAB(ZNF10), KRAB(ZFP28), KRAB(ZNF37A), KRAB(ZNF181), KRAB(ZNF208), KRAB(ZNF248), KRAB(ZNF334), KRAB(ZNF432), KRAB(ZNF510), KRAB(ZNF568), KRAB(ZNF862), KRAB(ZNF791), KRAB(ZNF10)_AW7EE, KRAB(ZNF10)_WSR8EEE, KRAB(ZNF791)_codopt1, KRAB(ZNF791)_codopt2, CBX1, CBX2, CBX3, CBX4, CBX5, CBX6, CBX7, CBX8, MPP8, CBX1VD, CBX2VD, CBX3VD, CBX4VD, CBX5VD, CBX6VD, CBX7VD, CBX8VD, RYBP, YAF2, ANM2, CREM, GSX2, MECP2, SUMO1, SUMO3, SUMOS, MBD2B, HPla, SALL1, SDS3, SSX2, and any combination thereof. In some embodiments, two or more repressors are fused in tandem. In some embodiments, two or more repressors are fused by linkers.

In some embodiments, the effector partner comprises bipartite repressors selected from the combination of CBX1VD-KRAB(ZIM3), CBX2VD-KRAB(ZIM3), CBX3VD-KRAB(ZIM3), CBX4VD-KRAB(ZIM3), CBX5VD-KRAB(ZIM3), CBX6VD-KRAB(ZIM3), CBX7VD-KRAB(ZIM3), CBX8VD-KRAB(ZIM3), CREM-KRAB(ZNF10), CREM-MPP8, KRAB(ZNF10)-ANM2, KRAB(ZNF10)-CBX1, KRAB(ZNF10)-CBX3, KRAB(ZNF10)-CBX4, KRAB(ZNF10)-CBX5, KRAB(ZNF10)-GSX2, KRAB(ZNF10)-KRAB(ZFP28), KRAB(ZNF10)-KRAB(ZNF37A), KRAB(ZNF10)-KRAB(ZNF181) KRAB(ZNF10)-KRAB(ZNF248), KRAB(ZNF10)-KRAB(ZNF334), KRAB(ZNF10)-KRAB(ZNF432), KRAB(ZNF10)-KRAB(ZNF510), KRAB(ZNF10)-KRAB(ZNF568), KRAB(ZNF10)-KRAB(ZNF862), KRAB(ZNF10)-MeCP2, KRAB(ZNF10)-MPP8, KRAB(ZNF10)-RYBP, KRAB(ZNF10)-SUMO1, KRAB(ZNF10)-SUMO3, KRAB(ZNF10)-SUMOS, KRAB(ZNF10)-YAF2, KRAB(ZNF791)_codopt1-KRAB(ZNF791)_codopt2, MBD2B-HP1a, MBD2B-KRAB(ZNF10), SALL1-SDS3, and SUMO3-SSX2. TABLE 4 provides amino acid sequences of the exemplary bipartite repressors.

In some embodiments, the effector partner comprises an H3K27 methylase. In general, an H3K27 methylase transfers a methyl group to Histone H3 lysine 27. A non-limiting example of an H3K27 methylase is EZH2. EZH2 is a catalytic subunit of the polycomb repressive complex 2 (PRC2). The effector partner may comprise one or more components of the PRC complex.

It is understood that an effector partner may include an entire protein, or in some embodiments, may include a fragment of the protein (e.g., a functional domain). In some embodiments, the functional domain binds or interacts with a nucleic acid, including intramolecular and/or intermolecular secondary structures thereof (e.g., hairpins, stem-loops, etc.). The functional domain may interact transiently or irreversibly, directly, or indirectly. In some embodiments, a functional domain comprises a region of one or more amino acids in a protein that is required for an activity of the protein, or the full extent of that activity, as measured in an in vitro assay. Activities include but are not limited to nucleic acid binding, nucleic acid modifying, protein binding or combinations thereof. The absence of the functional domain, including mutations of the functional domain, would abolish or reduce activity.

In some embodiments, effector partners inhibit or reduce expression of a target nucleic acid. In some embodiments, effector partners reduce expression of the target nucleic acid relative to its expression in the absence of the effector partners. Relative expression, including transcription and RNA levels, may be assessed, quantified, and compared, e.g., by RT-qPCR. In some embodiments, effector partners may comprise a transcriptional repressor. In some embodiments, the transcriptional repressors may inhibit transcription by: recruitment of other transcription factor proteins; modification of target DNA such as methylation; recruitment of a DNA modifier; modulation of histones associated with target DNA; recruitment of a histone modifier such as those that modify acetylation and/or methylation of histones; or a combination thereof.

In some embodiments, an effector partner provided herein comprises any one of the amino acid sequences recited in TABLE 4. Effector partners used in compositions, systems, and methods herein are further described throughout the application.

### Linkers

In some embodiments, effector proteins and effector partners are directly linked via a covalent bond. In some embodiments, In some embodiments, effector proteins and effector partners are linked via a molecule, also referred to as a “linker.” The linker may comprise or consist of a chemical group. In some embodiments, the linker comprises an amino acid. In some embodiments, a peptide linker comprises at least two amino acids linked by an amide bond. In general, the linker connects a terminus of the effector protein to a terminus of the fusion partner. In some embodiments, carboxy terminus of the effector protein is linked to the amino terminus of the fusion partner. In some embodiments, carboxy terminus of the fusion partner is linked to the amino terminus of the effector protein. In some embodiments, the effector protein and the fusion partner are directly linked by a covalent bond.

In some embodiments, linkers comprise one or more amino acids. In some embodiments, linker is a protein. In some embodiments, a terminus of the effector protein is linked to a terminus of the fusion partner through an amide bond. In some embodiments, a terminus of the effector protein is linked to a terminus of the fusion partner through a peptide bond. In some embodiments, linkers comprise an amino acid. In some embodiments, linkers comprise a peptide. In some embodiments, an effector protein is coupled to a fusion partner by a linker protein. In some embodiments, the linker may have any of a variety of amino acid sequences. In some embodiments, the linker may comprise a region of rigidity (e.g., beta sheet, alpha helix), a region of flexibility, or any combination thereof. In some embodiments, the linker comprises small amino acids, such as glycine and alanine, that impart high degrees of flexibility. The ordinarily skilled artisan will recognize that design of a peptide conjugated to any desired element may include linkers that are all or partially flexible, such that the linker may include a flexible linker as well as one or more portions that confer less flexible structure. Suitable linkers include proteins of 4 linked amino acids to 40 linked amino acids in length, or between 4 linked amino acids and 25 linked amino acids in length. In some embodiments, linked amino acids described herein comprise at least two amino acids linked by an amide bond.

Linkers may be produced by using synthetic, linker-encoding oligonucleotides to couple proteins, or may be encoded by a nucleic acid sequence encoding a fusion protein (e.g., an effector protein coupled to a fusion partner). In some embodiments, the linker is from 1 to 100 amino acids in length. In some embodiments, the linker is more 100 amino acids in length. In some embodiments, the linker is from 10 to 27 amino acids in length. In some embodiments, linker proteins include glycine polymers (G)n, glycine-serine polymers (including, for example, (GS)n, GSGGSn (SEQ ID NO:26), GGSGGSn (SEQ ID NO:27), and GGGSn (SEQ ID NO:28), where n is an integer of at least one), glycine-alanine polymers, and alanine-serine polymers. In some embodiments, linkers may comprise amino acid sequences including, but not limited to, GGSG (SEQ ID NO:29), GGSGG (SEQ ID NO:30), GSGSG (SEQ ID NO:31), GSGGG (SEQ ID NO:32), GGGSG (SEQ ID NO:33), and GSSSG (SEQ ID NO:34). In some embodiments, the linker comprises one or more repeats a tri-peptide GGS. In some embodiments, the linker is an XTEN linker. In some embodiments, the XTEN linker is an XTEN80 linker. In some embodiments, the XTEN linker is an XTEN20 linker.

In some embodiments, linkers do not comprise an amino acid. In some embodiments, linkers do not comprise a peptide. In some embodiments, linkers comprise a nucleotide, a polynucleotide, a polymer, or a lipid. In some embodiments, linker may be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacrylamide, polyacrylate, polycyanoacrylates, lipid polymers, chitins, hyaluronic acid, heparin, or an alkyl linker.

In some embodiments, a linker is recognized and cleaved by a protein described herein. In some embodiments, a linker comprises a recognition sequence that may be recognized and cleaved by the protein. In some embodiments, a guide nucleic acid comprises an aptamer, which may serve a similar function as a linker, bringing an effector protein and an effector partner protein into proximity. The aptamer can functionally connect two proteins (e.g., effector protein, effector partner) by interacting non-covalently with both, thereby bringing both proteins into proximity of the guide nucleic acid. In some embodiments, the first protein and/or the second protein comprise or is covalently linked to an aptamer binding moiety. In some embodiments, the aptamer is a short single stranded DNA (ssDNA) or RNA (ssRNA) molecule capable of being bound be the aptamer binding moiety. In some embodiments, the aptamer is a molecule that is capable of mimicking antibody binding activity and may be classified as a chemical antibody. In some instances, the aptamer described herein refers to artificial oligonucleotides that bind one or more specific molecules. In some embodiments, aptamers exhibit a range of affinities (KD in the pM to μM range) with little or no off-target binding.

In some embodiments, a peptide linker provided herein comprises any one of the amino acid sequences recited in TABLE 5. Linkers used in compositions, systems, and methods herein are further described throughout the application.

### C. Engineered Proteins

In some embodiments, proteins (e.g., effector protein, effector partner) described herein have been modified (also referred to as an engineered protein). In some embodiments, a modification of the proteins may include addition of one or more amino acids, deletion of one or more amino acids, substitution of one or more amino acids, or combinations thereof. In some embodiments, the proteins disclosed herein are engineered proteins. Unless otherwise indicated, reference to the proteins throughout the present disclosure include engineered proteins thereof.

In some embodiments, proteins (e.g., effector protein, effector partner) described herein can be modified with the addition of one or more heterologous peptides or heterologous polypeptides (referred to collectively herein as a heterologous polypeptide). In some embodiments, the protein modified with the addition of one or more heterologous peptides or heterologous polypeptides may be referred to herein as a fusion protein. Such fusion proteins are described herein and throughout.

In some embodiments, a heterologous peptide or heterologous polypeptide comprises a subcellular localization signal. In some embodiments, a subcellular localization signal can be a nuclear localization signal (NLS). In some embodiments, the NLS facilitates localization of a nucleic acid, protein, or small molecule to the nucleus, when present in a cell that contains a nuclear compartment. TABLE 2 lists exemplary NLS sequences. In some embodiments, the subcellular localization signal is a nuclear export signal (NES), a sequence to keep the protein retained in the cytoplasm, a mitochondrial localization signal for targeting to the mitochondria, a chloroplast localization signal for targeting to a chloroplast, an ER retention signal, and the like. In some embodiments, the protein described herein is not modified with a subcellular localization signal so that the protein is not targeted to the nucleus, which can be advantageous depending on the circumstance (e.g., when the target nucleic acid is an RNA that is present in the cytosol).

In some embodiments, a heterologous peptide or heterologous polypeptide comprises a protein tag. In some embodiments, the protein tag is referred to as purification tag or a fluorescent protein. The protein tag may be detectable for use in detection of the protein and/or purification of the protein. Accordingly, in some embodiments, compositions, systems and methods comprise a protein tag or use thereof. Any suitable protein tag may be used depending on the purpose of its use. Non-limiting examples of protein tags include a fluorescent protein, a histidine tag, e.g., a 6XHis tag; a hemagglutinin (HA) tag; a FLAG tag; a Myc tag; and maltose binding protein (MBP). In some embodiments, the protein tag is a portion of MBP that can be detected and/or purified. Non-limiting examples of fluorescent proteins include green fluorescent protein (GFP), yellow fluorescent protein (YFP), red fluorescent protein (RFP), cyan fluorescent protein (CFP), mCherry, and tdTomato.

A heterologous polypeptide may be located at or near the amino terminus (N-terminus) of the protein (e.g., effector protein, effector partner) disclosed herein. A heterologous polypeptide may be located at or near the carboxy terminus (C-terminus) of the proteins disclosed herein. In some embodiments, a heterologous polypeptide is located internally in the protein described herein (i.e., is not at the N- or C-terminus of the protein described herein) at a suitable insertion site.

In some embodiments, protein (e.g., effector protein, effector partner) described herein comprises 1, 2, 3, 4, 5, 6, 7, 8, 9, 10, or more heterologous polypeptides at or near the N-terminus, 1, 2, 3, 4, 5, 6, 7, 8, 9, 10, or more heterologous polypeptides at or near the C-terminus, or a combination of these (e.g., one or more heterologous polypeptides at the amino-terminus and one or more heterologous polypeptides at the carboxy terminus). When more than one heterologous polypeptide is present, each may be selected independently of the others, such that a single heterologous polypeptide may be present in more than one copy and/or in combination with one or more other heterologous polypeptides present in one or more copies. In some embodiments, a heterologous polypeptide is considered near the N- or C-terminus when the nearest amino acid of the heterologous polypeptide is within about 1, 2, 3, 4, 5, 10, 15, 20, 25, 30, 40, 50, or more amino acids along the polypeptide chain from the N- or C-terminus.

Effector proteins may comprise one or more modifications that reduce the activity of the effector protein relative to a naturally occurring effector protein. An effector protein may have a 100%, 90%, 80%, 70%, 60%, 50%, 40%, 30%, 20%, 10%, 5%, 1%, or less, decrease of the activity of a naturally occurring counterpart. Decreased activity may be decreased catalytic activity (e.g., nuclease activity) as compared to a naturally-occurring counterpart.

### D. Fusion Proteins

In some embodiments, the present disclosure provides a fusion protein comprising an effector protein described herein and one or more effector partners described herein. In some embodiments, the fusion protein comprises, from N-terminus to C-terminus, an effector protein and one or more effector partners. In some embodiments, the fusion protein comprises, from N-terminus to C-terminus, one or more effector partners and an effector protein. In some embodiments, the fusion protein comprises, from N-terminus to C-terminus, one or more effector partners, an effector protein, and another one or more effector partners. Exemplary fusion protein sequences are provided in TABLE 6.

In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence set forth in TABLE 1 and an effector partner comprising an amino acid sequence that is at least 90%, 91%, 92%, 93%, 94%, 95%, 96%, 97%, 98%, or 99% identical to an amino acid sequence set forth in TABLE 4. In some embodiments, the fusion protein described herein comprises an effector protein comprising or consisting of an amino acid sequence set forth in TABLE 1 and an effector partner comprising or consisting of an amino acid sequence set forth in TABLE 4.

In some embodiments, the fusion protein described herein comprises an effector protein comprising at least one amino acid alteration relative to the amino acid sequence of SEQ ID NO: 1, wherein the at least one amino acid substitution is selected from: L26R, L26K, A121Q, D369A, D369N, D658A, D658N, E567A, and E567Q, and a combination thereof. In some embodiments, the fusion protein described herein comprises an effector protein comprising two amino acid alterations relative to the amino acid sequence of SEQ ID NO: 1, wherein the amino acid substitutions are L26R and E567Q. In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence of SEQ ID NO: 2. In some embodiments, the fusion protein described herein comprises an effector protein comprising the amino acid sequence of SEQ ID NO: 2.

In some embodiments, the fusion protein described herein comprises an effector protein comprising at least one amino acid alteration relative to the amino acid sequence of SEQ ID NO: 35, wherein the at least one amino acid substitution is selected from: D220R, E335Q, and a combination thereof. In some embodiments, the fusion protein described herein comprises an effector protein comprising two amino acid alterations relative to the amino acid sequence of SEQ ID NO: 35, wherein the amino acid substitutions are D220R and E335Q. In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence of SEQ ID NO: 2950. In some embodiments, the fusion protein described herein comprises an effector protein comprising the amino acid sequence of SEQ ID NO: 2950. In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence of SEQ ID NO: 3010. In some embodiments, the fusion protein described herein comprises an effector protein comprising the amino acid sequence of SEQ ID NO: 3010. In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence of SEQ ID NO: 3011. In some embodiments, the fusion protein described herein comprises an effector protein comprising the amino acid sequence of SEQ ID NO: 3011. In some embodiments, the fusion protein described herein comprises an effector protein comprising an amino acid sequence that is at least 90%, 91%, 92%, 93%, 94%, 95%, 96%, 97%, 98%, or 99% identical to an amino acid sequence of SEQ ID NO: 3012. In some embodiments, the fusion protein described herein comprises an effector protein comprising the amino acid sequence of SEQ ID NO: 3012.

In some embodiments, the fusion protein comprises an amino acid sequence that is at least 90%, 91%, 92%, 93%, 94%, 95%, 96%, 97%, 98%, or 99% identical to an amino acid sequence set forth in TABLE 6. In some embodiments, the fusion protein comprises an amino acid sequence that is 100% identical to an amino acid sequence set forth in set forth in TABLE 6.

In some embodiments, the fusion protein comprises an effector protein described herein and one effector partner described herein. In some embodiments, the fusion protein comprises an effector protein comprising an amino acid sequence that is at least 90%, 91%, 92%, 93%, 94%, 95%, 96%, 97%, 98%, or 99% identical to an amino acid sequence set forth in TABLE 1 and one effector partner, wherein the effector partner comprises an amino acid sequence that is at least 90%, 91%, 92%, 93%, 94%, 95%, 96%, 97%, 98%, or 99% identical to an amino acid sequence set forth in TABLE 4. In some embodiments, the fusion protein comprises an effector protein comprising or consisting of an amino acid sequence set forth in TABLE 1 and one effector partner, wherein the effector partner comprises or consists of an amino acid sequence set forth in TABLE 4. In some embodiments, the fusion protein comprises an effector protein comprising an amino acid sequence that is at least 90%, 91%, 92%, 93%, 94%, 95%, 96%, 97%, 98%, or 99% identical to an amino acid sequence set forth in TABLE 1 and one effector partner, wherein the effector partner is selected from DNMT3L, DNMT3A, KRAB(ZNF10), and KRAB(ZIM3). In some embodiments, the effector partner is linked to the C-terminus or N-terminus of the effector protein.

In some embodiments, the fusion protein comprises an effector protein described herein and two effector partners described herein. In some embodiments, the fusion protein comprises an effector protein comprising an amino acid sequence that is at least 90%, 91%, 92%, 93%, 94%, 95%, 96%, 97%, 98%, or 99% identical to an amino acid sequence set forth in TABLE 1 and a first effector partner and a second effector partner, wherein the first effector partner and the second effector partner comprise an amino acid sequence that is at least 90%, 91%, 92%, 93%, 94%, 95%, 96%, 97%, 98%, or 99% identical to an amino acid sequence set forth in TABLE 4. In some embodiments, the fusion protein comprises an effector protein comprising or consisting of an amino acid sequence set forth in TABLE 1 and a first effector partner and a second effector partner, wherein the first effector partner and the second effector partner comprise or consist of an amino acid sequence set forth in TABLE 4. In some embodiments, the fusion protein comprises an effector protein comprising an amino acid sequence that is at least 90%, 91%, 92%, 93%, 94%, 95%, 96%, 97%, 98%, or 99% identical to an amino acid sequence set forth in TABLE 1 and a first effector partner and a second effector partner, wherein the first effector partner and the second effector partner are selected from DNMT3L, DNMT3A, KRAB(ZNF10), KRAB(ZIM3), YAF2, MPP8, RYBP, CBX3VD, and CBX5VD, and a combination thereof. In some embodiments, the fusion protein comprises an effector protein described herein, a first effector partner described herein and a second effector partner described herein, wherein the first effector partner is linked to the C-terminus of the effector protein, and wherein the second effector partner is linked to the N-terminus of the effector protein or the C-terminus of the first effector partner. In some embodiments, the fusion protein comprises an effector protein described herein, a first effector partner described herein and a second effector partner described herein, wherein the first effector partner is linked to the N-terminus of the effector protein, and wherein the second effector partner is linked to the N-terminus of the first effector partner.

In some embodiments, the fusion protein comprises an effector protein described herein and three effector partners described herein. In some embodiments, the fusion protein comprises an effector protein comprising an amino acid sequence that is at least 90%, 91%, 92%, 93%, 94%, 95%, 96%, 97%, 98%, or 99% identical to an amino acid sequence set forth in TABLE 1 and a first effector partner, a second effector partner and a third effector partner, wherein the first effector partner, the second effector partner and the third effector partner comprise an amino acid sequence that is at least 90%, 91%, 92%, 93%, 94%, 95%, 96%, 97%, 98%, or 99% identical to an amino acid sequence set forth in TABLE 4. In some embodiments, the fusion protein comprises an effector protein comprising or consisting of an amino acid sequence set forth in TABLE 1 and a first effector partner, a second effector partner and a third effector partner, wherein the first effector partner, the second effector partner and the third effector partner comprise or consist of an amino acid sequence set forth in TABLE 4. In some embodiments, the fusion protein comprises an effector protein comprising an amino acid sequence that is at least 90%, 91%, 92%, 93%, 94%, 95%, 96%, 97%, 98%, or 99% identical to an amino acid sequence set forth in TABLE 1 and a first effector partner, a second effector partner and a third effector partner, wherein the first effector partner, the second effector partner and the third effector partner are selected from DNMT3L, DNMT3A, KRAB(ZNF10), KRAB(ZIM3), YAF2, MPP8, RYBP, CBX3VD, and CBX5VD, and a combination thereof. In some embodiments, the fusion protein comprises an effector protein described herein, a first effector partner described herein, a second effector partner described herein and a third effector partner described herein, wherein the first effector partner is linked to the C-terminus of the effector protein, wherein the second effector partner is linked to the N-terminus of the effector protein, and wherein the third effector partner is linked to the C-terminus of the first effector partner or the N-terminus of the second effector partner. In some embodiments, the fusion protein comprises an effector protein described herein, a first effector partner described herein, a second effector partner described herein and a third effector partner described herein, wherein the first effector partner is linked to the C-terminus of the effector protein, wherein the second effector partner is linked to the C-terminus of the first effector partner, and wherein the third effector partner is linked to the C-terminus of the second effector partner. In some embodiments, the fusion protein comprises an effector protein described herein, a first effector partner described herein, a second effector partner described herein and a third effector partner described herein, wherein the first effector partner is linked to the N-terminus of the effector protein, wherein the second effector partner is linked to the N-terminus of the first effector partner, and wherein the third effector partner is linked to the N-terminus of the second effector partner.

In some embodiments, the fusion protein comprises an effector protein, a first effector partner, and a second effector partner. In some embodiments, the effector protein comprises dCasPhi12(L26R, E567Q) or a variant thereof described herein. In some embodiments, the first effector partner comprises DNMT3L or DNMT3A or a variant thereof described herein. In some embodiments, the second effector partner comprises bipartite repressors or a variant thereof described herein. In some embodiments, the first effector partner is linked to the C-terminus of the effector protein. In some embodiments, the second effector partner is linked to the C-terminus of the first effector partner. In some embodiments, the fusion protein comprises an effector protein comprising an amino acid sequence that is at least 90%, 91%, 92%, 93%, 94%, 95%, 96%, 97%, 98%, or 99% identical to an amino acid sequence of SEQ ID NO: 2, a first effector partner comprising an amino acid sequence that is at least 90%, 91%, 92%, 93%, 94%, 95%, 96%, 97%, 98%, or 99% identical to an amino acid sequence selected from SEQ ID NO: 10 and SEQ ID NO: 11, and a second effector partner comprising an amino acid sequence that is at least 90%, 91%, 92%, 93%, 94%, 95%, 96%, 97%, 98%, or 99% identical to an amino acid sequence of any of SEQ ID NOs: 12, 3005-3009. In some embodiments, the fusion protein comprises an effector protein comprising an amino acid sequence that is at least 90%, 91%, 92%, 93%, 94%, 95%, 96%, 97%, 98%, or 99% identical to an amino acid sequence of SEQ ID NO: 2, a first effector partner comprising an amino acid sequence that is at least 90%, 91%, 92%, 93%, 94%, 95%, 96%, 97%, 98%, or 99% identical to an amino acid sequence of SEQ ID NO: 11, and a second effector partner comprising an amino acid sequence that is at least 90%, 91%, 92%, 93%, 94%, 95%, 96%, 97%, 98%, or 99% identical to an amino acid sequence of SEQ ID NO: 3005. In some embodiments, the fusion protein comprises an effector protein comprising an amino acid sequence that is at least 90%, 91%, 92%, 93%, 94%, 95%, 96%, 97%, 98%, or 99% identical to an amino acid sequence of SEQ ID NO: 2, a first effector partner comprising an amino acid sequence that is at least 90%, 91%, 92%, 93%, 94%, 95%, 96%, 97%, 98%, or 99% identical to an amino acid sequence of SEQ ID NO: 11, and a second effector partner comprising an amino acid sequence that is at least 90%, 91%, 92%, 93%, 94%, 95%, 96%, 97%, 98%, or 99% identical to an amino acid sequence of SEQ ID NO: 3006. In some embodiments, the fusion protein comprises an effector protein comprising an amino acid sequence that is at least 90%, 91%, 92%, 93%, 94%, 95%, 96%, 97%, 98%, or 99% identical to an amino acid sequence of SEQ ID NO: 2, a first effector partner comprising an amino acid sequence that is at least 90%, 91%, 92%, 93%, 94%, 95%, 96%, 97%, 98%, or 99% identical to an amino acid sequence of SEQ ID NO: 11, and a second effector partner comprising an amino acid sequence that is at least 90%, 91%, 92%, 93%, 94%, 95%, 96%, 97%, 98%, or 99% identical to an amino acid sequence of SEQ ID NO: 3007. In some embodiments, the fusion protein comprises an effector protein comprising an amino acid sequence that is at least 90%, 91%, 92%, 93%, 94%, 95%, 96%, 97%, 98%, or 99% identical to an amino acid sequence of SEQ ID NO: 2, a first effector partner comprising an amino acid sequence that is at least 90%, 91%, 92%, 93%, 94%, 95%, 96%, 97%, 98%, or 99% identical to an amino acid sequence of SEQ ID NO: 11, and a second effector partner comprising an amino acid sequence that is at least 90%, 91%, 92%, 93%, 94%, 95%, 96%, 97%, 98%, or 99% identical to an amino acid sequence of SEQ ID NO: 3008. In some embodiments, the fusion protein comprises an effector protein comprising an amino acid sequence that is at least 90%, 91%, 92%, 93%, 94%, 95%, 96%, 97%, 98%, or 99% identical to an amino acid sequence of SEQ ID NO: 2, a first effector partner comprising an amino acid sequence that is at least 90%, 91%, 92%, 93%, 94%, 95%, 96%, 97%, 98%, or 99% identical to an amino acid sequence of SEQ ID NO: 11, and a second effector partner comprising an amino acid sequence that is at least 90%, 91%, 92%, 93%, 94%, 95%, 96%, 97%, 98%, or 99% identical to an amino acid sequence of SEQ ID NO: 3009. In some embodiments, the fusion protein comprises an effector protein comprising the amino acid sequence of SEQ ID NO: 2, a first effector partner comprising the amino acid sequence of SEQ ID NO: 11, and a second effector partner comprising an amino acid sequence of any of SEQ ID NOs: 3005-3009. In some embodiments, the fusion protein comprises an effector protein comprising the amino acid sequence of SEQ ID NO: 2, a first effector partner comprising the amino acid sequence of SEQ ID NO: 11, and a second effector partner comprising the amino acid sequence of SEQ ID NO: 3005. In some embodiments, the fusion protein comprises an effector protein comprising the amino acid sequence of SEQ ID NO: 2, a first effector partner comprising the amino acid sequence of SEQ ID NO: 11, and a second effector partner comprising the amino acid sequence of SEQ ID NO: 3006. In some embodiments, the fusion protein comprises an effector protein comprising the amino acid sequence of SEQ ID NO: 2, a first effector partner comprising the amino acid sequence of SEQ ID NO: 11, and a second effector partner comprising the amino acid sequence of SEQ ID NO: 3007. In some embodiments, the fusion protein comprises an effector protein comprising the amino acid sequence of SEQ ID NO: 2, a first effector partner comprising the amino acid sequence of SEQ ID NO: 11, and a second effector partner comprising the amino acid sequence of SEQ ID NO: 3008. In some embodiments, the fusion protein comprises an effector protein comprising the amino acid sequence of SEQ ID NO: 2, a first effector partner comprising the amino acid sequence of SEQ ID NO: 11, and a second effector partner comprising the amino acid sequence of SEQ ID NOs: 3009.

In some embodiments, the fusion protein comprises the effector protein comprising a Cas protein, the first effector partner comprising DNMT3L, and the second effector partner comprising bipartite repressors KRAB(ZNF10)-YAF2. In some embodiments, the fusion protein comprises the effector protein comprising a Cas protein, the first effector partner comprising an amino acid sequence that is at least 90%, 91%, 92%, 93%, 94%, 95%, 96%, 97%, 98%, or 99% identical to an amino acid sequence of SEQ ID NO: 11, and the second effector partner comprising an amino acid sequence that is at least 90%, 91%, 92%, 93%, 94%, 95%, 96%, 97%, 98%, or 99% identical to an amino acid sequence of any of SEQ ID NO: 3005. In some embodiments, the fusion protein comprises the effector protein comprising a Cas protein, the first effector partner comprising the amino acid sequence of SEQ ID NO: 11, and the second effector partner comprising an amino acid sequence of any of SEQ ID NO: 3005. In some embodiments, the fusion protein comprises the effector protein comprising a Cas protein, the first effector partner comprising DNMT3L, and the second effector partner comprising CBX3VD. In some embodiments, the second effector partner further comprises KRAB(ZIM3). In some embodiments, the fusion protein comprises the effector protein comprising a Cas protein, the first effector partner comprising DNMT3L, and the second effector partner comprising bipartite repressors CBX3VD-KRAB(ZIM3). In some embodiments, the fusion protein comprises the effector protein comprising a Cas protein, the first effector partner comprising an amino acid sequence that is at least 90%, 91%, 92%, 93%, 94%, 95%, 96%, 97%, 98%, or 99% identical to an amino acid sequence of SEQ ID NO: 11, and the second effector partner comprising an amino acid sequence that is at least 90%, 91%, 92%, 93%, 94%, 95%, 96%, 97%, 98%, or 99% identical to an amino acid sequence of any of SEQ ID NO: 3008. In some embodiments, the fusion protein comprises the effector protein comprising a Cas protein, the first effector partner comprising the amino acid sequence of SEQ ID NO: 11, and the second effector partner comprising an amino acid sequence of any of SEQ ID NO: 3008. In some embodiments, the fusion protein comprises the effector protein comprising a Cas protein, the first effector partner comprising DNMT3L, and the second effector partner comprising CBX5VD. In some embodiments, the second effector partner further comprises KRAB(ZIM3). In some embodiments, the fusion protein comprises the effector protein comprising a Cas protein, the first effector partner comprising DNMT3L, and the second effector partner comprising bipartite repressors CBX5VD-KRAB(ZIM3). In some embodiments, the fusion protein comprises the effector protein comprising a Cas protein, the first effector partner comprising an amino acid sequence that is at least 90%, 91%, 92%, 93%, 94%, 95%, 96%, 97%, 98%, or 99% identical to an amino acid sequence of SEQ ID NO: 11, and the second effector partner comprising an amino acid sequence that is at least 90%, 91%, 92%, 93%, 94%, 95%, 96%, 97%, 98%, or 99% identical to an amino acid sequence of any of SEQ ID NO: 3009. In some embodiments, the fusion protein comprises the effector protein comprising a Cas protein, the first effector partner comprising the amino acid sequence of SEQ ID NO: 11, and the second effector partner comprising an amino acid sequence of any of SEQ ID NO: 3009. In some embodiments, the Cas protein comprises an amino acid sequence that is at least 90%, 91%, 92%, 93%, 94%, 95%, 96%, 97%, 98%, or 99% identical to an amino acid sequence of SEQ ID NO: 1. In some embodiments, the Cas protein comprises the amino acid sequence of SEQ ID NO: 1. In some embodiments, the Cas protein comprises an amino acid sequence that is at least 90%, 91%, 92%, 93%, 94%, 95%, 96%, 97%, 98%, or 99% identical to an amino acid sequence of SEQ ID NO: 2. In some embodiments, the Cas protein comprises the amino acid sequence of SEQ ID NO: 2.

In some embodiments, the fusion protein comprises an amino acid sequence that is at least 90%, at least 95%, at least 97%, at least 98%, at least 99%, or 100% identical to a sequence selected from SEQ ID NOs: 16-21, 3004, 3013-3025.

### Synthesis, Isolation and Assaying

Effector proteins of the present disclosure may be synthesized, using any suitable method. In some embodiments, the effector proteins may be produced in vitro or by eukaryotic cells or by prokaryotic cells. In some embodiments, the effector proteins may be further processed by unfolding (e.g. heat denaturation, dithiothreitol reduction, etc.) and may be further refolded, using any suitable method. In some embodiments, the nucleic acid(s) encoding the effector proteins described herein, the recombinant nucleic acid(s) described herein, the vectors described herein may be produced in vitro or in vivo by eukaryotic cells or by prokaryotic cells.

Any suitable method of generating and assaying the effector proteins described herein may be used. Such methods include, but are not limited to, site-directed mutagenesis, random mutagenesis, combinatorial libraries, and other mutagenesis methods described herein (see, e.g., Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, MD (1999); Gillman et al., Directed Evolution Library Creation: Methods and Protocols (Methods in Molecular Biology) Springer, 2nd ed (2014)). One non-limiting example of a method for preparing an effector protein is to express recombinant nucleic acids encoding the effector protein in a suitable microbial organism, such as a bacterial cell, a yeast cell, or other suitable cell, using methods well known in the art. Exemplary methods are also described in the Examples provided herein.

In some embodiments, an effector protein provided herein is an isolated effector protein. In some embodiments, the effector proteins may be isolated and purified for use in compositions, systems, and/or methods described herein. In some embodiments, methods described here may include the step of isolating effector proteins described herein. Any suitable method to provide isolated effector proteins described herein may be used in the present disclosure, for example, recombinant expression systems, precipitation, gel filtration, ion-exchange, reverse-phase and affinity chromatography, and the like. Other well-known methods are described in Deutscher et al., Guide to Protein Purification: Methods in Enzymology, Vol. 182, (Academic Press, (1990)). Alternatively, the isolated polypeptides of the present disclosure can be obtained using well-known recombinant methods (see, e.g., Sambrook et al., Molecular Cloning: A Laboratory Manual, Third Ed., Cold Spring Harbor Laboratory, New York (2001); and Ausubel et al., Current Protocols in Molecular Biology, John Wiley and Sons, Baltimore, MD (1999)). The methods and conditions for biochemical purification of a polypeptide described herein can be chosen by those skilled in the art, and purification monitored, for example, by a functional assay.

In some embodiments, compositions, systems, and methods described herein may further comprise a purification tag that can be attached to an effector protein, or a nucleic acid encoding the purification tag that can be attached to a nucleic acid encoding the effector protein as described herein. In some embodiments, the purification tag may be an amino acid sequence which can attach or bind with high affinity to a separation substrate and assist in isolating the protein of interest from its environment, which may be its biological source, such as a cell lysate. Attachment of the purification tag may be at the N or C terminus of the effector protein. Furthermore, an amino acid sequence recognized by a protease or a nucleic acid encoding for an amino acid sequence recognized by a protease, such as TEV protease or the HRV3C protease may be inserted between the purification tag and the effector protein, such that biochemical cleavage of the sequence with the protease after initial purification liberates the purification tag. Purification and/or isolation may be performed through high performance liquid chromatography (HPLC), exclusion chromatography, gel electrophoresis, affinity chromatography, or other purification technique. Non-limiting examples of purification tags are as described herein.

In some embodiments, effector proteins described herein are isolated from cell lysate. In some embodiments, the compositions described herein may comprise 20% or more by weight, 75% or more by weight, 95% or more by weight, or 99.5% or more by weight of an effector protein, related to the method of preparation of compositions described herein and its purification thereof, wherein percentages may be upon total protein content in relation to contaminants. Thus, in some embodiments, the effector protein is at least 80% pure, at least 85% pure, at least 90% pure, at least 95% pure, at least 98% pure, or at least 99% pure (e.g., free of contaminants, non-engineered proteins or other macromolecules, etc.).

### E. Guide Nucleic Acids

The compositions, systems, and methods of the present disclosure may comprise a guide nucleic acid or a use thereof. Unless otherwise indicated, compositions, systems and methods comprising guide nucleic acids or uses thereof, as described herein and throughout, include DNA molecules, such as expression vectors, that encode a guide nucleic acid. Accordingly, compositions, systems, and methods of the present disclosure comprise a guide nucleic acid or a nucleotide sequence encoding the guide nucleic acid. Guide nucleic acids are also referred to herein as “guide RNA.” A guide nucleic acid, as well as any components thereof (e.g., spacer sequence, repeat sequence, linker nucleotide sequence, handle sequence, intermediary sequence etc.) may comprise one or more deoxyribonucleotides, ribonucleotides, biochemically or chemically modified nucleotides (e.g., one or more engineered modifications as described herein), or any combinations thereof. Such nucleotide sequences described herein may be described as a nucleotide sequence of either DNA or RNA, however, no matter the form the sequence is described, it is readily understood that such nucleotide sequences can be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein also discloses the complementary nucleotide sequence, the reverse nucleotide sequence, and the reverse complement nucleotide sequence, any one of which can be a nucleotide sequence for use in a guide nucleic acid as described herein. In some embodiments, a guide nucleic acid sequence(s) comprises one or more nucleotide alterations at one or more positions in any one of the sequences described herein. Alternative nucleotides can be any one or more of A, C, G, T or U, or a deletion, or an insertion.

A guide nucleic acid may comprise a naturally occurring sequence. A guide nucleic acid may comprise a non-naturally occurring sequence, wherein the sequence of the guide nucleic acid, or any portion thereof, may be different from the sequence of a naturally occurring guide nucleic acid. A guide nucleic acid of the present disclosure comprises one or more of the following: a) a single nucleic acid molecule; b) a DNA base; c) an RNA base; d) a modified base; e) a modified sugar; f) a modified backbone; and the like. Modifications are described herein and throughout the present disclosure (e.g., in the section entitled “Engineered Modifications”). A guide nucleic acid may be chemically synthesized or recombinantly produced by any suitable methods. Guide nucleic acids and portions thereof may be found in or identified from a CRISPR array present in the genome of a host organism or cell.

In general, the guide nucleic acid comprises a nucleotide sequence that is at least 80%, at least 85%, at least 90%, at least 95%, at least 98%, at least 99%, or 100% complementary to the target sequence. In some embodiments, the guide nucleic acid comprises at least 10 contiguous nucleotides that are complementary to the target sequence in the target nucleic acid. In some embodiments, guide nucleic acid comprises a spacer sequence that is at least 80%, at least 85%, at least 90%, at least 95%, at least 98%, at least 99%, or 100% complementary to the target sequence.

In general, a guide nucleic acid comprises a first region that is not complementary to a target nucleic acid (FR) and a second region is complementary to the target nucleic acid (SR), wherein the FR and the SR are heterologous to each other. In some embodiments, FR is located 5′ to SR (FR-SR). In some embodiments, SR is located 5′ to FR (SR-FR). In some embodiments, at least a portion of the FR interacts or binds to an effector protein. The portion of the guide nucleic acid that may be bound by an effector protein may be referred to as the “protein binding sequence.” The protein binding sequence may comprise a repeat sequence, an intermediary sequence, a handle sequence, or a combination thereof, all of which are described herein and throughout. In some embodiments, the SR comprises a spacer sequence, wherein the spacer sequence can interact in a sequence-specific manner with (e.g., has complementarity with, or can hybridize to a target sequence in) a target nucleic acid (e.g., the APOC3, PCSK9, or ANGPTL3 genes). In some embodiments, the spacer sequence comprises a sequence that is at least 65%, at least 70%, at least 75%, at least 80%, at least 85%, at least 90%, at least 95% or 100% complementary to a target sequence of a region from about 500 nucleotides upstream to about 500 nucleotides downstream of the transcriptional start site of a target nucleic acid (e.g., the APOC3, PCSK9, or ANGPTL3 genes).

In some embodiments, the first region, the second region, or both may be about 8 nucleic acids, about 10 nucleic acids, about 12 nucleic acids, about 14 nucleic acids, about 16 nucleic acids, about 18 nucleic acids, about 20 nucleic acids, about 22 nucleic acids, about 24 nucleic acids, about 26 nucleic acids, about 28 nucleic acids, about 30 nucleic acids, about 32 nucleic acids, about 34 nucleic acids, about 36 nucleic acids, about 38 nucleic acids, about 40 nucleic acids, about 42 nucleic acids, about 44 nucleic acids, about 46 nucleic acids, about 48 nucleic acids, or about 50 nucleic acids long.

In some embodiments, the first region, the second region, or both may be from about 8 to about 12, from about 8 to about 16, from about 8 to about 20, from about 8 to about 24, from about 8 to about 28, from about 8 to about 30, from about 8 to about 32, from about 8 to about 34, from about 8 to about 36, from about 8 to about 38, from about 8 to about 40, from about 8 to about 42, from about 8 to about 44, from about 8 to about 48, or from about 8 to about 50 nucleic acids long.

In some embodiments, the first region, the second region, or both may comprise a GC content of about 1%, about 5%, about 10%, about 15%, about 20%, about 25%, about 30%, about 40%, about 50%, about 60%, about 70%, about 80%, about 90%, about 95%, or about 99%. In some embodiments, the first region, the second region, or both may comprise a GC content of from about 1% to about 95%, from about 5% to about 90%, from about 10% to about 80%, from about 15% to about 70%, from about 20% to about 60%, from about 25% to about 50%, or from about 30% to about 40%.

In some embodiments, the first region, the second region, or both may have a melting temperature of about 38° C., about 40° C., about 42° C., about 44° C., about 46° C., about 48° C., about 50° C., about 52° C., about 54° C., about 56° C., about 58° C., about 60° C., about 62° C., about 64° C., about 66° C., about 68° C., about 70° C., about 72° C., about 74° C., about 76° C., about 78° C., about 80° C., about 82° C., about 84° C., about 86° C., about 88° C., about 90° C., or about 92° C. In some embodiments, the first region, the second region, or both may have a melting temperature of from about 35° C. to about 40° C., from about 35° C. to about 45° C., from about 35° C. to about 50° C., from about 35° C. to about 55° C., from about 35° C. to about 60° C., from about 35° C. to about 65° C., from about 35° C. to about 70° C., from about 35° C. to about 75° C., from about 35° C. to about 80° C., or from about 35° C. to about 85° C.

In some embodiments, the compositions, systems, and methods of the present disclosure further comprise an additional nucleic acid, wherein a portion of the additional nucleic acid at least partially hybridizes to the first region of the guide nucleic acid. In some embodiments, the additional nucleic acid is at least partially hybridized to the 5′ end of the second region of the guide nucleic acid. In some embodiments, an unhybridized portion of the additional nucleic acid, at least partially, interacts with an effector protein or polypeptide. In some embodiments, the compositions, systems, and methods of the present disclosure comprise a dual nucleic acid system comprising the guide nucleic acid and the additional nucleic acid as described herein.

The guide nucleic acid may also form complexes as described through herein. For example, a guide nucleic acid may hybridize to another nucleic acid, such as target nucleic acid, or a portion thereof. In another example, a guide nucleic acid may complex with an effector protein. In such embodiments, a guide nucleic acid-effector protein complex may be described herein as an RNP. In some embodiments, when in a complex, at least a portion of the complex may bind, recognize, and/or hybridize to a target nucleic acid. For example, when a guide nucleic acid and an effector protein are complexed to form an RNP, at least a portion of the guide nucleic acid hybridizes to a target sequence in a target nucleic acid. Those skilled in the art in reading the below specific examples of guide nucleic acids as used in RNPs described herein, will understand that in some embodiments, a RNP may hybridize to one or more target sequences in a target nucleic acid, thereby allowing the RNP to modify and/or recognize a target nucleic acid or sequence contained therein (e.g., PAM) or to modify and/or recognize non-target sequences depending on the guide nucleic acid, and in some embodiments, the effector protein, used.

In some embodiments, a guide nucleic acid may comprise or form intramolecular secondary structure (e.g., hairpins, stem-loops, etc.). In some embodiments, a guide nucleic acid comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the guide nucleic acid comprises a pseudoknot (e.g., a secondary structure comprising a stem, at least partially, hybridized to a second stem or half-stem secondary structure). An effector protein may recognize a guide nucleic acid comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the guide nucleic acid comprises at least 2, at least 3, at least 4, or at least 5 stem regions.

In some embodiments, a guide nucleic acid comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In general, a guide nucleic acid comprises at least: 10, 15, 20, 25, 30, 35, 40, 45, 50, 55, 60 linked nucleotides. In some embodiments, the guide nucleic acid has about 10 to about 60, about 20 to about 50, or about 30 to about 40 linked nucleotides.

In some embodiments, a guide nucleic acid comprises at least 10, 11, 12, 13, 14, 15, 16, 17, 18, 19, 20, 21, 22, 23, 24, 25, 26, 27, 28, 29, or 30 contiguous nucleotides that are complementary to a eukaryotic sequence. Such a eukaryotic sequence is a nucleotide sequence that is present in a host eukaryotic cell. Such a nucleotide sequence is distinguished from nucleotide sequences present in other host cells, such as prokaryotic cells, or viruses. Said sequences present in a eukaryotic cell can be located in a gene, an exon, an intron, a non-coding (e.g., promoter or enhancer) region, a selectable marker, tag, signal, and the like. In some embodiments, a target sequence is a eukaryotic sequence.

In some embodiments, a length of a guide nucleic acid is about 30 to about 120 linked nucleotides. In some embodiments, the length of a guide nucleic acid is about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a guide nucleic acid is about 40, about 45, about 50, about 55, about 60, about 65, about 70 or about 75 linked nucleotides. In some embodiments, the length of a guide nucleic acid is greater than about 15, about 20, about 25, about 30, about 35, about 40, about 45, about 50, about 55, about 60, about 65, about 70 or about 75 linked nucleotides. In some embodiments, the length of a guide nucleic acid is not greater than about 40, about 45, about 50, about 55, about 60, about 65, about 70, about 75, about 80, about 85, about 90, about 95, about 100, about 105, about 110, about 115, about 120, or about 125 linked nucleotides.

In some embodiments, guide nucleic acids comprise additional elements that contribute additional functionality (e.g., stability, heat resistance, etc.) to the guide nucleic acid. Such elements may be one or more nucleotide alterations, nucleotide sequences, intermolecular secondary structures, or intramolecular secondary structures (e.g., one or more hair pin regions, one or more bulges, etc.).

In some embodiments, guide nucleic acids comprise one or more linkers connecting different nucleotide sequences as described herein. A linker may comprise 1, 2, 3, 4, 5, 6, 7, 8, 9, 10, or more nucleotides. A linker may be any suitable linker, examples of which are described herein.

In some embodiments, guide nucleic acids comprise one or more nucleotide sequences as described herein (e.g., TABLES 7-9). Such nucleotide sequences described herein (e.g., TABLES 7-9) may be described as a nucleotide sequence of either DNA or RNA, however, no matter the form of the sequence described, it is readily understood that such nucleotide sequences may be revised to be RNA or DNA, as needed, for describing a sequence within a guide nucleic acid itself or the sequence that encodes a guide nucleic acid, such as a nucleotide sequence described herein for a vector. Similarly, disclosure of the nucleotide sequences described herein (e.g., TABLES 7-9) also discloses the complementary nucleotide sequence, the reverse nucleotide sequence, and the reverse complement nucleotide sequence, any one of which may be a nucleotide sequence for use in a guide nucleic acid as described herein. In some embodiments, guide nucleic acid sequence(s) comprises one or more nucleotide alterations at one or more positions in any one of the sequences described herein. Alternative nucleotides may be any one or more of A, C, G, T or U, or a deletion, or an insertion.

In some embodiments, the guide nucleic acid comprises a nucleotide sequence that is capable of hybridizing to a target sequence in a target nucleic acid, wherein the target nucleic acid is any one of: a naturally occurring eukaryotic sequence, a naturally occurring prokaryotic sequence, a naturally occurring viral sequence, a naturally occurring bacterial sequence, a naturally occurring fungal sequence, an engineered eukaryotic sequence, an engineered prokaryotic sequence, an engineered viral sequence, an engineered bacterial sequence, an engineered fungal sequence, a fragment of a naturally occurring sequence, a fragment of an engineered sequence, and combinations thereof. In some embodiments, the target sequence is only found in a eukaryote in nature. In some embodiments, the target sequence is only found in a human in nature.

In some embodiments, the guide nucleic acid is isolated from any one of: a naturally occurring cell, a eukaryotic cell, a prokaryotic cell, a plant cell, a fungal cell, an animal cell, cell of an invertebrate, a fly cell, a cell of a vertebrate, a mammalian cell, a primate cell, a non-human primate cell, a human cell, a living cell, a non-living cell, a modified cell, a derived cell, and a non-naturally occurring cell.

### Repeat Sequence

Guide nucleic acids described herein may comprise one or more repeat sequences. In some embodiments, a repeat sequence comprises a nucleotide sequence that is not complementary to a target sequence of a target nucleic acid. In some embodiments, a repeat sequence comprises a nucleotide sequence that may interact with an effector protein. In some embodiments, a repeat sequence is connected to another sequence of a guide nucleic acid, such as an intermediary sequence, that is capable of non-covalently interacting with an effector protein. In some embodiments, a repeat sequence includes a nucleotide sequence that is capable of forming a guide nucleic acid-effector protein complex (e.g., a RNP complex).

In some embodiments, the repeat sequence is between 10 and 50, 12 and 48, 14 and 46, 16 and 44, and 18 and 42 nucleotides in length.

In some embodiments, a repeat sequence is adjacent to a spacer sequence. In some embodiments, a repeat sequence is followed by a spacer sequence in the 5′ to 3′ direction. In some embodiments, a repeat sequence is preceded by a spacer sequence in the 5′ to 3′ direction. In some embodiments, a repeat sequence is adjacent to an intermediary sequence. In some embodiments, a repeat sequence is 3′ to an intermediary sequence. In some embodiments, an intermediary sequence is followed by a repeat sequence, which is followed by a spacer sequence in the 5′ to 3′ direction. In some embodiments, a repeat sequence is linked to a spacer sequence and/or an intermediary sequence. In some embodiments, a guide nucleic acid comprises a repeat sequence linked to a spacer sequence and/or to an intermediary sequence, which may be a direct link or by any suitable linker, examples of which are described herein.

In some embodiments, guide nucleic acids comprise more than one repeat sequence (e.g., two or more, three or more, or four or more repeat sequences). In some embodiments, a guide nucleic acid comprises more than one repeat sequence separated by another sequence of the guide nucleic acid. For example, in some embodiments, a guide nucleic acid comprises two repeat sequences, wherein the first repeat sequence is followed by a spacer sequence, and the spacer sequence is followed by a second repeat sequence in the 5′ to 3′ direction. In some embodiments, the more than one repeat sequences are identical. In some embodiments, the more than one repeat sequences are not identical.

In some embodiments, the repeat sequence comprises two sequences that are complementary to each other and hybridize to form a double stranded RNA duplex (dsRNA duplex). In some embodiments, the two sequences are not directly linked and hybridize to form a stem loop structure. In some embodiments, the dsRNA duplex comprises 5, 10, 15, 20 or 25 base pairs (bp). In some embodiments, not all nucleotides of the dsRNA duplex are paired, and therefore the duplex forming sequence may include a bulge. In some embodiments, the repeat sequence comprises a hairpin or stem-loop structure, optionally at the 5′ portion of the repeat sequence. In some embodiments, a strand of the stem portion comprises a sequence and the other strand of the stem portion comprises a sequence that is, at least partially, complementary. In some embodiments, such sequences may have 65% to 100% complementarity (e.g., 65%, 70%, 75%, 80%, 85%, 90%, 95%, or 100% complementarity). In some embodiments, a guide nucleic acid comprises nucleotide sequence that when involved in hybridization events may hybridize over one or more segments such that intervening or adjacent segments are not involved in the hybridization event (e.g., a bulge, a loop structure or hairpin structure, etc.).

In some embodiments, a repeat sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of any one of the repeat sequences in TABLE 7. In some embodiments, the repeat sequence is at least 85% identical to any one of sequences set forth in TABLE 7. In some embodiments, a repeat sequence comprises at least 5, at least 6, at least 7, at least 8, at least 9, at least 10, at least 11, at least 12, at least 13, at least 14, at least 15, at least 16, at least 17, at least 18, at least 19, at least 20, or at least 21 contiguous nucleotides of any one of the sequences recited in TABLE 7. In some embodiments, a repeat sequence comprises one or more nucleotide alterations at one or more positions in the sequence recited in TABLE 7. Alternative nucleotides can be any one or more of A, C, G, T or U, or a deletion, or an insertion.

In some embodiments, the effector protein is at least 90%, at least 95%, at least 98%, at least 99% or 100% identical to SEQ ID NO: 1 or 2, and the repeat sequence is at least 90%, at least 95%, at least 98%, at least 99% or 100% identical to any one of the nucleotide sequence of SEQ ID NOs: 5-9. In some embodiments, the effector protein is at least 90%, at least 95%, at least 98%, at least 99% or 100% identical to SEQ ID NO: 1, and the protein binding sequence is at least 90%, at least 95%, at least 98%, at least 99% or 100% identical to any one of the nucleotide sequence of SEQ ID NOs: 5-9.

### Spacer Sequence

Guide nucleic acids described herein may comprise one or more spacer sequences. In some embodiments, a spacer sequence is capable of hybridizing to a target sequence of a target nucleic acid. In some embodiments, a spacer sequence comprises a nucleotide sequence that is, at least partially, hybridizable to an equal length of a sequence (e.g., a target sequence) of a target nucleic acid. In some embodiments, a spacer sequence is at least 90% complementary to a target sequence in a eukaryotic genome, wherein the eukaryotic genome is a human genome. Exemplary hybridization conditions are described herein. In some embodiments, the spacer sequence may function to direct an RNP complex comprising the guide nucleic acid to the target nucleic acid for detection and/or modification. The spacer sequence may function to direct a RNP to the target nucleic acid for detection and/or modification. A spacer sequence may be complementary to a target sequence that is adjacent to a PAM that is recognizable by an effector protein described herein.

In some embodiments, a spacer sequence comprises at least 5 to about 50 contiguous nucleotides that are complementary to a target sequence in a target nucleic acid. In some embodiments, a spacer sequence comprises at least 5 to about 50 linked nucleotides. In some embodiments, a spacer sequence comprises at least 5 to about 50, at least 5 to about 25, at least about 10 to at least about 25, or at least about 15 to about 25 linked nucleotides. In some embodiments, the spacer sequence comprises 15-28 linked nucleotides. In some embodiments, a spacer sequence comprises 15-26, 15-24, 15-22, 15-20, 15-18, 16-28, 16-26, 16-24, 16-22, 16-20, 16-18, 17-26, 17-24, 17-22, 17-20, 17-18, 18-26, 18-24, or 18-22 linked nucleotides. In some embodiments, the spacer sequence comprises 10, 11, 12, 13, 14, 15, 16, 17, 18, 19, 20, 21, 22, 23, 24, 25, 26, 27, 28, 29, 30, or more nucleotides.

In some embodiments, a spacer sequence is adjacent to a repeat sequence. In some embodiments, a spacer sequence follows a repeat sequence in a 5′ to 3′ direction. In some embodiments, a spacer sequence precedes a repeat sequence in a 5′ to 3′ direction. In some embodiments, the spacer sequence(s) and the repeat sequence(s) of the guide nucleic acid are present within the same molecule. In some embodiments, the spacer(s) and repeat sequence(s) are linked directly to one another. In some embodiments, a linker is present between the spacer(s) and repeat sequences. Linkers may be any suitable linker. In some embodiments, the spacer sequence(s) and the repeat sequence(s) of the guide nucleic acid are present in separate molecules, which are joined to one another by base pairing interactions.

In some embodiments, a spacer sequence comprises a nucleotide sequence that is at least 65%, at least 70%, at least 75%, at least 80%, at least 85%, at least 90%, at least 95% or 100% complementary to a target sequence of a target nucleic acid. A spacer sequence is capable of hybridizing to an equal length portion of a target nucleic acid (e.g., a target sequence). In some embodiments, a target nucleic acid, such as DNA or RNA, may be a cancer gene or gene associated with a genetic disorder, or an amplicon thereof, as described herein.

In some embodiments, a spacer sequence comprises a sequence that is at least 65%, at least 70%, at least 75%, at least 80%, at least 85%, at least 90%, at least 95% or 100% complementary to a target sequence of a region from about 500 nucleotides upstream to about 500 nucleotides downstream of the transcriptional start site of a APOC3 gene. In some embodiments, a spacer sequence comprises a sequence that is at least 65%, at least 70%, at least 75%, at least 80%, at least 85%, at least 90%, at least 95% or 100% complementary to a target sequence of a region from about 500 nucleotides upstream to about 500 nucleotides downstream of the transcriptional start site of a PCKS9 gene. In some embodiments, a spacer sequence comprises a sequence that is at least 65%, at least 70%, at least 75%, at least 80%, at least 85%, at least 90%, at least 95% or 100% complementary to a target sequence of a region from about 500 nucleotides upstream to about 500 nucleotides downstream of the transcriptional start site of a ANGPTL3 gene.

TABLE 9 provides illustrative spacer sequences targeting human or mouse APOC3 for use with CasPhi.12 (SEQ ID NO: 1). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to any one of the nucleotide sequences of SEQ ID NOs: 37-109, and 360-456. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 37-109, and 360-456.

TABLE 9 provides illustrative spacer sequences targeting human or mouse PCSK9 for use with CasPhi.12 (SEQ ID NO: 1). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to any one of the nucleotide sequences of SEQ ID NOs: 110-177 and 457-528. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 110-177, or 457-528.

TABLE 9 provides illustrative spacer sequences targeting human ANGPTL3 for use with CasPhi.12 (SEQ ID NO: 1). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to any one of the nucleotide sequences of SEQ ID NOs: 178-359. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 178-359.

TABLE 10 provides illustrative spacer sequences targeting human or mouse APOC3 for use with CasM.265466 (SEQ ID NO: 35). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to any one of the nucleotide sequences of SEQ ID NOs: 1021-1202, and 1583-1800. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 1021-1202, and 1583-1800.

TABLE 10 provides illustrative spacer sequences targeting human or mouse PCSK9 for use with CasM.265466 (SEQ ID NO: 35). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to any one of the nucleotide sequences of SEQ ID NOs: 1203-1361 and 1801-1975. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 1203-1361 and 1801-1975.

TABLE 10 provides illustrative spacer sequences targeting human ANGPTL3 for use with CasM.265466 (SEQ ID NO: 35). In some embodiments, the spacer sequence comprises at least 70%, at least 80%, at least 90%, at least 91%, at least 92%, at least 93%, at least 94%, at least 95%, at least 96%, at least 97%, or at least 99%, or 100% sequence identity to any one of the nucleotide sequences of SEQ ID NOs: 1362-1582. In some embodiments, spacer sequences comprise at least 10, at least 11, at least 12, at least 13, at least 14, at least 15, at least 16, at least 17, at least 18, at least 19, or at least 20 contiguous nucleotides of a sequence selected from any one of SEQ ID NOs: 1362-1582.

In some embodiments, a target nucleic acid is a gene selected from TABLE 11. In some embodiments, a spacer sequence comprises a sequence that is at least 65%, at least 70%, at least 75%, at least 80%, at least 85%, at least 90%, at least 95% or 100% complementary to a target sequence of a target nucleic acid selected from TABLE 11. In some embodiments, a target nucleic acid is a nucleic acid associated with a disease or syndrome set forth in TABLE 12. In some embodiments, a spacer sequence comprises a sequence that is at least 65%, at least 70%, at least 75%, at least 80%, at least 85%, at least 90%, at least 95% or 100% complementary to a target sequence of a target nucleic acid associated with a disease or syndrome set forth in TABLE 12. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are capable of hybridizing to the target sequence. In some embodiments, the spacer sequence comprises at least 10, 11, 12, 13, 14, 15, 16, 17, 18, 19, 20, 21, 22, 23, 24, 25, 26, 27, 28, 29, or 30 contiguous nucleotides that are complementary to the target sequence.

It is understood that the spacer sequence of a spacer sequence need not be 100% complementary to that of a target sequence of a target nucleic acid to hybridize or hybridize specifically to the target sequence. For example, the spacer sequence may comprise at least one alteration, such as a substituted or modified nucleotide, that is not complementary to the corresponding nucleotide of the target sequence. Spacer sequences are further described throughout herein.

### Linker for Nucleic Acids

In some embodiments, a guide nucleic acid for use with compositions, systems, and methods described herein comprises one or more linkers, or a nucleic acid encoding one or more linkers. In some embodiments, the guide nucleic acid comprises at least one, at least two, at least three, at least four, at least five, at least six, at least seven, at least eight, at least nine, or at least ten linkers. In some embodiments, the guide nucleic acid comprises one, two, three, four, five, six, seven, eight, nine, or ten linkers. In some embodiments, the guide nucleic acid comprises more than one linker. In some embodiments, at least two of the more than one linker are the same. In some embodiments, at least two of the more than one linker are not same.

In some embodiments, a linker comprises one to ten, one to seven, one to five, one to three, two to ten, two to eight, two to six, two to four, three to ten, three to seven, three to five, four to ten, four to eight, four to six, five to ten, five to seven, six to ten, six to eight, seven to ten, or eight to ten linked nucleotides. In some embodiments, the linker comprises one, two, three, four, five, six, seven, eight, nine, or ten linked nucleotides. In some embodiments, a linker comprises a nucleotide sequence of 5′-GAAA-3′.

In some embodiments, a guide nucleic acid comprises one or more linkers connecting one or more repeat sequences. In some embodiments, the guide nucleic acid comprises one or more linkers connecting one or more repeat sequences and one or more spacer sequences. In some embodiments, the guide nucleic acid comprises at least two repeat sequences connected by a linker.

### Intermediary Sequence

Guide nucleic acids described herein may comprise one or more intermediary sequences. In general, an intermediary sequence used in the present disclosure is not transactivated or transactivating. An intermediary sequence may also be referred to as an intermediary RNA, although it may comprise deoxyribonucleotides instead of or in addition to ribonucleotides, and/or modified bases. In general, the intermediary sequence non-covalently binds to an effector protein. In some embodiments, the intermediary sequence forms a secondary structure, for example in a cell, and an effector protein binds the secondary structure.

In some embodiments, a length of the intermediary sequence is at least 30, 50, 70, 90, 110, 130, 150, 170, 190, or 210 linked nucleotides. In some embodiments, a length of the intermediary sequence is not greater than 30, 50, 70, 90, 110, 130, 150, 170, 190, or 210 linked nucleotides. In some embodiments, the length of the intermediary sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

An intermediary sequence may also comprise or form a secondary structure (e.g., one or more hairpin loops) that facilitates the binding of an effector protein to a guide nucleic acid and/or modification activity of an effector protein on a target nucleic acid (e.g., a hairpin region). An intermediary sequence may comprise from 5′ to 3′, a 5′ region, a hairpin region, and a 3′ region. In some embodiments, the 5′ region may hybridize to the 3′ region. In some embodiments, the 5′ region of the intermediary sequence does not hybridize to the 3′ region.

In some embodiments, the hairpin region may comprise a first sequence, a second sequence that is reverse complementary to the first sequence, and a stem-loop linking the first sequence and the second sequence. In some embodiments, an intermediary sequence comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, an intermediary sequence comprises a pseudoknot (e.g., a secondary structure comprising a stem at least partially hybridized to a second stem or half-stem secondary structure). An effector protein may interact with an intermediary sequence comprising a single stem region or multiple stem regions. In some embodiments, the nucleotide sequences of the multiple stem regions are identical to one another. In some embodiments, the nucleotide sequences of at least one of the multiple stem regions is not identical to those of the others. In some embodiments, an intermediary sequence comprises 1, 2, 3, 4, 5 or more stem regions.

### Handle Sequence

Guide nucleic acids described herein may comprise one or more handle sequences. In some embodiments, the handle sequence comprises an intermediary sequence. In such instances, at least a portion of an intermediary sequence non-covalently bonds with an effector protein. In some embodiments, the intermediary sequence is at the 3′-end of the handle sequence. In some embodiments, the intermediary sequence is at the 5′-end of the handle sequence. Additionally, or alternatively, in some embodiments, the handle sequence further comprises one or more of linkers and repeat sequences. In such instances, at least a portion of an intermediary sequence, or both of at least a portion of the intermediary sequence and at least a portion of repeat sequence, non-covalently interacts with an effector protein. In some embodiments, an intermediary sequence and repeat sequence are directly linked (e.g., covalently linked, such as through a phosphodiester bond). In some embodiments, the intermediary sequence and repeat sequence are linked by a suitable linker, examples of which are provided herein. In some embodiments, the linker comprises a sequence of 5′-GAAA-3′. In some embodiments, the intermediary sequence is 5′ to the repeat sequence. In some embodiments, the intermediary sequence is 5′ to the linker. In some embodiments, the intermediary sequence is 3′ to the repeat sequence. In some embodiments, the intermediary sequence is 3′ to the linker. In some embodiments, the repeat sequence is 3′ to the linker. In some embodiments, the repeat sequence is 5′ to the linker. In general, a single guide nucleic acid, also referred to as a single guide RNA (sgRNA), comprises a handle sequence comprising an intermediary sequence, and optionally one or more of a repeat sequence and a linker.

A handle sequence may comprise or form a secondary structure (e.g., one or more hairpin loops) that facilitates the binding of an effector protein to a guide nucleic acid and/or modification activity of an effector protein on a target nucleic acid (e.g., a hairpin region). In some embodiments, handle sequences comprise a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the handle sequence comprises a pseudoknot (e.g., a secondary structure comprising a stem at least partially hybridized to a second stem or half-stem secondary structure). An effector protein may recognize a handle sequence comprising multiple stem regions. In some embodiments, the nucleotide sequences of the multiple stem regions are identical to one another. In some embodiments, the nucleotide sequences of at least one of the multiple stem regions is not identical to those of the others. In some embodiments, the handle sequence comprises at least 2, at least 3, at least 4, or at least 5 stem regions.

In some embodiments, a length of the handle sequence is at least 30, 50, 70, 90, 100, 110, 130, 150, 170, 190, or 210 linked nucleotides. In some embodiments, a length of the handle sequence is not greater than 30, 50, 70, 90, 100, 110, 130, 150, 170, 190, or 210 linked nucleotides. In some embodiments, the length of the handle sequence is about 30 to about 210, about 60 to about 210, about 90 to about 210, about 120 to about 210, about 150 to about 210, about 180 to about 210, about 30 to about 180, about 60 to about 180, about 90 to about 180, about 120 to about 180, or about 150 to about 180 linked nucleotides.

In some embodiments, a handle sequence comprises a nucleotide sequence that is at least 65%, at least 70%, at least 75%, at least 80%, at least 85%, at least 90%, at least 95%, at least 97%, or at least 98%, at least 99%, or 100% identical to an equal length portion of any one of the repeat sequences in TABLE 8. In some embodiments, the handle sequence is at least 85% identical to any one of sequences set forth in TABLE 8. In some embodiments, a handle sequence comprises one or more nucleotide alterations at one or more positions in the sequence recited in TABLE 8. Alternative nucleotides can be any one or more of A, C, G, T or U, or a deletion, or an insertion.

In some embodiments, the effector protein is at least 90%, at least 95%, at least 98%, at least 99% or 100% identical to SEQ ID NO: 35, 2950, 3010, 3011, or 3012, and the handle sequence is at least 90%, at least 95%, at least 98%, at least 99% or 100% identical to the nucleotide sequence of SEQ ID NO: 36.

### Exemplary Guide Nucleic Acid

In some embodiments, the guide nucleic acids disclosed herein comprise a spacer sequence that is at least 70%, at least 75%, at least 80%, at least 85%, at least 90%, at least 95%, or 100% identical to any one of the sequences as set forth in TABLE 9 and a repeat sequence that is at least 70%, at least 75%, at least 80%, at least 85%, at least 90%, at least 95%, or 100% identical to any one of the sequences of TABLE 7.

In some embodiments, the guide nucleic acids disclosed herein comprise a spacer sequence that is at least 70%, at least 75%, at least 80%, at least 85%, at least 90%, at least 95%, or 100% identical to any one of the sequences as set forth in TABLE 10 and a handle sequence that is at least 70%, at least 75%, at least 80%, at least 85%, at least 90%, at least 95%, or 100% identical to any one of the sequences of TABLE 8.

Exemplary guide nucleic acid sequences useful for systems, compositions and methods described herein are provided in TABLES 9 and 10. In some embodiments, the guide nucleic acid comprises a sequence that is at least 70%, at least 75%, at least 80%, at least 85%, at least 90%, at least 95%, or 100% identical to any one of the sequences of TABLES 9 and 10. In some embodiments, the guide nucleic acid consists of a sequence that is at least 70%, at least 75%, at least 80%, at least 85%, at least 90%, at least 95%, or 100% identical to any one of the sequences of TABLES 9 and 10. In some embodiments, the guide nucleic acids provided in TABLES 9 and 10 comprise an additional “G” at the 5′ end of the sequence.

In some embodiments, a guide nucleic acid to be used in combination with CasPhi.12 for targeting the human APOC3 gene comprises a sequence that is at least 70%, at least 75%, at least 80%, at least 85%, at least 90%, at least 95%, or 100% identical to any one of the sequences of SEQ ID NOs: 529-601. In some embodiments, a guide nucleic acid to be used in combination with CasPhi.12 for targeting the mouse APOC3 gene comprises a sequence that is at least 70%, at least 75%, at least 80%, at least 85%, at least 90%, at least 95%, or 100% identical to any one of the sequences of SEQ ID NOs: 852-948.

In some embodiments, a guide nucleic acid to be used in combination with CasPhi.12 for targeting the human PCSK9 gene comprises a sequence that is at least 70%, at least 75%, at least 80%, at least 85%, at least 90%, at least 95%, or 100% identical to any one of the sequences of SEQ ID NOs: 602-669. In some embodiments, a guide nucleic acid to be used in combination with CasPhi.12 for targeting the mouse PCSK9 gene comprises a sequence that is at least 70%, at least 75%, at least 80%, at least 85%, at least 90%, at least 95%, or 100% identical to any one of the sequences of SEQ ID NOs: 949-1020.

In some embodiments, a guide nucleic acid to be used in combination with CasPhi.12 for targeting the human ANGPTL3 gene comprises a sequence that is at least 70%, at least 75%, at least 80%, at least 85%, at least 90%, at least 95%, or 100% identical to any one of the sequences of SEQ ID NOs: 670-851.

In some embodiments, a guide nucleic acid to be used in combination with CasM.265466 for targeting the human APOC3 gene comprises a sequence that is at least 70%, at least 75%, at least 80%, at least 85%, at least 90%, at least 95%, or 100% identical to any one of the sequences of SEQ ID NOs: 1976-2157. In some embodiments, a guide nucleic acid to be used in combination with CasM.265466 for targeting the mouse APOC3 gene comprises a sequence that is at least 70%, at least 75%, at least 80%, at least 85%, at least 90%, at least 95%, or 100% identical to any one of the sequences of SEQ ID NOs: 2538-2755.

In some embodiments, a guide nucleic acid to be used in combination with CasM.265466 for targeting the human PCSK9 gene comprises a sequence that is at least 70%, at least 75%, at least 80%, at least 85%, at least 90%, at least 95%, or 100% identical to any one of the sequences of SEQ ID NOs: 2158-2316. In some embodiments, a guide nucleic acid to be used in combination with CasM.265466 for targeting the mouse PCSK9 gene comprises a sequence that is at least 70%, at least 75%, at least 80%, at least 85%, at least 90%, at least 95%, or 100% identical to any one of the sequences of SEQ ID NOs: 2756-2930.

In some embodiments, a guide nucleic acid to be used in combination with CasM.265466 for targeting the human ANGPTL3 gene comprises a sequence that is at least 70%, at least 75%, at least 80%, at least 85%, at least 90%, at least 95%, or 100% identical to any one of the sequences of SEQ ID NOs: 2317-2537.

### A Single Nucleic Acid System

In some embodiments, compositions, systems and methods described herein comprise a single nucleic acid system comprising a guide nucleic acid or a nucleotide sequence encoding the guide nucleic acid, and one or more effector proteins or a nucleotide sequence encoding the one or more effector proteins. In some embodiments, a first region (FR) of the guide nucleic acid non-covalently interacts with the one or more polypeptides described herein. In some embodiments, a second region (SR) of the guide nucleic acid hybridizes with a target sequence of the target nucleic acid. In the single nucleic acid system having a complex of the guide nucleic acid and the effector protein, the effector protein is not transactivated by the guide nucleic acid. In other words, activity of effector protein does not require binding to a second non-target nucleic acid molecule. An exemplary guide nucleic acid for a single nucleic acid system is a crRNA or a sgRNA.

crRNA

In some embodiments, a guide nucleic acid comprises a crRNA. In some embodiments, the guide nucleic acid is the crRNA. In general, a crRNA comprises a first region (FR) and a second region (SR), wherein the FR of the crRNA comprises a repeat sequence, and the SR of the crRNA comprises a spacer sequence. In some embodiments, the repeat sequence and the spacer sequences are directly connected to each other (e.g., covalent bond (phosphodiester bond)). In some embodiments, the repeat sequence and the spacer sequence are connected by a linker.

In some embodiments, a crRNA is useful as a single nucleic acid system for compositions, methods, and systems described herein or as part of a single nucleic acid system for compositions, methods, and systems described herein. In some embodiments, a crRNA is useful as part of a single nucleic acid system for compositions, methods, and systems described herein. In such embodiments, a single nucleic acid system comprises a guide nucleic acid comprising a crRNA wherein, a repeat sequence of a crRNA is capable of connecting a crRNA to an effector protein. In some embodiments, a single nucleic acid system comprises a guide nucleic acid comprising a crRNA linked to another nucleotide sequence that is capable of being non-covalently bond by an effector protein. In such embodiments, a repeat sequence of a crRNA can be linked to an intermediary sequence. In some embodiments, a single nucleic acid system comprises a guide nucleic acid comprising a crRNA and an intermediary sequence.

A crRNA may include deoxyribonucleosides, ribonucleosides, chemically modified nucleosides, or any combination thereof. In some embodiments, a crRNA comprises about: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 linked nucleotides. In some embodiments, a crRNA comprises at least: 10, 15, 20, 25, 30, 35, 40, 45, 50, 55, 60 linked nucleotides. In some embodiments, the length of the crRNA is about 20 to about 120 linked nucleotides. In some embodiments, the length of a crRNA is about 20 to about 100, about 30 to about 100, about 40 to about 100, about 40 to about 90, about 40 to about 80, about 40 to about 70, about 40 to about 60, about 40 to about 50, about 50 to about 90, about 50 to about 80, about 50 to about 70, or about 50 to about 60 linked nucleotides. In some embodiments, the length of a crRNA is about 20, about 25, about 30, about 35, about 40, about 45, about 50, about 55, about 60, about 65, about 70 or about 75 linked nucleotides.

sgRNA

In some embodiments, a guide nucleic acid comprises a sgRNA. In some embodiments, a guide nucleic acid is a sgRNA. In some embodiments, a sgRNA comprises a first region (FR) and a second region (SR), wherein the FR comprises a handle sequence and the SR comprises a spacer sequence. In some embodiments, the handle sequence and the spacer sequences are directly connected to each other (e.g., covalent bond (phosphodiester bond)). In some embodiments, the handle sequence and the spacer sequence are connected by a linker.

In some embodiments, a sgRNA comprises one or more of a handle sequence, an intermediary sequence, a crRNA, a repeat sequence, a spacer sequence, a linker, or combinations thereof. For example, a sgRNA comprises a handle sequence and a spacer sequence; an intermediary sequence and an crRNA; an intermediary sequence, a repeat sequence and a spacer sequence; and the like.

In some embodiments, a sgRNA comprises an intermediary sequence and an crRNA. In some embodiments, an intermediary sequence is 5′ to a crRNA in an sgRNA. In some embodiments, a sgRNA comprises a linked intermediary sequence and crRNA. In some embodiments, an intermediary sequence and a crRNA are linked in an sgRNA directly (e.g., covalently linked, such as through a phosphodiester bond) In some embodiments, an intermediary sequence and a crRNA are linked in an sgRNA by any suitable linker, examples of which are provided herein.

In some embodiments, a sgRNA comprises a handle sequence and a spacer sequence. In some embodiments, a handle sequence is 5′ to a spacer sequence in an sgRNA. In some embodiments, a sgRNA comprises a linked handle sequence and spacer sequence. In some embodiments, a handle sequence and a spacer sequence are linked in an sgRNA directly (e.g., covalently linked, such as through a phosphodiester bond) In some embodiments, a handle sequence and a spacer sequence are linked in an sgRNA by any suitable linker, examples of which are provided herein.

In some embodiments, a sgRNA comprises an intermediary sequence, a repeat sequence, and a spacer sequence. In some embodiments, an intermediary sequence is 5′ to a repeat sequence in an sgRNA. In some embodiments, a sgRNA comprises a linked intermediary sequence and repeat sequence. In some embodiments, an intermediary sequence and a repeat sequence are linked in an sgRNA directly (e.g., covalently linked, such as through a phosphodiester bond) In some embodiments, an intermediary sequence and a repeat sequence are linked in an sgRNA by any suitable linker, examples of which are provided herein. In some embodiments, a repeat sequence is 5′ to a spacer sequence in an sgRNA. In some embodiments, a sgRNA comprises a linked repeat sequence and spacer sequence. In some embodiments, a repeat sequence and a spacer sequence are linked in an sgRNA directly (e.g. covalently linked, such as through a phosphodiester bond) In some embodiments, a repeat sequence and a spacer sequence are linked in an sgRNA by any suitable linker, examples of which are provided herein.

### Dual Nucleic Acid Systems

In some embodiments, compositions, systems and methods described herein comprise a dual nucleic acid system comprising a crRNA or a nucleotide sequence encoding the crRNA, a tracrRNA or a nucleotide sequence encoding the tracrRNA, and one or more effector protein or a nucleotide sequence encoding the one or more effector protein, wherein the crRNA and the tracrRNA are separate, unlinked molecules, wherein a repeat hybridization region of the tracrRNA is capable of hybridizing with an equal length portion of the crRNA to form a tracrRNA-crRNA duplex, wherein the equal length portion of the crRNA does not include a spacer sequence of the crRNA, and wherein the spacer sequence is capable of hybridizing to a target sequence of the target nucleic acid. In the dual nucleic acid system having a complex of the guide nucleic acid, tracrRNA, and the effector protein, the effector protein is transactivated by the tracrRNA. In other words, activity of effector protein requires binding to a tracrRNA molecule. In some embodiments, the dual nucleic acid system comprises a guide nucleic acid and a tracrRNA, wherein the tracrRNA is an additional nucleic acid capable of at least partially hybridizing to the first region of the guide nucleic acid. In some embodiments, the tracrRNA or additional nucleic acid is capable of at least partially hybridizing to the 5′ end of the second region of the guide nucleic acid.

In some embodiments, a repeat hybridization sequence is at the 3′ end of a tracrRNA. In some embodiments, a repeat hybridization sequence may have a length of about 1, about 2, about 3, about 4, about 5, about 6, about 7, about 8, about 9, about 10, about 12, about 14, about 16, about 18, or about 20 linked nucleotides. In some embodiments, the length of the repeat hybridization sequence is 1 to 20 linked nucleotides.

A tracrRNA and/or tracrRNA-crRNA duplex may form a secondary structure that facilitates the binding of an effector protein to a tracrRNA or a tracrRNA-crRNA. In some embodiments, the secondary structure modifies activity of the effector protein on a target nucleic acid. In some embodiments, the secondary structure comprises a stem-loop structure comprising a stem region and a loop region. In some embodiments, the stem region is 4 to 8 linked nucleotides in length. In some embodiments, the stem region is 5 to 6 linked nucleotides in length. In some embodiments, the stem region is 4 to 5 linked nucleotides in length. In some embodiments, the secondary structure comprises a pseudoknot (e.g., a secondary structure comprising a stem at least partially hybridized to a second stem or half-stem secondary structure). An effector protein may recognize a secondary structure comprising multiple stem regions. In some embodiments, nucleotide sequences of the multiple stem regions are identical to one another. In some embodiments, the nucleotide sequences of at least one of the multiple stem regions is not identical to those of the others. In some embodiments, the secondary structure comprises at least two, at least three, at least four, or at least five stem regions. In some embodiments, the secondary structure comprises one or more loops. In some embodiments, the secondary structure comprises at least one, at least two, at least three, at least four, or at least five loops.

### F. Engineered Modifications

Polypeptides (e.g., effector proteins) and nucleic acids (e.g., engineered guide nucleic acids) can be further modified as described herein. Examples are modifications that do not alter the primary sequence of the polypeptides or nucleic acids, such as chemical derivatization of polypeptides (e.g., acylation, acetylation, carboxylation, amidation, etc.), or modifications that do alter the primary sequence of the polypeptide or nucleic acid. Also included are polypeptides that have a modified glycosylation pattern (e.g., those made by: modifying the glycosylation patterns of a polypeptide during its synthesis and processing or in further processing steps; by exposing the polypeptide to enzymes which affect glycosylation, such as mammalian glycosylating or deglycosylating enzymes). Also embraced are polypeptides that have phosphorylated amino acid residues (e.g., phosphotyrosine, phosphoserine, or phosphothreonine).

Modifications disclosed herein can also include modification of described polypeptides and/or guide nucleic acids through any suitable method, such as molecular biological techniques and/or synthetic chemistry, to improve their resistance to proteolytic degradation, to change the target sequence specificity, to optimize solubility properties, to alter protein activity (e.g., transcription modulatory activity, enzymatic activity, etc.) or to render them more suitable for their intended purpose (e.g., in vivo administration, in vitro methods, or ex vivo applications). Analogs of such polypeptides include those containing residues other than naturally occurring L-amino acids, e.g. D-amino acids or non-naturally occurring synthetic amino acids. D-amino acids may be substituted for some or all of the amino acid residues. Modifications can also include modifications with non-naturally occurring unnatural amino acids. The particular sequence and the manner of preparation will be determined by convenience, economics, purity required, and the like.

Modifications can further include the introduction of various groups to polypeptides and/or guide nucleic acids described herein. For example, groups can be introduced during synthesis or during expression of a polypeptide (e.g., an effector protein), which allow for linking to other molecules or to a surface. Thus, e.g., cysteines may be used to make thioethers, histidines for linking to a metal ion complex, carboxyl groups for forming amides or esters, amino groups for forming amides, and the like.

Modifications can further include changing of nucleic acids described herein (e.g., engineered guide nucleic acids) to provide the nucleic acid with a new or enhanced feature, such as improved stability. Such modifications of a nucleic acid include a nucleobase base modification, abackbone modification, a sugar modification, or combinations thereof. In some embodiments, the modifications can be of one or more nucleotides, nucleosides, or nucleobases in a nucleic acid. In some embodiments, uridines can be exchanged for pseudouridines (e.g., 1N-Methyl-Pseudouridine). In some embodiments, all uridines can be exchanged for 1N-Methyl-Pseudouridine. In this application, U can represent uracil or 1N-Methyl-Pseudouridine.

In some embodiments, nucleic acids (e.g., nucleic acids encoding effector proteins, engineered guide nucleic acids, or nucleic acids encoding engineered guide nucleic acids) described herein comprise one or more modifications comprising: 2′O-methyl modified nucleotides (e.g., 2′-O-Methyl (2′OMe) sugar modifications); 2′ fluoro modified nucleotides (e.g., 2′-fluoro (2′-F) sugar modifications); locked nucleic acid (LNA) modified nucleotides; peptide nucleic acid (PNA) modified nucleotides; nucleotides with phosphorothioate linkages; a 5′ cap (e.g., a 7-methylguanylate cap (m7G)), phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkyl phosphoramidates, phosphorodiamidates, thionophosphor amidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage; phosphorothioate and/or heteroatom internucleoside linkages, such as —CH2-NH—O—CH2-, —CH2-N(CH3)-O—CH2- (known as a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-); morpholino linkages (formed in part from the sugar portion of a nucleoside); morpholino backbones; phosphorodiamidate or other non-phosphodiester internucleoside linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; other backbone modifications having mixed N, O, S and CH2 component parts; and combinations thereof.

### G. Expression Vectors

Compositions, systems, and methods described herein comprise a vector or a use thereof. A vector can comprise a nucleic acid of interest. In some embodiments, the nucleic acid of interest comprises one or more components of a composition or system described herein. In some embodiments, the nucleic acid of interest comprises a nucleotide sequence that encodes one or more components of the composition or system described herein. In some embodiments, one or more components comprises an effector protein, an effector partner, and a guide nucleic acid. In some embodiments, the component comprises a nucleic acid encoding an effector protein, nucleic acid encoding an effector partner, and a guide nucleic acid. In some embodiments, a vector may be part of a vector system. In some embodiments, components described herein (e.g., effector protein, effector partner, guide nucleic acid) are encoded by the same vector. In some embodiments, components described herein (e.g., effector protein, effector partner, guide nucleic acid) are encoded by different vectors. In some embodiments, the components are operably linked to a promoter that is operable in a target cell, such as a eukaryotic cell.

In some embodiments, a vector may comprise or encode one or more regulatory elements. Regulatory elements may refer to transcriptional and translational control sequences, such as promoters, enhancers, polyadenylation signals, terminators, protein degradation signals, and the like, that provide for and/or regulate transcription of a non-coding sequence or a coding sequence and/or regulate translation of an encoded polypeptide. In some embodiments, a vector may comprise or encode for one or more additional elements, such as, for example, replication origins, antibiotic resistance (or a nucleic acid encoding the same), a tag (or a nucleic acid encoding the same), selectable markers, and the like. In some embodiments, a vector comprises or encodes for one or more elements, such as, for example, ribosome binding sites, and RNA splice sites.

Vectors described herein can encode a promoter—a regulatory region on a nucleic acid, such as a DNA sequence, capable of initiating transcription of a downstream (3′ direction) coding or non-coding sequence. A promoter can be linked at its 3′ terminus to a nucleic acid, the expression or transcription of which is desired, and extends upstream (5′ direction) to include bases or elements necessary to initiate transcription or induce expression, which could be measured at a detectable level. A promoter can comprise a nucleotide sequence, referred to herein as a “promoter sequence”. The promoter sequence can include a transcription initiation site, and one or more protein binding domains responsible for the binding of transcription machinery, such as RNA polymerase. When eukaryotic promoters are used, such promoters can contain “TATA” boxes and “CAT” boxes. Various promoters, including inducible promoters, may be used to drive expression, i.e., transcriptional activation, of the nucleic acid of interest. Accordingly, in some embodiments, the nucleic acid of interest can be operably linked to a promoter.

Promotors may be any suitable type of promoter envisioned for the compositions, systems, and methods described herein. Examples include constitutively active promoters (e.g., CMV promoter), inducible promoters (e.g., heat shock promoter, tetracycline-regulated promoter, steroid-regulated promoter, metal-regulated promoter, estrogen receptor-regulated promoter, etc.), spatially restricted and/or temporally restricted promoters (e.g., a tissue specific promoter, a cell type specific promoter, etc.), etc. Suitable promoters include, but are not limited to: SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, and a human Hl promoter (Hl). By transcriptional activation, it is intended that transcription will be increased above basal levels in the target cell by 2 fold, 5 fold, 10 fold, 50 fold, by 100 fold, 500 fold, or by 1000 fold, or more. In addition, vectors used for providing a nucleic acid that, when transcribed, produces a guide nucleic acid and/or a nucleic acid that encodes an effector protein to a cell may include nucleic acid sequences that encode for selectable markers in the target cells, so as to identify cells that have taken up the guide nucleic acid and/or the effector protein.

In general, vectors provided herein comprise at least one promotor or a combination of promoters driving expression or transcription of an effector protein, effector partner, guide nucleic acid, or combination thereof. In some embodiments, the vector comprises a nucleotide sequence of a promoter. In some embodiments, the vector comprises two promoters. In some embodiments, the vector comprises three promoters. In some embodiments, a length of the promoter is less than about 500, less than about 400, less than about 300, or less than about 200 linked nucleotides. In some embodiments, a length of the promoter is at least 100, at least 200, at least 300, at least 400, or at least 500 linked nucleotides. Non-limiting examples of promoters include CMV, 7SK, EFla, RPBSA, hPGK, EFS, SV40, PGK1, Ubc, human beta actin, TRE, UAS, Ac5, Polyhedrin, CaMKIIa, GALl-10, H1, TEF1, GDS, ADH1, CaMV35S, HSV TK, Ubi, U6, MNDU3, MSCV, MND, and CAG. In some embodiments, some promoters (e.g., U6, enhanced U6, Hl and 7SK) prefers the nucleic acid being transcribed having “g” nucleotide at the 5′ end of the coding sequence. Accordingly, when such coding sequence is expressed, it comprises an additional “g” nucleotide at 5′ end. In some embodiments, vectors provided herein comprise a promotor driving expression or transcription of any one of the guide nucleic acids described herein (e.g., TABLEs 7-10) further comprises “g” nucleotide at 5′ end of the guide nucleic acid, wherein the promotor is selected from U6, enhanced U6, Hl and 7SK.

In some embodiments, the promoter is a constitutive promoter. In some embodiments, the promoter is an inducible promoter. In some embodiments, the inducible promoter only drives expression of its corresponding coding sequence (e.g., polypeptide or guide nucleic acid) when a signal is present, e.g., a hormone, a small molecule, a peptide. Non-limiting examples of inducible promoters are the T7 RNA polymerase promoter, the T3 RNA polymerase promoter, the Isopropyl-beta-D-thiogalactopyranoside (IPTG)-regulated promoter, a lactose induced promoter, a heat shock promoter, a tetracycline-regulated promoter (tetracycline-inducible or tetracycline-repressible), a steroid regulated promoter, a metal-regulated promoter, and an estrogen receptor-regulated promoter. In some embodiments, the promoter is an activation-inducible promoter, such as a CD69 promoter. In some embodiments, the promoter for expressing effector protein is a ubiquitous promoter. In some embodiments, the ubiquitous promoter comprises MND or CAG promoter sequence.

In some embodiments, the promoters are prokaryotic promoters (e.g., drive expression of a gene in a prokaryotic cell). In some embodiments, the promoters are eukaryotic promoters, (e.g., drive expression of a gene in a eukaryotic cell). In some embodiments, the promoter is EF1a. In some embodiments, the promoter is ubiquitin. In some embodiments, vectors are bicistronic or polycistronic vector (e.g., having or involving two or more loci responsible for generating a protein) having an internal ribosome entry site (IRES) is for translation initiation in a cap-independent manner.

In some embodiments, a vector described herein is a nucleic acid expression vector. In some embodiments, a vector described herein is a recombinant expression vector. In some embodiments, a vector described herein is a messenger RNA. In some embodiments, a vector comprising the recombinant nucleic acid as described herein, wherein the vector is a viral vector, an adeno associated viral (AAV) vector, a retroviral vector, or a lentiviral vector. In some embodiments, a vector described herein or a recombinant nucleic acid described herein is comprised in a cell. In some embodiments, a recombinant nucleic acid integrated into a genomic DNA sequence of the cell, wherein the cell is a eukaryotic cell or a prokaryotic cell.

In some embodiments, a vector described herein is a delivery vector. In some embodiments, the delivery vector is a eukaryotic vector, a prokaryotic vector (e.g., a bacterial vector) a viral vector, or any combination thereof. In some embodiments, the delivery vehicle is a non-viral vector. In some embodiments, the delivery vector is a plasmid. In some embodiments, the plasmid comprises DNA. In some embodiments, the plasmid comprises RNA. In some embodiments, the plasmid comprises circular double-stranded DNA. In some embodiments, the plasmid is linear. In some embodiments, the plasmid comprises one or more coding sequences of interest and one or more regulatory elements. In some embodiments, the plasmid comprises a bacterial backbone containing an origin of replication and an antibiotic resistance gene or other selectable marker for plasmid amplification in bacteria. In some embodiments, the plasmid is a minicircle plasmid. In some embodiments, the plasmid contains one or more genes that provide a selective marker to induce a target cell to retain the plasmid. In some examples, the plasmids are engineered through synthetic or other suitable means known in the art. For example, in some embodiments, the genetic elements are assembled by restriction digest of the desired genetic sequence from a donor plasmid or organism to produce ends of the DNA which is then be readily ligated to another genetic sequence.

In some embodiments, vectors comprise an enhancer. Enhancers are nucleotide sequences that have the effect of enhancing promoter activity. In some embodiments, enhancers augment transcription regardless of the orientation of their sequence. In some embodiments, enhancers activate transcription from a distance of several kilo base pairs. Furthermore, enhancers are located optionally upstream or downstream of a gene region to be transcribed, and/or located within the gene, to activate the transcription. Exemplary enhancers include, but are not limited to, WPRE; CMV enhancers; the R—U5′ segment in LTR of HTLV-I.

### Administration of a Non-Viral Vector

In some embodiments, an administration of a non-viral vector comprises contacting a cell, such as a host cell, with the non-viral vector. In some embodiments, a physical method or a chemical method is employed for delivering the vector into the cell. Exemplary physical methods include electroporation, gene gun, sonoporation, magnetofection, or hydrodynamic delivery. Exemplary chemical methods include delivery of the recombinant polynucleotide by liposomes such as, cationic lipids or neutral lipids; lipofection; dendrimers; lipid nanoparticle (LNP); or cell-penetrating peptides.

### Lipid Particles and Non-Viral Vectors

In some embodiments, compositions and systems provided herein comprise a lipid particle. In some embodiments, a lipid particle is a lipid nanoparticle (LNP). In some embodiments, a lipid or a lipid nanoparticle can encapsulate an expression vector as described herein. LNPs are a non-viral delivery system for delivery of the composition and/or system components described herein. LNPs are particularly effective for delivery of nucleic acids. Beneficial properties of LNP include ease of manufacture, low cytotoxicity and immunogenicity, high efficiency of nucleic acid encapsulation and cell transfection, multi-dosing capabilities and flexibility of design (Kulkarni et al., (2018) Nucleic Acid Therapeutics, 28(3):146-157). In some embodiments, compositions and methods comprise a lipid, polymer, nanoparticle, or a combination thereof, or use thereof, to introduce one or more effector proteins, one or more guide nucleic acids, one or more effector partners, or any combinations thereof to a cell. Non-limiting examples of lipids and polymers are cationic polymers, cationic lipids, ionizable lipids, or bio-responsive polymers. In some embodiments, the ionizable lipids exploits chemical-physical properties of the endosomal environment (e.g., pH) offering improved delivery of nucleic acids. In some embodiments, the ionizable lipids are neutral at physiological pH. In some embodiments, the ionizable lipids are protonated under acidic pH. In some embodiments, the bio-responsive polymer exploits chemical-physical properties of the endosomal environment (e.g., pH) to preferentially release the genetic material in the intracellular space.

In some embodiments, a LNP comprises an outer shell and an inner core. In some embodiments, the outer shell comprises lipids. In some embodiments, the lipids comprise modified lipids. In some embodiments, the modified lipids comprise pegylated lipids. In some embodiments, the lipids comprise one or more of cationic lipids, anionic lipids, ionizable lipids, and non-ionic lipids. In some embodiments, the LNP comprises one or more of N1,N3,N5-tris(3-(didodecylamino)propyl)benzene-1,3,5-tricarboxamide (TT3), 2-dioleoyl-sn-glycero-3-phosphoethanolamine (DOPE), 1-palmitoyl-2-oleoylsn-glycero-3-phosphoethanolamine (POPE), 1,2-distearoyl-sn-glycero-3-phosphocholine (DSPC), cholesterol (Chol), 1,2-dimyristoyl-sn-glycerol, and methoxypolyethylene glycol (DMG-PEChooo), derivatives, analogs, or variants thereof. In some embodiments, the LNP has a negative net overall charge prior to complexation with one or more of a guide nucleic acid, effector protein, effector partner, and nucleic acid encoding the same. In some embodiments, the inner core is a hydrophobic core. In some embodiments, the guide nucleic acid, effector protein, effector partner, or nucleic acid encoding the same forms a complex with one or more of the cationic lipids and the ionizable lipids. In some embodiments, the nucleic acid encoding the effector protein or the nucleic acid encoding the guide nucleic acid is self-replicating.

In some embodiments, a LNP comprises one or more of cationic lipids, ionizable lipids, and modified versions thereof. In some embodiments, the ionizable lipid comprises TT3 or a derivative thereof. Accordingly, in some embodiments, the LNP comprises one or more of TT3 and pegylated TT3. The publication WO2016187531 is hereby incorporated by reference in its entirety, which describes representative LNP formulations in Table 2 and Table 3, and representative methods of delivering LNP formulations in Example 7.

In some embodiments, a LNP comprises a lipid composition targeting to a specific organ. In some embodiments, the lipid composition comprises lipids having a specific alkyl chain length that controls accumulation of the LNP in the specific organ (e.g., liver or spleen). In some embodiments, the lipid composition comprises a biomimetic lipid that controls accumulation of the LNP in the specific organ (e.g., brain). In some embodiments, the lipid composition comprises lipid derivatives (e.g., cholesterol derivatives) that controls accumulation of the LNP in a specific cell (e.g., liver endothelial cells, Kupffer cells, hepatocytes).

### Delivery of Viral Vectors

In some embodiments, a vector described herein comprises a viral vector. In some embodiments, the viral vector comprises a nucleic acid to be delivered into a host cell by a recombinantly produced virus or viral particle. The nucleic acid may be single-stranded or double stranded, linear or circular, segmented or non-segmented. The nucleic acid may comprise DNA, RNA, or a combination thereof. In some embodiments, the vector is an adeno-associated viral vector. There are a variety of viral vectors that are associated with various types of viruses, including but not limited to retroviruses (e.g., lentiviruses and γ-retroviruses), adenoviruses, arenaviruses, alphaviruses, adeno-associated viruses (AAVs), baculoviruses, vaccinia viruses, herpes simplex viruses and poxviruses. In some embodiments, the vector is an adeno-associated viral (AAV) vector. In some embodiments, the viral vector is a recombinant viral vector. In some embodiments, the vector is a retroviral vector. In some embodiments, the retroviral vector is a lentiviral vector. In some embodiments, the retroviral vector comprises gamma-retroviral vector. A viral vector provided herein may be derived from or based on any such virus. For example, in some embodiments, the gamma-retroviral vector is derived from a Moloney Murine Leukemia Virus (MoMLV, MMLV, MuLV, or MLV) or a Murine Stem cell Virus (MSCV) genome. In some embodiments, the lentiviral vector is derived from the human immunodeficiency virus (HIV) genome. In some embodiments, the viral vector is a chimeric viral vector. In some embodiments, the chimeric viral vector comprises viral portions from two or more viruses. In some embodiments, the viral vector corresponds to a virus of a specific serotype.

In some embodiments, a viral vector is an adeno-associated viral vector (AAV vector). In some embodiments, a viral particle that delivers a viral vector described herein is an AAV. In some embodiments, the AAV comprises any AAV known in the art. In some embodiments, the viral vector corresponds to a virus of a specific AAV serotype. In some embodiments, the AAV serotype is selected from an AAV1 serotype, an AAV2 serotype, AAV3 serotype, an AAV4 serotype, AAV5 serotype, an AAV6 serotype, AAV7 serotype, an AAV8 serotype, an AAV9 serotype, an AAV10 serotype, an AAV11 serotype, an AAV12 serotype, an AAV-rh10 serotype, and any combination, derivative, or variant thereof. In some embodiments, the AAV vector is a recombinant vector, a hybrid AAV vector, a chimeric AAV vector, a self-complementary AAV (scAAV) vector, a single-stranded AAV, or any combination thereof. scAAV genomes are generally known in the art and contain both DNA strands which can anneal together to form double-stranded DNA.

In some embodiments, an AAV vector described herein is a chimeric AAV vector. In some embodiments, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some embodiments, AAV vector described herein comprises two inverted terminal repeats (ITRs). According, in some embodiments, the viral vector provided herein comprises two inverted terminal repeats of AAV. A nucleotide sequence between the ITRs of an AAV vector (also referred to as a transgene) provided herein comprises a sequence an effector protein, effector partner, guide nucleic acid, a nucleic acid encoding the same, or combination thereof. In some embodiments, the transgene comprises a nucleic acid encoding one or more effector proteins (and/or effector partners), a nucleic acid encoding one or more fusion proteins (e.g., a nuclear localization signal (NLS), polyA tail), one or more guide nucleic acids, a nucleic acid encoding the one or more guide nucleic acids, respective promoter(s), or any combinations thereof. In some embodiments, viral vectors provided herein comprise at least one promotor or a combination of promoters driving expression or transcription of an effector protein, effector partner, guide nucleic acid, a nucleic acid encoding the same, or combination thereof. In some embodiments, a coding region of the AAV vector forms an intramolecular double-stranded DNA template thereby generating the AAV vector that is a self-complementary AAV (scAAV) vector. In some embodiments, the scAAV vector comprises the sequence (encoding an effector protein, effector partner, guide nucleic acid, a nucleic acid encoding the same, or combination thereof) that has a length of about 2 kb to about 3 kb. In some embodiments, the AAV vector provided herein is a self-inactivating AAV vector. In some embodiments, the AAV vector provided herein comprises a modification, such as an insertion, deletion, chemical alteration, or synthetic modification, relative to a wild-type AAV vector.

### Producing AAV Delivery Vectors

In some embodiments, methods of producing AAV delivery vectors herein comprise packaging a nucleic acid encoding an effector protein and a guide nucleic acid, or a combination thereof, into an AAV vector. In some embodiments, methods of producing the delivery vector comprises, (a) contacting a cell with at least one nucleic acid encoding: (i) a guide nucleic acid; (ii) a Replication (Rep) gene; and (iii) a Capsid (Cap) gene that encodes an AAV capsid protein; (b) expressing the AAV capsid protein in the cell; (c) assembling an AAV particle; and (d) packaging an effector encoding nucleic acid into the AAV particle, thereby generating an AAV delivery vector. In some embodiments, promoters, stuffer sequences, and any combination thereof may be packaged in the AAV vector. In some examples, the AAV vector may package 1, 2, 3, 4, or 5 guide nucleic acids or copies thereof. In some embodiments, the AAV vector comprises inverted terminal repeats, e.g., a 5′ inverted terminal repeat and a 3′ inverted terminal repeat. In some embodiments, the AAV vector comprises a mutated inverted terminal repeat that lacks a terminal resolution site.

In some embodiments, a hybrid AAV vector is produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV9), wherein the first and second AAV serotypes may be not the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

### Producing AAV Particles

In some embodiments, AAV particles described herein are recombinant AAV (rAAV). In some embodiments, rAAV particles are generated by transfecting AAV producing cells with an AAV-containing plasmid carrying the sequence encoding an effector protein, effector partner, guide nucleic acid, a nucleic acid encoding the same, or combination thereof, a plasmid that carries viral encoding regions, i.e., Rep and Cap gene regions; and a plasmid that provides the helper genes such as ElA, E1B, E2A, E40RF6 and VA. In some embodiments, the AAV producing cells are mammalian cells. In some embodiments, host cells for rAAV viral particle production are mammalian cells. In some embodiments, a mammalian cell for rAAV viral particle production is a COS cell, a HEK293T cell, a HeLa cell, a KB cell, a variant thereof, or a combination thereof. In some embodiments, rAAV virus particles can be produced in the mammalian cell culture system by providing the rAAV plasmid to the mammalian cell. In some embodiments, producing rAAV virus particles in a mammalian cell comprises transfecting vectors that express the rep protein, the capsid protein, and the gene-of-interest expression construct flanked by the ITR sequence on the 5′ and 3′ ends. Methods of such processes are provided in, for example, Naso et al., BioDrugs, 2017 August; 31(4):317-334 and Benskey et al., (2019), Methods Mol Biol., 1937:3-26, each of which is incorporated by reference in their entireties.

In some embodiments, rAAV is produced in a non-mammalian cell. In some embodiments, rAAV is produced in an insect cell. In some embodiments, the insect cell for producing rAAV viral particles comprises a Sf9 cell. In some embodiments, production of rAAV virus particles in insect cells may comprise baculovirus. In some embodiments, production of rAAV virus particles in insect cells may comprise infecting the insect cells with three recombinant baculoviruses, one carrying the cap gene, one carrying the rep gene, and one carrying the gene-of-interest expression construct enclosed by an ITR on both the 5′ and 3′ end. In some embodiments, rAAV virus particles are produced by the One Bac system. In some embodiments, rAAV virus particles can be produced by the Two Bac system. In some embodiments, in the Two Bac system, the rep gene and the cap gene of the AAV is integrated into one baculovirus virus genome, and the ITR sequence and the gene-of-interest expression construct is integrated into another baculovirus virus genome. In some embodiments, in the One Bac system, an insect cell line that expresses both the rep protein and the capsid protein is established and infected with a baculovirus virus integrated with the ITR sequence and the gene-of-interest expression construct. Details of such processes are provided in, for example, Smith et. al., (1983), Mol. Cell. Biol., 3(12):2156-65; Urabe et al., (2002), Hum. Gene. Ther., 1;13(16):1935-43; and Benskey et al., (2019), Methods Mol Biol., 1937:3-26, each of which is incorporated by reference in its entirety.

### H. Target Nucleic Acids

Disclosed herein are compositions, systems and methods for modifying expression of a target nucleic acid. In some embodiments, the target nucleic acid is a double stranded nucleic acid. In some embodiments, the target nucleic acid is a single stranded nucleic acid. Alternatively, or in combination, the target nucleic acid is a double stranded nucleic acid and is prepared into single stranded nucleic acids before or upon contacting an RNP. In some embodiments, the single stranded nucleic acid comprises a RNA, wherein the RNA comprises a mRNA, a rRNA, a tRNA, a non-coding RNA, a long non-coding RNA, a microRNA (miRNA), and a single-stranded RNA (ssRNA). In some embodiments, the target nucleic acid is complementary DNA (cDNA) synthesized from a single-stranded RNA template in a reaction catalyzed by a reverse transcriptase. In some embodiments, the target nucleic acid comprises an RNA, a DNA, or combination thereof. In some embodiments, guide nucleic acids described herein hybridize to a portion of the target nucleic acid. In some embodiments, the target nucleic acid is from a virus, a parasite, or a bacterium described herein. In some embodiments, the target sequence is only found in a eukaryote in nature. In some embodiments, the target sequence is only found in a human in nature.

In some embodiments, a target nucleic acid comprising a target sequence comprises a PAM sequence. In some embodiments, the PAM sequence is adjacent to the target sequence. In some embodiments, the PAM sequence is 3′ to the target sequence. In some embodiments, the PAM sequence is directly 3′ to the target sequence. In some embodiments, the PAM sequence 5′ to the target sequence. In some embodiments, the PAM sequence is directly 5′ to the target sequence. In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by an effector protein system.

In some embodiments, a target nucleic acid comprises 5 to 100, 5 to 90, 5 to 80, 5 to 70, 5 to 60, 5 to 50, 5 to 40, 5 to 30, 5 to 25, 5 to 20, 5 to 15, or 5 to 10 linked nucleotides. In some embodiments, the target nucleic acid comprises 10 to 90, 20 to 80, 30 to 70, or 40 to 60 linked nucleotides. In some embodiments, the target nucleic acid comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 linked nucleotides. In some embodiments, the target nucleic acid comprises at least 10, at least 20, at least 30, at least 40, at least 50, at least 60, at least 70, at least 80, at least 90, or at least 100 linked nucleotides. In some embodiments, the target sequence in the target nucleic acid comprises at least 10 contiguous nucleotides that are complementary to the guide nucleic acid or engineered guide nucleic acid.

In some embodiments, compositions, systems, and methods described herein comprise a target nucleic acid may be responsible for a disease, contain a mutation (e.g., single strand polymorphism, point mutation, insertion, or deletion), be contained in an amplicon, or be uniquely identifiable from the surrounding nucleic acids (e.g., contain a unique sequence of nucleotides). In some embodiments, the target nucleic acid has undergone a modification after contacting with an RNP. In some embodiments, the modification is a change in the sequence of the target nucleic acid. In some embodiments, the modification comprises an epigenetic modification.

In some embodiments, the target nucleic acid comprises a nucleic acid sequence from a pathogen responsible for a disease. Non-limiting examples of pathogens are bacteria, a virus and a fungus. In some embodiments, the target nucleic acid comprises a nucleic acid sequence of a virus, a bacterium, or other pathogen responsible for a disease in a plant (e.g., a crop).

In some embodiments, a target nucleic acid comprises a portion or a specific region of a nucleic acid from a genomic locus, any DNA amplicon of, a reverse transcribed mRNA, or a cDNA from a gene described herein. In some embodiments, the target nucleic acid is an amplicon of at least a portion of a gene. Non-limiting examples of genes are recited in TABLE 11. Nucleic acid sequences of target nucleic acids and/or corresponding genes are readily available in public databases as known and used in the art. In some embodiments, the target nucleic acid is selected from TABLE 11. In some embodiments, the target nucleic acid comprises one or more target sequences. In some embodiments, the one or more target sequence is within any one of the target nucleic acids set forth in TABLE 11. In some embodiments, the one or more target sequence is from about 500 nucleotides upstream to about 500 nucleotides downstream of the transcriptional start site of the target nucleic acids set forth in TABLE 11.

In some embodiments, a target sequence is from a region from about 500 nucleotides upstream to about 500 nucleotides downstream of the transcriptional start site of the apolipoprotein C3 (APOC3) gene. In general, guide nucleic acids described herein comprise a sequence that is complementary to and/or hybridizes to the target sequence of the APOC3 gene.

In some embodiments, a target sequence is from a region from about 500 nucleotides upstream to about 500 nucleotides downstream of the transcriptional start site of the Proprotein convertase subtilisin/kexin type 9 (PCSK9) gene. In general, guide nucleic acids described herein comprise a sequence that is complementary to and/or hybridizes to a target sequence of the PCSK9 gene.

In some embodiments, a target sequence is from a region from about 500 nucleotides upstream to about 500 nucleotides downstream of the transcriptional start site of the Angiopoietin-like 3 (ANGPTL3) gene. In general, guide nucleic acids described herein comprise a sequence that is complementary to and/or hybridizes to a target sequence of the ANGPTL3 gene.

### Mutations

In some embodiments, target nucleic acids described herein comprise a mutation. In some embodiments, a composition, system or method described herein can be used to modify the expression of a target nucleic acid comprising a mutation. In some embodiments, a composition, system or method described herein can be used to reduce, prevent or abolish the expression of a target nucleic acid comprising a mutation. A mutation may result in the insertion of at least one amino acid in a protein encoded by the target nucleic acid. A mutation may result in the deletion of at least one amino acid in a protein encoded by the target nucleic acid. A mutation may result in the substitution of at least one amino acid in a protein encoded by the target nucleic acid. A mutation that results in the deletion, insertion, or substitution of one or more amino acids of a protein encoded by the target nucleic acid may result in misfolding of a protein encoded by the target nucleic acid. A mutation may result in a premature stop codon, thereby resulting in a truncation of the encoded protein.

Non-limiting examples of mutations are insertion-deletion (indel), a point mutation, single nucleotide polymorphism (SNP), a chromosomal mutation, a copy number mutation or variation, and frameshift mutations. In some embodiments, an indel mutation is an insertion or deletion of one or more nucleotides. In some embodiments, a point mutation comprises a substitution, insertion, or deletion. In some embodiments, a frameshift mutation occurs when the number of nucleotides in the insertion/deletion is not divisible by three, and it occurs in a protein coding region. In some embodiments, a chromosomal mutation can comprise an inversion, a deletion, a duplication, or a translocation of one or more nucleotides. In some embodiments, a copy number variation can comprise a gene amplification or an expanding trinucleotide repeat. In some embodiments, an SNP is associated with a phenotype of the sample or a phenotype of the organism from which the sample was taken. In some embodiments, an SNP is associated with altered phenotype from wild type phenotype. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution.

In some embodiments, a target nucleic acid described herein comprises a mutation of one or more nucleotides. In some embodiments, the one or more nucleotides comprises 1, 2, 3, 4, 5, 6, 7, 8, 9, 10, 11, 12, 13, 14, 15, 16, 17, 18, 19, 20 or more nucleotides. In some embodiments, the mutation comprises a deletion, insertion, and/or substitution of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. In some embodiments, the mutation comprises a deletion, insertion, and/or substitution of 1 to 5, 5 to 10, 10 to 15, 15 to 20, 20 to 25, 25 to 30, 30 to 35, 35 to 40, 40 to 45, 45 to 50, 50 to 55, 55 to 60, 60 to 65, 65 to 70, 70 to 75, 75 to 80, 80 to 85, 85 to 90, 90 to 95, 95 to 100, 100 to 200, 200 to 300, 300 to 400, 400 to 500, 500 to 600, 600 to 700, 700 to 800, 800 to 900, 900 to 1000, 1 to 50, 1 to 100, 25 to 50, 25 to 100, 50 to 100, 100 to 500, 100 to 1000, or 500 to 1000 nucleotides. The mutation may be located in a non-coding region or a coding region of a gene, wherein the gene is a target nucleic acid. A mutation may be in an open reading frame of a target nucleic acid. In some embodiments, guide nucleic acids described herein hybridize to a portion of the target nucleic acid comprising or adjacent to the mutation.

In some embodiments, the target nucleic acid comprises one or more mutations. In some embodiments, the target nucleic acid comprises one or more, two or more, three or more, four or more, five or more, six or more, seven or more, eight or more, nine or more, or ten or more mutations as compared to the unmutated target nucleic acid. In some embodiments, the target nucleic acid comprises a sequence comprising one or more, two or more, three or more, four or more, five or more, six or more, seven or more, eight or more, nine or more, or ten or more mutations as compared to the wildtype sequence. In some embodiments, the target nucleic acid comprises a mutation associated with a disease or disorder.

In some embodiments, target nucleic acids comprise a mutation, wherein the mutation is a SNP. In some embodiments, the single nucleotide mutation or SNP is associated with a phenotype of the sample or a phenotype of the organism from which the sample was taken. In some embodiments, the SNP is associated with altered phenotype from wild type phenotype. In some embodiments, a single nucleotide mutation, SNP, or deletion described herein is associated with a disease, such as a genetic disease. In some embodiments, the SNP is a synonymous substitution or a nonsynonymous substitution. In some embodiments, the nonsynonymous substitution is a missense substitution or a nonsense point mutation. In some embodiments, the synonymous substitution is a silent substitution. In some embodiments, the mutation is a deletion of one or more nucleotides. In some embodiments, the single nucleotide mutation, SNP, or deletion is associated with a disease such as a genetic disorder. In some embodiments, the mutation, such as a single nucleotide mutation, a SNP, or a deletion, may be encoded in the sequence of a target nucleic acid from the germline of an organism or may be encoded in a target nucleic acid from a diseased cell.

In some embodiments, the mutation is associated with a disease, such as a genetic disorder. In some embodiments, the mutation may be encoded in the sequence of a target nucleic acid from the germline of an organism or may be encoded in a target nucleic acid from a diseased cell. In some embodiments, a target nucleic acid described herein comprises a mutation associated with a disease. In some examples, a mutation associated with a disease refers to a mutation whose presence in a subject indicates that the subject is susceptible to or suffers from, a disease, disorder, condition, or syndrome. In some examples, a mutation associated with a disease refers to a mutation which causes, contributes to the development of, or indicates the existence of the disease, disorder, condition, or syndrome. A mutation associated with a disease may also refer to any mutation which generates transcription or translation products at an abnormal level, or in an abnormal form, in cells affected by a disease relative to a control without the disease. In some examples, a mutation associated with a disease refers to a mutation whose presence in a subject indicates that the subject is susceptible to, or suffers from, a disease, disorder, or pathological state. In some embodiments, a mutation associated with a disease, comprises the co-occurrence of a mutation and the phenotype of a disease. The mutation may occur in a gene, wherein transcription or translation products from the gene occur at a significantly abnormal level or in an abnormal form in a cell or subject harboring the mutation as compared to a non-disease control subject not having the mutation. In some embodiments, a target nucleic acid described herein comprises a mutation associated with a disease, wherein the target nucleic acid is any one of the target nucleic acids set forth in TABLE 11. In some embodiments, a target nucleic acid described herein comprises a mutation associated with a disease, wherein the disease is any one of the diseases set forth in TABLE 12.

### II. Pharmaceutical Compositions

Described herein are formulations of introducing compositions or components of a system described herein to a host. In some embodiments, compositions described herein are pharmaceutical compositions. In some embodiments, the pharmaceutical compositions comprise compositions described herein or systems described herein. In some embodiments, the pharmaceutical composition comprises a pharmaceutically acceptable salt, one or more of a vehicle, adjuvant, excipient, or carrier, such as a filler, disintegrant, a surfactant, a binder, a lubricant, or combinations thereof. Remington: The Science and Practice of Pharmacy, 23rd edition, 2020, ed. D. B. Troy, Lippincott Williams & Wilkins, Philadelphia; Encyclopedia of Pharmaceutical Technology, eds. J. Swarbrick and J. C. Boylan, 1988-2001, Marcel Dekker, New York; and Encyclopedia of Pharmaceutical Technology, 3rd edition, 2016, eds. J. Swarbrick, CRC Press, Boca Raton, as well as more recent editions of the aforementioned publications, disclose various carriers used in formulating pharmaceutically acceptably compositions and known techniques for the preparation thereof. Non-limiting examples of pharmaceutically acceptable carriers and diluents suitable for the pharmaceutical compositions disclosed herein include buffers (e.g., neutral buffered saline, phosphate buffered saline); carbohydrates (e.g., glucose, mannose, sucrose, dextran, mannitol); polypeptides or amino acids (e.g., glycine); antioxidants; chelating agents (e.g., EDTA, glutathione); adjuvants (e.g., aluminum hydroxide); surfactants (Polysorbate 80, Polysorbate 20, or Pluronic F68); glycerol; sorbitol; mannitol; polyethyleneglycol; and preservatives. In some embodiments, the vector is formulated for delivery through injection by a needle carrying syringe. In some embodiments, the composition is formulated for delivery by electroporation. In some embodiments, the composition is formulated for delivery by chemical method. In some embodiments, the pharmaceutical compositions comprise a virus vector or a non-viral vector.

Pharmaceutical compositions described herein comprise a salt. In some embodiments, the salt is a sodium salt. In some embodiments, the salt is a potassium salt. In some embodiments, the salt is a magnesium salt. In some embodiments, the salt is NaCl. In some embodiments, the salt is KNO3. In some embodiments, the salt is Mg2+SO42-.

Pharmaceutical compositions described herein are in the form of a solution (e.g., a liquid). In some embodiments, the solution is formulated for injection, e.g., intravenous or subcutaneous injection. In some embodiments, the pH of the solution is about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9. In some embodiments, the pH is 7 to 7.5, 7.5 to 8, 8 to 8.5, 8.5 to 9, or 7 to 8.5. In some cases, the pH of the solution is less than 7. In some cases, the pH is greater than 7.

### III. Methods of Modifying Nucleic Acid Expression

Disclosed herein, in some aspects, are methods for modifying the expression of a target nucleic acid in a cell or subject. In some embodiments, methods comprise preventing, reducing, or abolishing expression of a target nucleic acid. Methods may comprise introducing an epigenetic modification (e.g., methylation, demethylation, acetylation, or deacetylation) to a target nucleic acid.

Methods may comprise contacting a target nucleic acid with one or more components, compositions or systems described herein. In some embodiments, methods comprise contacting a target nucleic acid with at least one of: (a) an effector protein, or a nucleic acid encoding the effector protein; (b) an effector partner or nucleic acid encoding the effector partner; and (c) a guide nucleic acid or a nucleic acid encoding a guide nucleic acid. In some embodiments, the subject is a mammal. In some embodiments, the subject is a human. In some embodiment, a cell includes the progeny of the cell which has been modified by the methods described herein. It is understood that the progeny of a single cell may not necessarily be completely identical in morphology or in genomic or total DNA complement as the original parent, due to natural, accidental, or deliberate mutation.

Methods of modifying nucleic acid expression may comprise contacting a cell or subject with a composition, system or component thereof, via a method selected from: viral infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al. Adv Drug Deliv Rev. 2012 Sep. 13. pii: S0169-409X(12)00283-9. doi: 10.1016/j.addr.2012.09.023). In some embodiments, the nucleic acid and/or protein(s) are introduced into a disease cell comprised in a pharmaceutical composition comprising the guide nucleic acid, the effector protein, a pharmaceutically acceptable excipient, or combinations thereof. In some embodiments, methods comprise contacting a cell or subject with one or more nucleic acids, such as a nucleic acid encoding an effector protein. Any suitable method may be used to introduce a nucleic acid into a cell. Suitable methods include, for example, viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like. Further methods are described throughout. Introducing one or more nucleic acids into a cell may occur in any culture media and under any culture conditions that promote the survival of the cells. Introducing one or more nucleic acids into a cell may be carried out in vivo or ex vivo. Introducing one or more nucleic acids into a cell may be carried out in vitro.

In some embodiments, methods comprise contacting a cell with an RNA encoding an effector protein. In some embodiments, methods comprise administering an RNA encoding an effector protein to a subject. In some embodiments, methods comprise contacting a cell with an RNA encoding an effector partner. In some embodiments, methods comprise administering an RNA encoding an effector partner to a subject. The RNA may be provided by direct chemical synthesis or may be transcribed in vitro from a DNA (e.g., encoding the effector protein). Once synthesized, the RNA may be introduced into a cell by way of any suitable technique for introducing nucleic acids into cells (e.g., microinjection, electroporation, transfection, etc.).

Methods may comprise contacting a cell or subject with at least one expression vector. Methods for contacting cells with vectors include but are not limited to electroporation, calcium chloride transfection, microinjection, lipofection, micro-injection, contact with the cell or particle that comprises a molecule of interest, or a package of cells or particles that comprise molecules of interest. In some embodiments, the nucleic acid expression vector is a viral vector. Viral vectors include, but are not limited to, retroviruses, adenoviruses, adeno-associated viruses, and herpes simplex viruses. In some embodiments, the viral vector is a replication-defective viral vector, comprising an insertion of a therapeutic gene inserted in genes essential to the lytic cycle, preventing the virus from replicating and exerting cytotoxic effects. In some embodiments, the viral vector is an adeno associated viral (AAV) vector. In some embodiments, the nucleic acid expression vector is a non-viral vector.

### IV. Methods of Treating a Disease or Disorder

Described herein are methods for treating a disease in a subject by contacting a target nucleic acid with a composition or system described herein, wherein the target nucleic acid is associated with a gene or expression of a gene related to the disease. In some embodiments, methods comprise treating, preventing, or inhibiting a disease or disorder associated with a mutation or aberrant expression of a gene. In some embodiments, methods for treating a disease or disorder comprise methods of modifying expression of a nucleic acid described herein.

Methods may comprise administration of a composition(s) or component(s) of a system described herein. In some embodiments, the composition(s) or component(s) of the system comprises use of a recombinant nucleic acid (DNA or RNA), administered for the purpose of modifying the expression of a target nucleic acid. In some embodiments, the composition or component of the system comprises use of a vector to introduce a functional gene or transgene. In some embodiments, vectors comprise nonviral vectors, including cationic polymers, cationic lipids, or bio-responsive polymers. In some embodiments, the bio-responsive polymer exploits chemical-physical properties of the endosomal environment (e.g., pH) to preferentially release the genetic material in the intracellular space. In some embodiments, vectors comprise viral vectors, including retroviruses, adenoviruses, adeno-associated viruses, and herpes simplex viruses. In some embodiments, the vector comprises a replication-defective viral vector, comprising an insertion of a therapeutic gene inserted in genes essential to the lytic cycle, preventing the virus from replicating and exerting cytotoxic effects. By way of non-limiting example, the composition(s) may comprise pharmaceutical compositions described herein. Methods of gene therapy that are applicable to the compositions and systems described herein are described in more detail in Ingusci et al., “Gene Therapy Tools for Brain Diseases”, Front. Pharmacol. 10:724 (2019), which is hereby incorporated by reference in its entirety.

In some embodiments, treating, preventing, or inhibiting disease or disorder in a subject may comprise contacting a target nucleic acid associated with a particular ailment with a composition described herein. In some embodiments, the methods of treating, preventing, or inhibiting a disease or disorder comprise modulating gene expression of a target nucleic acid. In some embodiments, methods comprise increasing gene expression. In some embodiments, methods comprise activating gene expression. In some embodiments, methods comprise reducing gene expression. In some embodiments, methods comprise abolishing gene expression.

In some embodiments, the systems and compositions described herein are for use in therapy. In some embodiments, the compositions described herein are for use in treating a disease or condition described herein. Also provided is the use of the compositions described herein in the manufacture of a medicament. Also provided is the use of the compositions described herein in the manufacture of a medicament for therapeutic and/or prophylactic treatment of a disease or condition described herein.

The compositions and methods described herein may be used to treat, prevent, or inhibit a disease or syndrome in a subject. In some embodiments, the disease is a liver disease, a lung disease, an eye disease, or a muscle disease. Exemplary diseases and syndromes include but are not limited to the diseases and syndromes listed in TABLE 12.

In some embodiments, compositions and methods modify the expression of at least one gene associated with a disease described herein or the expression thereof. In some embodiments, the disease is Alzheimer's disease and the gene is selected from APP, BACE-1, PSD95, MAPT, PSEN1, PSEN2, and APOEe4. In some embodiments, the disease is Parkinson's disease and the gene is selected from SNCA, GDNF, and LRRK2. In some embodiments, the disease is congenital muscular dystrophy 1A (MDC1A) and the gene is LAMA1 or LAMA2. In some embodiments, the disease is Ullrich Congenital Muscular Dystrophy (UCMD) and the gene is selected from COL6A1, COL6A2 and COL6A3. In some embodiments, the disease is Limb Girdle Muscular Dystrophies (LGMD1B, LGMD2A, LGMD2B) and the gene is selected from LMNA, DYSF, and CAPN3. In some embodiments, the disease is Nemaline Myopathy and the gene is selected from ACTA1, NEB, TPM2, TPM3, TNNT1, TNNT3, TNNI2 and LMOD3. In some embodiments, the disease comprises Centronuclear myopathy and the gene is DNM2. In some embodiments, the disease is Huntington's disease and the gene is HTT. In some embodiments, the disease is Alpha-1 antitrypsin deficiency (AATD) and the gene is SERPINA1. In some embodiments, the disease is amyotrophic lateral sclerosis (ALS) and the gene is selected from SOD1, FUS, C90RF72, ATXN2, TARDBP, and CHCHD10. In some embodiments, the disease comprises Alexander Disease and the gene is GFAP. In some embodiments, the disease comprises anaplastic large cell lymphoma and the gene is CD30. In some embodiments, the disease comprises Angelman Syndrome and the gene is UBE3A. In some embodiments, the disease comprises calcific aortic stenosis and the gene is Apo(a). In some embodiments, the disease comprises CD3Z-associated primary T-cell immunodeficiency and the gene is CD3Z or CD247. In some embodiments, the disease comprises CD18 deficiency and the gene is ITGB2. In some embodiments, the disease comprises CD40L deficiency and the gene is CD40L. In some embodiments, the disease is congenital adrenal hyperplasia and the gene is CAH1. In some embodiments, the disease comprises CNS trauma and the gene is VEGF. In some embodiments, the disease comprises coronary heart disease and the gene is selected from FGA, FGB, and FGG. In some embodiments, the disease comprises MECP2 Duplication syndrome and Rett syndrome and the gene is MECP2. In some embodiments, the disease comprises a bleeding disorder (coagulation) and the gene is FXI. In some embodiments, the disease comprises fragile X syndrome and the gene is FMR1. In some embodiments, the disease comprises Fuchs corneal dystrophy and the gene is selected from ZEB1, SLC4A11, and LOXHD1. In some embodiments, the disease comprises GM2-Gangliosidoses (e.g., Tay Sachs Disease, Sandhoff disease) and the gene is selected from HEXA and HEXB. In some embodiments, the disease comprises Hearing loss disorders and the gene is DFNA36. In some embodiments, the disease is Pompe disease, including infantile onset Pompe disease (IOPD) and late onset Pompe disease (LOPD) and the gene is GAA. In some embodiments, the disease is Retinitis pigmentosa and the gene is selected from PDE6B, RHO, RP1, RP2, RPGR, PRPH2, IMPDHI, PRPF31, CRB1, PRPF8, TULPI, CA4, HPRPF3, ABCA4, EYS, CERKL, FSCN2, TOPORS, SNRNP200, PRCD, NR2E3, MERTK, USH2A, PROM1, KLHL7, CNGBI, TTC8, ARL6, DHDDS, BEST1, LRAT, SPARA7, CRX, CLRN1, RPE65, and WDR19. In some embodiments, the disease comprises Leber Congenital Amaurosis Type 10 and the gene is CEP290. In some embodiments, the disease is cardiovascular disease and/or lipodystrophies and the gene is selected from ABCG5, ABCG8, AGT, ANGPTL3, APOCIII, APOA1, APOL1, ARH, CDKN2B, CFB, CXCL12, FXI, FXII, GATA-4, MIA3, MKL2, MTHFDIL, MYH7, NKX2-5, NOTCH1, PKK, PCSK9, PSRC1, SMAD3, and TTR. In some embodiments, the disease is cardiovascular disease and/or lipodystrophies and the gene is ANGPTL3. In some embodiments, the disease is cardiovascular disease and/or lipodystrophies and the gene is PCSK9. In some embodiments, the disease is cardiovascular disease and/or lipodystrophies and the gene is TTR. In some embodiments, the disease is severe hypertriglyceridemia (SHTG) and the gene is APOCIII or ANGPTL4. In some embodiments, the disease comprises acromegaly and the gene is GHR. In some embodiments, the disease comprises acute myeloid leukemia and the gene is CD22. In some embodiments, the disease is diabetes and the gene is GCGR. In some embodiments, the disease is NAFLD/NASH and the gene is selected from HSD17B13, PSD3, GPAM, CIDEB, DGAT2 and PNPLA3. In some embodiments, the disease is NASH/cirrhosis and the gene is MARC1. In some embodiments, the disease is cancer and the gene is selected from STAT3, YAP1, FOXP3, AR (Prostate cancer), and IRF4 (multiple myeloma). In some embodiments, the disease is cystic fibrosis and the gene is CFTR. In some embodiments, the disease is Duchenne muscular dystrophy and the gene is DMD. In some embodiments, the disease is ornithine transcarbamylase deficiency (OTCD) and the gene is OTC. In some embodiments, the disease is congenital adrenal hyperplasia (CAH) and the gene is CYP21A2. In some embodiments, the disease is atherosclerotic cardiovascular disease (ASCVD) and the gene is LPA. In some embodiments, the disease is hepatitis B virus infection (CHB) and the gene is HBV covalently closed circular DNA (cccDNA). In some embodiments, the disease is citrullinemia type I and the gene is ASS1. In some embodiments, the disease is citrullinemia type I and the gene is SLC25A13. In some embodiments, the disease is citrullinemia type I and the gene is ASS1. In some embodiments, the disease is arginase-1 deficiency and the gene is ARG1. In some embodiments, the disease is carbamoyl phosphate synthetase I deficiency and the gene is CPS1. In some embodiments, the disease is argininosuccinic aciduria and the gene is ASL. In some embodiments, the disease comprises angioedema and the gene is PKK. In some embodiments, the disease comprises thalassemia and the gene is TMPRSS6. In some embodiments, the disease comprises achondroplasia and the gene is FGFR3. In some embodiments, the disease comprises Cri du chat syndrome and the gene is selected from CTNND2. In some embodiments, the disease comprises sickle cell anemia and the gene is Beta globin gene. In some embodiments, the disease comprises Alagille Syndrome and the gene is selected from JAG1 and NOTCH2. In some embodiments, the disease comprises Charcot-Marie-Tooth disease and the gene is selected from PMP22 and MFN2. In some embodiments, the disease comprises Crouzon syndrome and the gene is selected from FGFR2, FGFR3, and FGFR3. In some embodiments, the disease comprises Dravet Syndrome and the gene is selected from SCN1A and SCN2A. In some embodiments, the disease comprises Emery-Dreifuss syndrome and the gene is selected from EMD, LMNA, SYNE1, SYNE2, FHL1, and TMEM43. In some embodiments, the disease comprises Factor V Leiden thrombophilia and the gene is F5. In some embodiments, the disease is fabry disease and the gene is GLA. In some embodiments, the disease is facioscapulohumeral muscular dystrophy (FSHD) and the gene is FSHD1. In some embodiments, the disease is facioscapulohumeral muscular dystrophy (FSHD) and the gene is DUX4. In some embodiments, the disease comprises Fanconi anemia and the gene is selected from FANCA, FANCB, FANCC, FANCD1, FANCD2, FANCE, FANCF, FANCG, FANCI, FANCJ, FANCL, FANCM, FANCN, FANCP, FANCS, RAD51C, and XPF. In some embodiments, the disease comprises Familial Creutzfeld-Jakob disease and the gene is PRNP. In some embodiments, the disease comprises Familial Mediterranean Fever and the gene is MEFV. In some embodiments, the disease comprises Friedreich's ataxia and the gene is FXN. In some embodiments, the disease comprises Gaucher disease and the gene is GBA. In some embodiments, the disease comprises human papilloma virus (HPV) infection and the gene is HPV E7. In some embodiments, the disease comprises hemochromatosis and the gene is HFE, optionally comprising a C282Y mutation. In some embodiments, the disease comprises Hemophilia A and the gene is FVIII. In some embodiments, the disease is hereditary angioedema and the gene is SERPING1 or KLKB1. In some embodiments, the disease comprises histiocytosis and the gene is CD1. In some embodiments, the disease comprises immunodeficiency 17 and the gene is CD3D. In some embodiments, the disease comprises immunodeficiency 13 and the gene is CD4. In some embodiments, the disease comprises Common Variable Immunodeficiency and the gene is selected from CD19 and CD81. In some embodiments, the disease comprises Joubert syndrome and the gene is selected from INPP5E, TMEM216, AHII, NPHPI, CEP290, TMEM67, RPGRIPIL, ARL13B, CC2D2A, OFD1, TMEM138, TCTN3, ZNF423, and AMRC9. In some embodiments, the disease comprises leukocyte adhesion deficiency and the gene is CD18. In some embodiments, the disease comprises Li-Fraumeni syndrome and the gene is TP53. In some embodiments, the disease comprises lymphoproliferative syndrome and the gene is CD27. In some embodiments, the disease comprises Lynch syndrome and the gene is selected from MSH2, MLH1, MSH6, PMS2, PMS1, TGFBR2, and MLH3. In some embodiments, the disease comprises mantle cell lymphoma and the gene is CD5. In some embodiments, the disease comprises Marfan syndrome and the gene is FBN1. In some embodiments, the disease comprises mastocytosis and the gene is CD2. In some embodiments, the disease comprises methylmalonic acidemia and the gene is selected from MMAA, MMAB, and MUT. In some embodiments, the disease is mycosis fungoides and the gene is CD7. In some embodiments, the disease is myotonic dystrophy and the gene is selected from CNBP and DMPK. In some embodiments, the disease comprises neurofibromatosis and the gene is selected from NF1, and NF2. In some embodiments, the disease comprises osteogenesis imperfecta and the gene is selected from COL1A1, COL1A2, and IFITM5. In some embodiments, the disease is non-small cell lung cancer and the gene is selected from KRAS, EGFR, ALK, METex14, BRAF V600E, ROSI, RET, and NTRK. In some embodiments, the disease comprises Peutz-Jeghers syndrome and the gene is STKI1. In some embodiments, the disease comprises polycystic kidney disease and the gene is selected from PKD1 and PKD2. In some embodiments, the disease comprises Severe Combined Immune Deficiency and the gene is selected from IL7R, RAG1, and JAK3. In some embodiments, the disease comprises PRKAG2 cardiac syndrome and the gene is PRKAG2. In some embodiments, the disease comprises spinocerebellar ataxia and the gene is selected from ATXN1, ATXN2, ATXN3, PLEKHG4, SPTBN2, CACNAIA, ATXN7, ATXN8OS, ATXN10, TTBK2, PPP2R2B, KCNC3, PRKCG, ITPRI, TBP, KCND3, and FGF14. In some embodiments, the disease is thrombophilia due to antithrombin III deficiency and the gene is SERPINC1. In some embodiments the disease is spinal muscular atrophy and the gene is SMN1. In some embodiments, the disease comprises Usher Syndrome and the gene is selected from MYO7A, USHIC, CDH23, PCDH15, USHIG, USH2A, GPR98, DFNB31, and CLRN1. In some embodiments, the disease comprises von Willebrand disease and the gene is VWF. In some embodiments, the disease comprises Waardenburg syndrome and the gene is selected from PAX3, MITF, WS2B, WS2C, SNAI2, EDNRB, EDN3, and SOX10. In some embodiments, the disease comprises Wiskott-Aldrich Syndrome and the gene is WAS. In some embodiments, the disease comprises von Hippel-Lindau disease and the gene is VHL. In some embodiments, the disease comprises Wilson disease and the gene is ATP7B. In some embodiments, the disease comprises Zellweger syndrome and the gene is selected from PEX1, PEX2, PEX3, PEX5, PEX6, PEX10, PEX12, PEX13, PEX14, PEX16, PEX19, and PEX26. In some embodiments, the disease comprises infantile myofibromatosis and the gene is CD34. In some embodiments, the disease comprises platelet glycoprotein IV deficiency and the gene is CD36. In some embodiments, the disease comprises immunodeficiency with hyper-IgM type 3 and the gene is CD40. In some embodiments, the disease comprises hemolytic uremic syndrome and the gene is CD46. In some embodiments, the disease comprises complement hyperactivation, angiopathic thrombosis, or protein-losing enteropathy and the gene is CD55. In some embodiments, the disease comprises hemolytic anemia and the gene is CD59. In some embodiments, the disease comprises calcification of joints and arteries and the gene is CD73. In some embodiments, the disease comprises immunoglobulin alpha deficiency and the gene is CD79A. In some embodiments, the disease comprises C syndrome and the gene is CD96. In some embodiments, the disease comprises hairy cell leukemia and the gene is CD123. In some embodiments, the disease comprises histiocytic sarcoma and the gene is CD163. In some embodiments, the disease comprises autosomal dominant deafness and the gene is CD164. In some embodiments, the disease comprises immunodeficiency 25 and the gene is CD247. In some embodiments, the disease comprises methymalonic acidemia due to transcobalamin receptor defect and the gene is CD320.

### Cancer

In some embodiments, the disease is cancer. Non-limiting examples of cancers include: acute lymphoblastic leukemia; acute lymphoblastic lymphoma; acute lymphocytic leukemia; acute myelogenous leukemia; acute myeloid leukemia (adult/childhood); adrenocortical carcinoma; AIDS-related cancers; AIDS-related lymphoma; anal cancer; appendix cancer; astrocytoma; atypical teratoid/rhabdoid tumor; basal-cell carcinoma; bile duct cancer; extrahepatic (cholangiocarcinoma); bladder cancer; bone osteosarcoma/malignant fibrous histiocytoma; brain cancer (adult/childhood); brain tumor; cerebellar astrocytoma (adult/childhood); brain tumor, cerebral astrocytoma/malignant glioma brain tumor; brain tumor, ependymoma; brain tumor, medulloblastoma; brain tumor, supratentorial primitive neuroectodermal tumors; brain tumor, visual pathway and hypothalamic glioma; brainstem glioma; breast cancer; bronchial adenomas/carcinoids; bronchial tumor; Burkitt lymphoma; cancer of childhood; carcinoid gastrointestinal tumor; carcinoid tumor; carcinoma of adult, unknown primary site; carcinoma of unknown primary; central nervous system embryonal tumor; central nervous system lymphoma, primary; cervical cancer; childhood adrenocortical carcinoma; childhood cancers; childhood cerebral astrocytoma; chordoma, childhood; chronic lymphocytic leukemia; chronic myelogenous leukemia; chronic myeloid leukemia; chronic myeloproliferative disorders; colon cancer; colorectal cancer; craniopharyngioma; cutaneous T-cell lymphoma; desmoplastic small round cell tumor; emphysema; endometrial cancer; ependymoblastoma; ependymoma; esophageal cancer; Ewing sarcoma in the Ewing family of tumors; extracranial germ cell tumor; extragonadal germ cell tumor; extrahepatic bile duct cancer; gallbladder cancer; gastric (stomach) cancer; gastric carcinoid; gastrointestinal carcinoid tumor; gastrointestinal stromal tumor; germ cell tumor: extracranial, extragonadal, or ovarian gestational trophoblastic tumor; gestational trophoblastic tumor, unknown primary site; glioma; glioma of the brain stem; glioma, childhood visual pathway and hypothalamic; hairy cell leukemia; head and neck cancer; heart cancer; hepatocellular (liver cancer); Hodgkin's lymphoma; hypopharyngeal cancer; hypothalamic and visual pathway glioma; intraocular melanoma; islet cell carcinoma (endocrine pancreas); Kaposi Sarcoma; kidney cancer (renal cell cancer); Langerhans cell histiocytosis; laryngeal cancer; lip and oral cavity cancer; liposarcoma; liver cancer (primary); lung cancer, non-small cell; lung cancer, small cell; lymphoma, primary central nervous system; macroglobulinemia, Waldenstrom; male breast cancer; malignant fibrous histiocytoma of bone/osteosarcoma; medulloblastoma; medulloepithelioma; melanoma; melanoma, intraocular (eye); Merkel cell cancer; Merkel cell skin carcinoma; mesothelioma; mesothelioma, adult malignant; metastatic squamous neck cancer with occult primary; mouth cancer; multiple endocrine neoplasia syndrome; multiple myeloma/plasma cell neoplasm; mycosis fungoides, myelodysplastic syndromes; myelodysplastic/myeloproliferative diseases; myelogenous leukemia, chronic; myeloid leukemia, adult acute; myeloid leukemia, childhood acute; myeloma, multiple (cancer of the bone-marrow); myeloproliferative disorders, chronic; nasal cavity and paranasal sinus cancer; nasopharyngeal carcinoma; neuroblastoma, non-small cell lung cancer; non-Hodgkin's lymphoma; oligodendroglioma; oral cancer; oral cavity cancer; oropharyngeal cancer; osteosarcoma/malignant fibrous histiocytoma of bone; ovarian cancer; ovarian epithelial cancer (surface epithelial-stromal tumor); ovarian germ cell tumor; ovarian low malignant potential tumor; pancreatic cancer; pancreatic cancer; pituitary tumor, islet cell; papillomatosis; paranasal sinus and nasal cavity cancer; parathyroid cancer; penile cancer; pharyngeal cancer; pheochromocytoma; pineal astrocytoma; pineal germinoma; pineal parenchymal tumors of intermediate differentiation; pineoblastoma and supratentorial primitive neuroectodermal tumors; pituitary tumor; pituitary adenoma; plasma cell neoplasia/multiple myeloma; pleuropulmonary blastoma; primary central nervous system lymphoma; prostate cancer; rectal cancer; renal cell carcinoma (kidney cancer); renal pelvis and ureter, transitional cell cancer; NUT midline carcinoma; retinoblastoma; rhabdomyosarcoma, childhood; salivary gland cancer; sarcoma, Ewing family of tumors; Sèzary syndrome; skin cancer (melanoma); skin cancer (non-melanoma); small cell lung cancer; small intestine cancer soft tissue sarcoma; soft tissue sarcoma; spinal cord tumor; squamous cell carcinoma; squamous neck cancer with occult primary, metastatic; stomach (gastric) cancer; supratentorial primitive neuroectodermal tumor; T-cell lymphoma, cutaneous (Mycosis Fungoides and Sèzary syndrome); testicular cancer; throat cancer; thymoma; thymoma and thymic carcinoma; thyroid cancer; thyroid cancer, childhood; transitional cell cancer of the renal pelvis and ureter; urethral cancer; uterine cancer, endometrial; uterine sarcoma; vaginal cancer; vulvar cancer; and Wilms Tumor.

In some embodiments, mutations are associated with cancer or are causative of cancer. The target nucleic acid, in some embodiments, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, a gene associated with cell cycle, or combinations thereof. Non-limiting examples of genes comprising a mutation associated with cancer are ABL, ACE, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CCR5, CDC73, CDHL, CDK4, CDKN1B, CDKN1C, CDKN2A, CEBPA, CHEK2, CREBBP, CTNNA1, DBL, DEK/CAN, DICER1, DIS3L2, E2A/PBX1, EGFR, ENI/HRX, EPCAM, ERG/TLS, ERBB, ERBB-2, ETS-1, EWS/FLI-1, FH, FKRP, FLCN, FMS, FOS, FPS, GATA2, GCG, GLI, GPC3, GPGSP, GREM1, HER2/neu, HOX11, HOXB13, HRAS, HST, IL-3, INT-2, JAK1, JUN, KIT, KS3, K-SAM, LBC, LCK, LMO1, LMO2, L-MYC, LYL-1, LYT-10, LYT-10/Cal, MAS, MAX, MDM-2, MEN1, MET, MITF, MLH1, MLL, MOS, MSHL, MSH2, MSH3, MSH6, MTG8/AML1, MUTYH, MYB, MYH11/CBFB, NBN, NEU, NFL, NF2, N-MYC, NTHL1, OST, PALB2, PAX-5, PBX1/E2A, PCDCl, PDGFRA, PHOX2B, PIM-1, PMS2, POLD1, POLE, POT1, PPARG, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHAF2, SDHB, SDHC, SDHD, SET/CAN, SIS, SKI, SMAD4, SMARCA4, SMARCB1, SMARCEL, SRC, STKI1, SUFU, TAL1, TAL2, TAN-1, TIAM1, TERC, TERT, TIMP3, TMEM127, TNF, TP53, TRAC, TSCL, TSC2, TRK, VHL, WRN, and WTI. Non-limiting examples of oncogenes are KRAS, NRAS, BRAF, MYC, CTNNB1, and EGFR. In some embodiments, the oncogene is a gene that encodes a cyclin dependent kinase (CDK). Non-limiting examples of CDKs are Cdk1, Cdk4, Cdk5, Cdk7, Cdk8, Cdk9, Cdk11 and Cdk20. Non-limiting examples of tumor suppressor genes are TP53, RB1, and PTEN.

### Infections

Described herein are compositions, systems, and methods for treating an infection in a subject. Infections may be caused by a pathogen (e.g., bacteria, viruses, fungi, and parasites). Compositions, systems, and methods may modify a target nucleic acid associated with the pathogen or parasite causing the infection. In some embodiments, the target nucleic acid may be in the pathogen or parasite itself or in a cell, tissue or organ of the subject that the pathogen or parasite infects. In some embodiments, the methods described herein include treating an infection caused by one or more bacterial pathogens. Non-limiting examples of bacterial pathogens include Acholeplasma laidlawii, Brucella abortus, Chlamydia psittaci, Chlamydia trachomatis, Cryptococcus neoformans, Escherichia coli, Legionella pneumophila, Lyme disease spirochetes, methicillin-resistant Staphylococcus aureus, Mycobacterium leprae, Mycobacterium tuberculosis, Mycoplasma arginini, Mycoplasma arthritidis, Mycoplasma genitalium, Mycoplasma hyorhinis, Mycoplasma orale, Mycoplasma pneumoniae, Mycoplasma salivarium, Neisseria gonorrhoeae, Neisseria meningitidis, Pneumococcus, Pseudomonas aeruginosa, sexually transmitted infection, Streptococcus agalactiae, Streptococcus pyogenes, and Treponema pallidum.

In some embodiments, compositions, systems or methods described herein may treat an infection caused by one or more viral pathogens. Non-limiting examples of viral pathogens include adenovirus, blue tongue virus, chikungunya, coronavirus (e.g., SARS-CoV-2), cytomegalovirus, Dengue virus, Ebola, Epstein-Barr virus, feline leukemia virus, Hemophilus influenzae B, Hepatitis Virus A, Hepatitis Virus B, Hepatitis Virus C, herpes simplex virus I, herpes simplex virus II, human papillomavirus (HPV) including HPV16 and HPV18, human serum parvo-like virus, human T-cell leukemia viruses, immunodeficiency virus (e.g., HIV), influenza virus, lymphocytic choriomeningitis virus, measles virus, mouse mammary tumor virus, mumps virus, murine leukemia virus, polio virus, rabies virus, Reovirus, respiratory syncytial virus (RSV), rubella virus, Sendai virus, simian virus 40, Sindbis virus, varicella-zoster virus, vesicular stomatitis virus, wart virus, West Nile virus, yellow fever virus, or any combination thereof.

In some embodiments, compositions, systems or methods described herein may treat an infection caused by one or more parasites. Non-limiting examples of parasites include helminths, annelids, platyhelminthes, nematodes, and thorny-headed worms. In some embodiments, parasitic pathogens comprise, without limitation, Babesia bovis, Echinococcus granulosus, Eimeria tenella, Leishmania tropica, Mesocestoides corti, Onchocerca volvulus, Plasmodium falciparum, Plasmodium vivax, Schistosoma japonicum, Schistosoma mansoni, Schistosoma spp., Taenia hydatigena, Taenia ovis, Taenia saginata, Theileria parva, Toxoplasma gondii, Toxoplasma spp., Trichinella spiralis, Trichomonas vaginalis, Trypanosoma brucei, Trypanosoma cruzi, Trypanosoma rangeli, Trypanosoma rhodesiense, Balantidium coli, Entamoeba histolytica, Giardia spp., Isospora spp., Trichomonas spp., or any combination thereof.

### Sequences and Tables

TABLE 1 provides illustrative amino acid sequences of effector proteins that are useful in the compositions, systems and methods described herein.

TABLE 1
EXEMPLARY AMINO ACID SEQUENCE(S) OF EFFECTOR PROTEIN(S)

| | SEQ | |
|---|---|---|
| | ID | |
| Name | NO: | Sequence |
| CasPhi.12 | 1 | MIKPTVSQFLTPGFKLIRNHSRTAGLKLKNEGEEA |
| | | CKKFVRENEIPKDECPNFQGGPAIANIIAKSREFT |
| | | EWEIYQSSLAIQEVIFTLPKDKLPEPILKEEWRAQ |
| | | WLSEHGLDTVPYKEAAGLNLIIKNAVNTYKGVQVK |
| | | VDNKNKNNLAKINRKNEIAKLNGEQEISFEEIKAF |
| | | DDKGYLLQKPSPNKSIYCYQSVSPKPFITSKYHNV |
| | | NLPEEYIGYYRKSNEPIVSPYQFDRLRIPIGEPGY |
| | | VPKWQYTFLSKKENKRRKLSKRIKNVSPILGIICI |
| | | KKDWCVFDMRGLLRTNHWKKYHKPTDSINDLFDYF |
| | | TGDPVIDTKANVVRFRYKMENGIVNYKPVREKKGK |
| | | ELLENICDQNGSCKLATVDVGQNNPVAIGLFELKK |
| | | VNGELTKTLISRHPTPIDFCNKITAYRERYDKLES |
| | | SIKLDAIKQLTSEQKIEVDNYNNNFTPQNTKQIVC |
| | | SKLNINPNDLPWDKMISGTHFISEKAQVSNKSEIY |
| | | FTSTDKGKTKDVMKSDYKWFQDYKPKLSKEVRDAL |
| | | SDIEWRLRRESLEFNKLSKSREQDARQLANWISSM |
| | | CDVIGIENLVKKNNFFGGSGKREPGWDNFYKPKKE |
| | | NRWWINAIHKALTELSQNKGKRVILLPAMRTSITC |
| | | PKCKYCDSKNRNGEKFNCLKCGIELNADIDVATEN |
| | | LATVAITAQSMPKPTCERSGDAKKPVRARKAKAPE |
| | | FHDKLAPSYTVVLREAV |
| dCasPhi12 | 2 | MIKPTVSQFLTPGFKLIRNHSRTAGRKLKNEGEEA |
| (L26R_E567Q) | | CKKFVRENEIPKDECPNFQGGPAIANIIAKSREFT |
| | | EWEIYQSSLAIQEVIFTLPKDKLPEPILKEEWRAQ |
| | | WLSEHGLDTVPYKEAAGLNLIIKNAVNTYKGVQVK |
| | | VDNKNKNNLAKINRKNEIAKLNGEQEISFEEIKAF |
| | | DDKGYLLQKPSPNKSIYCYQSVSPKPFITSKYHNV |
| | | NLPEEYIGYYRKSNEPIVSPYQFDRLRIPIGEPGY |
| | | VPKWQYTFLSKKENKRRKLSKRIKNVSPILGIICI |
| | | KKDWCVFDMRGLLRTNHWKKYHKPTDSINDLFDYF |
| | | TGDPVIDTKANVVRFRYKMENGIVNYKPVREKKGK |
| | | ELLENICDQNGSCKLATVDVGQNNPVAIGLFELKK |
| | | VNGELTKTLISRHPTPIDFCNKITAYRERYDKLES |
| | | SIKLDAIKQLTSEQKIEVDNYNNNFTPQNTKQIVC |
| | | SKLNINPNDLPWDKMISGTHFISEKAQVSNKSEIY |
| | | FTSTDKGKTKDVMKSDYKWFQDYKPKLSKEVRDAL |
| | | SDIEWRLRRESLEFNKLSKSREQDARQLANWISSM |
| | | CDVIGIQNLVKKNNFFGGSGKREPGWDNFYKPKKE |
| | | NRWWINAIHKALTELSQNKGKRVILLPAMRTSITC |
| | | PKCKYCDSKNRNGEKFNCLKCGIELNADIDVATEN |
| | | LATVAITAQSMPKPTCERSGDAKKPVRARKAKAPE |
| | | FHDKLAPSYTVVLREAV |
| CasM.265466 | 35 | MSVLTRKVQLIPVGDKEERDRVYKYLRDGIEAQNR |
| | | AMNLYMSGLYFAAINEASKEDRKELNQLYSRIATS |
| | | SKGSAYTTDIEFPTGLASTSTLSMAVRQDFTKSLK |
| | | DGLMYGRVSLPTYRKDNPLFVDVRFVALRGTKQKY |
| | | NGLYHEYKSHTEFLDNLYSSDLKVYIKFANDITFQ |
| | | VIFGNPRKSSALRSEFQNIFEEYYKVCQSSIQFSG |
| | | TKIILNMAMDIPDKEIELDEDVCVGVDLGIAIPAV |
| | | CALNKNRYSRVSIGSKEDFLRVRTKIRNQRKRLQT |
| | | NLKSSNGGHGRKKKMKPMDRFRDYEANWVQNYNHY |
| | | VSRQVVDFAVKNKAKYINLENLEGIRDDVKNEWLL |
| | | SNWSYYQLQQYITYKAKTYGIEVRKINPYHTSQRC |
| | | SCCGYEDAGNRPKKEKGQAYFKCLKCGEEMNADFN |
| | | AARNIAMSTEFQSGKKTKKQKKEQHENK |
| dCasM.265466 | 2950 | MSVLTRKVQLIPVGDKEERDRVYKYLRDGIEAQNR |
| (D220R_E335Q) | | AMNLYMSGLYFAAINEASKEDRKELNQLYSRIATS |
| | | SKGSAYTTDIEFPTGLASTSTLSMAVRQDFTKSLK |
| | | DGLMYGRVSLPTYRKDNPLFVDVRFVALRGTKQKY |
| | | NGLYHEYKSHTEFLDNLYSSDLKVYIKFANDITFQ |
| | | VIFGNPRKSSALRSEFQNIFEEYYKVCQSSIQFSG |
| | | TKIILNMAMRIPDKEIELDEDVCVGVDLGIAIPAV |
| | | CALNKNRYSRVSIGSKEDFLRVRTKIRNQRKRLQT |
| | | NLKSSNGGHGRKKKMKPMDRFRDYEANWVQNYNHY |
| | | VSRQVVDFAVKNKAKYINLQNLEGIRDDVKNEWLL |
| | | SNWSYYQLQQYITYKAKTYGIEVRKINPYHTSQRC |
| | | SCCGYEDAGNRPKKEKGQAYFKCLKCGEEMNADFN |
| | | AARNIAMSTEFQSGKKTKKQKKEQHENK |
| dCasM.288886 | 3010 | MSLITRKIQIYVSEEDKELKKQFVHTIYDWRNQIR |
| | | RAANIVVSHKFTQENIKDFVYLKDEIKEKFYVKDI |
| | | LKEGKGMSEQNTTYRVLADLLKGKIPSDIYSCLNQ |
| | | AVAKTFKETKSEIFKGETALRSYKNNIPVPFSSDS |
| | | IRNLHWEEKDKRFYFTLFGIPFATALGADRSNNKV |
| | | IIERVLSGEYKLCQSSIQIDDRKKKMFLLLCVDIP |
| | | KKEVKLNPDKKLYAFLGVFNPIVCTTEITDDLDAK |
| | | NPYKVWEIGTEEEFNHRRRQIQEAVKRCQINSRYT |
| | | EGGKGIKKKTKAIDRFHEKEKNYIDTKLHTYSRML |
| | | VDLAIKNECSEIVLMKQKEREEMAKEDNQNGKPFV |
| | | LRNWSYYGLKDKISYKAKMVGIGITVE |
| dCasM.288094 | 3011 | MIITRKIEIYVNESDKDKKAEYYDRLFKILYTVHR |
| | | FANLCASHLYFADNIADFVYLTTDARDKVKFSENL |
| | | LTDSKKKDEGILISSYDNLVYELSKTFPEEERKGI |
| | | TKILADTGNRIIKAYKKKTDGESERLMIKRGKRSV |
| | | KNYKLNIPIPFSKQSFKIEKSGNDYIFDFYHIPFK |
| | | TKCGKDKSGNRIIINRLLAGEYDLSDSSIQYAKKY |
| | | DRETGKAVSKWMLQLCVNIPETRLQAKKGVKVKAD |
| | | LNLVVPIIATCGKITREFGTKEEFLYQRIQIQSKL |
| | | KNLQKSLRWANGGNGRKAKLQAIERFKLKEKNYIK |
| | | TKLHAYSRALVNFAIKERAEKIELVNQKLKEEMAK |
| | | DLEPVLRNWSYYGLKQMIEYKAMRVGINVELN |
| dCasM.2401960 | 3012 | MPTVTRKIQLFFDIGDNPKELKALFKKWYEWQRIV |
| | | RKAANQISTHLYIQDNIKDFFYITEDVKIKLKKSA |
| | | DDVAGILNTSKDNTTYQVLSKLFKGDCPMGMLSGL |
| | | SSVIGNTYKKEKKEMFLGNKSLRSYKKDIPLPMRV |
| | | RDSSNWTKHEDGNYSFSVYGTPFKTWFGHEKKKVT |
| | | NDTHTKEYMLDMAMKGEVKFCDSSIQIKDNKFFLL |
| | | AVESWEKDVIKVDTENVADCYLSIKYPIIIKEGKD |
| | | RMYTIGSEEEYLHGRNSIKAALQRVQTGAKYNQGG |
| | | DGRELKLQSIERFKAAEKNFIENRMHVYARELIKY |
| | | CLKHGKGKIVLNNYNEAKEQTHEGTEEANLLLASW |
| | | SYYNLSDKIANKAAYHSIVVEKN |

TABLE 2 provides illustrative sequences of exemplary heterologous polypeptide modifications of effector protein(s) that are useful in the compositions, systems and methods described herein.

TABLE 2
EXEMPLARY NUCLEAR LOCALIZATOIN SIGNALS

| SEQ ID NO: | Description | Sequence* |
|---|---|---|
| 3 | SV40 NLS | MAPKKKRKVGIHGVPAA |
| 4 | Nucleoplasm NLS | KRPAATKKAGQAKKKKEF |

TABLE 3 provides illustrative PAM sequences that are useful in the compositions, systems and methods described herein.

TABLE 3
EXEMPLARY PAM SEQUENCES

| Effector Protein | PAM Sequence* |
|---|---|
| SEQ ID NO: | (5′→3′) |
| 1 | NTTN |
| 3 | NNTR, NNTN |

*wherein each N is independently any one of A, G, C, or T

TABLE 4 provides exemplary effector partners.

TABLE 4
EXEMPLARY EFFECTOR PARTNERS

| | SEQ ID | |
|---|---|---|
| Name | NO: | Sequence |
| DNMT3A | 10 | MNHDQEFDPPKVYPPVPAEKRKPIRVLSLFDGIATGLLVLKDLGIQVDRYIASEVCEDSITVGMV |
| | | RHQGKIMYVGDVRSVTQKHIQEWGPFDLVIGGSPCNDLSIVNPARKGLYEGTGRLFFEFYRLLH |
| | | DARPKEGDDRPFFWLFENVVAMGVSDKRDISRFLESNPVMIDAKEVSAAHRARYFWGNLPGM |
| | | NRPLASTVNDKLELQECLEHGRIAKFSKVRTITTRSNSIKQGKDQHFPVFMNEKEDILWCTEMER |
| | | VFGFPVHYTDVSNMSRLARQRLLGRSWSVPVIRHLFAPLKEYFACV |
| DNMT3L | 11 | MGPMEIYKTVSAWKRQPVRVLSLFRNIDKVLKSLGFLESGSGSGGGTLKYVEDVTNVVRRDVE |
| | | KWGPFDLVYGSTQPLGSSCDRCPGWYMFQFHRILQYALPRQESQRPFFWIFMDNLLLTEDDQET |
| | | TTRFLQTEAVTLQDVRGRDYQNAMRVWSNIPGLKSKHAPLTPKEEEYLQAQVRSRSKLDAPKV |
| | | DLLVKNCLLPLREYFKYFSQNSLPL |
| KRAB | 12 | MDAKSLTAWSRTLVTFKDVFVDFTREEWKLLDTAQQIVYRNVMLENYKNLVSLGYQLTKPDV |
| (ZNF10) | | ILRLEKGEEPWLV |
| KRAB | 3005 | MDAKSLTAWSRTLVTFKDVFVDFTREEWKLLDTAQQIVYRNVMLENYKNLVSLGYQLTKPDV |
| (ZNF10)- | | ILRLEKGEEPWLVESGPGTSTEPSEGSAPGSPAMKDKVEKEKSEKETTSKKNSHKKTRPRLK |
| YAF2 | | NVDRSSAQHLEVTVGDLTVIITDFKEKTKSPPASSAASADQHSQSGSSSDNT |
| | | wherein bold represents KRAB(ZNF10), |
| | | italics represent YAF2 (Yaf2/RYBP C-terminal binding motif), |
| | | and regular font represents a linker |
| KRAB | 3006 | MDAKSLTAWSRTLVTFKDVFVDFTREEWKLLDTAQQIVYRNVMLENYKNLVSLGYQLTKPDV |
| (ZNF10)- | | ILRLEKGEEPWLVESGPGTSTEPSEGSAPGSPAMAEAFGDSEEDGEDVFEVEKILDMKTEGG |
| MPP8 | | KVLYKVRWKGYTSDDDTWEPEIHLEDCKEVLLEFRKKIAENKAKAVRKDIQR |
| | | wherein bold represents KRAB(ZNF10), |
| | | italics represent MPP8 chromodomain, and regular font |
| | | represents a linker |
| KRAB | 3007 | MDAKSLTAWSRTLVTFKDVFVDFTREEWKLLDTAQQIVYRNVMLENYKNLVSLGYQLTKPDV |
| (ZNF10)- | | ILRLEKGEEPWLVESGPGTSTEPSEGSAPGSPAMPSEANSIQSANATTKTSETNHTSRPRLK |
| RYBP | | NVDRSTAQQLAVTVGNVTVIITDFKEKTRSSSTSSSTVTSSAGSEQQNQSSS |
| | | wherein bold represents KRAB(ZNF10), |
| | | italics represent RYBP (Yaf2/RYBP C-terminal binding motif), |
| | | and regular font represents a linker |
| CBX3VD | 3008 | MEEAEPDEFVVEKVLDRRVVNGKVEYFLKWEGFTDADNTWEPEENLDCPELIEAFLNSQKSR |
| -KRAB | | SSGGGSGGSGSMNNSQGRVTFEDVTVNFTQGEWQRLNPEQRNLYRDVMLENYSNLVSVGQGE |
| (ZIM3) | | TTKPDVILRLEQGKEPWLEEEEVLGSGRAEKNGDIGGQIWKPKDVKESL |
| | | wherein bold represents CBX3 (chromodomain, Q9D, K33E), |
| | | italics represent ZIM3 (KRAB domain), |
| | | and regular font represents a linker |
| CBX5VD | 3009 | MEDEYVVEKVLDRRVVKGQVEYLLKWEGFSEEHNTWEPEKNLDCPELISEFMKKYKKMKEGE |
| -KRAB | | NSRSSGGGSGGSGSMNNSQGRVTFEDVTVNFTQGEWQRLNPEQRNLYRDVMLENYSNLVSVG |
| (ZIM3) | | QGETTKPDVILRLEQGKEPWLEEEEVLGSGRAEKNGDIGGQIWKPKDVKESL |
| | | wherein bold represents CBX5 (chromodomain, Q9D, K33E), |
| | | italics represent ZIM3 (KRAB domain), |
| | | and regular font represents a linker |

TABLE 5 provides exemplary peptide linkers.

TABLE 5
EXEMPLARY PEPTIDE LINKERS

| Name | SEQ ID NO | Sequence |
|---|---|---|
| XTEN20 | 13 | GSPAGSPTSTEEGTSESATP |
| XTEN54 | 14 | GGPSSGAPPPSGGSPAGSPTST |
| | | EEGTSESATPESGPGTSTEPSE |
| | | GSAPGSPAGS |
| DNMT linker | 15 | SSGNSNANSRGPSFSSGLVPLS |
| | | LRGSH |

TABLE 6 provides exemplary fusion protein sequences.

TABLE 6
FUSION CONSTRUCTS TESTED FOR THE REPRESSION OF GFP EXPRESSION

| | | SEQ |
|---|---|---|
| | | ID |
| Description | Amino Acid Sequence | NO |
| DNMT3A- | MNHDQEFDPPKVYPPVPAEKRKPIRVLSLFDGIATGLLVLKDLGIQVDRYIASEV | 16 |
| DNMT3L- | CEDSITVGMVRHQGKIMYVGDVRSVTQKHIQEWGPFDLVIGGSPCNDLSIVNPAR | |
| NLS- | KGLYEGTGRLFFEFYRLLHDARPKEGDDRPFFWLFENVVAMGVSDKRDISRFLES | |
| dCasPhi12( | NPVMIDAKEVSAAHRARYFWGNLPGMNRPLASTVNDKLELQECLEHGRIAKFSKV | |
| L26R_E567 | RTITTRSNSIKQGKDQHFPVFMNEKEDILWCTEMERVFGFPVHYTDVSNMSRLAR | |
| Q)-NLS- | QRLLGRSWSVPVIRHLFAPLKEYFACVSSGNSNANSRGPSFSSGLVPLSLRGSHM | |
| KRAB | GPMEIYKTVSAWKRQPVRVLSLFRNIDKVLKSLGFLESGSGSGGGTLKYVEDVTN | |
| | VVRRDVEKWGPFDLVYGSTQPLGSSCDRCPGWYMFQFHRILQYALPRQESQRPFF | |
| | WIFMDNLLLTEDDQETTTRFLQTEAVTLQDVRGRDYQNAMRVWSNIPGLKSKHAP | |
| | LTPKEEEYLQAQVRSRSKLDAPKVDLLVKNCLLPLREYFKYFSQNSLPLGGPSSG | |
| | APPPSGGSPAGSPTSTEEGTSESATPESGPGTSTEPSEGSAPGSPAGSMAPKKKR | |
| | KVGIHGVPAAMIKPTVSQFLTPGFKLIRNHSRTAGRKLKNEGEEACKKFVRENEI | |
| | PKDECPNFQGGPAIANIIAKSREFTEWEIYQSSLAIQEVIFTLPKDKLPEPILKE | |
| | EWRAQWLSEHGLDTVPYKEAAGLNLIIKNAVNTYKGVQVKVDNKNKNNLAKINRK | |
| | NEIAKLNGEQEISFEEIKAFDDKGYLLQKPSPNKSIYCYQSVSPKPFITSKYHNV | |
| | NLPEEYIGYYRKSNEPIVSPYQFDRLRIPIGEPGYVPKWQYTFLSKKENKRRKLS | |
| | KRIKNVSPILGIICIKKDWCVFDMRGLLRTNHWKKYHKPTDSINDLFDYFTGDPV | |
| | IDTKANVVRFRYKMENGIVNYKPVREKKGKELLENICDQNGSCKLATVDVGQNNP | |
| | VAIGLFELKKVNGELTKTLISRHPTPIDFCNKITAYRERYDKLESSIKLDAIKQL | |
| | TSEQKIEVDNYNNNFTPQNTKQIVCSKLNINPNDLPWDKMISGTHFISEKAQVSN | |
| | KSEIYFTSTDKGKTKDVMKSDYKWFQDYKPKLSKEVRDALSDIEWRLRRESLEFN | |
| | KLSKSREQDARQLANWISSMCDVIGIQNLVKKNNFFGGSGKREPGWDNFYKPKKE | |
| | NRWWINAIHKALTELSQNKGKRVILLPAMRTSITCPKCKYCDSKNRNGEKFNCLK | |
| | CGIELNADIDVATENLATVAITAQSMPKPTCERSGDAKKPVRARKAKAPEFHDKL | |
| | APSYTVVLREAVKRPAATKKAGQAKKKKEFGSPAGSPTSTEEGTSESATPMDAKS | |
| | LTAWSRTLVTFKDVFVDFTREEWKLLDTAQQIVYRNVMLENYKNLVSLGYQLTKP | |
| | DVILRLEKGEEPWLV | |
| KRAB- | MDAKSLTAWSRTLVTFKDVFVDFTREEWKLLDTAQQIVYRNVMLENYKNLVSLGY | 17 |
| NLS- | QLTKPDVILRLEKGEEPWLVGSPAGSPTSTEEGTSESATPMAPKKKRKVGIHGVP | |
| dCasPhi12( | AAMIKPTVSQFLTPGFKLIRNHSRTAGRKLKNEGEEACKKFVRENEIPKDECPNF | |
| L26R_E567 | QGGPAIANIIAKSREFTEWEIYQSSLAIQEVIFTLPKDKLPEPILKEEWRAQWLS | |
| Q)-NLS- | EHGLDTVPYKEAAGLNLIIKNAVNTYKGVQVKVDNKNKNNLAKINRKNEIAKLNG | |
| DNMT3L- | EQEISFEEIKAFDDKGYLLQKPSPNKSIYCYQSVSPKPFITSKYHNVNLPEEYIG | |
| DNMT3A | YYRKSNEPIVSPYQFDRLRIPIGEPGYVPKWQYTFLSKKENKRRKLSKRIKNVSP | |
| | ILGIICIKKDWCVFDMRGLLRTNHWKKYHKPTDSINDLFDYFTGDPVIDTKANVV | |
| | RFRYKMENGIVNYKPVREKKGKELLENICDQNGSCKLATVDVGQNNPVAIGLFEL | |
| | KKVNGELTKTLISRHPTPIDFCNKITAYRERYDKLESSIKLDAIKQLTSEQKIEV | |
| | DNYNNNFTPQNTKQIVCSKLNINPNDLPWDKMISGTHFISEKAQVSNKSEIYFTS | |
| | TDKGKTKDVMKSDYKWFQDYKPKLSKEVRDALSDIEWRLRRESLEFNKLSKSREQ | |
| | DARQLANWISSMCDVIGIQNLVKKNNFFGGSGKREPGWDNFYKPKKENRWWINAI | |
| | HKALTELSQNKGKRVILLPAMRTSITCPKCKYCDSKNRNGEKFNCLKCGIELNAD | |
| | IDVATENLATVAITAQSMPKPTCERSGDAKKPVRARKAKAPEFHDKLAPSYTVVL | |
| | REAVKRPAATKKAGQAKKKKEFGGPSSGAPPPSGGSPAGSPTSTEEGTSESATPE | |
| | SGPGTSTEPSEGSAPGSPAGSMGPMEIYKTVSAWKRQPVRVLSLFRNIDKVLKSL | |
| | GFLESGSGSGGGTLKYVEDVTNVVRRDVEKWGPFDLVYGSTQPLGSSCDRCPGWY | |
| | MFQFHRILQYALPRQESQRPFFWIFMDNLLLTEDDQETTTRFLQTEAVTLQDVRG | |
| | RDYQNAMRVWSNIPGLKSKHAPLTPKEEEYLQAQVRSRSKLDAPKVDLLVKNCLL | |
| | PLREYFKYFSQNSLPLSSGNSNANSRGPSFSSGLVPLSLRGSHMNHDQEFDPPKV | |
| | YPPVPAEKRKPIRVLSLFDGIATGLLVLKDLGIQVDRYIASEVCEDSITVGMVRH | |
| | QGKIMYVGDVRSVTQKHIQEWGPFDLVIGGSPCNDLSIVNPARKGLYEGTGRLFF | |
| | EFYRLLHDARPKEGDDRPFFWLFENVVAMGVSDKRDISRFLESNPVMIDAKEVSA | |
| | AHRARYFWGNLPGMNRPLASTVNDKLELQECLEHGRIAKFSKVRTITTRSNSIKQ | |
| | GKDQHFPVFMNEKEDILWCTEMERVFGFPVHYTDVSNMSRLARQRLLGRSWSVPV | |
| | IRHLFAPLKEYFACV | |
| KRAB- | MDAKSLTAWSRTLVTFKDVFVDFTREEWKLLDTAQQIVYRNVMLENYKNLVSLGY | 18 |
| NLS- | QLTKPDVILRLEKGEEPWLVGSPAGSPTSTEEGTSESATPMAPKKKRKVGIHGVP | |
| dCasPhi12 | AAMIKPTVSQFLTPGFKLIRNHSRTAGRKLKNEGEEACKKFVRENEIPKDECPNF | |
| (L26R_E567 | QGGPAIANIIAKSREFTEWEIYQSSLAIQEVIFTLPKDKLPEPILKEEWRAQWLS | |
| Q)-NLS- | EHGLDTVPYKEAAGLNLIIKNAVNTYKGVQVKVDNKNKNNLAKINRKNEIAKLNG | |
| DNMT3L | EQEISFEEIKAFDDKGYLLQKPSPNKSIYCYQSVSPKPFITSKYHNVNLPEEYIG | |
| | YYRKSNEPIVSPYQFDRLRIPIGEPGYVPKWQYTFLSKKENKRRKLSKRIKNVSP | |
| | ILGIICIKKDWCVFDMRGLLRTNHWKKYHKPTDSINDLFDYFTGDPVIDTKANVV | |
| | RFRYKMENGIVNYKPVREKKGKELLENICDQNGSCKLATVDVGQNNPVAIGLFEL | |
| | KKVNGELTKTLISRHPTPIDFCNKITAYRERYDKLESSIKLDAIKQLTSEQKIEV | |
| | DNYNNNFTPQNTKQIVCSKLNINPNDLPWDKMISGTHFISEKAQVSNKSEIYFTS | |
| | TDKGKTKDVMKSDYKWFQDYKPKLSKEVRDALSDIEWRLRRESLEFNKLSKSREQ | |
| | DARQLANWISSMCDVIGIQNLVKKNNFFGGSGKREPGWDNFYKPKKENRWWINAI | |
| | HKALTELSQNKGKRVILLPAMRTSITCPKCKYCDSKNRNGEKFNCLKCGIELNAD | |
| | IDVATENLATVAITAQSMPKPTCERSGDAKKPVRARKAKAPEFHDKLAPSYTVVL | |
| | REAVKRPAATKKAGQAKKKKEFGGPSSGAPPPSGGSPAGSPTSTEEGTSESATPE | |
| | SGPGTSTEPSEGSAPGSPAGSMGPMEIYKTVSAWKRQPVRVLSLFRNIDKVLKSL | |
| | GFLESGSGSGGGTLKYVEDVTNVVRRDVEKWGPFDLVYGSTQPLGSSCDRCPGWY | |
| | MFQFHRILQYALPRQESQRPFFWIFMDNLLLTEDDQETTTRFLQTEAVTLQDVRG | |
| | RDYQNAMRVWSNIPGLKSKHAPLTPKEEEYLQAQVRSRSKLDAPKVDLLVKNCLL | |
| | PLREYFKYFSQNSLPL | |
| NLS- | MAPKKKRKVGIHGVPAAMIKPTVSQFLTPGFKLIRNHSRTAGRKLKNEGEEACKK | 19 |
| dCasPhi12 | FVRENEIPKDECPNFQGGPAIANIIAKSREFTEWEIYQSSLAIQEVIFTLPKDKL | |
| (L26R_E567 | PEPILKEEWRAQWLSEHGLDTVPYKEAAGLNLIIKNAVNTYKGVQVKVDNKNKNN | |
| Q)-NLS- | LAKINRKNEIAKLNGEQEISFEEIKAFDDKGYLLQKPSPNKSIYCYQSVSPKPFI | |
| DNMT3L- | TSKYHNVNLPEEYIGYYRKSNEPIVSPYQFDRLRIPIGEPGYVPKWQYTFLSKKE | |
| KRAB | NKRRKLSKRIKNVSPILGIICIKKDWCVFDMRGLLRTNHWKKYHKPTDSINDLFD | |
| | YFTGDPVIDTKANVVRFRYKMENGIVNYKPVREKKGKELLENICDQNGSCKLATV | |
| | DVGQNNPVAIGLFELKKVNGELTKTLISRHPTPIDFCNKITAYRERYDKLESSIK | |
| | LDAIKQLTSEQKIEVDNYNNNFTPQNTKQIVCSKLNINPNDLPWDKMISGTHFIS | |
| | EKAQVSNKSEIYFTSTDKGKTKDVMKSDYKWFQDYKPKLSKEVRDALSDIEWRLR | |
| | RESLEFNKLSKSREQDARQLANWISSMCDVIGIQNLVKKNNFFGGSGKREPGWDN | |
| | FYKPKKENRWWINAIHKALTELSQNKGKRVILLPAMRTSITCPKCKYCDSKNRNG | |
| | EKFNCLKCGIELNADIDVATENLATVAITAQSMPKPTCERSGDAKKPVRARKAKA | |
| | PEFHDKLAPSYTVVLREAVKRPAATKKAGQAKKKKEFGGPSSGAPPPSGGSPAGS | |
| | PTSTEEGTSESATPESGPGTSTEPSEGSAPGSPAGSMGPMEIYKTVSAWKRQPVR | |
| | VLSLFRNIDKVLKSLGFLESGSGSGGGTLKYVEDVTNVVRRDVEKWGPFDLVYGS | |
| | TQPLGSSCDRCPGWYMFQFHRILQYALPRQESQRPFFWIFMDNLLLTEDDQETTT | |
| | RFLQTEAVTLQDVRGRDYQNAMRVWSNIPGLKSKHAPLTPKEEEYLQAQVRSRSK | |
| | LDAPKVDLLVKNCLLPLREYFKYFSQNSLPLGSPAGSPTSTEEGTSESATPMDAK | |
| | SLTAWSRTLVTFKDVFVDFTREEWKLLDTAQQIVYRNVMLENYKNLVSLGYQLTK | |
| | PDVILRLEKGEEPWLV | |
| DNMT3L- | MGPMEIYKTVSAWKRQPVRVLSLFRNIDKVLKSLGFLESGSGSGGGTLKYVEDVT | 20 |
| KRAB- | NVVRRDVEKWGPFDLVYGSTQPLGSSCDRCPGWYMFQFHRILQYALPRQESQRPF | |
| NLS- | FWIFMDNLLLTEDDQETTTRFLQTEAVTLQDVRGRDYQNAMRVWSNIPGLKSKHA | |
| dCasPhi12 | PLTPKEEEYLQAQVRSRSKLDAPKVDLLVKNCLLPLREYFKYFSQNSLPLGGPSS | |
| (L26R_E567 | GAPPPSGGSPAGSPTSTEEGTSESATPESGPGTSTEPSEGSAPGSPAGSMDAKSL | |
| Q)-NLS | TAWSRTLVTFKDVFVDFTREEWKLLDTAQQIVYRNVMLENYKNLVSLGYQLTKPD | |
| | VILRLEKGEEPWLVGSPAGSPTSTEEGTSESATPMAPKKKRKVGIHGVPAAMIKP | |
| | TVSQFLTPGFKLIRNHSRTAGRKLKNEGEEACKKFVRENEIPKDECPNFQGGPAI | |
| | ANIIAKSREFTEWEIYQSSLAIQEVIFTLPKDKLPEPILKEEWRAQWLSEHGLDT | |
| | VPYKEAAGLNLIIKNAVNTYKGVQVKVDNKNKNNLAKINRKNEIAKLNGEQEISF | |
| | EEIKAFDDKGYLLQKPSPNKSIYCYQSVSPKPFITSKYHNVNLPEEYIGYYRKSN | |
| | EPIVSPYQFDRLRIPIGEPGYVPKWQYTFLSKKENKRRKLSKRIKNVSPILGIIC | |
| | IKKDWCVFDMRGLLRTNHWKKYHKPTDSINDLFDYFTGDPVIDTKANVVRFRYKM | |
| | ENGIVNYKPVREKKGKELLENICDQNGSCKLATVDVGQNNPVAIGLFELKKVNGE | |
| | LTKTLISRHPTPIDFCNKITAYRERYDKLESSIKLDAIKQLTSEQKIEVDNYNNN | |
| | FTPQNTKQIVCSKLNINPNDLPWDKMISGTHFISEKAQVSNKSEIYFTSTDKGKT | |
| | KDVMKSDYKWFQDYKPKLSKEVRDALSDIEWRLRRESLEFNKLSKSREQDARQLA | |
| | NWISSMCDVIGIQNLVKKNNFFGGSGKREPGWDNFYKPKKENRWWINAIHKALTE | |
| | LSQNKGKRVILLPAMRTSITCPKCKYCDSKNRNGEKFNCLKCGIELNADIDVATE | |
| | NLATVAITAQSMPKPTCERSGDAKKPVRARKAKAPEFHDKLAPSYTVVLREAVKR | |
| | PAATKKAGQAKKKKEF | |
| KRAB- | MDAKSLTAWSRTLVTFKDVFVDFTREEWKLLDTAQQIVYRNVMLENYKNLVSLGY | 21 |
| NLS- | QLTKPDVILRLEKGEEPWLVGSPAGSPTSTEEGTSESATPMAPKKKRKVGIHGVP | |
| dCasPhi12- | AAMIKPTVSQFLTPGFKLIRNHSRTAGRKLKNEGEEACKKFVRENEIPKDECPNF | |
| NLS | QGGPAIANIIAKSREFTEWEIYQSSLAIQEVIFTLPKDKLPEPILKEEWRAQWLS | |
| | EHGLDTVPYKEAAGLNLIIKNAVNTYKGVQVKVDNKNKNNLAKINRKNEIAKLNG | |
| | EQEISFEEIKAFDDKGYLLQKPSPNKSIYCYQSVSPKPFITSKYHNVNLPEEYIG | |
| | YYRKSNEPIVSPYQFDRLRIPIGEPGYVPKWQYTFLSKKENKRRKLSKRIKNVSP | |
| | ILGIICIKKDWCVFDMRGLLRTNHWKKYHKPTDSINDLFDYFTGDPVIDTKANVV | |
| | RFRYKMENGIVNYKPVREKKGKELLENICDQNGSCKLATVDVGQNNPVAIGLFEL | |
| | KKVNGELTKTLISRHPTPIDFCNKITAYRERYDKLESSIKLDAIKQLTSEQKIEV | |
| | DNYNNNFTPQNTKQIVCSKLNINPNDLPWDKMISGTHFISEKAQVSNKSEIYFTS | |
| | TDKGKTKDVMKSDYKWFQDYKPKLSKEVRDALSDIEWRLRRESLEFNKLSKSREQ | |
| | DARQLANWISSMCDVIGIQNLVKKNNFFGGSGKREPGWDNFYKPKKENRWWINAI | |
| | HKALTELSQNKGKRVILLPAMRTSITCPKCKYCDSKNRNGEKFNCLKCGIELNAD | |
| | IDVATENLATVAITAQSMPKPTCERSGDAKKPVRARKAKAPEFHDKLAPSYTVVL | |
| | REAVKRPAATKKAGQAKKKKEF | |
| dCasPhi.12- | MAPKKKRKVGIHGVPAAMIKPTVSQFLTPGFKLIRNHSRTAGRKLKNEGEEACKK | 3004 |
| DNMT3L | FVRENEIPKDECPNFQGGPAIANIIAKSREFTEWEIYQSSLAIQEVIFTLPKDKL | |
| | PEPILKEEWRAQWLSEHGLDTVPYKEAAGLNLIIKNAVNTYKGVQVKVDNKNKNN | |
| | LAKINRKNEIAKLNGEQEISFEEIKAFDDKGYLLQKPSPNKSIYCYQSVSPKPFI | |
| | TSKYHNVNLPEEYIGYYRKSNEPIVSPYQFDRLRIPIGEPGYVPKWQYTFLSKKE | |
| | NKRRKLSKRIKNVSPILGIICIKKDWCVFDMRGLLRTNHWKKYHKPTDSINDLFD | |
| | YFTGDPVIDTKANVVRFRYKMENGIVNYKPVREKKGKELLENICDQNGSCKLATV | |
| | DVGQNNPVAIGLFELKKVNGELTKTLISRHPTPIDFCNKITAYRERYDKLESSIK | |
| | LDAIKQLTSEQKIEVDNYNNNFTPQNTKQIVCSKLNINPNDLPWDKMISGTHFIS | |
| | EKAQVSNKSEIYFTSTDKGKTKDVMKSDYKWFQDYKPKLSKEVRDALSDIEWRLR | |
| | RESLEFNKLSKSREQDARQLANWISSMCDVIGIQNLVKKNNFFGGSGKREPGWDN | |
| | FYKPKKENRWWINAIHKALTELSQNKGKRVILLPAMRTSITCPKCKYCDSKNRNG | |
| | EKFNCLKCGIELNADIDVATENLATVAITAQSMPKPTCERSGDAKKPVRARKAKA | |
| | PEFHDKLAPSYTVVLREAVKRPAATKKAGQAKKKKEFGGPSSGAPPPSGGSPAGS | |
| | PTSTEEGTSESATPESGPGTSTEPSEGSAPGSPAGSMGPMEIYKTVSAWKRQPVR | |
| | VLSLFRNIDKVLKSLGFLESGSGSGGGTLKYVEDVTNVVRRDVEKWGPFDLVYGS | |
| | TQPLGSSCDRCPGWYMFQFHRILQYALPRQESQRPFFWIFMDNLLLTEDDQETTT | |
| | RFLQTEAVTLQDVRGRDYQNAMRVWSNIPGLKSKHAPLTPKEEEYLQAQVRSRSK | |
| | LDAPKVDLLVKNCLLPLREYFKYFSQNSLPLGSPAGSPTSTEEGTSESATP | |
| | wherein italics represent NLSs, | |
| | bold represents dCasPhi.12(L26R, E567Q), | |
| | regular font represents mouse Dnmt31 | |
| | (C terminus domain), and | |
| | double underline represents linker | |

TABLE 7 provides illustrative repeat sequences for use in guide nucleic acids that are useful in the compositions, systems and methods described herein.

TABLE 7
EXEMPLARY REPEAT SEQUENCES FOR USE
IN GUIDE NUCLEIC ACIDS

| Effector | | Repeat |
|---|---|---|
| Protein | Repeat Sequence | Sequence |
| SEQ | (5′ → 3′), | SEQ ID |
| ID NO: | shown as RNA | NO: |
| 1 | AUUGCUCCUUACGAGGAGAC | 5 |
| 1 | GAUUGCUCCUUACGAGGAGAC | 6 |
| 1 | AGAUUGCUCCUUACGAGGAGAC | 7 |
| 1 | UAGAUUGCUCCUUACGAGGAGAC | 8 |
| 1 | AUAGAUUGCUCCUUACGAGGAGAC | 9 |

TABLE 8 provides illustrative handle sequences for use in guide nucleic acids that are useful in the compositions, systems and methods described herein.

TABLE 8
EXEMPLARY HANDLE SEQUENCES FOR USE IN
GUIDE NUCLEIC ACIDS

| Effector | Handle Sequence | Repeat |
|---|---|---|
| Protein SEQ | (5′ → 3′), | Sequence |
| ID NO: | shown as RNA | SEQ ID NO: |
| 3 | ACAGCUUAUUUGGAAGC | 36 |
| | UGAAAUGUGAGGUUUAU | |
| | AACACUCACAAGAAUCC | |
| | UGAAAAAGGAUGCCAAA | |
| | C | |

TABLE 9 provides exemplary spacer sequences and full guide nucleic acid sequences for targeting human APOC3, human PCSK9, human ANGPTL3, mouse Apoc3, and mouse Pcsk9 for CasPhi.12 effector protein.

TABLE 9
EXEMPLARY SPACER SEQUENCES AND FULL GUIDE
NUCLEIC ACID SEQUENCES FOR CASPHI.12

| | | Spacer | gRNA |
|---|---|---|---|
| | | Sequence | SEQ ID |
| Spacer ID | Target | SEQ ID NO: | NO: |
| 132842 | human APOC3 | 37 | 529 |
| 132843 | human APOC3 | 38 | 530 |
| 132844 | human APOC3 | 39 | 531 |
| 132845 | human APOC3 | 40 | 532 |
| 132846 | human APOC3 | 41 | 533 |
| 132847 | human APOC3 | 42 | 534 |
| 132848 | human APOC3 | 43 | 535 |
| 132849 | human APOC3 | 44 | 536 |
| 132850 | human APOC3 | 45 | 537 |
| 132851 | human APOC3 | 46 | 538 |
| 132852 | human APOC3 | 47 | 539 |
| 132853 | human APOC3 | 48 | 540 |
| 132854 | human APOC3 | 49 | 541 |
| 132855 | human APOC3 | 50 | 542 |
| 132856 | human APOC3 | 51 | 543 |
| 132857 | human APOC3 | 52 | 544 |
| 132858 | human APOC3 | 53 | 545 |
| 132859 | human APOC3 | 54 | 546 |
| 132860 | human APOC3 | 55 | 547 |
| 132861 | human APOC3 | 56 | 548 |
| 132862 | human APOC3 | 57 | 549 |
| 132863 | human APOC3 | 58 | 550 |
| 132864 | human APOC3 | 59 | 551 |
| 132865 | human APOC3 | 60 | 552 |
| 132866 | human APOC3 | 61 | 553 |
| 132867 | human APOC3 | 62 | 554 |
| 132868 | human APOC3 | 63 | 555 |
| 132869 | human APOC3 | 64 | 556 |
| 132870 | human APOC3 | 65 | 557 |
| 132871 | human APOC3 | 66 | 558 |
| 132872 | human APOC3 | 67 | 559 |
| 132873 | human APOC3 | 68 | 560 |
| 132874 | human APOC3 | 69 | 561 |
| 132875 | human APOC3 | 70 | 562 |
| 132876 | human APOC3 | 71 | 563 |
| 132877 | human APOC3 | 72 | 564 |
| 132878 | human APOC3 | 73 | 565 |
| 132879 | human APOC3 | 74 | 566 |
| 132880 | human APOC3 | 75 | 567 |
| 132881 | human APOC3 | 76 | 568 |
| 132882 | human APOC3 | 77 | 569 |
| 132883 | human APOC3 | 78 | 570 |
| 132884 | human APOC3 | 79 | 571 |
| 132885 | human APOC3 | 80 | 572 |
| 132886 | human APOC3 | 81 | 573 |
| 132887 | human APOC3 | 82 | 574 |
| 132888 | human APOC3 | 83 | 575 |
| 132889 | human APOC3 | 84 | 576 |
| 132890 | human APOC3 | 85 | 577 |
| 132891 | human APOC3 | 86 | 578 |
| 132892 | human APOC3 | 87 | 579 |
| 132893 | human APOC3 | 88 | 580 |
| 132894 | human APOC3 | 89 | 581 |
| 132895 | human APOC3 | 90 | 582 |
| 132896 | human APOC3 | 91 | 583 |
| 132897 | human APOC3 | 92 | 584 |
| 132898 | human APOC3 | 93 | 585 |
| 132899 | human APOC3 | 94 | 586 |
| 132900 | human APOC3 | 95 | 587 |
| 132901 | human APOC3 | 96 | 588 |
| 132902 | human APOC3 | 97 | 589 |
| 132903 | human APOC3 | 98 | 590 |
| 132904 | human APOC3 | 99 | 591 |
| 132905 | human APOC3 | 100 | 592 |
| 132906 | human APOC3 | 101 | 593 |
| 132907 | human APOC3 | 102 | 594 |
| 132908 | human APOC3 | 103 | 595 |
| 132909 | human APOC3 | 104 | 596 |
| 132910 | human APOC3 | 105 | 597 |
| 132911 | human APOC3 | 106 | 598 |
| 132912 | human APOC3 | 107 | 599 |
| 132913 | human APOC3 | 108 | 600 |
| 132914 | human APOC3 | 109 | 601 |
| 132915 | human PCSK9 | 110 | 602 |
| 132916 | human PCSK9 | 111 | 603 |
| 132917 | human PCSK9 | 112 | 604 |
| 132918 | human PCSK9 | 113 | 605 |
| 132919 | human PCSK9 | 114 | 606 |
| 132920 | human PCSK9 | 115 | 607 |
| 132921 | human PCSK9 | 116 | 608 |
| 132922 | human PCSK9 | 117 | 609 |
| 132923 | human PCSK9 | 118 | 610 |
| 132924 | human PCSK9 | 119 | 611 |
| 132925 | human PCSK9 | 120 | 612 |
| 132926 | human PCSK9 | 121 | 613 |
| 132927 | human PCSK9 | 122 | 614 |
| 132928 | human PCSK9 | 123 | 615 |
| 132929 | human PCSK9 | 124 | 616 |
| 132930 | human PCSK9 | 125 | 617 |
| 132931 | human PCSK9 | 126 | 618 |
| 132932 | human PCSK9 | 127 | 619 |
| 132933 | human PCSK9 | 128 | 620 |
| 132934 | human PCSK9 | 129 | 621 |
| 132935 | human PCSK9 | 130 | 622 |
| 132936 | human PCSK9 | 131 | 623 |
| 132937 | human PCSK9 | 132 | 624 |
| 132938 | human PCSK9 | 133 | 625 |
| 132939 | human PCSK9 | 134 | 626 |
| 132940 | human PCSK9 | 135 | 627 |
| 132941 | human PCSK9 | 136 | 628 |
| 132942 | human PCSK9 | 137 | 629 |
| 132943 | human PCSK9 | 138 | 630 |
| 132944 | human PCSK9 | 139 | 631 |
| 132945 | human PCSK9 | 140 | 632 |
| 132946 | human PCSK9 | 141 | 633 |
| 132947 | human PCSK9 | 142 | 634 |
| 132948 | human PCSK9 | 143 | 635 |
| 132949 | human PCSK9 | 144 | 636 |
| 132950 | human PCSK9 | 145 | 637 |
| 132951 | human PCSK9 | 146 | 638 |
| 132952 | human PCSK9 | 147 | 639 |
| 132953 | human PCSK9 | 148 | 640 |
| 132954 | human PCSK9 | 149 | 641 |
| 132955 | human PCSK9 | 150 | 642 |
| 132956 | human PCSK9 | 151 | 643 |
| 132957 | human PCSK9 | 152 | 644 |
| 132958 | human PCSK9 | 153 | 645 |
| 132959 | human PCSK9 | 154 | 646 |
| 132960 | human PCSK9 | 155 | 647 |
| 132961 | human PCSK9 | 156 | 648 |
| 132962 | human PCSK9 | 157 | 649 |
| 132963 | human PCSK9 | 158 | 650 |
| 132964 | human PCSK9 | 159 | 651 |
| 132965 | human PCSK9 | 160 | 652 |
| 132966 | human PCSK9 | 161 | 653 |
| 132967 | human PCSK9 | 162 | 654 |
| 132968 | human PCSK9 | 163 | 655 |
| 132969 | human PCSK9 | 164 | 656 |
| 132970 | human PCSK9 | 165 | 657 |
| 132971 | human PCSK9 | 166 | 658 |
| 132972 | human PCSK9 | 167 | 659 |
| 132973 | human PCSK9 | 168 | 660 |
| 132974 | human PCSK9 | 169 | 661 |
| 132975 | human PCSK9 | 170 | 662 |
| 132976 | human PCSK9 | 171 | 663 |
| 132977 | human PCSK9 | 172 | 664 |
| 132978 | human PCSK9 | 173 | 665 |
| 132979 | human PCSK9 | 174 | 666 |
| 132980 | human PCSK9 | 175 | 667 |
| 132981 | human PCSK9 | 176 | 668 |
| 132982 | human PCSK9 | 177 | 669 |
| 132983 | human ANGPTL3 | 178 | 670 |
| 132984 | human ANGPTL3 | 179 | 671 |
| 132985 | human ANGPTL3 | 180 | 672 |
| 132986 | human ANGPTL3 | 181 | 673 |
| 132987 | human ANGPTL3 | 182 | 674 |
| 132988 | human ANGPTL3 | 183 | 675 |
| 132989 | human ANGPTL3 | 184 | 676 |
| 132990 | human ANGPTL3 | 185 | 677 |
| 132991 | human ANGPTL3 | 186 | 678 |
| 132992 | human ANGPTL3 | 187 | 679 |
| 132993 | human ANGPTL3 | 188 | 680 |
| 132994 | human ANGPTL3 | 189 | 681 |
| 132995 | human ANGPTL3 | 190 | 682 |
| 132996 | human ANGPTL3 | 191 | 683 |
| 132997 | human ANGPTL3 | 192 | 684 |
| 132998 | human ANGPTL3 | 193 | 685 |
| 132999 | human ANGPTL3 | 194 | 686 |
| 133000 | human ANGPTL3 | 195 | 687 |
| 133001 | human ANGPTL3 | 196 | 688 |
| 133002 | human ANGPTL3 | 197 | 689 |
| 133003 | human ANGPTL3 | 198 | 690 |
| 133004 | human ANGPTL3 | 199 | 691 |
| 133005 | human ANGPTL3 | 200 | 692 |
| 133006 | human ANGPTL3 | 201 | 693 |
| 133007 | human ANGPTL3 | 202 | 694 |
| 133008 | human ANGPTL3 | 203 | 695 |
| 133009 | human ANGPTL3 | 204 | 696 |
| 133010 | human ANGPTL3 | 205 | 697 |
| 133011 | human ANGPTL3 | 206 | 698 |
| 133012 | human ANGPTL3 | 207 | 699 |
| 133013 | human ANGPTL3 | 208 | 700 |
| 133014 | human ANGPTL3 | 209 | 701 |
| 133015 | human ANGPTL3 | 210 | 702 |
| 133016 | human ANGPTL3 | 211 | 703 |
| 133017 | human ANGPTL3 | 212 | 704 |
| 133018 | human ANGPTL3 | 213 | 705 |
| 133019 | human ANGPTL3 | 214 | 706 |
| 133020 | human ANGPTL3 | 215 | 707 |
| 133021 | human ANGPTL3 | 216 | 708 |
| 133022 | human ANGPTL3 | 217 | 709 |
| 133023 | human ANGPTL3 | 218 | 710 |
| 133024 | human ANGPTL3 | 219 | 711 |
| 133025 | human ANGPTL3 | 220 | 712 |
| 133026 | human ANGPTL3 | 221 | 713 |
| 133027 | human ANGPTL3 | 222 | 714 |
| 133028 | human ANGPTL3 | 223 | 715 |
| 133029 | human ANGPTL3 | 224 | 716 |
| 133030 | human ANGPTL3 | 225 | 717 |
| 133031 | human ANGPTL3 | 226 | 718 |
| 133032 | human ANGPTL3 | 227 | 719 |
| 133033 | human ANGPTL3 | 228 | 720 |
| 133034 | human ANGPTL3 | 229 | 721 |
| 133035 | human ANGPTL3 | 230 | 722 |
| 133036 | human ANGPTL3 | 231 | 723 |
| 133037 | human ANGPTL3 | 232 | 724 |
| 133038 | human ANGPTL3 | 233 | 725 |
| 133039 | human ANGPTL3 | 234 | 726 |
| 133040 | human ANGPTL3 | 235 | 727 |
| 133041 | human ANGPTL3 | 236 | 728 |
| 133042 | human ANGPTL3 | 237 | 729 |
| 133043 | human ANGPTL3 | 238 | 730 |
| 133044 | human ANGPTL3 | 239 | 731 |
| 133045 | human ANGPTL3 | 240 | 732 |
| 133046 | human ANGPTL3 | 241 | 733 |
| 133047 | human ANGPTL3 | 242 | 734 |
| 133048 | human ANGPTL3 | 243 | 735 |
| 133049 | human ANGPTL3 | 244 | 736 |
| 133050 | human ANGPTL3 | 245 | 737 |
| 133051 | human ANGPTL3 | 246 | 738 |
| 133052 | human ANGPTL3 | 247 | 739 |
| 133053 | human ANGPTL3 | 248 | 740 |
| 133054 | human ANGPTL3 | 249 | 741 |
| 133055 | human ANGPTL3 | 250 | 742 |
| 133056 | human ANGPTL3 | 251 | 743 |
| 133057 | human ANGPTL3 | 252 | 744 |
| 133058 | human ANGPTL3 | 253 | 745 |
| 133059 | human ANGPTL3 | 254 | 746 |
| 133060 | human ANGPTL3 | 255 | 747 |
| 133061 | human ANGPTL3 | 256 | 748 |
| 133062 | human ANGPTL3 | 257 | 749 |
| 133063 | human ANGPTL3 | 258 | 750 |
| 133064 | human ANGPTL3 | 259 | 751 |
| 133065 | human ANGPTL3 | 260 | 752 |
| 133066 | human ANGPTL3 | 261 | 753 |
| 133067 | human ANGPTL3 | 262 | 754 |
| 133068 | human ANGPTL3 | 263 | 755 |
| 133069 | human ANGPTL3 | 264 | 756 |
| 133070 | human ANGPTL3 | 265 | 757 |
| 133071 | human ANGPTL3 | 266 | 758 |
| 133072 | human ANGPTL3 | 267 | 759 |
| 133073 | human ANGPTL3 | 268 | 760 |
| 133074 | human ANGPTL3 | 269 | 761 |
| 133075 | human ANGPTL3 | 270 | 762 |
| 133076 | human ANGPTL3 | 271 | 763 |
| 133077 | human ANGPTL3 | 272 | 764 |
| 133078 | human ANGPTL3 | 273 | 765 |
| 133079 | human ANGPTL3 | 274 | 766 |
| 133080 | human ANGPTL3 | 275 | 767 |
| 133081 | human ANGPTL3 | 276 | 768 |
| 133082 | human ANGPTL3 | 277 | 769 |
| 133083 | human ANGPTL3 | 278 | 770 |
| 133084 | human ANGPTL3 | 279 | 771 |
| 133085 | human ANGPTL3 | 280 | 772 |
| 133086 | human ANGPTL3 | 281 | 773 |
| 133087 | human ANGPTL3 | 282 | 774 |
| 133088 | human ANGPTL3 | 283 | 775 |
| 133089 | human ANGPTL3 | 284 | 776 |
| 133090 | human ANGPTL3 | 285 | 777 |
| 133091 | human ANGPTL3 | 286 | 778 |
| 133092 | human ANGPTL3 | 287 | 779 |
| 133093 | human ANGPTL3 | 288 | 780 |
| 133094 | human ANGPTL3 | 289 | 781 |
| 133095 | human ANGPTL3 | 290 | 782 |
| 133096 | human ANGPTL3 | 291 | 783 |
| 133097 | human ANGPTL3 | 292 | 784 |
| 133098 | human ANGPTL3 | 293 | 785 |
| 133099 | human ANGPTL3 | 294 | 786 |
| 133100 | human ANGPTL3 | 295 | 787 |
| 133101 | human ANGPTL3 | 296 | 788 |
| 133102 | human ANGPTL3 | 297 | 789 |
| 133103 | human ANGPTL3 | 298 | 790 |
| 133104 | human ANGPTL3 | 299 | 791 |
| 133105 | human ANGPTL3 | 300 | 792 |
| 133106 | human ANGPTL3 | 301 | 793 |
| 133107 | human ANGPTL3 | 302 | 794 |
| 133108 | human ANGPTL3 | 303 | 795 |
| 133109 | human ANGPTL3 | 304 | 796 |
| 133110 | human ANGPTL3 | 305 | 797 |
| 133111 | human ANGPTL3 | 306 | 798 |
| 133112 | human ANGPTL3 | 307 | 799 |
| 133113 | human ANGPTL3 | 308 | 800 |
| 133114 | human ANGPTL3 | 309 | 801 |
| 133115 | human ANGPTL3 | 310 | 802 |
| 133116 | human ANGPTL3 | 311 | 803 |
| 133117 | human ANGPTL3 | 312 | 804 |
| 133118 | human ANGPTL3 | 313 | 805 |
| 133119 | human ANGPTL3 | 314 | 806 |
| 133120 | human ANGPTL3 | 315 | 807 |
| 133121 | human ANGPTL3 | 316 | 808 |
| 133122 | human ANGPTL3 | 317 | 809 |
| 133123 | human ANGPTL3 | 318 | 810 |
| 133124 | human ANGPTL3 | 319 | 811 |
| 133125 | human ANGPTL3 | 320 | 812 |
| 133126 | human ANGPTL3 | 321 | 813 |
| 133127 | human ANGPTL3 | 322 | 814 |
| 133128 | human ANGPTL3 | 323 | 815 |
| 133129 | human ANGPTL3 | 324 | 816 |
| 133130 | human ANGPTL3 | 325 | 817 |
| 133131 | human ANGPTL3 | 326 | 818 |
| 133132 | human ANGPTL3 | 327 | 819 |
| 133133 | human ANGPTL3 | 328 | 820 |
| 133134 | human ANGPTL3 | 329 | 821 |
| 133135 | human ANGPTL3 | 330 | 822 |
| 133136 | human ANGPTL3 | 331 | 823 |
| 133137 | human ANGPTL3 | 332 | 824 |
| 133138 | human ANGPTL3 | 333 | 825 |
| 133139 | human ANGPTL3 | 334 | 826 |
| 133140 | human ANGPTL3 | 335 | 827 |
| 133141 | human ANGPTL3 | 336 | 828 |
| 133142 | human ANGPTL3 | 337 | 829 |
| 133143 | human ANGPTL3 | 338 | 830 |
| 133144 | human ANGPTL3 | 339 | 831 |
| 133145 | human ANGPTL3 | 340 | 832 |
| 133146 | human ANGPTL3 | 341 | 833 |
| 133147 | human ANGPTL3 | 342 | 834 |
| 133148 | human ANGPTL3 | 343 | 835 |
| 133149 | human ANGPTL3 | 344 | 836 |
| 133150 | human ANGPTL3 | 345 | 837 |
| 133151 | human ANGPTL3 | 346 | 838 |
| 133152 | human ANGPTL3 | 347 | 839 |
| 133153 | human ANGPTL3 | 348 | 840 |
| 133154 | human ANGPTL3 | 349 | 841 |
| 133155 | human ANGPTL3 | 350 | 842 |
| 133156 | human ANGPTL3 | 351 | 843 |
| 133157 | human ANGPTL3 | 352 | 844 |
| 133158 | human ANGPTL3 | 353 | 845 |
| 133159 | human ANGPTL3 | 354 | 846 |
| 133160 | human ANGPTL3 | 355 | 847 |
| 133161 | human ANGPTL3 | 356 | 848 |
| 133162 | human ANGPTL3 | 357 | 849 |
| 133163 | human ANGPTL3 | 358 | 850 |
| 133164 | human ANGPTL3 | 359 | 851 |
| 143961 | mouse Apoc3 | 360 | 852 |
| 143962 | mouse Apoc3 | 361 | 853 |
| 143963 | mouse Apoc3 | 362 | 854 |
| 143964 | mouse Apoc3 | 363 | 855 |
| 143965 | mouse Apoc3 | 364 | 856 |
| 143966 | mouse Apoc3 | 365 | 857 |
| 143967 | mouse Apoc3 | 366 | 858 |
| 143968 | mouse Apoc3 | 367 | 859 |
| 143969 | mouse Apoc3 | 368 | 860 |
| 143970 | mouse Apoc3 | 369 | 861 |
| 143971 | mouse Apoc3 | 370 | 862 |
| 143972 | mouse Apoc3 | 371 | 863 |
| 143973 | mouse Apoc3 | 372 | 864 |
| 143974 | mouse Apoc3 | 373 | 865 |
| 143975 | mouse Apoc3 | 374 | 866 |
| 143976 | mouse Apoc3 | 375 | 867 |
| 143977 | mouse Apoc3 | 376 | 868 |
| 143978 | mouse Apoc3 | 377 | 869 |
| 143979 | mouse Apoc3 | 378 | 870 |
| 143980 | mouse Apoc3 | 379 | 871 |
| 143981 | mouse Apoc3 | 380 | 872 |
| 143982 | mouse Apoc3 | 381 | 873 |
| 143983 | mouse Apoc3 | 382 | 874 |
| 143984 | mouse Apoc3 | 383 | 875 |
| 143985 | mouse Apoc3 | 384 | 876 |
| 143986 | mouse Apoc3 | 385 | 877 |
| 143987 | mouse Apoc3 | 386 | 878 |
| 143988 | mouse Apoc3 | 387 | 879 |
| 143989 | mouse Apoc3 | 388 | 880 |
| 143990 | mouse Apoc3 | 389 | 881 |
| 143991 | mouse Apoc3 | 390 | 882 |
| 143992 | mouse Apoc3 | 391 | 883 |
| 143993 | mouse Apoc3 | 392 | 884 |
| 143994 | mouse Apoc3 | 393 | 885 |
| 143995 | mouse Apoc3 | 394 | 886 |
| 143996 | mouse Apoc3 | 395 | 887 |
| 143997 | mouse Apoc3 | 396 | 888 |
| 143998 | mouse Apoc3 | 397 | 889 |
| 143999 | mouse Apoc3 | 398 | 890 |
| 144000 | mouse Apoc3 | 399 | 891 |
| 144001 | mouse Apoc3 | 400 | 892 |
| 144002 | mouse Apoc3 | 401 | 893 |
| 144003 | mouse Apoc3 | 402 | 894 |
| 144004 | mouse Apoc3 | 403 | 895 |
| 144005 | mouse Apoc3 | 404 | 896 |
| 144006 | mouse Apoc3 | 405 | 897 |
| 144007 | mouse Apoc3 | 406 | 898 |
| 144008 | mouse Apoc3 | 407 | 899 |
| 144009 | mouse Apoc3 | 408 | 900 |
| 144010 | mouse Apoc3 | 409 | 901 |
| 144011 | mouse Apoc3 | 410 | 902 |
| 144012 | mouse Apoc3 | 411 | 903 |
| 144013 | mouse Apoc3 | 412 | 904 |
| 144014 | mouse Apoc3 | 413 | 905 |
| 144015 | mouse Apoc3 | 414 | 906 |
| 144016 | mouse Apoc3 | 415 | 907 |
| 144017 | mouse Apoc3 | 416 | 908 |
| 144018 | mouse Apoc3 | 417 | 909 |
| 144019 | mouse Apoc3 | 418 | 910 |
| 144020 | mouse Apoc3 | 419 | 911 |
| 144021 | mouse Apoc3 | 420 | 912 |
| 144022 | mouse Apoc3 | 421 | 913 |
| 144023 | mouse Apoc3 | 422 | 914 |
| 144024 | mouse Apoc3 | 423 | 915 |
| 144025 | mouse Apoc3 | 424 | 916 |
| 144026 | mouse Apoc3 | 425 | 917 |
| 144027 | mouse Apoc3 | 426 | 918 |
| 144028 | mouse Apoc3 | 42 | 919 |
| 144029 | mouse Apoc3 | 428 | 920 |
| 144030 | mouse Apoc3 | 429 | 921 |
| 144031 | mouse Apoc3 | 430 | 922 |
| 144032 | mouse Apoc3 | 431 | 923 |
| 144033 | mouse Apoc3 | 432 | 924 |
| 144034 | mouse Apoc3 | 433 | 925 |
| 144035 | mouse Apoc3 | 434 | 926 |
| 144036 | mouse Apoc3 | 435 | 927 |
| 144037 | mouse Apoc3 | 436 | 928 |
| 144038 | mouse Apoc3 | 437 | 929 |
| 144039 | mouse Apoc3 | 438 | 930 |
| 144040 | mouse Apoc3 | 439 | 931 |
| 144041 | mouse Apoc3 | 440 | 932 |
| 144042 | mouse Apoc3 | 441 | 933 |
| 144043 | mouse Apoc3 | 442 | 934 |
| 144044 | mouse Apoc3 | 443 | 935 |
| 144045 | mouse Apoc3 | 444 | 936 |
| 144046 | mouse Apoc3 | 445 | 937 |
| 144047 | mouse Apoc3 | 446 | 938 |
| 144048 | mouse Apoc3 | 447 | 939 |
| 144049 | mouse Apoc3 | 448 | 940 |
| 144050 | mouse Apoc3 | 449 | 941 |
| 144051 | mouse Apoc3 | 450 | 942 |
| 144052 | mouse Apoc3 | 451 | 943 |
| 144053 | mouse Apoc3 | 452 | 944 |
| 144054 | mouse Apoc3 | 453 | 945 |
| 144055 | mouse Apoc3 | 454 | 946 |
| 144056 | mouse Apoc3 | 455 | 947 |
| 144057 | mouse Apoc3 | 456 | 948 |
| 144058 | mouse Pcsk9 | 457 | 949 |
| 144059 | mouse Pcsk9 | 458 | 950 |
| 144060 | mouse Pcsk9 | 459 | 951 |
| 144061 | mouse Pcsk9 | 460 | 952 |
| 144062 | mouse Pcsk9 | 461 | 953 |
| 144063 | mouse Pcsk9 | 462 | 954 |
| 144064 | mouse Pcsk9 | 463 | 955 |
| 144065 | mouse Pcsk9 | 464 | 956 |
| 144066 | mouse Pcsk9 | 465 | 957 |
| 144067 | mouse Pcsk9 | 466 | 958 |
| 144068 | mouse Pcsk9 | 467 | 959 |
| 144069 | mouse Pcsk9 | 468 | 960 |
| 144070 | mouse Pcsk9 | 469 | 961 |
| 144071 | mouse Pcsk9 | 470 | 962 |
| 144072 | mouse Pcsk9 | 471 | 963 |
| 144073 | mouse Pcsk9 | 472 | 964 |
| 144074 | mouse Pcsk9 | 473 | 965 |
| 144075 | mouse Pcsk9 | 474 | 966 |
| 144076 | mouse Pcsk9 | 475 | 967 |
| 144077 | mouse Pcsk9 | 476 | 968 |
| 144078 | mouse Pcsk9 | 477 | 969 |
| 144079 | mouse Pcsk9 | 478 | 970 |
| 144080 | mouse Pcsk9 | 479 | 971 |
| 144081 | mouse Pcsk9 | 480 | 972 |
| 144082 | mouse Pcsk9 | 481 | 973 |
| 144083 | mouse Pcsk9 | 482 | 974 |
| 144084 | mouse Pcsk9 | 483 | 975 |
| 144085 | mouse Pcsk9 | 484 | 976 |
| 144086 | mouse Pcsk9 | 485 | 977 |
| 144087 | mouse Pcsk9 | 486 | 978 |
| 144088 | mouse Pcsk9 | 487 | 979 |
| 144089 | mouse Pcsk9 | 488 | 980 |
| 144090 | mouse Pcsk9 | 489 | 981 |
| 144091 | mouse Pcsk9 | 490 | 982 |
| 144092 | mouse Pcsk9 | 491 | 983 |
| 144093 | mouse Pcsk9 | 492 | 984 |
| 144094 | mouse Pcsk9 | 493 | 985 |
| 144095 | mouse Pcsk9 | 494 | 986 |
| 144096 | mouse Pcsk9 | 495 | 987 |
| 144097 | mouse Pcsk9 | 496 | 988 |
| 144098 | mouse Pcsk9 | 497 | 989 |
| 144099 | mouse Pcsk9 | 498 | 990 |
| 144100 | mouse Pcsk9 | 499 | 991 |
| 144101 | mouse Pcsk9 | 500 | 992 |
| 144102 | mouse Pcsk9 | 501 | 993 |
| 144103 | mouse Pcsk9 | 502 | 994 |
| 144104 | mouse Pcsk9 | 503 | 995 |
| 144105 | mouse Pcsk9 | 504 | 996 |
| 144106 | mouse Pcsk9 | 505 | 997 |
| 144107 | mouse Pcsk9 | 506 | 998 |
| 144108 | mouse Pcsk9 | 507 | 999 |
| 144109 | mouse Pcsk9 | 508 | 1000 |
| 144110 | mouse Pcsk9 | 509 | 1001 |
| 144111 | mouse Pcsk9 | 510 | 1002 |
| 144112 | mouse Pcsk9 | 511 | 1003 |
| 144113 | mouse Pcsk9 | 512 | 1004 |
| 144114 | mouse Pcsk9 | 513 | 1005 |
| 144115 | mouse Pcsk9 | 514 | 1006 |
| 144116 | mouse Pcsk9 | 515 | 1007 |
| 144117 | mouse Pcsk9 | 516 | 1008 |
| 144118 | mouse Pcsk9 | 517 | 1009 |
| 144119 | mouse Pcsk9 | 518 | 1010 |
| 144120 | mouse Pcsk9 | 519 | 1011 |
| 144121 | mouse Pcsk9 | 520 | 1012 |
| 144122 | mouse Pcsk9 | 521 | 1013 |
| 144123 | mouse Pcsk9 | 522 | 1014 |
| 144124 | mouse Pcsk9 | 523 | 1015 |
| 144125 | mouse Pcsk9 | 524 | 1016 |
| 144126 | mouse Pcsk9 | 525 | 1017 |
| 144127 | mouse Pcsk9 | 526 | 1018 |
| 144128 | mouse Pcsk9 | 527 | 1019 |
| 144129 | mouse Pcsk9 | 528 | 1020 |

TABLE 10 provides exemplary spacer sequences and full guide nucleic acid sequences for targeting human APOC3, human PCSK9, human ANGPTL3, mouse Apoc3, and mouse Pcsk9 for CasM.265466 effector protein.

TABLE 10
EXEMPLARY SPACER SEQUENCES AND FULL GUIDE
NUCLEIC ACID SEQUENCES FOR CASM.265466

| | | | gRNA |
|---|---|---|---|
| | | Spacer SEQ | SEQ ID |
| Spacer ID | Target | ID NO: | NO: |
| 133653 | human APOC3 | 1021 | 1976 |
| 133654 | human APOC3 | 1022 | 1977 |
| 133655 | human APOC3 | 1023 | 1978 |
| 133656 | human APOC3 | 1024 | 1979 |
| 133657 | human APOC3 | 1025 | 1980 |
| 133658 | human APOC3 | 1026 | 1981 |
| 133659 | human APOC3 | 1027 | 1982 |
| 133660 | human APOC3 | 1028 | 1983 |
| 133661 | human APOC3 | 1029 | 1984 |
| 133662 | human APOC3 | 1030 | 1985 |
| 133663 | human APOC3 | 1031 | 1986 |
| 133664 | human APOC3 | 1032 | 1987 |
| 133665 | human APOC3 | 1033 | 1988 |
| 133666 | human APOC3 | 1034 | 1989 |
| 133667 | human APOC3 | 1035 | 1990 |
| 133668 | human APOC3 | 1036 | 1991 |
| 133669 | human APOC3 | 1037 | 1992 |
| 133670 | human APOC3 | 1038 | 1993 |
| 133671 | human APOC3 | 1039 | 1994 |
| 133672 | human APOC3 | 1040 | 1995 |
| 133673 | human APOC3 | 1041 | 1996 |
| 133674 | human APOC3 | 1042 | 1997 |
| 133675 | human APOC3 | 1043 | 1998 |
| 133676 | human APOC3 | 1044 | 1999 |
| 133677 | human APOC3 | 1045 | 2000 |
| 133678 | human APOC3 | 1046 | 2001 |
| 133679 | human APOC3 | 1047 | 2002 |
| 133680 | human APOC3 | 1048 | 2003 |
| 133681 | human APOC3 | 1049 | 2004 |
| 133682 | human APOC3 | 1050 | 2005 |
| 133683 | human APOC3 | 1051 | 2006 |
| 133684 | human APOC3 | 1052 | 2007 |
| 133685 | human APOC3 | 1053 | 2008 |
| 133686 | human APOC3 | 1054 | 2009 |
| 133687 | human APOC3 | 1055 | 2010 |
| 133688 | human APOC3 | 1056 | 2011 |
| 133689 | human APOC3 | 1057 | 2012 |
| 133690 | human APOC3 | 1058 | 2013 |
| 133691 | human APOC3 | 1059 | 2014 |
| 133692 | human APOC3 | 1060 | 2015 |
| 133693 | human APOC3 | 1061 | 2016 |
| 133694 | human APOC3 | 1062 | 2017 |
| 133695 | human APOC3 | 1063 | 2018 |
| 133696 | human APOC3 | 1064 | 2019 |
| 133697 | human APOC3 | 1065 | 2020 |
| 133698 | human APOC3 | 1066 | 2021 |
| 133699 | human APOC3 | 1067 | 2022 |
| 133700 | human APOC3 | 1068 | 2023 |
| 133701 | human APOC3 | 1069 | 2024 |
| 133702 | human APOC3 | 1070 | 2025 |
| 133703 | human APOC3 | 1071 | 2026 |
| 133704 | human APOC3 | 1072 | 2027 |
| 133705 | human APOC3 | 1073 | 2028 |
| 133706 | human APOC3 | 1074 | 2029 |
| 133707 | human APOC3 | 1075 | 2030 |
| 133708 | human APOC3 | 1076 | 2031 |
| 133709 | human APOC3 | 1077 | 2032 |
| 133710 | human APOC3 | 1078 | 2033 |
| 133711 | human APOC3 | 1079 | 2034 |
| 133712 | human APOC3 | 1080 | 2035 |
| 133713 | human APOC3 | 1081 | 2036 |
| 133714 | human APOC3 | 1082 | 2037 |
| 133715 | human APOC3 | 1083 | 2038 |
| 133716 | human APOC3 | 1084 | 2039 |
| 133717 | human APOC3 | 1085 | 2040 |
| 133718 | human APOC3 | 1086 | 2041 |
| 133719 | human APOC3 | 1087 | 2042 |
| 133720 | human APOC3 | 1088 | 2043 |
| 133721 | human APOC3 | 1089 | 2044 |
| 133722 | human APOC3 | 1090 | 2045 |
| 133723 | human APOC3 | 1091 | 2046 |
| 133724 | human APOC3 | 1092 | 2047 |
| 133725 | human APOC3 | 1093 | 2048 |
| 133726 | human APOC3 | 1094 | 2049 |
| 133727 | human APOC3 | 1095 | 2050 |
| 133728 | human APOC3 | 1096 | 2051 |
| 133729 | human APOC3 | 1097 | 2052 |
| 133730 | human APOC3 | 1098 | 2053 |
| 133731 | human APOC3 | 1099 | 2054 |
| 133732 | human APOC3 | 1100 | 2055 |
| 133733 | human APOC3 | 1101 | 2056 |
| 133734 | human APOC3 | 1102 | 2057 |
| 133735 | human APOC3 | 1103 | 2058 |
| 133736 | human APOC3 | 1104 | 2059 |
| 133737 | human APOC3 | 1105 | 2060 |
| 133738 | human APOC3 | 1106 | 2061 |
| 133739 | human APOC3 | 1107 | 2062 |
| 133740 | human APOC3 | 1108 | 2063 |
| 133741 | human APOC3 | 1109 | 2064 |
| 133742 | human APOC3 | 1110 | 2065 |
| 133743 | human APOC3 | 1111 | 2066 |
| 133744 | human APOC3 | 1112 | 2067 |
| 133745 | human APOC3 | 1113 | 2068 |
| 133746 | human APOC3 | 1114 | 2069 |
| 133747 | human APOC3 | 1115 | 2070 |
| 133748 | human APOC3 | 1116 | 2071 |
| 133749 | human APOC3 | 1117 | 2072 |
| 133750 | human APOC3 | 1118 | 2073 |
| 133751 | human APOC3 | 1119 | 2074 |
| 133752 | human APOC3 | 1120 | 2075 |
| 133753 | human APOC3 | 1121 | 2076 |
| 133754 | human APOC3 | 1122 | 2077 |
| 133755 | human APOC3 | 1123 | 2078 |
| 133756 | human APOC3 | 1124 | 2079 |
| 133757 | human APOC3 | 1125 | 2080 |
| 133758 | human APOC3 | 1126 | 2081 |
| 133759 | human APOC3 | 1127 | 2082 |
| 133760 | human APOC3 | 1128 | 2083 |
| 133761 | human APOC3 | 1129 | 2084 |
| 133762 | human APOC3 | 1130 | 2085 |
| 133763 | human APOC3 | 1131 | 2086 |
| 133764 | human APOC3 | 1132 | 2087 |
| 133765 | human APOC3 | 1133 | 2088 |
| 133766 | human APOC3 | 1134 | 2089 |
| 133767 | human APOC3 | 1135 | 2090 |
| 133768 | human APOC3 | 1136 | 2091 |
| 133769 | human APOC3 | 1137 | 2092 |
| 133770 | human APOC3 | 1138 | 2093 |
| 133771 | human APOC3 | 1139 | 2094 |
| 133772 | human APOC3 | 1140 | 2095 |
| 133773 | human APOC3 | 1141 | 2096 |
| 133774 | human APOC3 | 1142 | 2097 |
| 133775 | human APOC3 | 1143 | 2098 |
| 133776 | human APOC3 | 1144 | 2099 |
| 133777 | human APOC3 | 1145 | 2100 |
| 133778 | human APOC3 | 1146 | 2101 |
| 133779 | human APOC3 | 1147 | 2102 |
| 133780 | human APOC3 | 1148 | 2103 |
| 133781 | human APOC3 | 1149 | 2104 |
| 133782 | human APOC3 | 1150 | 2105 |
| 133783 | human APOC3 | 1151 | 2106 |
| 133784 | human APOC3 | 1152 | 2107 |
| 133785 | human APOC3 | 1153 | 2108 |
| 133786 | human APOC3 | 1154 | 2109 |
| 133787 | human APOC3 | 1155 | 2110 |
| 133788 | human APOC3 | 1156 | 2111 |
| 133789 | human APOC3 | 1157 | 2112 |
| 133790 | human APOC3 | 1158 | 2113 |
| 133791 | human APOC3 | 1159 | 2114 |
| 133792 | human APOC3 | 1160 | 2115 |
| 133793 | human APOC3 | 1161 | 2116 |
| 133794 | human APOC3 | 1162 | 2117 |
| 133795 | human APOC3 | 1163 | 2118 |
| 133796 | human APOC3 | 1164 | 2119 |
| 133797 | human APOC3 | 1165 | 2120 |
| 133798 | human APOC3 | 1166 | 2121 |
| 133799 | human APOC3 | 1167 | 2122 |
| 133800 | human APOC3 | 1168 | 2123 |
| 133801 | human APOC3 | 1169 | 2124 |
| 133802 | human APOC3 | 1170 | 2125 |
| 133803 | human APOC3 | 1171 | 2126 |
| 133804 | human APOC3 | 1172 | 2127 |
| 133805 | human APOC3 | 1173 | 2128 |
| 133806 | human APOC3 | 1174 | 2129 |
| 133807 | human APOC3 | 1175 | 2130 |
| 133808 | human APOC3 | 1176 | 2131 |
| 133809 | human APOC3 | 1177 | 2132 |
| 133810 | human APOC3 | 1178 | 2133 |
| 133811 | human APOC3 | 1179 | 2134 |
| 133812 | human APOC3 | 1180 | 2135 |
| 133813 | human APOC3 | 1181 | 2136 |
| 133814 | human APOC3 | 1182 | 2137 |
| 133815 | human APOC3 | 1183 | 2138 |
| 133816 | human APOC3 | 1184 | 2139 |
| 133817 | human APOC3 | 1185 | 2140 |
| 133818 | human APOC3 | 1186 | 2141 |
| 133819 | human APOC3 | 1187 | 2142 |
| 133820 | human APOC3 | 1188 | 2143 |
| 133821 | human APOC3 | 1189 | 2144 |
| 133822 | human APOC3 | 1190 | 2145 |
| 133823 | human APOC3 | 1191 | 2146 |
| 133824 | human APOC3 | 1192 | 2147 |
| 133825 | human APOC3 | 1193 | 2148 |
| 133826 | human APOC3 | 1194 | 2149 |
| 133827 | human APOC3 | 1195 | 2150 |
| 133828 | human APOC3 | 1196 | 2151 |
| 133829 | human APOC3 | 1197 | 2152 |
| 133830 | human APOC3 | 1198 | 2153 |
| 133831 | human APOC3 | 1199 | 2154 |
| 133832 | human APOC3 | 1200 | 2155 |
| 133833 | human APOC3 | 1201 | 2156 |
| 133834 | human APOC3 | 1202 | 2157 |
| 133835 | human PCSK9 | 1203 | 2158 |
| 133836 | human PCSK9 | 1204 | 2159 |
| 133837 | human PCSK9 | 1205 | 2160 |
| 133838 | human PCSK9 | 1206 | 2161 |
| 133839 | human PCSK9 | 1207 | 2162 |
| 133840 | human PCSK9 | 1208 | 2163 |
| 133841 | human PCSK9 | 1209 | 2164 |
| 133842 | human PCSK9 | 1210 | 2165 |
| 133843 | human PCSK9 | 1211 | 2166 |
| 133844 | human PCSK9 | 1212 | 2167 |
| 133845 | human PCSK9 | 1213 | 2168 |
| 133846 | human PCSK9 | 1214 | 2169 |
| 133847 | human PCSK9 | 1215 | 2170 |
| 133848 | human PCSK9 | 1216 | 2171 |
| 133849 | human PCSK9 | 1217 | 2172 |
| 133850 | human PCSK9 | 1218 | 2173 |
| 133851 | human PCSK9 | 1219 | 2174 |
| 133852 | human PCSK9 | 1220 | 2175 |
| 133853 | human PCSK9 | 1221 | 2176 |
| 133854 | human PCSK9 | 1222 | 2177 |
| 133855 | human PCSK9 | 1223 | 2178 |
| 133856 | human PCSK9 | 1224 | 2179 |
| 133857 | human PCSK9 | 1225 | 2180 |
| 133858 | human PCSK9 | 1226 | 2181 |
| 133859 | human PCSK9 | 1227 | 2182 |
| 133860 | human PCSK9 | 1228 | 2183 |
| 133861 | human PCSK9 | 1229 | 2184 |
| 133862 | human PCSK9 | 1230 | 2185 |
| 133863 | human PCSK9 | 1231 | 2186 |
| 133864 | human PCSK9 | 1232 | 2187 |
| 133865 | human PCSK9 | 1233 | 2188 |
| 133866 | human PCSK9 | 1234 | 2189 |
| 133867 | human PCSK9 | 1235 | 2190 |
| 133868 | human PCSK9 | 1236 | 2191 |
| 133869 | human PCSK9 | 1237 | 2192 |
| 133870 | human PCSK9 | 1238 | 2193 |
| 133871 | human PCSK9 | 1239 | 2194 |
| 133872 | human PCSK9 | 1240 | 2195 |
| 133873 | human PCSK9 | 1241 | 2196 |
| 133874 | human PCSK9 | 1242 | 2197 |
| 133875 | human PCSK9 | 1243 | 2198 |
| 133876 | human PCSK9 | 1244 | 2199 |
| 133877 | human PCSK9 | 1245 | 2200 |
| 133878 | human PCSK9 | 1246 | 2201 |
| 133879 | human PCSK9 | 1247 | 2202 |
| 133880 | human PCSK9 | 1248 | 2203 |
| 133881 | human PCSK9 | 1249 | 2204 |
| 133882 | human PCSK9 | 1250 | 2205 |
| 133883 | human PCSK9 | 1251 | 2206 |
| 133884 | human PCSK9 | 1252 | 2207 |
| 133885 | human PCSK9 | 1253 | 2208 |
| 133886 | human PCSK9 | 1254 | 2209 |
| 133887 | human PCSK9 | 1255 | 2210 |
| 133888 | human PCSK9 | 1256 | 2211 |
| 133889 | human PCSK9 | 1257 | 2212 |
| 133890 | human PCSK9 | 1258 | 2213 |
| 133891 | human PCSK9 | 1259 | 2214 |
| 133892 | human PCSK9 | 1260 | 2215 |
| 133893 | human PCSK9 | 1261 | 2216 |
| 133894 | human PCSK9 | 1262 | 2217 |
| 133895 | human PCSK9 | 1263 | 2218 |
| 133896 | human PCSK9 | 1264 | 2219 |
| 133897 | human PCSK9 | 1265 | 2220 |
| 133898 | human PCSK9 | 1266 | 2221 |
| 133899 | human PCSK9 | 1267 | 2222 |
| 133900 | human PCSK9 | 1268 | 2223 |
| 133901 | human PCSK9 | 1269 | 2224 |
| 133902 | human PCSK9 | 1270 | 2225 |
| 133903 | human PCSK9 | 1271 | 2226 |
| 133904 | human PCSK9 | 1272 | 2227 |
| 133905 | human PCSK9 | 1273 | 2228 |
| 133906 | human PCSK9 | 1274 | 2229 |
| 133907 | human PCSK9 | 1275 | 2230 |
| 133908 | human PCSK9 | 1276 | 2231 |
| 133909 | human PCSK9 | 1277 | 2232 |
| 133910 | human PCSK9 | 1278 | 2233 |
| 133911 | human PCSK9 | 1279 | 2234 |
| 133912 | human PCSK9 | 1280 | 2235 |
| 133913 | human PCSK9 | 1281 | 2236 |
| 133914 | human PCSK9 | 1282 | 2237 |
| 133915 | human PCSK9 | 1283 | 2238 |
| 133916 | human PCSK9 | 1284 | 2239 |
| 133917 | human PCSK9 | 1285 | 2240 |
| 133918 | human PCSK9 | 1286 | 2241 |
| 133919 | human PCSK9 | 1287 | 2242 |
| 133920 | human PCSK9 | 1288 | 2243 |
| 133921 | human PCSK9 | 1289 | 2244 |
| 133922 | human PCSK9 | 1290 | 2245 |
| 133923 | human PCSK9 | 1291 | 2246 |
| 133924 | human PCSK9 | 1292 | 2247 |
| 133925 | human PCSK9 | 1293 | 2248 |
| 133926 | human PCSK9 | 1294 | 2249 |
| 133927 | human PCSK9 | 1295 | 2250 |
| 133928 | human PCSK9 | 1296 | 2251 |
| 133929 | human PCSK9 | 1297 | 2252 |
| 133930 | human PCSK9 | 1298 | 2253 |
| 133931 | human PCSK9 | 1299 | 2254 |
| 133932 | human PCSK9 | 1300 | 2255 |
| 133933 | human PCSK9 | 1301 | 2256 |
| 133934 | human PCSK9 | 1302 | 2257 |
| 133935 | human PCSK9 | 1303 | 2258 |
| 133936 | human PCSK9 | 1304 | 2259 |
| 133937 | human PCSK9 | 1305 | 2260 |
| 133938 | human PCSK9 | 1306 | 2261 |
| 133939 | human PCSK9 | 1307 | 2262 |
| 133940 | human PCSK9 | 1308 | 2263 |
| 133941 | human PCSK9 | 1309 | 2264 |
| 133942 | human PCSK9 | 1310 | 2265 |
| 133943 | human PCSK9 | 1311 | 2266 |
| 133944 | human PCSK9 | 1312 | 2267 |
| 133945 | human PCSK9 | 1313 | 2268 |
| 133946 | human PCSK9 | 1314 | 2269 |
| 133947 | human PCSK9 | 1315 | 2270 |
| 133948 | human PCSK9 | 1316 | 2271 |
| 133949 | human PCSK9 | 1317 | 2272 |
| 133950 | human PCSK9 | 1318 | 2273 |
| 133951 | human PCSK9 | 1319 | 2274 |
| 133952 | human PCSK9 | 1320 | 2275 |
| 133953 | human PCSK9 | 1321 | 2276 |
| 133954 | human PCSK9 | 1322 | 2277 |
| 133955 | human PCSK9 | 1323 | 2278 |
| 133956 | human PCSK9 | 1324 | 2279 |
| 133957 | human PCSK9 | 1325 | 2280 |
| 133958 | human PCSK9 | 1326 | 2281 |
| 133959 | human PCSK9 | 1327 | 2282 |
| 133960 | human PCSK9 | 1328 | 2283 |
| 133961 | human PCSK9 | 1329 | 2284 |
| 133962 | human PCSK9 | 1330 | 2285 |
| 133963 | human PCSK9 | 1331 | 2286 |
| 133964 | human PCSK9 | 1332 | 2287 |
| 133965 | human PCSK9 | 1333 | 2288 |
| 133966 | human PCSK9 | 1334 | 2289 |
| 133967 | human PCSK9 | 1335 | 2290 |
| 133968 | human PCSK9 | 1336 | 2291 |
| 133969 | human PCSK9 | 1337 | 2292 |
| 133970 | human PCSK9 | 1338 | 2293 |
| 133971 | human PCSK9 | 1339 | 2294 |
| 133972 | human PCSK9 | 1340 | 2295 |
| 133973 | human PCSK9 | 1341 | 2296 |
| 133974 | human PCSK9 | 1342 | 2297 |
| 133975 | human PCSK9 | 1343 | 2298 |
| 133976 | human PCSK9 | 1344 | 2299 |
| 133977 | human PCSK9 | 1345 | 2300 |
| 133978 | human PCSK9 | 1346 | 2301 |
| 133979 | human PCSK9 | 1347 | 2302 |
| 133980 | human PCSK9 | 1348 | 2303 |
| 133981 | human PCSK9 | 1349 | 2304 |
| 133982 | human PCSK9 | 1350 | 2305 |
| 133983 | human PCSK9 | 1351 | 2306 |
| 133984 | human PCSK9 | 1352 | 2307 |
| 133985 | human PCSK9 | 1353 | 2308 |
| 133986 | human PCSK9 | 1354 | 2309 |
| 133987 | human PCSK9 | 1355 | 2310 |
| 133988 | human PCSK9 | 1356 | 2311 |
| 133989 | human PCSK9 | 1357 | 2312 |
| 133990 | human PCSK9 | 1358 | 2313 |
| 133991 | human PCSK9 | 1359 | 2314 |
| 133992 | human PCSK9 | 1360 | 2315 |
| 133993 | human PCSK9 | 1361 | 2316 |
| 133994 | human ANGPTL3 | 1362 | 2317 |
| 133995 | human ANGPTL3 | 1363 | 2318 |
| 133996 | human ANGPTL3 | 1364 | 2319 |
| 133997 | human ANGPTL3 | 1365 | 2320 |
| 133998 | human ANGPTL3 | 1366 | 2321 |
| 133999 | human ANGPTL3 | 1367 | 2322 |
| 134000 | human ANGPTL3 | 1368 | 2323 |
| 134001 | human ANGPTL3 | 1369 | 2324 |
| 134002 | human ANGPTL3 | 1370 | 2325 |
| 134003 | human ANGPTL3 | 1371 | 2326 |
| 134004 | human ANGPTL3 | 1372 | 2327 |
| 134005 | human ANGPTL3 | 1373 | 2328 |
| 134006 | human ANGPTL3 | 1374 | 2329 |
| 134007 | human ANGPTL3 | 1375 | 2330 |
| 134008 | human ANGPTL3 | 1376 | 2331 |
| 134009 | human ANGPTL3 | 1377 | 2332 |
| 134010 | human ANGPTL3 | 1378 | 2333 |
| 134011 | human ANGPTL3 | 1379 | 2334 |
| 134012 | human ANGPTL3 | 1380 | 2335 |
| 134013 | human ANGPTL3 | 1381 | 2336 |
| 134014 | human ANGPTL3 | 1382 | 2337 |
| 134015 | human ANGPTL3 | 1383 | 2338 |
| 134016 | human ANGPTL3 | 1384 | 2339 |
| 134017 | human ANGPTL3 | 1385 | 2340 |
| 134018 | human ANGPTL3 | 1386 | 2341 |
| 134019 | human ANGPTL3 | 1387 | 2342 |
| 134020 | human ANGPTL3 | 1388 | 2343 |
| 134021 | human ANGPTL3 | 1389 | 2344 |
| 134022 | human ANGPTL3 | 1390 | 2345 |
| 134023 | human ANGPTL3 | 1391 | 2346 |
| 134024 | human ANGPTL3 | 1392 | 2347 |
| 134025 | human ANGPTL3 | 1393 | 2348 |
| 134026 | human ANGPTL3 | 1394 | 2349 |
| 134027 | human ANGPTL3 | 1395 | 2350 |
| 134028 | human ANGPTL3 | 1396 | 2351 |
| 134029 | human ANGPTL3 | 1397 | 2352 |
| 134030 | human ANGPTL3 | 1398 | 2353 |
| 134031 | human ANGPTL3 | 1399 | 2354 |
| 134032 | human ANGPTL3 | 1400 | 2355 |
| 134033 | human ANGPTL3 | 1401 | 2356 |
| 134034 | human ANGPTL3 | 1402 | 2357 |
| 134035 | human ANGPTL3 | 1403 | 2358 |
| 134036 | human ANGPTL3 | 1404 | 2359 |
| 134037 | human ANGPTL3 | 1405 | 2360 |
| 134038 | human ANGPTL3 | 1406 | 2361 |
| 134039 | human ANGPTL3 | 1407 | 2362 |
| 134040 | human ANGPTL3 | 1408 | 2363 |
| 134041 | human ANGPTL3 | 1409 | 2364 |
| 134042 | human ANGPTL3 | 1410 | 2365 |
| 134043 | human ANGPTL3 | 1411 | 2366 |
| 134044 | human ANGPTL3 | 1412 | 2367 |
| 134045 | human ANGPTL3 | 1413 | 2368 |
| 134046 | human ANGPTL3 | 1414 | 2369 |
| 134047 | human ANGPTL3 | 1415 | 2370 |
| 134048 | human ANGPTL3 | 1416 | 2371 |
| 134049 | human ANGPTL3 | 1417 | 2372 |
| 134050 | human ANGPTL3 | 1418 | 2373 |
| 134051 | human ANGPTL3 | 1419 | 2374 |
| 134052 | human ANGPTL3 | 1420 | 2375 |
| 134053 | human ANGPTL3 | 1421 | 2376 |
| 134054 | human ANGPTL3 | 1422 | 2377 |
| 134055 | human ANGPTL3 | 1423 | 2378 |
| 134056 | human ANGPTL3 | 1424 | 2379 |
| 134057 | human ANGPTL3 | 1425 | 2380 |
| 134058 | human ANGPTL3 | 1426 | 2381 |
| 134059 | human ANGPTL3 | 1427 | 2382 |
| 134060 | human ANGPTL3 | 1428 | 2383 |
| 134061 | human ANGPTL3 | 1429 | 2384 |
| 134062 | human ANGPTL3 | 1430 | 2385 |
| 134063 | human ANGPTL3 | 1431 | 2386 |
| 134064 | human ANGPTL3 | 1432 | 2387 |
| 134065 | human ANGPTL3 | 1433 | 2388 |
| 134066 | human ANGPTL3 | 1434 | 2389 |
| 134067 | human ANGPTL3 | 1435 | 2390 |
| 134068 | human ANGPTL3 | 1436 | 2391 |
| 134069 | human ANGPTL3 | 1437 | 2392 |
| 134070 | human ANGPTL3 | 1438 | 2393 |
| 134071 | human ANGPTL3 | 1439 | 2394 |
| 134072 | human ANGPTL3 | 1440 | 2395 |
| 134073 | human ANGPTL3 | 1441 | 2396 |
| 134074 | human ANGPTL3 | 1442 | 2397 |
| 134075 | human ANGPTL3 | 1443 | 2398 |
| 134076 | human ANGPTL3 | 1444 | 2399 |
| 134077 | human ANGPTL3 | 1445 | 2400 |
| 134078 | human ANGPTL3 | 1446 | 2401 |
| 134079 | human ANGPTL3 | 1447 | 2402 |
| 134080 | human ANGPTL3 | 1448 | 2403 |
| 134081 | human ANGPTL3 | 1449 | 2404 |
| 134082 | human ANGPTL3 | 1450 | 2405 |
| 134083 | human ANGPTL3 | 1451 | 2406 |
| 134084 | human ANGPTL3 | 1452 | 2407 |
| 134085 | human ANGPTL3 | 1453 | 2408 |
| 134086 | human ANGPTL3 | 1454 | 2409 |
| 134087 | human ANGPTL3 | 1455 | 2410 |
| 134088 | human ANGPTL3 | 1456 | 2411 |
| 134089 | human ANGPTL3 | 1457 | 2412 |
| 134090 | human ANGPTL3 | 1458 | 2413 |
| 134091 | human ANGPTL3 | 1459 | 2414 |
| 134092 | human ANGPTL3 | 1460 | 2415 |
| 134093 | human ANGPTL3 | 1461 | 2416 |
| 134094 | human ANGPTL3 | 1462 | 2417 |
| 134095 | human ANGPTL3 | 1463 | 2418 |
| 134096 | human ANGPTL3 | 1464 | 2419 |
| 134097 | human ANGPTL3 | 1465 | 2420 |
| 134098 | human ANGPTL3 | 1466 | 2421 |
| 134099 | human ANGPTL3 | 1467 | 2422 |
| 134100 | human ANGPTL3 | 1468 | 2423 |
| 134101 | human ANGPTL3 | 1469 | 2424 |
| 134102 | human ANGPTL3 | 1470 | 2425 |
| 134103 | human ANGPTL3 | 1471 | 2426 |
| 134104 | human ANGPTL3 | 1472 | 2427 |
| 134105 | human ANGPTL3 | 1473 | 2428 |
| 134106 | human ANGPTL3 | 1474 | 2429 |
| 134107 | human ANGPTL3 | 1475 | 2430 |
| 134108 | human ANGPTL3 | 1476 | 2431 |
| 134109 | human ANGPTL3 | 1477 | 2432 |
| 134110 | human ANGPTL3 | 1478 | 2433 |
| 134111 | human ANGPTL3 | 1479 | 2434 |
| 134112 | human ANGPTL3 | 1480 | 2435 |
| 134113 | human ANGPTL3 | 1481 | 2436 |
| 134114 | human ANGPTL3 | 1482 | 2437 |
| 134115 | human ANGPTL3 | 1483 | 2438 |
| 134116 | human ANGPTL3 | 1484 | 2439 |
| 134117 | human ANGPTL3 | 1485 | 2440 |
| 134118 | human ANGPTL3 | 1486 | 2441 |
| 134119 | human ANGPTL3 | 1487 | 2442 |
| 134120 | human ANGPTL3 | 1488 | 2443 |
| 134121 | human ANGPTL3 | 1489 | 2444 |
| 134122 | human ANGPTL3 | 1490 | 2445 |
| 134123 | human ANGPTL3 | 1491 | 2446 |
| 134124 | human ANGPTL3 | 1492 | 2447 |
| 134125 | human ANGPTL3 | 1493 | 2448 |
| 134126 | human ANGPTL3 | 1494 | 2449 |
| 134127 | human ANGPTL3 | 1495 | 2450 |
| 134128 | human ANGPTL3 | 1496 | 2451 |
| 134129 | human ANGPTL3 | 1497 | 2452 |
| 134130 | human ANGPTL3 | 1498 | 2453 |
| 134131 | human ANGPTL3 | 1499 | 2454 |
| 134132 | human ANGPTL3 | 1500 | 2455 |
| 134133 | human ANGPTL3 | 1501 | 2456 |
| 134134 | human ANGPTL3 | 1502 | 2457 |
| 134135 | human ANGPTL3 | 1503 | 2458 |
| 134136 | human ANGPTL3 | 1504 | 2459 |
| 134137 | human ANGPTL3 | 1505 | 2460 |
| 134138 | human ANGPTL3 | 1506 | 2461 |
| 134139 | human ANGPTL3 | 1507 | 2462 |
| 134140 | human ANGPTL3 | 1508 | 2463 |
| 134141 | human ANGPTL3 | 1509 | 2464 |
| 134142 | human ANGPTL3 | 1510 | 2465 |
| 134143 | human ANGPTL3 | 1511 | 2466 |
| 134144 | human ANGPTL3 | 1512 | 2467 |
| 134145 | human ANGPTL3 | 1513 | 2468 |
| 134146 | human ANGPTL3 | 1514 | 2469 |
| 134147 | human ANGPTL3 | 1515 | 2470 |
| 134148 | human ANGPTL3 | 1516 | 2471 |
| 134149 | human ANGPTL3 | 1517 | 2472 |
| 134150 | human ANGPTL3 | 1518 | 2473 |
| 134151 | human ANGPTL3 | 1519 | 2474 |
| 134152 | human ANGPTL3 | 1520 | 2475 |
| 134153 | human ANGPTL3 | 1521 | 2476 |
| 134154 | human ANGPTL3 | 1522 | 2477 |
| 134155 | human ANGPTL3 | 1523 | 2478 |
| 134156 | human ANGPTL3 | 1524 | 2479 |
| 134157 | human ANGPTL3 | 1525 | 2480 |
| 134158 | human ANGPTL3 | 1526 | 2481 |
| 134159 | human ANGPTL3 | 1527 | 2482 |
| 134160 | human ANGPTL3 | 1528 | 2483 |
| 134161 | human ANGPTL3 | 1529 | 2484 |
| 134162 | human ANGPTL3 | 1530 | 2485 |
| 134163 | human ANGPTL3 | 1531 | 2486 |
| 134164 | human ANGPTL3 | 1532 | 2487 |
| 134165 | human ANGPTL3 | 1533 | 2488 |
| 134166 | human ANGPTL3 | 1534 | 2489 |
| 134167 | human ANGPTL3 | 1535 | 2490 |
| 134168 | human ANGPTL3 | 1536 | 2491 |
| 134169 | human ANGPTL3 | 1537 | 2492 |
| 134170 | human ANGPTL3 | 1538 | 2493 |
| 134171 | human ANGPTL3 | 1539 | 2494 |
| 134172 | human ANGPTL3 | 1540 | 2495 |
| 134173 | human ANGPTL3 | 1541 | 2496 |
| 134174 | human ANGPTL3 | 1542 | 2497 |
| 134175 | human ANGPTL3 | 1543 | 2498 |
| 134176 | human ANGPTL3 | 1544 | 2499 |
| 134177 | human ANGPTL3 | 1545 | 2500 |
| 134178 | human ANGPTL3 | 1546 | 2501 |
| 134179 | human ANGPTL3 | 1547 | 2502 |
| 134180 | human ANGPTL3 | 1548 | 2503 |
| 134181 | human ANGPTL3 | 1549 | 2504 |
| 134182 | human ANGPTL3 | 1550 | 2505 |
| 134183 | human ANGPTL3 | 1551 | 2506 |
| 134184 | human ANGPTL3 | 1552 | 2507 |
| 134185 | human ANGPTL3 | 1553 | 2508 |
| 134186 | human ANGPTL3 | 1554 | 2509 |
| 134187 | human ANGPTL3 | 1555 | 2510 |
| 134188 | human ANGPTL3 | 1556 | 2511 |
| 134189 | human ANGPTL3 | 1557 | 2512 |
| 134190 | human ANGPTL3 | 1558 | 2513 |
| 134191 | human ANGPTL3 | 1559 | 2514 |
| 134192 | human ANGPTL3 | 1560 | 2515 |
| 134193 | human ANGPTL3 | 1561 | 2516 |
| 134194 | human ANGPTL3 | 1562 | 2517 |
| 134195 | human ANGPTL3 | 1563 | 2518 |
| 134196 | human ANGPTL3 | 1564 | 2519 |
| 134197 | human ANGPTL3 | 1565 | 2520 |
| 134198 | human ANGPTL3 | 1566 | 2521 |
| 134199 | human ANGPTL3 | 1567 | 2522 |
| 134200 | human ANGPTL3 | 1568 | 2523 |
| 134201 | human ANGPTL3 | 1569 | 2524 |
| 134202 | human ANGPTL3 | 1570 | 2525 |
| 134203 | human ANGPTL3 | 1571 | 2526 |
| 134204 | human ANGPTL3 | 1572 | 2527 |
| 134205 | human ANGPTL3 | 1573 | 2528 |
| 134206 | human ANGPTL3 | 1574 | 2529 |
| 134207 | human ANGPTL3 | 1575 | 2530 |
| 134208 | human ANGPTL3 | 1576 | 2531 |
| 134209 | human ANGPTL3 | 1577 | 2532 |
| 134210 | human ANGPTL3 | 1578 | 2533 |
| 134211 | human ANGPTL3 | 1579 | 2534 |
| 134212 | human ANGPTL3 | 1580 | 2535 |
| 134213 | human ANGPTL3 | 1581 | 2536 |
| 134214 | human ANGPTL3 | 1582 | 2537 |
| 144542 | mouse Apoc3 | 1583 | 2538 |
| 144543 | mouse Apoc3 | 1584 | 2539 |
| 144544 | mouse Apoc3 | 1585 | 2540 |
| 144545 | mouse Apoc3 | 1586 | 2541 |
| 144546 | mouse Apoc3 | 1587 | 2542 |
| 144547 | mouse Apoc3 | 1588 | 2543 |
| 144548 | mouse Apoc3 | 1589 | 2544 |
| 144549 | mouse Apoc3 | 1590 | 2545 |
| 144550 | mouse Apoc3 | 1591 | 2546 |
| 144551 | mouse Apoc3 | 1592 | 2547 |
| 144552 | mouse Apoc3 | 1593 | 2548 |
| 144553 | mouse Apoc3 | 1594 | 2549 |
| 144554 | mouse Apoc3 | 1595 | 2550 |
| 144555 | mouse Apoc3 | 1596 | 2551 |
| 144556 | mouse Apoc3 | 1597 | 2552 |
| 144557 | mouse Apoc3 | 1598 | 2553 |
| 144558 | mouse Apoc3 | 1599 | 2554 |
| 144559 | mouse Apoc3 | 1600 | 2555 |
| 144560 | mouse Apoc3 | 1601 | 2556 |
| 144561 | mouse Apoc3 | 1602 | 2557 |
| 144562 | mouse Apoc3 | 1603 | 2558 |
| 144563 | mouse Apoc3 | 1604 | 2559 |
| 144564 | mouse Apoc3 | 1605 | 2560 |
| 144565 | mouse Apoc3 | 1606 | 2561 |
| 144566 | mouse Apoc3 | 1607 | 2562 |
| 144567 | mouse Apoc3 | 1608 | 2563 |
| 144568 | mouse Apoc3 | 1609 | 2564 |
| 144569 | mouse Apoc3 | 1610 | 2565 |
| 144570 | mouse Apoc3 | 1611 | 2566 |
| 144571 | mouse Apoc3 | 1612 | 2567 |
| 144572 | mouse Apoc3 | 1613 | 2568 |
| 144573 | mouse Apoc3 | 1614 | 2569 |
| 144574 | mouse Apoc3 | 1615 | 2570 |
| 144575 | mouse Apoc3 | 1616 | 2571 |
| 144576 | mouse Apoc3 | 1617 | 2572 |
| 144577 | mouse Apoc3 | 1618 | 2573 |
| 144578 | mouse Apoc3 | 1619 | 2574 |
| 144579 | mouse Apoc3 | 1620 | 2575 |
| 144580 | mouse Apoc3 | 1621 | 2576 |
| 144581 | mouse Apoc3 | 1622 | 2577 |
| 144582 | mouse Apoc3 | 1623 | 2578 |
| 144583 | mouse Apoc3 | 1624 | 2579 |
| 144584 | mouse Apoc3 | 1625 | 2580 |
| 144585 | mouse Apoc3 | 1626 | 2581 |
| 144586 | mouse Apoc3 | 1627 | 2582 |
| 144587 | mouse Apoc3 | 1628 | 2583 |
| 144588 | mouse Apoc3 | 1629 | 2584 |
| 144589 | mouse Apoc3 | 1630 | 2585 |
| 144590 | mouse Apoc3 | 1631 | 2586 |
| 144591 | mouse Apoc3 | 1632 | 2587 |
| 144592 | mouse Apoc3 | 1633 | 2588 |
| 144593 | mouse Apoc3 | 1634 | 2589 |
| 144594 | mouse Apoc3 | 1635 | 2590 |
| 144595 | mouse Apoc3 | 1636 | 2591 |
| 144596 | mouse Apoc3 | 1637 | 2592 |
| 144597 | mouse Apoc3 | 1638 | 2593 |
| 144598 | mouse Apoc3 | 1639 | 2594 |
| 144599 | mouse Apoc3 | 1640 | 2595 |
| 144600 | mouse Apoc3 | 1641 | 2596 |
| 144601 | mouse Apoc3 | 1642 | 2597 |
| 144602 | mouse Apoc3 | 1643 | 2598 |
| 144603 | mouse Apoc3 | 1644 | 2599 |
| 144604 | mouse Apoc3 | 1645 | 2600 |
| 144605 | mouse Apoc3 | 1646 | 2601 |
| 144606 | mouse Apoc3 | 1647 | 2602 |
| 144607 | mouse Apoc3 | 1648 | 2603 |
| 144608 | mouse Apoc3 | 1649 | 2604 |
| 144609 | mouse Apoc3 | 1650 | 2605 |
| 144610 | mouse Apoc3 | 1651 | 2606 |
| 144611 | mouse Apoc3 | 1652 | 2607 |
| 144612 | mouse Apoc3 | 1653 | 2608 |
| 144613 | mouse Apoc3 | 1654 | 2609 |
| 144614 | mouse Apoc3 | 1655 | 2610 |
| 144615 | mouse Apoc3 | 1656 | 2611 |
| 144616 | mouse Apoc3 | 1657 | 2612 |
| 144617 | mouse Apoc3 | 1658 | 2613 |
| 144618 | mouse Apoc3 | 1659 | 2614 |
| 144619 | mouse Apoc3 | 1660 | 2615 |
| 144620 | mouse Apoc3 | 1661 | 2616 |
| 144621 | mouse Apoc3 | 1662 | 2617 |
| 144622 | mouse Apoc3 | 1663 | 2618 |
| 144623 | mouse Apoc3 | 1664 | 2619 |
| 144624 | mouse Apoc3 | 1665 | 2620 |
| 144625 | mouse Apoc3 | 1666 | 2621 |
| 144626 | mouse Apoc3 | 1667 | 2622 |
| 144627 | mouse Apoc3 | 1668 | 2623 |
| 144628 | mouse Apoc3 | 1669 | 2624 |
| 144629 | mouse Apoc3 | 1670 | 2625 |
| 144630 | mouse Apoc3 | 1671 | 2626 |
| 144631 | mouse Apoc3 | 1672 | 2627 |
| 144632 | mouse Apoc3 | 1673 | 2628 |
| 144633 | mouse Apoc3 | 1674 | 2629 |
| 144634 | mouse Apoc3 | 1675 | 2630 |
| 144635 | mouse Apoc3 | 1676 | 2631 |
| 144636 | mouse Apoc3 | 1677 | 2632 |
| 144637 | mouse Apoc3 | 1678 | 2633 |
| 144638 | mouse Apoc3 | 1679 | 2634 |
| 144639 | mouse Apoc3 | 1680 | 2635 |
| 144640 | mouse Apoc3 | 1681 | 2636 |
| 144641 | mouse Apoc3 | 1682 | 2637 |
| 144642 | mouse Apoc3 | 1683 | 2638 |
| 144643 | mouse Apoc3 | 1684 | 2639 |
| 144644 | mouse Apoc3 | 1685 | 2640 |
| 144645 | mouse Apoc3 | 1686 | 2641 |
| 144646 | mouse Apoc3 | 1687 | 2642 |
| 144647 | mouse Apoc3 | 1688 | 2643 |
| 144648 | mouse Apoc3 | 1689 | 2644 |
| 144649 | mouse Apoc3 | 1690 | 2645 |
| 144650 | mouse Apoc3 | 1691 | 2646 |
| 144651 | mouse Apoc3 | 1692 | 2647 |
| 144652 | mouse Apoc3 | 1693 | 2648 |
| 144653 | mouse Apoc3 | 1694 | 2649 |
| 144654 | mouse Apoc3 | 1695 | 2650 |
| 144655 | mouse Apoc3 | 1696 | 2651 |
| 144656 | mouse Apoc3 | 1697 | 2652 |
| 144657 | mouse Apoc3 | 1698 | 2653 |
| 144658 | mouse Apoc3 | 1699 | 2654 |
| 144659 | mouse Apoc3 | 1700 | 2655 |
| 144660 | mouse Apoc3 | 1701 | 2656 |
| 144661 | mouse Apoc3 | 1702 | 2657 |
| 144662 | mouse Apoc3 | 1703 | 2658 |
| 144663 | mouse Apoc3 | 1704 | 2659 |
| 144664 | mouse Apoc3 | 1705 | 2660 |
| 144665 | mouse Apoc3 | 1706 | 2661 |
| 144666 | mouse Apoc3 | 1707 | 2662 |
| 144667 | mouse Apoc3 | 1708 | 2663 |
| 144668 | mouse Apoc3 | 1709 | 2664 |
| 144669 | mouse Apoc3 | 1710 | 2665 |
| 144670 | mouse Apoc3 | 1711 | 2666 |
| 144671 | mouse Apoc3 | 1712 | 2667 |
| 144672 | mouse Apoc3 | 1713 | 2668 |
| 144673 | mouse Apoc3 | 1714 | 2669 |
| 144674 | mouse Apoc3 | 1715 | 2670 |
| 144675 | mouse Apoc3 | 1716 | 2671 |
| 144676 | mouse Apoc3 | 1717 | 2672 |
| 144677 | mouse Apoc3 | 1718 | 2673 |
| 144678 | mouse Apoc3 | 1719 | 2674 |
| 144679 | mouse Apoc3 | 1720 | 2675 |
| 144680 | mouse Apoc3 | 1721 | 2676 |
| 144681 | mouse Apoc3 | 1722 | 2677 |
| 144682 | mouse Apoc3 | 1723 | 2678 |
| 144683 | mouse Apoc3 | 1724 | 2679 |
| 144684 | mouse Apoc3 | 1725 | 2680 |
| 144685 | mouse Apoc3 | 1726 | 2681 |
| 144686 | mouse Apoc3 | 1727 | 2682 |
| 144687 | mouse Apoc3 | 1728 | 2683 |
| 144688 | mouse Apoc3 | 1729 | 2684 |
| 144689 | mouse Apoc3 | 1730 | 2685 |
| 144690 | mouse Apoc3 | 1731 | 2686 |
| 144691 | mouse Apoc3 | 1732 | 2687 |
| 144692 | mouse Apoc3 | 1733 | 2688 |
| 144693 | mouse Apoc3 | 1734 | 2689 |
| 144694 | mouse Apoc3 | 1735 | 2690 |
| 144695 | mouse Apoc3 | 1736 | 2691 |
| 144696 | mouse Apoc3 | 1737 | 2692 |
| 144697 | mouse Apoc3 | 1738 | 2693 |
| 144698 | mouse Apoc3 | 1739 | 2694 |
| 144699 | mouse Apoc3 | 1740 | 2695 |
| 144700 | mouse Apoc3 | 1741 | 2696 |
| 144701 | mouse Apoc3 | 1742 | 2697 |
| 144702 | mouse Apoc3 | 1743 | 2698 |
| 144703 | mouse Apoc3 | 1744 | 2699 |
| 144704 | mouse Apoc3 | 1745 | 2700 |
| 144705 | mouse Apoc3 | 1746 | 2701 |
| 144706 | mouse Apoc3 | 1747 | 2702 |
| 144707 | mouse Apoc3 | 1748 | 2703 |
| 144708 | mouse Apoc3 | 1749 | 2704 |
| 144709 | mouse Apoc3 | 1750 | 2705 |
| 144710 | mouse Apoc3 | 1751 | 2706 |
| 144711 | mouse Apoc3 | 1752 | 2707 |
| 144712 | mouse Apoc3 | 1753 | 2708 |
| 144713 | mouse Apoc3 | 1754 | 2709 |
| 144714 | mouse Apoc3 | 1755 | 2710 |
| 144715 | mouse Apoc3 | 1756 | 2711 |
| 144716 | mouse Apoc3 | 1757 | 2712 |
| 144717 | mouse Apoc3 | 1758 | 2713 |
| 144718 | mouse Apoc3 | 1759 | 2714 |
| 144719 | mouse Apoc3 | 1760 | 2715 |
| 144720 | mouse Apoc3 | 1761 | 2716 |
| 144721 | mouse Apoc3 | 1762 | 2717 |
| 144722 | mouse Apoc3 | 1763 | 2718 |
| 144723 | mouse Apoc3 | 1764 | 2719 |
| 144724 | mouse Apoc3 | 1765 | 2720 |
| 144725 | mouse Apoc3 | 1766 | 2721 |
| 144726 | mouse Apoc3 | 1767 | 2722 |
| 144727 | mouse Apoc3 | 1768 | 2723 |
| 144728 | mouse Apoc3 | 1769 | 2724 |
| 144729 | mouse Apoc3 | 1770 | 2725 |
| 144730 | mouse Apoc3 | 1771 | 2726 |
| 144731 | mouse Apoc3 | 1772 | 2727 |
| 144732 | mouse Apoc3 | 1773 | 2728 |
| 144733 | mouse Apoc3 | 1774 | 2729 |
| 144734 | mouse Apoc3 | 1775 | 2730 |
| 144735 | mouse Apoc3 | 1776 | 2731 |
| 144736 | mouse Apoc3 | 1777 | 2732 |
| 144737 | mouse Apoc3 | 1778 | 2733 |
| 144738 | mouse Apoc3 | 1779 | 2734 |
| 144739 | mouse Apoc3 | 1780 | 2735 |
| 144740 | mouse Apoc3 | 1781 | 2736 |
| 144741 | mouse Apoc3 | 1782 | 2737 |
| 144742 | mouse Apoc3 | 1783 | 2738 |
| 144743 | mouse Apoc3 | 1784 | 2739 |
| 144744 | mouse Apoc3 | 1785 | 2740 |
| 144745 | mouse Apoc3 | 1786 | 2741 |
| 144746 | mouse Apoc3 | 1787 | 2742 |
| 144747 | mouse Apoc3 | 1788 | 2743 |
| 144748 | mouse Apoc3 | 1789 | 2744 |
| 144749 | mouse Apoc3 | 1790 | 2745 |
| 144750 | mouse Apoc3 | 1791 | 2746 |
| 144751 | mouse Apoc3 | 1792 | 2747 |
| 144752 | mouse Apoc3 | 1793 | 2748 |
| 144753 | mouse Apoc3 | 1794 | 2749 |
| 144754 | mouse Apoc3 | 1795 | 2750 |
| 144755 | mouse Apoc3 | 1796 | 2751 |
| 144756 | mouse Apoc3 | 1797 | 2752 |
| 144757 | mouse Apoc3 | 1798 | 2753 |
| 144758 | mouse Apoc3 | 1799 | 2754 |
| 144759 | mouse Apoc3 | 1800 | 2755 |
| 144760 | mouse Pcsk9 | 1801 | 2756 |
| 144761 | mouse Pcsk9 | 1802 | 2757 |
| 144762 | mouse Pcsk9 | 1803 | 2758 |
| 144763 | mouse Pcsk9 | 1804 | 2759 |
| 144764 | mouse Pcsk9 | 1805 | 2760 |
| 144765 | mouse Pcsk9 | 1806 | 2761 |
| 144766 | mouse Pcsk9 | 1807 | 2762 |
| 144767 | mouse Pcsk9 | 1808 | 2763 |
| 144768 | mouse Pcsk9 | 1809 | 2764 |
| 144769 | mouse Pcsk9 | 1810 | 2765 |
| 144770 | mouse Pcsk9 | 1811 | 2766 |
| 144771 | mouse Pcsk9 | 1812 | 2767 |
| 144772 | mouse Pcsk9 | 1813 | 2768 |
| 144773 | mouse Pcsk9 | 1814 | 2769 |
| 144774 | mouse Pcsk9 | 1815 | 2770 |
| 144775 | mouse Pcsk9 | 1816 | 2771 |
| 144776 | mouse Pcsk9 | 1817 | 2772 |
| 144777 | mouse Pcsk9 | 1818 | 2773 |
| 144778 | mouse Pcsk9 | 1819 | 2774 |
| 144779 | mouse Pcsk9 | 1820 | 2775 |
| 144780 | mouse Pcsk9 | 1821 | 2776 |
| 144781 | mouse Pcsk9 | 1822 | 2777 |
| 144782 | mouse Pcsk9 | 1823 | 2778 |
| 144783 | mouse Pcsk9 | 1824 | 2779 |
| 144784 | mouse Pcsk9 | 1825 | 2780 |
| 144785 | mouse Pcsk9 | 1826 | 2781 |
| 144786 | mouse Pcsk9 | 1827 | 2782 |
| 144787 | mouse Pcsk9 | 1828 | 2783 |
| 144788 | mouse Pcsk9 | 1829 | 2784 |
| 144789 | mouse Pcsk9 | 1830 | 2785 |
| 144790 | mouse Pcsk9 | 1831 | 2786 |
| 144791 | mouse Pcsk9 | 1832 | 2787 |
| 144792 | mouse Pcsk9 | 1833 | 2788 |
| 144793 | mouse Pcsk9 | 1834 | 2789 |
| 144794 | mouse Pcsk9 | 1835 | 2790 |
| 144795 | mouse Pcsk9 | 1836 | 2791 |
| 144796 | mouse Pcsk9 | 1837 | 2792 |
| 144797 | mouse Pcsk9 | 1838 | 2793 |
| 144798 | mouse Pcsk9 | 1839 | 2794 |
| 144799 | mouse Pcsk9 | 1840 | 2795 |
| 144800 | mouse Pcsk9 | 1841 | 2796 |
| 144801 | mouse Pcsk9 | 1842 | 2797 |
| 144802 | mouse Pcsk9 | 1843 | 2798 |
| 144803 | mouse Pcsk9 | 1844 | 2799 |
| 144804 | mouse Pcsk9 | 1845 | 2800 |
| 144805 | mouse Pcsk9 | 1846 | 2801 |
| 144806 | mouse Pcsk9 | 1847 | 2802 |
| 144807 | mouse Pcsk9 | 1848 | 2803 |
| 144808 | mouse Pcsk9 | 1849 | 2804 |
| 144809 | mouse Pcsk9 | 1850 | 2805 |
| 144810 | mouse Pcsk9 | 1851 | 2806 |
| 144811 | mouse Pcsk9 | 1852 | 2807 |
| 144812 | mouse Pcsk9 | 1853 | 2808 |
| 144813 | mouse Pcsk9 | 1854 | 2809 |
| 144814 | mouse Pcsk9 | 1855 | 2810 |
| 144815 | mouse Pcsk9 | 1856 | 2811 |
| 144816 | mouse Pcsk9 | 1857 | 2812 |
| 144817 | mouse Pcsk9 | 1858 | 2813 |
| 144818 | mouse Pcsk9 | 1859 | 2814 |
| 144819 | mouse Pcsk9 | 1860 | 2815 |
| 144820 | mouse Pcsk9 | 1861 | 2816 |
| 144821 | mouse Pcsk9 | 1862 | 2817 |
| 144822 | mouse Pcsk9 | 1863 | 2818 |
| 144823 | mouse Pcsk9 | 1864 | 2819 |
| 144824 | mouse Pcsk9 | 1865 | 2820 |
| 144825 | mouse Pcsk9 | 1866 | 2821 |
| 144826 | mouse Pcsk9 | 1867 | 2822 |
| 144827 | mouse Pcsk9 | 1868 | 2823 |
| 144828 | mouse Pcsk9 | 1869 | 2824 |
| 144829 | mouse Pcsk9 | 1870 | 2825 |
| 144830 | mouse Pcsk9 | 1871 | 2826 |
| 144831 | mouse Pcsk9 | 1872 | 2827 |
| 144832 | mouse Pcsk9 | 1873 | 2828 |
| 144833 | mouse Pcsk9 | 1874 | 2829 |
| 144834 | mouse Pcsk9 | 1875 | 2830 |
| 144835 | mouse Pcsk9 | 1876 | 2831 |
| 144836 | mouse Pcsk9 | 1877 | 2832 |
| 144837 | mouse Pcsk9 | 1878 | 2833 |
| 144838 | mouse Pcsk9 | 1879 | 2834 |
| 144839 | mouse Pcsk9 | 1880 | 2835 |
| 144840 | mouse Pcsk9 | 1881 | 2836 |
| 144841 | mouse Pcsk9 | 1882 | 2837 |
| 144842 | mouse Pcsk9 | 1883 | 2838 |
| 144843 | mouse Pcsk9 | 1884 | 2839 |
| 144844 | mouse Pcsk9 | 1885 | 2840 |
| 144845 | mouse Pcsk9 | 1886 | 2841 |
| 144846 | mouse Pcsk9 | 1887 | 2842 |
| 144847 | mouse Pcsk9 | 1888 | 2843 |
| 144848 | mouse Pcsk9 | 1889 | 2844 |
| 144849 | mouse Pcsk9 | 1890 | 2845 |
| 144850 | mouse Pcsk9 | 1891 | 2846 |
| 144851 | mouse Pcsk9 | 1892 | 2847 |
| 144852 | mouse Pcsk9 | 1893 | 2848 |
| 144853 | mouse Pcsk9 | 1894 | 2849 |
| 144854 | mouse Pcsk9 | 1895 | 2850 |
| 144855 | mouse Pcsk9 | 1896 | 2851 |
| 144856 | mouse Pcsk9 | 1897 | 2852 |
| 144857 | mouse Pcsk9 | 1898 | 2853 |
| 144858 | mouse Pcsk9 | 1899 | 2854 |
| 144859 | mouse Pcsk9 | 1900 | 2855 |
| 144860 | mouse Pcsk9 | 1901 | 2856 |
| 144861 | mouse Pcsk9 | 1902 | 2857 |
| 144862 | mouse Pcsk9 | 1903 | 2858 |
| 144863 | mouse Pcsk9 | 1904 | 2859 |
| 144864 | mouse Pcsk9 | 1905 | 2860 |
| 144865 | mouse Pcsk9 | 1906 | 2861 |
| 144866 | mouse Pcsk9 | 1907 | 2862 |
| 144867 | mouse Pcsk9 | 1908 | 2863 |
| 144868 | mouse Pcsk9 | 1909 | 2864 |
| 144869 | mouse Pcsk9 | 1910 | 2865 |
| 144870 | mouse Pcsk9 | 1911 | 2866 |
| 144871 | mouse Pcsk9 | 1912 | 2867 |
| 144872 | mouse Pcsk9 | 1913 | 2868 |
| 144873 | mouse Pcsk9 | 1914 | 2869 |
| 144874 | mouse Pcsk9 | 1915 | 2870 |
| 144875 | mouse Pcsk9 | 1916 | 2871 |
| 144876 | mouse Pcsk9 | 1917 | 2872 |
| 144877 | mouse Pcsk9 | 1918 | 2873 |
| 144878 | mouse Pcsk9 | 1919 | 2874 |
| 144879 | mouse Pcsk9 | 1920 | 2875 |
| 144880 | mouse Pcsk9 | 1921 | 2876 |
| 144881 | mouse Pcsk9 | 1922 | 2877 |
| 144882 | mouse Pcsk9 | 1923 | 2878 |
| 144883 | mouse Pcsk9 | 1924 | 2879 |
| 144884 | mouse Pcsk9 | 1925 | 2880 |
| 144885 | mouse Pcsk9 | 1926 | 2881 |
| 144886 | mouse Pcsk9 | 1927 | 2882 |
| 144887 | mouse Pcsk9 | 1928 | 2883 |
| 144888 | mouse Pcsk9 | 1929 | 2884 |
| 144889 | mouse Pcsk9 | 1930 | 2885 |
| 144890 | mouse Pcsk9 | 1931 | 2886 |
| 144891 | mouse Pcsk9 | 1932 | 2887 |
| 144892 | mouse Pcsk9 | 1933 | 2888 |
| 144893 | mouse Pcsk9 | 1934 | 2889 |
| 144894 | mouse Pcsk9 | 1935 | 2890 |
| 144895 | mouse Pcsk9 | 1936 | 2891 |
| 144896 | mouse Pcsk9 | 1937 | 2892 |
| 144897 | mouse Pcsk9 | 1938 | 2893 |
| 144898 | mouse Pcsk9 | 1939 | 2894 |
| 144899 | mouse Pcsk9 | 1940 | 2895 |
| 144900 | mouse Pcsk9 | 1941 | 2896 |
| 144901 | mouse Pcsk9 | 1942 | 2897 |
| 144902 | mouse Pcsk9 | 1943 | 2898 |
| 144903 | mouse Pcsk9 | 1944 | 2899 |
| 144904 | mouse Pcsk9 | 1945 | 2900 |
| 144905 | mouse Pcsk9 | 1946 | 2901 |
| 144906 | mouse Pcsk9 | 1947 | 2902 |
| 144907 | mouse Pcsk9 | 1948 | 2903 |
| 144908 | mouse Pcsk9 | 1949 | 2904 |
| 144909 | mouse Pcsk9 | 1950 | 2905 |
| 144910 | mouse Pcsk9 | 1951 | 2906 |
| 144911 | mouse Pcsk9 | 1952 | 2907 |
| 144912 | mouse Pcsk9 | 1953 | 2908 |
| 144913 | mouse Pcsk9 | 1954 | 2909 |
| 144914 | mouse Pcsk9 | 1955 | 2910 |
| 144915 | mouse Pcsk9 | 1956 | 2911 |
| 144916 | mouse Pcsk9 | 1957 | 2912 |
| 144917 | mouse Pcsk9 | 1958 | 2913 |
| 144918 | mouse Pcsk9 | 1959 | 2914 |
| 144919 | mouse Pcsk9 | 1960 | 2915 |
| 144920 | mouse Pcsk9 | 1961 | 2916 |
| 144921 | mouse Pcsk9 | 1962 | 2917 |
| 144922 | mouse Pcsk9 | 1963 | 2918 |
| 144923 | mouse Pcsk9 | 1964 | 2919 |
| 144924 | mouse Pcsk9 | 1965 | 2920 |
| 144925 | mouse Pcsk9 | 1966 | 2921 |
| 144926 | mouse Pcsk9 | 1967 | 2922 |
| 144927 | mouse Pcsk9 | 1968 | 2923 |
| 144928 | mouse Pcsk9 | 1969 | 2924 |
| 144929 | mouse Pcsk9 | 1970 | 2925 |
| 144930 | mouse Pcsk9 | 1971 | 2926 |
| 144931 | mouse Pcsk9 | 1972 | 2927 |
| 144932 | mouse Pcsk9 | 1973 | 2928 |
| 144933 | mouse Pcsk9 | 1974 | 2929 |
| 144934 | mouse Pcsk9 | 1975 | 2930 |

TABLE 11 provides illustrative target nucleic acids that are useful in the compositions, systems and methods described herein.

TABLE 11
EXEMPLARY TARGET NUCLEIC ACIDS
Exemplary targets

AAVS1, ABCA4, ABCB11, ABCC8, ABCD1, ABCG5, ABCG8, ACAD9, ACADM, ACADVL, ACAT1, ACTA1, ACOX1, ACSF3,
ADA, ADAMTS2, ADGRG1, AGA, AGL, AGPS, AGXT, AHI1, AIRE, ALDH3A2, ALDOB, ALG6, ALK, ALKBH5, ALMS1, ALPL,
AMRC9, AMT, ANAPC10, ANAPC11, ANGPTL3, ANGPTL4, APC, Apo(a), APOCIII (APOC3), APOEε4, APOL1, APP, AQP2, AR,
ARFRP1, ARG1, ARH, ARL13B, ARL6, ARSA, ARSB, ASL, ASNS, ASPA, ASS1, ATM, ATP6V1B1, ATP7A, ATP7B, ATRX, ATXN1,
ATXN10, ATXN2, ATXN3, ATXN7, ATXN8OS, AXIN1, AXIN2, B2M, BACE-1, BAK1, BAP1, BARD1, BAX2, BBS1, BBS10, BBS12,
BBS2, BCKDHA, BCKDHB, BCL2L2, BCS1L, BEST1, Betaglobin gene, BLM, BMPR1A, BRAF, BRAFV600E, BRCA1, BRCA2,
BRIP1, BSND, C9ORF72, CA4, CACNA1A, CAH1, CAPN3, CASR, CBS, CCNB1 CC2D2A, CCR5, CD1, CD2, CD3, CD3D,
CD3Z, CD4, CD5, CD6, CD7, CD8A, CD8B, CD9, CD14, CD18, CD19, CD21, CD22, CD23, CD27, CD28, CD30, CD33, CD34,
CD36, CD38, CD40, CD40L, CD44, CD46, CD47, CD48, CD52, CD55, CD57, CD58, CD59, CD68, CD69, CD72, CD73, CD74,
CD79A, CD80, CD81, CD83, CD84, CD86, CD90, CD93, CD96, CD99, CD100, CD123, CD160, CD163, CD164, CD164L2,
CD166, CD200, CD204, CD207, CD209, CD226, CD244, CD247, CD274, CD276, CD300, CD320, CDC73, CDH1, CDH23,
CDK11, CDK4, CDKN1A, CDKN1B, CDKN1C, CDKN2A, CDKN2B, CEBPA, CELA3B, CEP290, CERKL, CFB, CFTR,
CHCHD10, CHEK2, CHM, CHRNE, CIDEB, CIITA, CLN3, CLN5, CLN6, CLN8, CLRN1, CLTA, CMT1A, CNBP, CNGB1,
CNGB3, COL1A1, COL1A2, COL27A1, COL4A3, COL4A4, COL4A5, COL6A1, COL6A2, COL6A3, COL7A1, CPS1, CPT1A,
CPT2, CRB1, CREBBP, CRX, CRYAA, CTNNA1, CTNNB1, CTNND2, CTNS, CTSK, CXCL12, CYBA, CYBB, CYP11B1, CYP11B2,
CYP17A1, CYP19A1, CYP21A2, CYP27A1, DBT, DCC, DCLRE1C, DERL2, DFNA36, DFNB31, DGAT2, DHCR7, DHDDS,
DICER1, DIS3L2, DLD, DMD, DMPK, DNAH5, DNAI1, DNAI2, DNM2, DNMT1, DPC4, DUX4, DYSF, EDA, EDN3, EDNRB,
EGFR, EIF2B5, EMC2, EMC3, EMD, EMX1, EN1, EPCAM, ERCC6, ERCC8, ESCO2, ETFA, ETFDH, ETHE1, EVC, EVC2, EYS,
F5, F9, FX1, FAH, FAM161A, FANCA, FANCB, FANCC, FANCD1, FANCD2, FANCE, FANCF, FANCG, FANC1, FANCJ,
FANCL, FANCM, FANCN, FANCP, FANCS, FBN1, FGF14, FGFR2, FGFR3, FGA, FGB, FGG, FH, FHL1, FIX, FKRP, FKTN,
FLCN, FMR1, FOXP3, FSCN2, FSHD1, FUS, FUT8, FVIII, FXII, FXN, G6PC, GAA, GALC, GALK1, GALT, GAMT, GATA2,
GATA-4, GBA, GBE1, GCDH, GCGR, GDNF, GFAP, GFM1, GHR, GJB1, GJB2, GLA, GLB1, GLDC, GLE1, GNE, GNPTAB,
GNPTG, GNS, GPAM, GPC3, GPR98, GREM1, GRHPR, GRIN2B, H2AFX, H2AX, HADHA, HAX1, HBA1, HBA2, HBB, HBV
cccDNA, HER2, HEXA, HEXB, HFE, HGSNAT, HLCS, HMGCL, HAO1, HOGA1, HOXB13, HPRPF3, HPRT1, HPS1, HPS3,
HRAS, HRD1, HSD3B2, HSD17B4, HSD17B13, HTT, HUS1, HYAL1, HYLS1, IDS, IDUA, IFITM5, IKBKAP, IL2RG, IL7R,
IMPDH1, INPP5E, IRF4, ITGB2, ITPR1, IVD, JAG1, JAK1, JAK3, KCNC3, KCND3, KCNJ11, KLKB1, KLHL7, KRAS, LAMA1,
LAMA2, LAMA3, LAMB3, LAMC2, LCA5, LDHA, LDLR, LDLRAP1, LHX3, LIFR, LIPA, LMNA, LMOD3, LOR, LOXHD1, LPA,
LPL, LRAT, LRP6, LRPPRC, LRRK2, MADR2, MAN2B1, MAPT, MARC1, MAX, MCM6, MCOLN1, MECP2, MED17, MEFV,
MEN1, MERTK, MESP2, MET, METex14, MFN2, MFSD8, MIA3, MITF, MKL2, MKS1, MLC1, MLH1, MLH3, MMAA, MMAB,
MMACHC, MMADHC, MMD, MP1, MPL, MPV17, MSH2, MSH3, MSH6, MTHFD1L, MTHFR, MTM1, MTRR, MTTP, MUT,
MUTYH, MYC, MYH7, MYO7A, NAGLU, NAGS, NBN, NDRG1, NDUFAF5, NDUFS6, NEB, NF1, NF2, NKX2-5, NOG, NOTCH1,
NOTCH2, NPC1, NPC2, NPHP1, NPHS1, NPHS2, NRAS, NR2E3, NTHL1, NTRK, NTRK1, OAT, OCT4, OFD1, OPA3, OTC,
PAH, PALB2, PAQR8, PAX3, PC, PCCA, PCCB, PCDH15, PCSK9, PD1, PDCD1, PDE6B, PDGFRA, PDHA1, PDHB, PEX1,
PEX10, PEX12, PEX13, PEX14, PEX16, PEX19, PEX2, PEX26, PEX3, PEX5, PEX6, PEX7, PFKM, PHGDH, PHOX2B, PKD1,
PKD2, PKHD1, PKK, PLEKHG4, PMM2, PMP22, PMS1, PMS2, PNPLA3, POLD1, POLE, POMGNT1, POT1, POU5F1, PPM1A,
PPP2R2B, PPT1, PRCD, PRKAG2, PRKAR1A, PRKCG, PRNP, PROM1, PROP1, PRPF31, PRPF8, PRPH2, PRPS1, PSAP,
PSD3, PSD95, PSEN1, PSEN2, PSRC1, PTCH1, PTEN, PTS, PUS1, PYGM, RAB23, RAD50, RAD51C, RAD51D, RAG1, RAG2,
RAPSN, RARS2, RB1, RDH12, RECQL4, RET, RHO, RICTOR, RMRP, ROS1, RP1, RP2, RPE65, RPGR, RPGRIP1L, RPL32P3,
RS1, RTCA, RTEL1, RUNX1, SACS, SAMHD1, SCN1A, SCN2A, SDHA, SDHAF2, SDHB, SDHC, SDHD, SEL1L, SEPSECS,
SERPINA1, SERPINC1, SERPING1, SGCA, SGCB, SGCG, SGSH, SIRT1, SLC12A3, SLC12A6, SLC17A5, SLC22A5, SLC25A13,
SLC25A15, SLC26A2, SLC26A4, SLC35A3, SLC35B4, SLC37A4, SLC39A4, SLC4A11, SLC6A8, SLC7A7, SMAD3, SMAD4,
SMARCA4, SMARCAL1, SMARCB1, SMARCE1, SMN1, SMPD1, SNAI2, SNCA, SNRNP200, SOD1, SOX10, SPARA7, SPTBN2,
STAR, STAT3, STK11, SUFU, SUMF1, SYNE1, SYNE2, SYS1, TARDBP, TAT, TBK1, TBP, TCIRG1, TCTN3, TECPR2, TERC,
TERT, TFR2, TGFBR2, TGM1, TH, TLE3, TMEM127, TMEM138, TMEM216, TMEM43, TMEM67, TMPRSS6, TNNI2, TNNT1,
TNNT3, TOP1, TOPORS, TP53, TPM2, TPM3, TPP1, TRAC, TRMU, TSC1, TSC2, TSFM, TSPAN14, TTBK2, TTC8, TTPA, TTR,
TULP1, TYMP, UBE2G2, UBE2J1, UBE3A, USH1C, USH1G, USH2A, VEGF, VHL, VPS13A, VPS13B, VPS35, VPS45, VRK1,
VSX2, VWF, WAS, WDR19, WDR48, WNT10A, WRN, WS2B, WS2C, WT1, XPA, XPC, XPF, XRCC3, YAP1, ZAC1, ZEB1,
ZFYVE26, and ZNF423

TABLE 12 provides illustrative diseases and syndromes for compositions, systems and methods described herein.

TABLE 12
DISEASES AND SYNDROMES
Exemplary Diseases and Syndromes

11-hydroxylase deficiency; 17,20-desmolase deficiency; 17-hydroxylase deficiency; 3-hydroxyisobutyrate aciduria; 3-
hydroxysteroid dehydrogenase deficiency; 46,XY gonadal dysgenesis; AAA syndrome; ABCA3 deficiency; ABCC8-associated
hyperinsulinism; aceruloplasminemia; acromegaly; achondrogenesis type 2; acral peeling skin syndrome; acrodermatitis
enteropathica; acute bacterial infection; adrenocortical micronodular hyperplasia; adrenoleukodystrophies;
adrenomyeloneuropathies; Aicardi-Goutieres syndrome; AIDS; Alagille disease (also called Alagille Syndrome); Alexander
Disease; Alpers syndrome; alpha-1 antitrypsin deficiency (AATD); alpha-mannosidosis; Alstrom syndrome; Alzheimer's disease;
amebic dysentery; amelogenesis imperfecta; amish type microcephaly; amyotrophic lateral sclerosis (ALS); anaplastic large cell
lymphoma; anauxetic dysplasia; androgen insensitivity syndrome; angiopathic thrombosis; antiphospholipid syndrome; Antley-
Bixler syndrome; APECED; Apert syndrome; aplasia of lacrimal and salivary glands; arginase-1 deficiency; argininosuccinic
aciduria; argininemia; arrhythmogenic right ventricular dysplasia; Arts syndrome; ARVD2; arylsulfatase deficiency type
metachromatic leukodystrophy; ataxia telangiectasia; atherosclerotic cardiovascular disease; autoimmune lymphoproliferative
syndrome; autoimmune polyglandular syndrome type 1; autosomal dominant anhidrotic ectodermal dysplasia; autosomal dominant
deafness; autosomal dominant polycystic kidney disease; autosomal recessive microtia; autosomal recessive renal glucosuria;
autosomal visceral heterotaxy; babesiosis; bacterial vaginosis; balantidial dysentery; Bardet-Biedl syndrome; Bartter syndrome;
basal cell nevus syndrome; Batten disease; benign recurrent intrahepatic cholestasis; beta-mannosidosis; β-thalassemia; Bethlem
myopathy; Blackfan-Diamond anemia; bleeding disorder (coagulation); blepharophimosis; Byler disease; C syndrome; CADASIL;
calcific aortic stenosis; calcification of joints and arteries; carbamoyl phosphate synthetase I deficiency; carcinoid syndrome
diarrhea; cardiofaciocutaneous syndrome; cardiovascular disease (CVD); Carney triad; carnitine palmitoyltransferase deficiencies;
cartilage-hair hypoplasia; cblC type of combined methylmalonic aciduria; CD18 deficiency; CD3Z-associated primary T-cell
immunodeficiency; CD40L deficiency; CDAGS syndrome; CDG1A; CDG1B; CDG1M; CDG2C; CEDNIK syndrome; central core
disease; centronuclear myopathy; cerebral capillary malformation; cerebrooculofacioskeletal syndrome type 4;
cerebrooculogacioskeletal syndrome; cerebrotendinous xanthomatosis; Chaga's Disease; Charcot Marie Tooth Disesase;
chemotherapy; cherubism; CHILD syndrome; chronic granulomatous disease; chronic recurrent multifocal osteomyelitis; cirrhosis;
citrin deficiency; citrullinemia type I; citrullinemia type II; classic hemochromatosis; CNPPB syndrome; cobalamin C disease;
Cockayne syndrome; coenzyme Q10 deficiency; Coffin-Lowry syndrome; Cohen syndrome; combined deficiency of coagulation
factors V; common variable immune deficiency 3; complement hyperactivation; complete androgen insentivity; cone rod
dystrophies; conformational diseases; congenital adrenal hyperplasia; congenital bile adid synthesis defect type 1; congenital bile
adid synthesis defect type 2; congenital defect in bile acid synthesis type; congenital erythropoietic porphyria; congenital
generalized osteosclerosis; congenital hyperplasia (CAH); congenital muscular dystrophy 1A (MDC1A); Cornelia de Lange
syndrome; coronary heart disease; Cousin syndrome; Cowden disease; COX deficiency; Cri du chat syndrome; Crigler-Najjar
disease; Crigler-Najjar syndrome type 1; Crisponi syndrome; Crouzon syndrome; Currarino syndrome; Curth-Macklin type
ichthyosis hystrix; cutaneous T-cell lymphoma; cutis laxa; cystic fibrosis; cystinosis; d-2-hydroxyglutaric aciduria; DDP syndrome;
Dejerine-Sottas disease; Denys-Drash syndrome; Dercum disease; desmin cardiomyopathy; desmin myopathy; DGUOK-associated
mitochondrial DNA depletion; diabetes Type I; diabetes Type II; disorders of glutamate metabolism; distal spinal muscular atrophy
type 5; DNA repair diseases; dominant optic atrophy; Doyne honeycomb retinal dystrophy; Dravet Syndrome; Duchenne muscular
dystrophy; dyskeratosis congenita; Ehlers-Danlos syndrome type 4; Ehlers-Danlos syndromes; Elejalde disease; Ellis-van Creveld
disease; Emery-Dreifuss muscular dystrophies; encephalomyopathic mtDNA depletion syndrome; encephalitis; enzymatic diseases;
EPCAM-associated congenital tufting enteropathy; epidermolysis bullosa with pyloric atresia; epilepsy; fabry disease;
facioscapulohumeral muscular dystrophy; Factor V Leiden thrombophilia; Faisalabad histiocytosis; familial atypical
mycobacteriosis; familial capillary malformation-arteriovenous; Familial Creutzfeld-Jakob disease; familial esophageal achalasia;
familial glomuvenous malformation; familial hemophagocytic lymphohistiocytosis; familial mediterranean fever; familial
megacalyces; familial schwannomatosis; familial spina bifida; familial splenic asplenia/hypoplasia; familial thrombotic
thrombocytopenia purpura; Fanconi disease (Fanconi anemia); Feingold syndrome; FENIB; fibrodysplasia ossificans progressiva;
FKTN; Fragile X syndrome; Francois-Neetens fleck corneal dystrophy; Frasier syndrome; Friedreich's ataxia; FTDP-17; Fuchs
corneal dystrophy; fucosidosis; G6PD deficiency; galactosialidosis; Galloway syndrome; Gardner syndrome; Gaucher disease;
Gitelman syndrome; glaucoma; GLUT1 deficiency; GM2- Gangliosidoses (e.g., Tay Sachs Disease, Sandhoff Disease) glycogen
storage disease type 1b; glycogen storage disease type 2; glycogen storage disease type 3; glycogen storage disease type 4; glycogen
storage disease type 9a; glycogen storage diseases; GM1-gangliosidosis; Greenberg syndrome; Greig cephalopolysyndactyly
syndrome; hair genetic diseases; hairy cell leukemia; HANAC syndrome; harlequin type ichtyosis congenita; HDR syndrome;
hearing loss; heart failure; hemochromatosis type 3; hemochromatosis type 4; hemolytic anemia; hemolytic uremic syndrome;
hemophilia A; hemophilia B; hepatitis C infection; hereditary angioedema type 3; hereditary angioedemas; hereditary hemorrhagic
telangiectasia; hereditary hypofibrinogenemia; hereditary intraosseous vascular malformation; hereditary leiomyomatosis and renal
cell cancer; hereditary neuralgic amyotrophy; hereditary orotic aciduria; hereditary sensory and autonomic neuropathy type;
Hermansky-Pudlak disease; HHH syndrome; HHT2; hidrotic ectodermal dysplasia type 1; hidrotic ectodermal dysplasias;
histiocytic sarcoma; HNF4A-associated hyperinsulinism; HNPCC; homozygous familial hypercholesterolemia; hormone refractory
prostate cancer; human immunodeficiency with microcephaly; Human monkeypox (MPX); human papilloma virus (HPV) infection;
Huntington's disease; hyper-IgD syndrome; hyperinsulinism-hyperammonemia syndrome; hypercholesterolemia; hypertension;
hypertrophy of the retinal pigment epithelium; hypochondrogenesis; hypohidrotic ectodermal dysplasia; hypotension; ICF
syndrome; idiopathic congenital intestinal pseudo-obstruction; immunodeficiency 13; immunodeficiency 17; immunodeficiency 25;
immunodeficiency with hyper-IgM type 1; immunodeficiency with hyper-IgM type 3; immunodeficiency with hyper-IgM type 4;
immunodeficiency with hyper-IgM type 5; immunoglobulin alpha deficiency; inborn errors of thyroid metabolism; infantile
myofibromatosis; infantile visceral myopathy; infantile X-linked spinal muscular atrophy; influenza A; influenza B; intradialytic
hypotension; intrahepatic cholestasis of pregnancy; invasive aspergillosis; invasive mucormycosis; IPEX syndrome; IRAK4
deficiency; isolated congenital asplenia; Jeune syndrome; Johanson-Blizzard syndrome; Joubert syndrome; JP-HHT syndrome;
juvenile hemochromatosis; juvenile hyalin fibromatosis; juvenile nephronophthisis; Kabuki mask syndrome; Kallmann syndromes;
Kartagener syndrome; KCNJ11-associated hyperinsulinism; Kearns-Sayre syndrome; Kostmann disease; Kozlowski type of
spondylometaphyseal dysplasia; Krabbe disease; LADD syndrome; late infantile-onset neuronal ceroid lipofuscinosis; LCK
deficiency; LDHCP syndrome; Leber Congenital Amaurosis Teyp 10; Legius syndrome; Leigh syndrome; lethal congenital
contracture syndrome 2; lethal congenital contracture syndromes; lethal contractural syndrome type 3; lethal neonatal CPT
deficiency type 2; lethal osteosclerotic bone dysplasia; leukocyte adhesion deficiency; Li Fraumeni syndrome; LIG4 syndrome;
limb girdle muscular dystrophies (LGMD1B, LGMD2A, LGMD2B); lipodystrophy; lissencephaly type 1; lissencephaly type 3;
Loeys-Dietz syndrome; low phospholipid-associated cholelithiasis; Lynch Syndrome; lysinuric protein intolerance; a lysosomal
storage disease (e.g., Hunter syndrome, Hurler syndrome); macular dystrophy; Maffucci syndrome; Majeed syndrome; malaria;
mannose-binding protein deficiency; mantle cell lymphoma; Marfan disease; Marshall syndrome; MASA syndrome; mastocytosis;
MCAD deficiency; McCune-Albright syndrome; MCKD2; Meckel syndrome; MECP2 Duplication Syndrome; Meesmann corneal
dystrophy; megacystis-microcolon-intestinal hypoperistalsis; megaloblastic anemia type 1; MEHMO; MELAS; Melnick-Needles
syndrome; MEN2s; meningitis; Menkes disease; metachromatic leukodystrophies; methymalonic acidemia due to transcobalamin
receptor defect; methylmalonic acidurias; methylvalonic aciduria; microcoria-congenital nephrosis syndrome; microvillous atrophy;
migraine; mitochondrial neurogastrointestinal encephalomyopathy; monilethrix; monosomy X; mosaic trisomy 9 syndrome; Mowat-
Wilson syndrome; mucolipidosis type 2; mucolipidosis type Ma; mucolipidosis type IV; mucopolysaccharidoses;
mucopolysaccharidosis type 3A; mucopolysaccharidosis type 3C; mucopolysaccharidosis type 4B; multiminicore disease; multiple
acyl-CoA dehydrogenation deficiency; multiple cutaneous and mucosal venous malformations; multiple endocrine neoplasia type 1;
multiple myeloma; multiple sclerosis; multiple sulfatase deficiency; mycosis fungoides; myotonic dystrophy; NAIC; nail-patella
syndrome; nemaline myopathies; neonatal diabetes mellitus; neonatal surfactant deficiency; nephronophtisis; Netherton disease;
neurofibromatoses; neurofibromatosis type 1; Niemann-Pick disease type A; Niemann-Pick disease type B; Niemann-Pick disease
type C; NKX2E; non-alcoholic fatty liver disease (NAFLD); non-alcoholic steatohepatitis (NASH); Noonan syndrome; North
American Indian childhood cirrhosis; NROB1 duplication-associated DSD; ocular genetic diseases; oculo-auricular syndrome;
OLEDAID; oligomeganephronia; oligomeganephronic renal hypolasia; Ollier disease; Opitz-Kaveggia syndrome; ornithine
transcarbamylase deficiency (OTCD); orofaciodigital syndrome type 1; orofaciodigital syndrome type 2; osseous Paget disease;
osteogenesis imperfecta; otopalatodigital syndrome type 2; orthostatic hypotension; overactive bladder; OXPHOS diseases;
palmoplantar hyperkeratosis; panlobar nephroblastomatosis; Parkes-Weber syndrome; Parkinson's disease; partial deletion of
21q22.2-q22.3; Pearson syndrome; Pelizaeus-Merzbacher disease; Pendred syndrome; pentalogy of Cantrell; peroxisomal acyl-
CoA-oxidase deficiency; Peutz-Jeghers syndrome; Pfeiffer syndrome; Pierson syndrome; pigmented nodular adrenocortical disease;
pipecolic acidemia; Pitt-Hopkins syndrome; plasmalogens deficiency; platelet glycoprotein IV deficiency; pleuropulmonary
blastoma and cystic nephroma; pneumonia; polycystic kidney disease; polycystic ovarian disease; polycystic lipomembranous
osteodysplasia; Pompe disease; including infantile onset Pompe disease (IOPD) and late onset Pompe disease (LOPD); porphyrias;
post-herpetic neuralgia; PRKAG2 cardiac syndrome; premature ovarian failure; primary erythermalgia; primary hemochromatoses;
primary hyperoxaluria; progressive familial intrahepatic cholestasis; propionic acidemia; prostate cancer; protein-losing
enteropathy; pulmonary arterial hypertension; pyruvate decarboxylase deficiency; RAPADILINO syndrome; renal cystinosis;
restless leg syndrome; retinitis pigmentosa; Rett Syndrome; rhabdoid tumor predisposition syndrome; Rieger syndrome; ring
chromosome 4; Roberts syndrome; Robinow-Sorauf syndrome; Rothmund-Thomson syndrome; severe combined
immunodeficiency disorder (SCID); Saethre-Chotzen syndrome; Sandhoff disease; SC phocomelia syndrome; SCAS; Schinzel
phocomelia syndrome; schizophrenia; severe hypertriglyceridemia; short rib-polydactyly syndrome type 1; short rib-polydactyly
syndrome type 4; short-rib polydactyly syndrome type 2; short-rib polydactyly syndrome type 3; Shwachman disease; Shwachman-
Diamond disease; sickle cell anemia; Silver-Russell syndrome; Simpson-Golabi-Behmel syndrome; skin infection; Smith-Lemli-
Opitz syndrome; SPG7-associated hereditary spastic paraplegia; spherocytosis; spinocerebellar ataxia; spinal muscular atrophy;
split-hand/foot malformation with long bone deficiencies; spondylocostal dysostosis; sporadic amyotrophic lateral sclerosis;
sporadic visceral myopathy with inclusion bodies; storage diseases; Stargardt macular dystrophy; STRA6-associated syndrome;
stroke; tardive dyskinesia; Tay-Sachs disease; thanatophoric dysplasia; thromboembolism; thrombosis; thrombophilia due to
antithrombin III deficiency; thyroid metabolism diseases; Tourette syndrome; transcarbamylase deficiency; transthyretin-associated
amyloidosis; trisomy 13; trisomy 22; trisomy 2p syndrome; tuberous sclerosis; tufting enteropathy; ullrich congenital muscular
dystrophy (UCMD); urea cycle diseases; Usher Syndrome; Van Den Ende-Gupta syndrome; Van der Woude syndrome; variegated
mosaic aneuploidy syndrome; VLCAD deficiency; von Hippel-Lindau disease; von Willebrand disease; Waardenburg syndrome;
WAGR syndrome; Walker-Warburg syndrome; Werner syndrome; Wilson's disease; Wiskott-Aldrich Syndrome; Wolcott-Rallison
syndrome; Wolfram syndrome; X-linked agammaglobulinemia; X-linked chronic idiopathic intestinal pseudo-obstruction; X-linked
cleft palate with ankyloglossia; X-linked dominant chondrodysplasia punctata; X-linked ectodermal dysplasia; X-linked Emery-
Dreifuss muscular dystrophy; X-linked lissencephaly; X-linked lymphoproliferative disease; X-linked visceral heterotaxy;
xanthinuria type 1; xanthinuria type 2; xeroderma pigmentosum; XPV; and Zellweger disease.

### EXAMPLES

The following examples are included for illustrative purposes only and are not intended to limit the scope of the invention.

### Example 1. DCasPhi.12—Methyltransferase Fusion Protein Represses Gene Expression in Human Cells

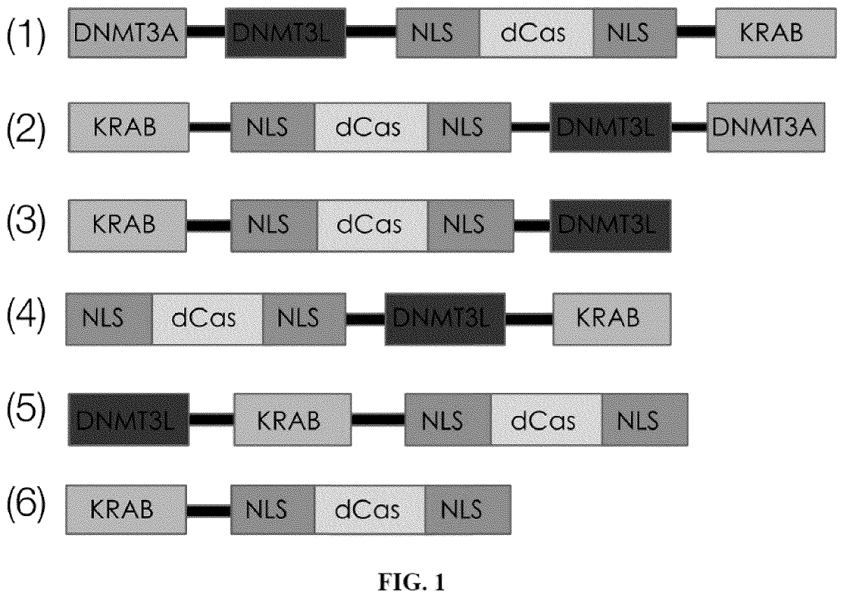

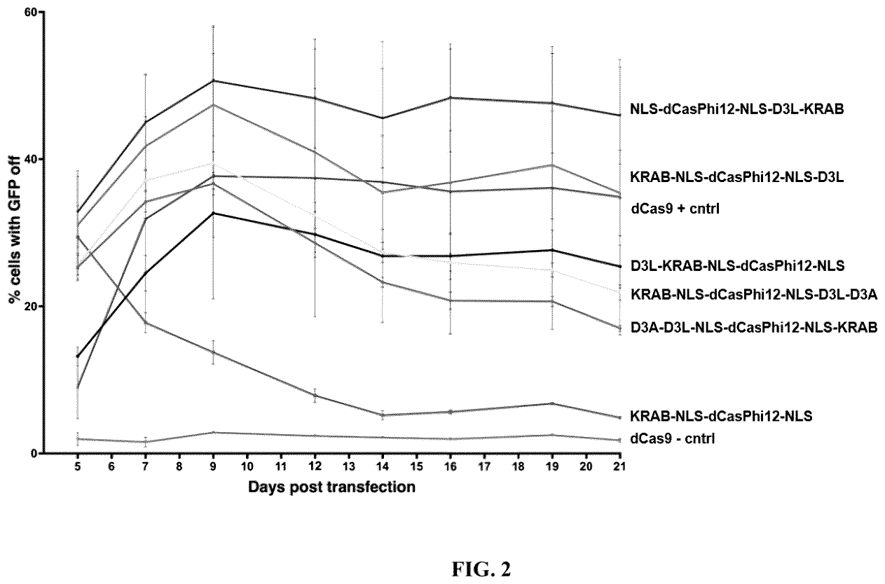

Plasmids encoding fusion constructs comprising dCasPhi.12 (L26R, E567Q) (SEQ ID NO: 2) were generated. These fusion constructs are described in TABLE 6 and represented in FIG. 1. HEK293T cells were transfected with one of these fusion constructs, a pool of two guide nucleic acids, and a GFP reporter. The guide nucleic acids were designed to target the GFP reporter, such that a complex of a fusion construct and the guide nucleic acid would bind the GFP reporter and repress its expression. The guide nucleic acids had a repeat sequence comprising nucleotide sequence: 5′-AUUGCUCCUUACGAGGAGAC-3′ (SEQ ID NO: 5). The full guide sequences were: 5′-AUUGCUCCUUACGAGGAGACCGGCAAGACUAGCGCAGAGA-3′ (SEQ ID NO: 23) and 5′-AUUGCUCCUUACGAGGAGACGUCGCGCGGCUCCCUACGCA-3′ (SEQ ID NO: 24). Experiments were performed in triplicate. GFP fluorescence was measured by flow cytometry starting at day 5 after transfection, every 2-3 days, ending at day 21. Results are shown in FIG. 2. KRAB(ZNF10)-NLS-dCasPhi12-NLS showed transient repression of GFP as expected. NLS-dCasPhi12-NLS-DNMT3L-KRAB(ZNF10) demonstrated stable repression of GFP reporter through day 21. dCas9 (D10A_H840A) (SEQ ID NO: 22) served as a control with a guide sequence of: 5′-CUCCUCAGAACCAAGCGUCGUUUAAGAGCUAUGCUGGAAACAGCAUAGCAAGU UUAAAUAAGGCUAGUCCGUUAUCAACUUGAAAAAGUGGCACCGAGUCGGUGC-3′ (SEQ ID NO: 25).

### Example 2. In Vitro Screening of CasPhi.12 Guide Nucleic Acids for Repressing Gene Expression in Human Cells

To screen for CasPhi12 guide nucleic acids for repressing activity, a total of 44 guide nucleic acids were tested with an activated CasPhi12 nuclease to determine if indels were generated in target sequences. Plasmid 1 encoding CasPhi.12 (SEQ ID NO: 1) and plasmid 2 encoding one of the 44 guide nucleic acids were generated. HEK293T cells with a synthetic GFP reporter were transfected with plasmid 1 and plasmid 2. The guide nucleic acids were designed to target the GFP reporter, such that a complex of the CasPhi.12 construct and the guide nucleic acid would bind the GFP reporter and repress its expression. The negative control is CasPhi12 with a non-targeting (NT) guide nucleic acid and the positive control is CasPhi12 with a validated guide nucleic acid. 3 days after transfection, cells were harvested. Next-generation sequencing (NGS) was used to detect indels.

The study also aimed to evaluate the gene silencing capabilities of the 44 guide nucleic acids when paired with dCasPhi.12(L26R, E567Q) (SEQ ID NO: 2). Plasmid 1 encoding dCasPhi.12(L26R and E567Q)-DNMT3L-KRAB(ZNF10) (SEQID NO: 19) and plasmid 2 encoding one of the 44 guide nucleic acids were generated. The negative control is dCas9 (CRISPROff) (SEQ ID NO: 22) with a NT guide nucleic acid and the positive control is dCasPhi.12 (L26R and E567Q)-DNMT3L-KRAB(ZNF10) (SEQID NO: 19) with a validated guide nucleic acid (−13) and dCas9 (SEQ ID NO: 22) with a targeting guide nucleic acid. The GFP flow cytometry analysis began on day 5 and concluded on day 21. Measurements were taken on day 5, 7, 9, 12, 15, 19 and 21.

Based on the day 3 Indel results and day 21 GFP repression data, 5 out of the 44 guide nucleic acids (shown in TABLE 13) demonstrated about 20% or greater repressive activity.

TABLE 13
Spacer sequences and full sequences for
the 5 guide nucleic acids

| Distance | | | SEQ | | SEQ |
|---|---|---|---|---|---|
| from | | Spacer | ID | | ID |
| TSS | PL# | Sequence | NO: | Full Sequence | NO: |
| −143 | P134337 | GUAGCUGCC | 2931 | AUUGCUCCUUACGAGG | 2936 |
| | | UUUUGGCAG | | AGACGUAGCUGCCUUU | |
| | | GA | | UGGCAGGA | |
| −118 | P134341 | CGGUCAGAG | 2932 | AUUGCUCCUUACGAGG | 2937 |
| | | GGACAGAGA | | AGACCGGUCAGAGGGA | |
| | | CC | | CAGAGACC | |
| −173 | P134346 | CGCAGUAGG | 2933 | AUUGCUCCUUACGAGG | 2938 |
| | | AAUGCUCAA | | AGACCGCAGUAGGAAU | |
| | | GC | | GCUCAAGC | |
| −220 | P134358 | ACGGGUGCA | 2934 | AUUGCUCCUUACGAGG | 2939 |
| | | CGUAGCUCA | | AGACACGGGUGCACGU | |
| | | GG | | AGCUCAGG | |
| −193 | P134364 | GAGAGAGGC | 2935 | AUUGCUCCUUACGAGG | 2940 |
| | | CCAGCUACU | | AGACGAGAGAGGCCCA | |
| | | CG | | GCUACUCG | |

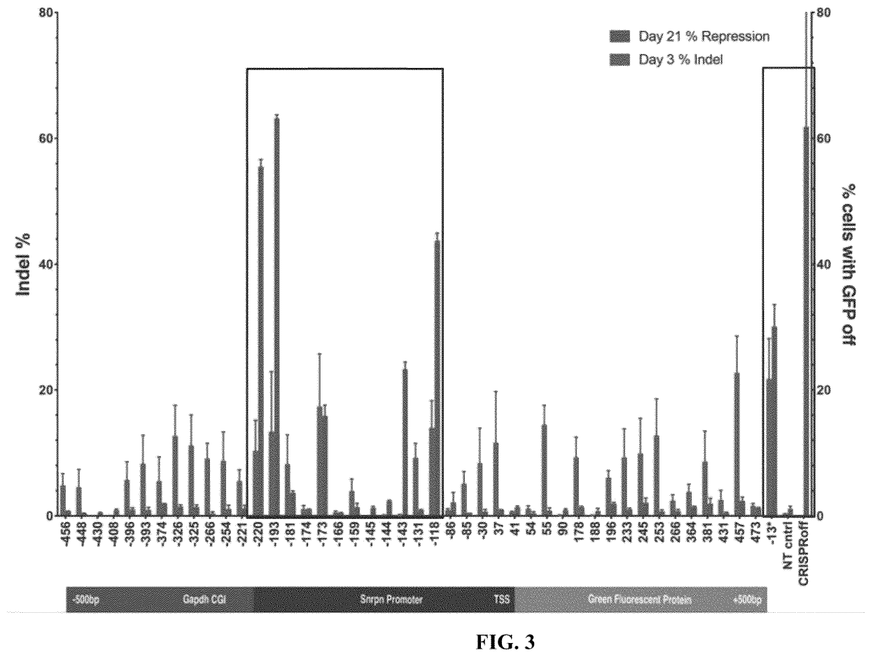

FIG. 3 illustrates that the majority of guide nucleic acids with repressive activity contain a spacer sequence that is complementary to and/or hybridizes to a target sequence situated upstream of the transcription start site (TSS).

### Example 3. In Vitro Screening of dCasPhi.12—Methyltransferase Fusion Proteins for Repressing Gene Expression in Human Cells

This study aimed to screen for fusion proteins with improved potency, durability and targeting. A number of plasmids encoding fusion proteins that contain CasPhi.12 mutants (dCasPhi.12) and at least one effector partner selected from DNMT3L, DNMT3A, KRAB(ZNF10) and KRAB(ZIM3) in different orientations were generated. These fusion protein constructs are listed as follows:
(a) dCasPhi.12 (L26K, A121Q, and E567Q)-DNMT3L-KRAB(ZNF10)
(b) dCasPhi.12 (L26K and E567Q)-DNMT3L-KRAB(ZNF10)
(c) dCasPhi.12 (L26R and E567Q)-DNMT3L-KRAB(ZNF10)
(d) dCasPhi.12 (L26R and E567Q)-KRAB(ZNF10)-DNMT3L
(e) KRAB(ZNF10)-DNMT3L-dCasPhi.12 (L26R and E567Q)
(f) DNMT3L-KRAB(ZNF10)-dCasPhi.12 (L26R and E567Q)
(g) DNMT3L-dCasPhi.12 (L26R and E567Q)-KRAB(ZNF10)
(h) KRAB(ZNF10)-dCasPhi.12 (L26R and E567Q)-DNMT3L
(i) dCasPhi.12 (L26R and E567Q)-DNMT3L-K(ZIM3)
(j) dCasPhi.12 (L26R and E567Q)-DNMT3L-KRAB(ZNF10)-K(ZIM3)
(k) K(ZIM3)-dCasPhi.12 (L26R and E567Q)-DNMT3L-KRAB(ZNF10)
(l) K(ZIM3)-KRAB(ZNF10)-DNMT3L-dCasPhi.12 (L26R and E567Q)
(m) K(ZIM3)-KRAB(ZNF10)-dCasPhi.12 (L26R and E567Q)-DNMT3L
(n) DNMT3L-dCasPhi.12 (L26R and E567Q)-KRAB(ZNF10)-K(ZIM3)

Two exemplary guide nucleic acids (gTR079 TSS-13 and gKD010 TSS-118) were used for the evaluation. TABLE 14 provides the spacer sequences and full sequences of the two exemplary guide nucleic acids.

TABLE 14
Spacer sequences and full sequences
of the two exemplary guide nucleic
acids

| | Dis- | | | | | |
|---|---|---|---|---|---|---|
| | tance | | Spacer | SEQ | Full | SEQ |
| gRNA | from | | Se- | ID | Se | NO: |
| Name | TSS | PL# | quence | NO: | quence | ID |
| gTR079 | −13 | PL25402 | CGGCA | 294 | AUUGCUC | 2942 |
| | | | AGACU | 1 | CUUACGA | |
| | | | AGCGC | | GGAGACC | |
| | | | AGAGA | | GGCAAGA | |
| | | | | | CUAGCGC | |
| | | | | | AGAGA | |
| gKD010 | −118 | PL34341 | CGGUC | 293 | AUUGCUC | 2937 |
| | | | AGAGG | 2 | CUUACGA | |
| | | | GACAG | | GGAGACC | |
| | | | AGACC | | GGUCAGA | |
| | | | | | GGGACAG | |
| | | | | | AGACC | |

HEK293T cells with a synthetic GFP reporter were transfected with plasmid 1 encoding a fusion protein construct as described above and plasmid 2 encoding a guide nucleic acid as provided in TABLE 14. The GFP flow cytometry analysis began on day 5 and concluded on day 21. Measurements were taken on day 5, 7,9, 12,15, 19 and 21.

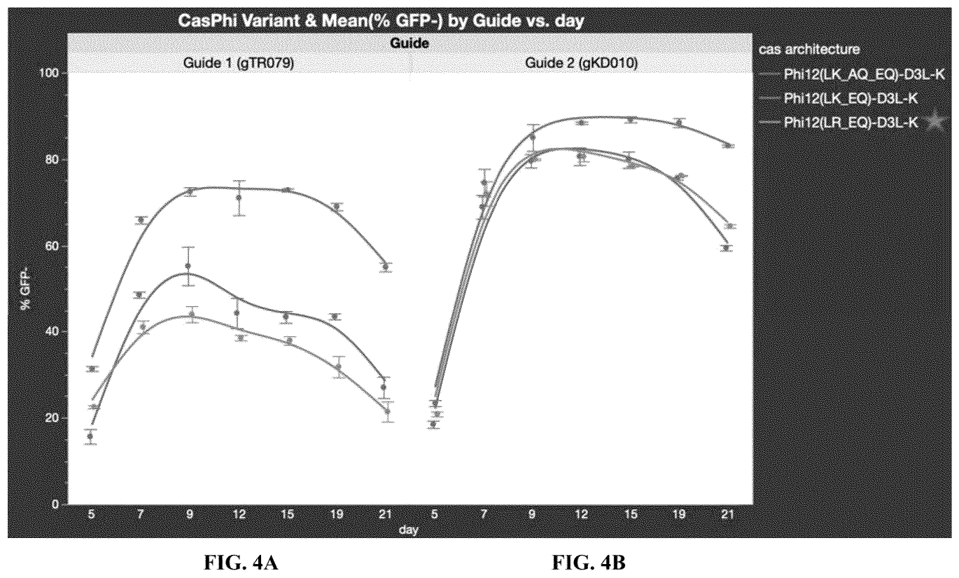

FIGS. 4A and 4B show the effects of different CasPhi.12 mutants on the repressive activity of the fusion constructs. The results show that the fusion protein dCasPhi.12(L26K and E567Q)-DNMT3L-KRAB(ZNF10) exhibits better repressive activity than dCasPhi.12(L26K, A121Q, and E567Q)-DNMT3L-KRAB(ZNF10) and dCasPhi.12 (L26R and E567Q)-DNMT3L-KRAB(ZNF10) when paired with the guide nucleic acid gTR-79 (FIG. 4A) or gKD010 (FIG. 4B).

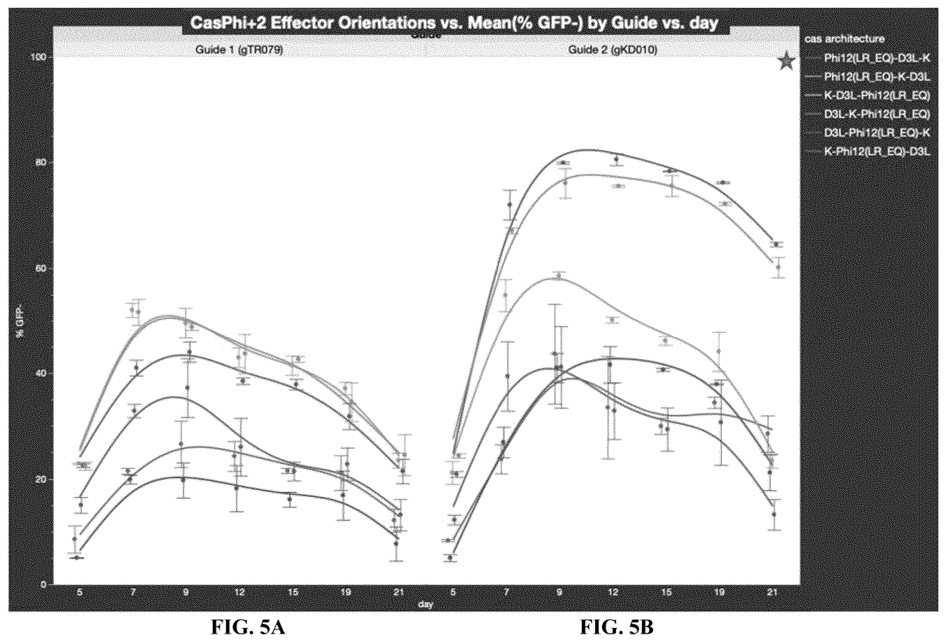

FIGS. 5A and 5B depict the effects of the different orientations of dCasPhi.12 in conjunction with effector partners DNMT3L and KRAB(ZNF10) on the performance of the fusion constructs. The results show that dCasPhi.12 (L26R and E567Q)-DNMT3L-KRAB(ZNF10) emerged as the most effective fusion protein when paired with the guide nucleic acid gTR-79 (FIG. 5A) or gKD010 (FIG. 5B). dCasPhi.12 (L26R and E567Q)-KRAB(ZNF10)-DNMT3L and KRAB(ZNF10)-DNMT3L-dCasPhi.12 (L26R and E567Q) have similar performance.

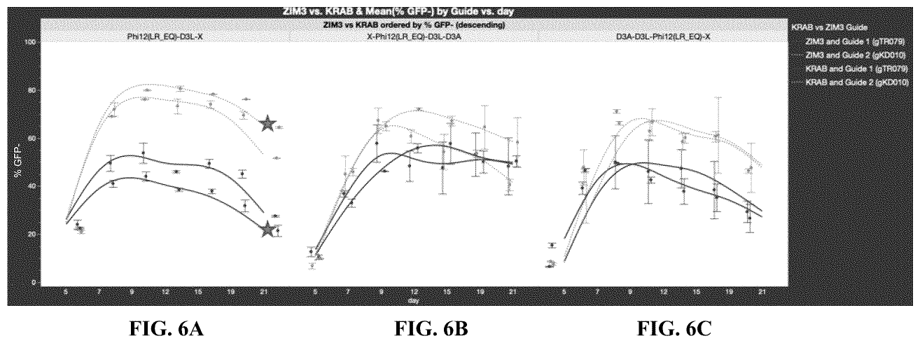

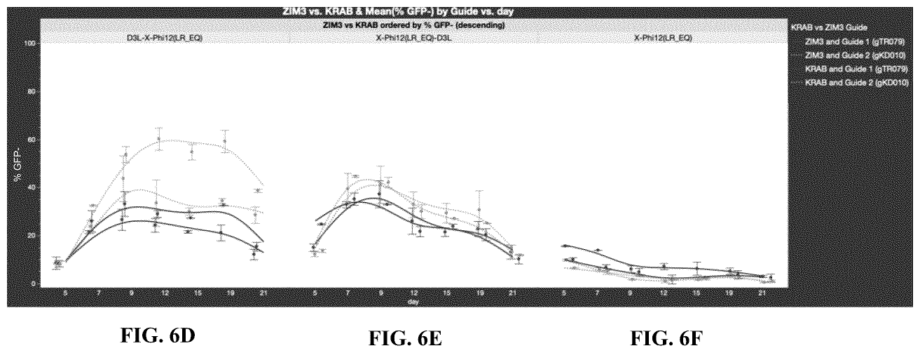

FIGS. 6A-6F present a side-by-side comparison of KRAB(ZNF10) and KRAB(ZIM3) in the fusion proteins. When swapping KRAB(ZNF10) with KRAB(ZIM3), the fusion proteins, including dCasPhi12(L26R and E567Q)-DNMT3L-X (FIG. 6A), X-dCasPhi12(L26R and E567Q)-DNMT3L-DNMT3A (FIG. 6B), DNMT3A-DNMT3L-dCasPhi12(L26R and E567Q)-X (FIG. 6C), DNMT3L-X-dCasPhi12(L26R and E567Q) (FIG. 6D), X-dCasPhi12(L26R and E567Q)-DNMT3L (FIG. 6E), and X-dCasPhi12(L26R and E567Q) (FIG. 6F), wherein X is selected from KRAB(ZNF10) and KRAB(ZIM3), display similar repressive activity. The results indicate that, compared to KRAB(ZNF10), KRAB(ZIM3) imparts a comparable repressive activity to the fusion proteins.

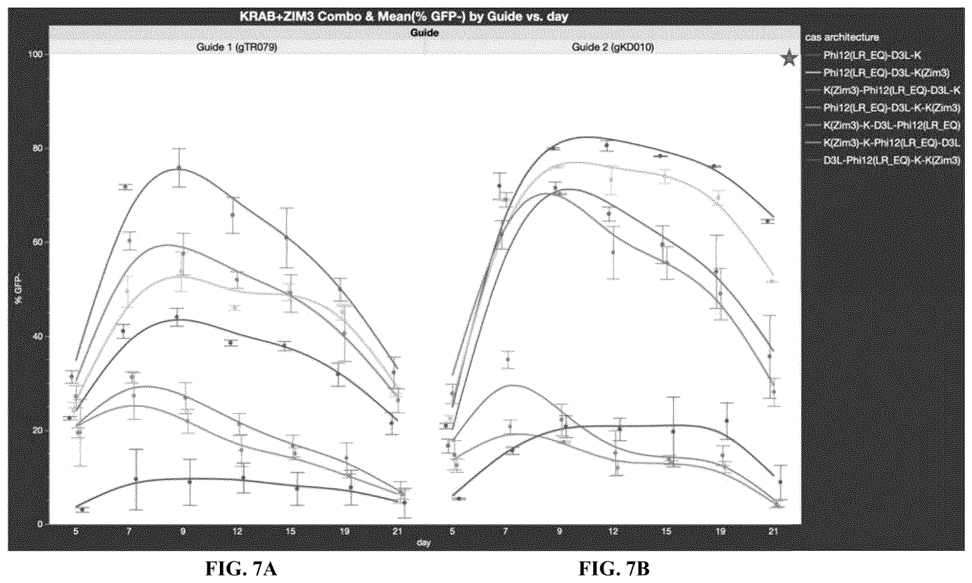

FIGS. 7A and 7B illustrate the effect of combining KRAB(ZNF10) and KRAB(ZIM3) in a single fusion protein on the performance of the fusion constructs. The results show that when paired with the guide nucleic acid gTR-79 (FIG. 7A) or gKD010 (FIG. 7B), the fusion proteins with both KRAB(ZNF10) and KRAB(ZIM3) display repressive activity comparable to those containing either KRAB(ZNF10) or KRAB(ZIM3) alone.

### Example 4. In Vitro Screening of CasM.265466 Guide Nucleic Acids and CasM.265466—Methyltransferase Fusion Proteins for Repressing Gene Expression in Human Cells

To screen for CasM.265466 guide nucleic acids for repressing activity, a total of 40 guide nucleic acids were generated and tested with an activated nuclease to determine if indels were generated in target sequences. HEK293T cells with a synthetic GFP reporter were transfected with Plasmid 1 encoding CasM.265466 (SEQ ID NO: 35) and plasmid 2 encoding one of the 40 guide nucleic acids. The guide nucleic acids were designed to target the GFP reporter, such that a complex of the CasM.265466 construct and the guide nucleic acid would bind the GFP reporter and repress its expression. The negative control is CasM.265466 with a non-targeting (NT) guide nucleic acid and the positive control is CasM.265466 with a validated guide nucleic acid. 3 days after transfection, cells were harvested. Next-generation sequencing (NGS) was used to detect Indels.

The Day 3 Indel data led to the identification of 23 promising guide nucleic acids. The Indel results indicate that that the majority of guide nucleic acids achieving higher Indel rates contain a spacer sequence that is complementary to and/or hybridizes to a target sequence situated upstream of the transcription start site (TSS).

The study also aimed to evaluate the gene silencing capabilities of a fusion protein that includes a CasM.265466 mutant, dCasM.265466 (D220R and E335Q), when paired with one of the 23 guide nucleic acids. The fusion constructs including CasM.265466 mutants are listed as follows:
(a) KRAB(ZNF10)-dCasM.265466 (D220R and E335Q)-DMNT3L-DMNT3A
(b) KRAB(ZNF10)-dCasM.265466 (D220R and E335Q)-DMNT3L
(c) DNMT3L-KRAB(ZNF10)-dCasM.265466 (D220R and E335Q)
(d) DNMT3A-DNMT3L-dCasM.265466 (D220R and E335Q)-KRAB(ZNF10)
(e) dCasM.265466 (D220R and E335Q)-DNMT3L-KRAB(ZNF10)
(f) KRAB(ZNF10)-dCasM.265466 (D220R and E335Q)

HEK293T cells with a synthetic GFP reporter were transfected with plasmid 1 encoding one of the fusion constructs and plasmid 2 encoding one of the 23 guide nucleic acids. The negative control is dCas9 (CRISPROff) (SEQ ID NO: 22) with a non-targeting guide nucleic acid (NT guide) and the positive control is dCasPhi(L26R, E567Q)-DNMT3L-KRAB(ZNF10) (EpiCasPhi) with a validated guide nucleic acid. The GFP flow cytometry analysis began on day 5 and concluded on day 21. Measurements were taken on day 5, 7, 9, 12,15, 19 and 21.

Based on the day 21 GFP repression data, 6 out of the 23 guide nucleic acids were identified to show repressive activity. TABLE 15 provides the spacer sequences and full sequences of the 6 guide nucleic acids paired with dCasM.265466 fusion proteins for targeting GFP.

TABLE 15
Spacer sequences and full sequences of the 6 guide nucleic acids

| Distance | | | SEQ | | SEQ |
|---|---|---|---|---|---|
| from | | Spacer | ID | | ID |
| TSS | PI# | Sequence | NO: | Full Sequence | NO: |
| −398 | P134398 | CAGUGCC | 3000 | ACAGCUUAUUUGGAAGCUGA | 2944 |
| | | AGGUGA | | AAUGUGAGGUUUAUAACACU | |
| | | AAAUCG | | CACAAGAAUCCUGAAAAGGA | |
| | | C | | UGCCAAACCAGUGCCAGGUG | |
| | | | | AAAAUCGC | |
| −373 | P134386 | GAACAG | 3001 | ACAGCUUAUUUGGAAGCUGA | 2945 |
| | | GGAGGA | | AAUGUGAGGUUUAUAACACU | |
| | | GCAGAG | | CACAAGAAUCCUGAAAAGGA | |
| | | AG | | UGCCAAACGAACAGGGAGGA | |
| | | | | GCAGAGAG | |
| −324 | P134403 | UAGGAA | 3002 | ACAGCUUAUUUGGAAGCUGA | 2946 |
| | | CCCGGAU | | AAUGUGAGGUUUAUAACACU | |
| | | GGUGGG | | CACAAGAAUCCUGAAAAGGA | |
| | | G | | UGCCAAACUAGGAACCCGGA | |
| | | | | UGGUGGGG | |
| −221 | P134388 | CGGGUG | 3003 | ACAGCUUAUUUGGAAGCUGA | 2947 |
| | | CACGUA | | AAUGUGAGGUUUAUAACACU | |
| | | GCUCAG | | CACAAGAAUCCUGAAAAGGA | |
| | | GC | | UGCCAAACCGGGUGCACGUA | |
| | | | | GCUCAGGC | |
| −182 | P134392 | AGCGUC | 2999 | ACAGCUUAUUUGGAAGCUGA | 2948 |
| | | AAUUGG | | AAUGUGAGGUUUAUAACACU | |
| | | AGAGAG | | CACAAGAAUCCUGAAAAGGA | |
| | | GC | | UGCCAAACAGCGUCAAUUGG | |
| | | | | AGAGAGGC | |
| −76 | P134380 | CGCAUG | 2943 | ACAGCUUAUUUGGAAGCUGA | 2949 |
| | | UGCAGCC | | AAUGUGAGGUUUAUAACACU | |
| | | AUUGCC | | CACAAGAAUCCUGAAAAGGA | |
| | | U | | UGCCAAACCGCAUGUGCAGC | |
| | | | | CAUUGCCU | |

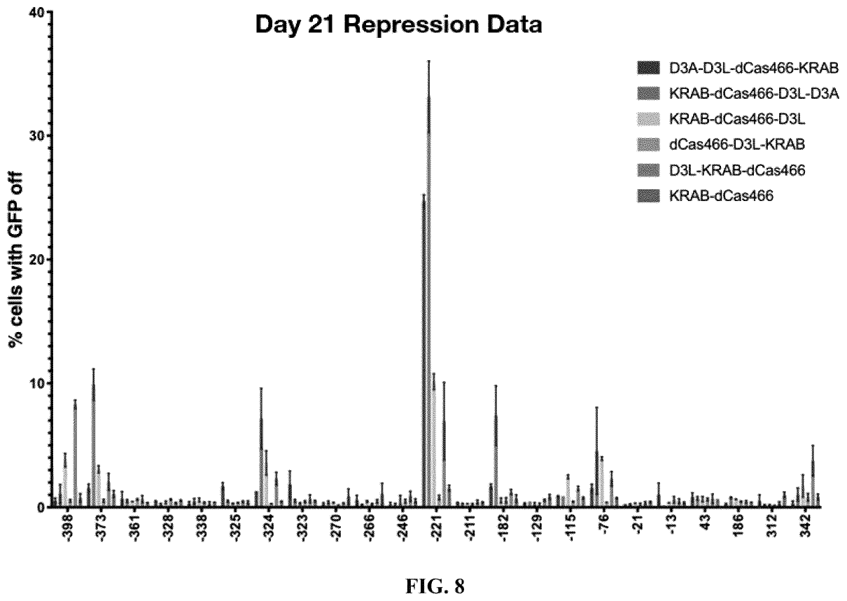

FIG. 8 illustrates that the majority of guide nucleic acids with repressive activity contain a spacer sequence that is complementary to and/or hybridizes to a target sequence situated upstream of the transcription start site (TSS). Compared to dCasPhi.12, the candidate guides for dCasM.265466 extend even further into the GAPDH CpG island. FIG. 8 also indicates that the fusion constructs, specifically KRAB(ZNF10)-dCasM.265466 (D220R and E335Q)-DMNT3L-DMNT3A, KRAB(ZNF10)-dCasM.265466 (D220R and E335Q)-DMNT3L, DNMT3L-KRAB(ZNF10)-dCasM.265466 (D220R and E335Q), and DNMT3A-DNMT3L-dCasM.265466 (D220R and E335Q)-KRAB(ZNF10), when paired with the selected guide nucleic acids, display repressive activity. KRAB(ZNF10)-dCasM.265466 (D220R and E335Q)-DMNT3L-DMNT3A demonstrated the highest activity.

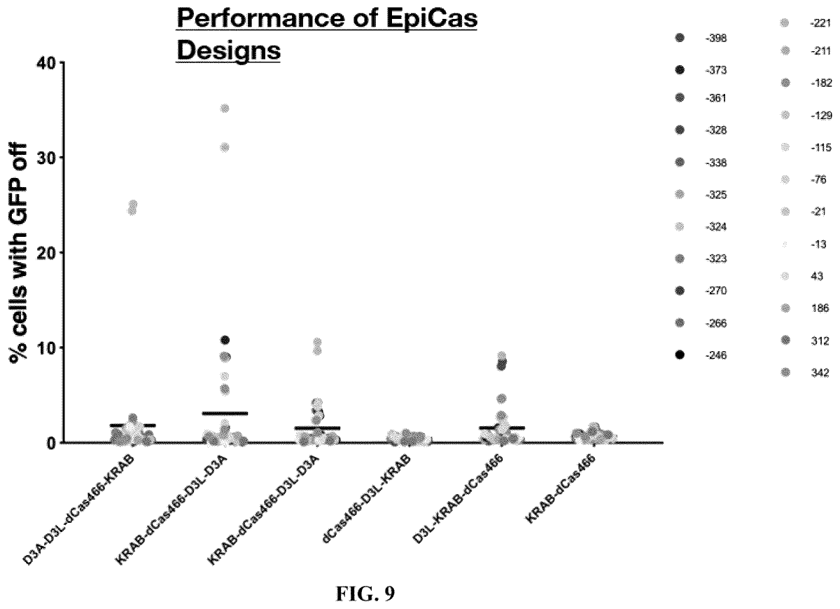

The results also indicate that a triple-effector partner design outperformed the other designs. As depicted in FIG. 9, the fusion construct KRAB(ZNF10)-dCasM.265466 (D220R and E335Q)-DMNT3L-DMNT3A exhibited the highest repressive activity.

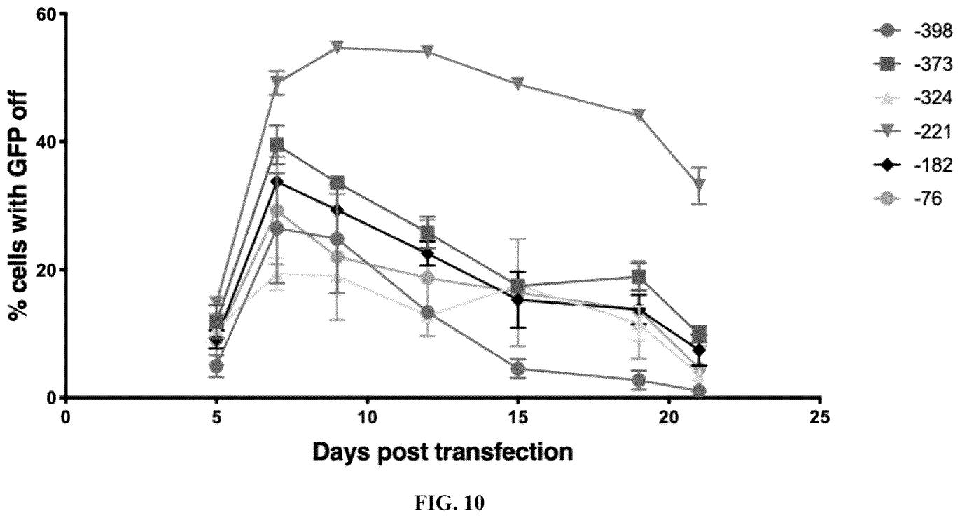

As shown in FIG. 10, when paired with the best performing fusion protein design KRAB(ZNF10)-dCasM.265466 (D220R and E335Q)-DMNT3L-DMNT3A, the guide nucleic acid with a spacer sequence that is complementary to and/or hybridizes to a target sequence situated 221 nucleotides upstream of the TSS (guide −221) exhibited the most potent repressive activity over the 21-day period.

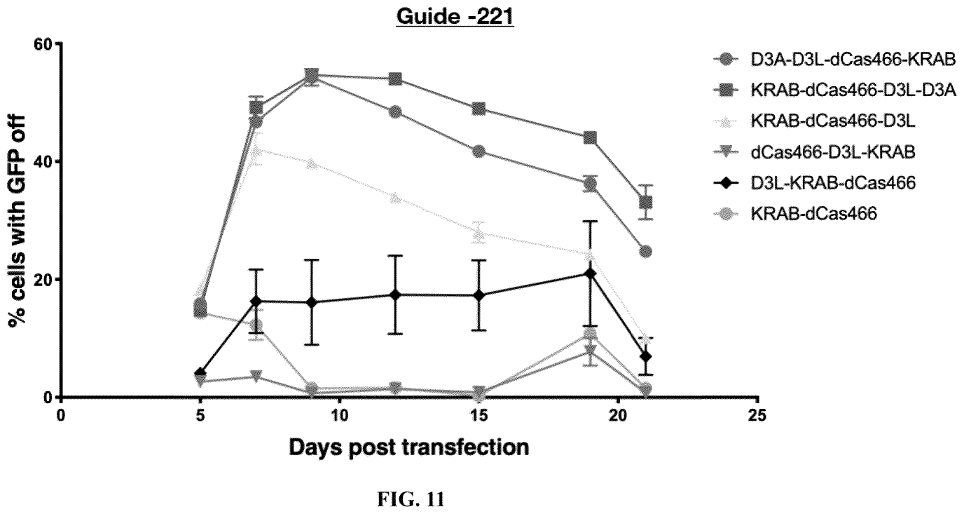

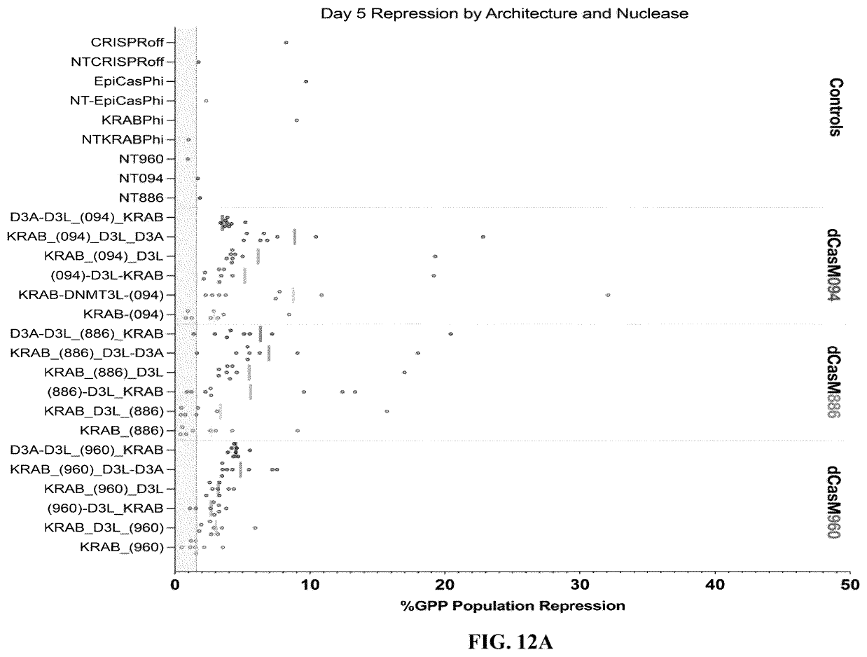

As shown in FIG. 11, when paired with the best performing guide −221, the triple-effector partner design KRAB(ZNF10)-dCasM.265466-DMNT3L-DMNT3A exhibited the highest repressive activity outperformed other fusion protein designs over the 21-day period.

### Example 5. Repression Validation of Type V Group 9 dCasM Proteins

This study aimed to evaluate the epigenetic repressive activity of the hypercompact deactivated Cas protein family type V Group 9 dCasM (G9 dCasM) effector proteins including dCasM.288094 (dCasM.094) (SEQ ID NO: 3011), dCasM.288886 (dCasM.886) (SEQ ID NO: 3010), and dCasM.2401960 (dCasM.960) (SEQ ID NO: 3012). Each G9 dCasM (dCasM.094/886/960) has six architectural variations when combined with one or more effector partners selected from NDMT3L, DNMT3A, and KRAB(ZNF10) to constitute a single or multiple appended chromatin effector fusion protein. These fusion constructs are listed as follows:
(a) DNMT3A-DNMT3L-dCasM.094/886/960-KRAB(ZNF10)
(b) KRAB(ZNF10)-dCasM.094/886/960IMT3L-DNMT3A
(c) KRAB(ZNF1)-dCasM.094/886/960-DNMT3L
(d) dCasM.094/886/960-DNMT3L-KRAB(ZNF10)
(e) KRAB(ZNF16)-DNMT3L-dCasM.094/886/960
(f) KRAB(ZNF1)-dCasM.094/886/960

HEK293T cells with a synthetic GFP reporter were transfected with plasmid 1 encoding a fusion construct and plasmid 2 encoding a guide nucleic acid. The GFP flow cytometry analysis began on day 5 and concluded on day 15. Measurements were taken on day 5, 7, 9, 12, and 15.

For each G9 dCasM, eight guide nucleic acids were generated for evaluation. These guide nucleic acids comprise spacer sequences that target regions upstream or downstream of the GFP transcription start site (TSS). TABLE 16 provides the spacer sequences and full sequences of the guide nucleic acids paired with each G9 dCasM for targeting GFP.

TABLE 16

| | | | SEQ | | SEQ |
|---|---|---|---|---|---|
| | | Spacer | ID | | ID |
| PL# | Nuclease | Sequence | NO: | Full Sequence | NO: |
| PL32677 | dCasM. | CGUUGCA | 2957 | gUCGGGGCGGCUGUACGCCC | 2981 |
| | 288886 | AAUCACU | | UUAAGUCGAGGCGGAGGCAC | |
| | | CCUCAG | | UCGAUAAAUACCUGAAAAGG | |
| | | | | UAUAUACAACCGUUGCAAAU | |
| | | | | CACUCCUCAG | |
| PL32686 | dCasM. | CGUCGCC | 2958 | gUCGGGGCGGCUGUACGCCC | 2982 |
| | 288886 | GUCCAGC | | UUAAGUCGAGGCGGAGGCAC | |
| | | UCGACC | | UCGAUAAAUACCUGAAAAGG | |
| | | | | UAUAUACAACCGUCGCCGUC | |
| | | | | CAGCUCGACC | |
| PL32691 | dCasM. | UGGAUUC | 2959 | gUAGGGGUGGUUGUGCACCC | 2983 |
| | 288094 | UGCGGCC | | UGAGCGGGAGGUUUUUAAGC | |
| | | GCGCCA | | ACUCCUGAAUUACCCUAUUU | |
| | | | | GAAAAGGUAAACACAACUGG | |
| | | | | AUUCUGCGGCCGCGCCA | |
| PL32692 | dCasM. | CCUGGGA | 2960 | gUAGGGGUGGUUGUGCACCC | 2984 |
| | 288094 | CGCAUGC | | UGAGCGGGAGGUUUUUAAGC | |
| | | GUAGGG | | ACUCCUGAAUUACCCUAUUU | |
| | | | | GAAAAGGUAAACACAACCCU | |
| | | | | GGGACGCAUGCGUAGGG | |
| PL32707 | dCasM. | CCGCAAU | 2961 | gUAGGGGUGGUUGUGCACCC | 2985 |
| | 288094 | GGCUCAG | | UGAGCGGGAGGUUUUUAAGC | |
| | | GUUUGU | | ACUCCUGAAUUACCCUAUUU | |
| | | | | GAAAAGGUAAACACAACCCG | |
| | | | | CAAUGGCUCAGGUUUGU | |
| PL32715 | dCasM. | CCGUAGG | 2962 | gUAGGGGUGGUUGUGCACCC | 2986 |
| | 288094 | UGGCAUC | | UGAGCGGGAGGUUUUUAAGC | |
| | | GCCCUC | | ACUCCUGAAUUACCCUAUUU | |
| | | | | GAAAAGGUAAACACAACCCG | |
| | | | | UAGGUGGCAUCGCCCUC | |
| PL32716 | dCasM. | CUCACCA | 2963 | gUAGGGGUGGUUGUGCACCC | 2987 |
| | 288094 | UGGUGGC | | UGAGCGGGAGGUUUUUAAGC | |
| | | GCGGCC | | ACUCCUGAAUUACCCUAUUU | |
| | | | | GAAAAGGUAAACACAACCUC | |
| | | | | ACCAUGGUGGCGCGGCC | |
| PL32723 | dCasM. | UCGCGCG | 2964 | gUAGGGGUGGUUGUGCACCC | 2988 |
| | 288094 | GCUCCCU | | UGAGCGGGAGGUUUUUAAGC | |
| | | ACGCAU | | ACUCCUGAAUUACCCUAUUU | |
| | | | | GAAAAGGUAAACACAACUCG | |
| | | | | CGCGGCUCCCUACGCAU | |
| PL32728 | dCasM. | CGUUGCA | 2965 | gUAGGGGUGGUUGUGCACCC | 2989 |
| | 288094 | AAUCACU | | UGAGCGGGAGGUUUUUAAGC | |
| | | CCUCAG | | ACUCCUGAAUUACCCUAUUU | |
| | | | | GAAAAGGUAAACACAACCGU | |
| | | | | UGCAAAUCACUCCUCAG | |
| PL32737 | dCasM. | CGUCGCC | 2966 | gUAGGGGUGGUUGUGCACCC | 2990 |
| | 288094 | GUCCAGC | | UGAGCGGGAGGUUUUUAAGC | |
| | | UCGACC | | ACUCCUGAAUUACCCUAUUU | |
| | | | | GAAAAGGUAAACACAACCGU | |
| | | | | CGCCGUCCAGCUCGACC | |
| PL32742 | dCasM. | UGGAUUC | 2967 | gAGGGGCCACUUGUAUUGGC | 2991 |
| | 2401960 | UGCGGCC | | CUGAGCAGGAGGGUGUAAUU | |
| | | GCGCCA | | GCCACUCCUGAAUGACCUGA | |
| | | | | AAAGGUUGAUACAACUGGAU | |
| | | | | UCUGCGGCCGCGCCA | |
| PL32743 | dCasM. | CCUGGGA | 2968 | gAGGGGCCACUUGUAUUGGC | 2992 |
| | 2401960 | CGCAUGC | | CUGAGCAGGAGGGUGUAAUU | |
| | | GUAGGG | | GCCACUCCUGAAUGACCUGA | |
| | | | | AAAGGUUGAUACAACCCUGG | |
| | | | | GACGCAUGCGUAGGG | |
| PL32758 | dCasM. | CCGCAAU | 2969 | gAGGGGCCACUUGUAUUGGC | 299 |
| | 2401960 | GGCUCAG | | CUGAGCAGGAGGGUGUAAUU | |
| | | GUUUGU | | GCCACUCCUGAAUGACCUGA | |
| | | | | AAAGGUUGAUACAACCCGCA | |
| | | | | AUGGCUCAGGUUUGU | 3 |
| PL32766 | dCasM. | CCGUAGG | 2970 | gAGGGGCCACUUGUAUUGGC | 2994 |
| | 2401960 | UGGCAUC | | CUGAGCAGGAGGGUGUAAUU | |
| | | GCCCUC | | GCCACUCCUGAAUGACCUGA | |
| | | | | AAAGGUUGAUACAACCCGUA | |
| | | | | GGUGGCAUCGCCCUC | |
| PL32767 | dCasM. | CUCACCA | 2971 | gAGGGGCCACUUGUAUUGGC | 2995 |
| | 2401960 | UGGUGGC | | CUGAGCAGGAGGGUGUAAUU | |
| | | GCGGCC | | GCCACUCCUGAAUGACCUGA | |
| | | | | AAAGGUUGAUACAACCUCAC | |
| | | | | CAUGGUGGCGCGGCC | |
| PL32774 | dCasM. | UCGCGCG | 2972 | gAGGGGCCACUUGUAUUGGC | 2996 |
| | 2401960 | GCUCCCU | | CUGAGCAGGAGGGUGUAAUU | |
| | | ACGCAU | | GCCACUCCUGAAUGACCUGA | |
| | | | | AAAGGUUGAUACAACUCGCG | |
| | | | | CGGCUCCCUACGCAU | |
| PL32779 | dCasM. | CGUUGCA | 2973 | gAGGGGCCACUUGUAUUGGC | 2997 |
| | 2401960 | AAUCACU | | CUGAGCAGGAGGGUGUAAUU | |
| | | CCUCAG | | GCCACUCCUGAAUGACCUGA | |
| | | | | AAAGGUUGAUACAACCGUUG | |
| | | | | CAAAUCACUCCUCAG | |
| PL32788 | dCasM. | CGUCGCC | 2974 | gAGGGGCCACUUGUAUUGGC | 2998 |
| | 2401960 | GUCCAGC | | CUGAGCAGGAGGGUGUAAUU | |
| | | UCGACC | | GCCACUCCUGAAUGACCUGA | |
| | | | | AAAGGUUGAUACAACCGUCG | |
| | | | | CCGUCCAGCUCGACC | |

Eight control constructs include dCasM.094/886/960 with a NT guide. KRAB(ZNF10)-CasPhi.12, dCasPhi(L26R and E567Q)-DNMT3L-KRAB (EpiCasPhi) with a NT guide, EpiCasPhi, CRISPROff with a NT guide, and CRISPROff.

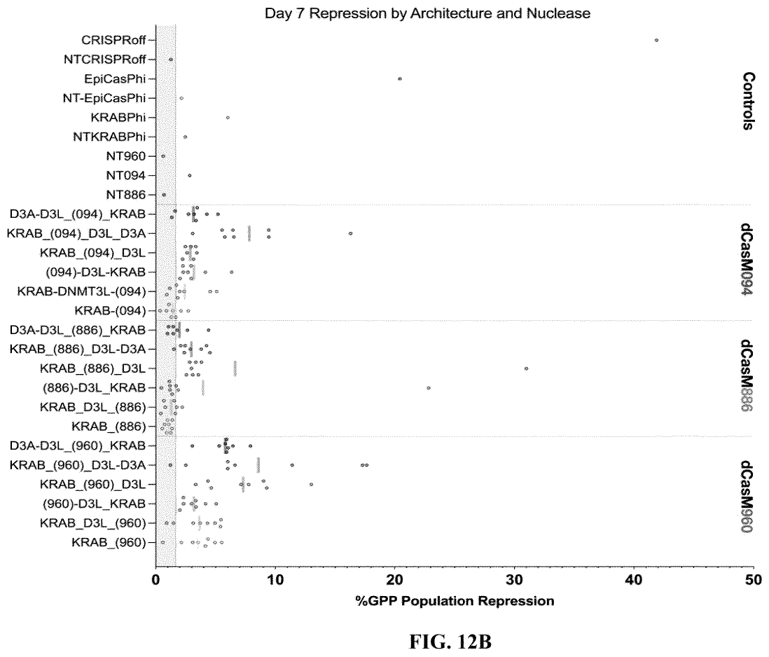

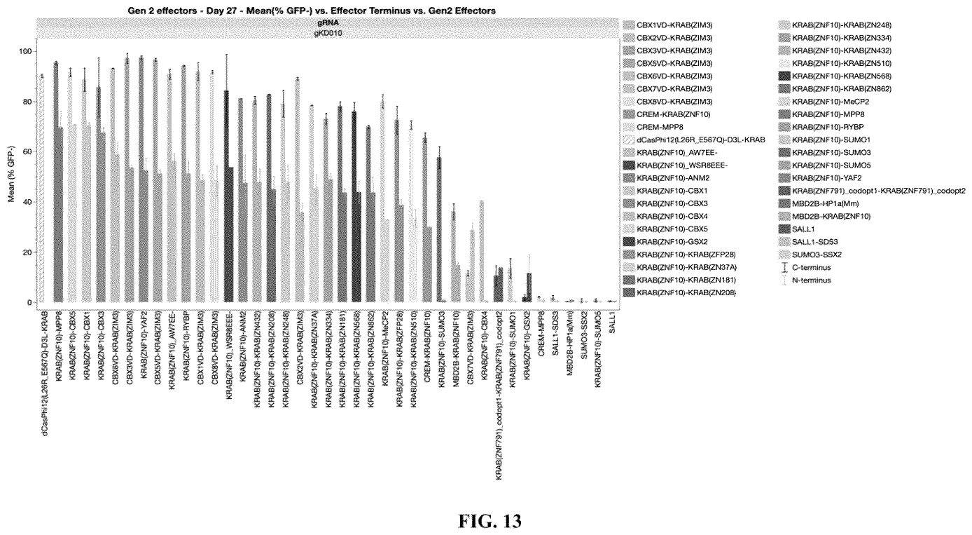

FIGS. 12A-12B depict that triple-effector partner designs demonstrate better activity in repressing GFP+ cells than dual-effector partner designs. Further, KRAB(ZNF10)-dCasM.094/886/960-DNMT3L-DNMT3A and KIRAB(ZNF10)-dCasM.094/886/960-DNMT3L repressed GFP+ cells more efficiently and stably than other designs.

### Example 6. Multiple Repressors and Stabilizers Provide Durable Silencing by dCasPhi.12

Without being bound by theory, KRAB zinc finger domains described herein may serve as a repressor of target expression when fused to a Gas effector protein, while methyltransferase(s) (e.g., DNMT3A and DNMT3L) serve as a stabilizer. In this example, a larger screen of repressors to replace KRAB(ZNF10) was performed, including 2 stability engineered KRAB(ZNF10), 5 wholly non-KRAB mono/bipartite repressor, 22 KRAB+non-KRAB bipartite repressors, and 11 KRAB+KRAB bipartite repressors. Repressors were fused in two orientations on dCasPhi12(L26R, E567Q): dCasPhi.12-D3L-Repressor1(-Repressor 2) dubbed “C-terminus effectors,” and Repressor1(-Repressor2)-dCas-DNMT3L dubbed “N-terminus effectors.” HEK293T cells were transfected with plasmids encoding a GFP reporter, a fusion protein, and a guide nucleic acid, such that if the fusion protein and guide nucleic acid form a complex that binds the GFP reporter, GFP expression will be repressed. Repression was tracked over 27 days post-transfection with flow cytometry.

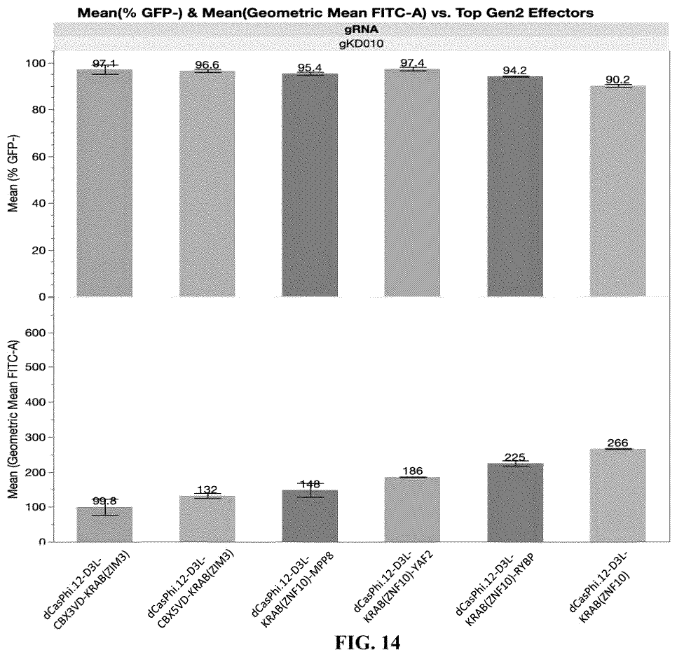

FIG. 13 shows that the C-terminus effectors generally have stronger repression than N-terminus effectors. FIG. 14 shows the C-terminus effectors that provided the greatest GFP repression at day 27. TABLE 17 provides the sequences of dCasPhi12(L26R, E567Q)-DNMT3L and the repressors used to generate the data shown in FIG. 14.

TABLE 17
Sequences of dcasphi12(126r, e567q)-dnmt3l and the repressors

| | | SEQ |
|---|---|---|
| Protein | | ID |
| Description | Amino Acid Sequence | NO |
| dCasPhi.12- | MAPKKKRKVGIHGVPAAMIKPTVSQFLTPGFKLIRNHSRTAGR | 3004 |
| DNMT3L | KLKNEGEEACKKFVRENEIPKDECPNFQGGPAIANIIAKSREF | |
| | TEWEIYQSSLAIQEVIFTLPKDKLPEPILKEEWRAQWLSEHG | |
| | LDTVPYKEAAGLNLIIKNAVNTYKGVQVKVDNKNKNNLAKI | |
| | NRKNEIAKLNGEQEISFEEIKAFDDKGYLLQKPSPNKSIYCYQ | |
| | SVSPKPFITSKYHNVNLPEEYIGYYRKSNEPIVSPYQFDRLRIP | |
| | IGEPGYVPKWQYTFLSKKENKRRKLSKRIKNVSPILGIICIKK | |
| | DWCVFDMRGLLRTNHWKKYHKPTDSINDLFDYFTGDPVIDT | |
| | KANVVRFRYKMENGIVNYKPVREKKGKELLENICDQNGSCK | |
| | LATVDVGQNNPVAIGLFELKKVNGELTKTLISRHPTPIDFCN | |
| | KITAYRERYDKLESSIKLDAIKQLTSEQKIEVDNYNNNFTPQN | |
| | TKQIVCSKLNINPNDLPWDKMISGTHFISEKAQVSNKSEIYFT | |
| | STDKGKTKDVMKSDYKWFQDYKPKLSKEVRDALSDIEWRL | |
| | RRESLEFNKLSKSREQDARQLANWISSMCDVIGIQNLVKKNN | |
| | FFGGSGKREPGWDNFYKPKKENRWWINAIHKALTELSQNK | |
| | GKRVILLPAMRTSITCPKCKYCDSKNRNGEKFNCLKCGIELN | |
| | ADIDVATENLATVAITAQSMPKPTCERSGDAKKPVRARKAK | |
| | APEFHDKLAPSYTVVLREAVKRPAATKKAGQAKKKKEFGGPSS | |
| | GAPPPSGGSPAGSPTSTEEGTSESATPESGPGTSTEPSEGSAPGSP | |
| | AGSMGPMEIYKTVSAWKRQPVRVLSLFRNIDKVLKSLGFLESGS | |
| | GSGGGTLKYVEDVTNVVRRDVEKWGPFDLVYGSTQPLGSSCDR | |
| | CPGWYMFQFHRILQYALPRQESQRPFFWIFMDNLLLTEDDQETT | |
| | TRFLQTEAVTLQDVRGRDYQNAMRVWSNIPGLKSKHAPLTPKE | |
| | EEYLQAQVRSRSKLDAPKVDLLVKNCLLPLREYFKYFSQNSLPL | |
| | GSPAGSPTSTEEGTSESATP-(Repressor Sequences in rows below) | |
| | wherein italics represent NLSs, bold represents dCasPhi.12(L26R, | |
| | E567Q), regular font represents mouse Dnmt31 (C terminus domain), | |
| | and double underline represents linker | |
| KRAB | MDAKSLTAWSRTLVTFKDVFVDFTREEWKLLDTAQQIVYRN | 3005 |
| (ZNF10)- | VMLENYKNLVSLGYQLTKPDVILRLEKGEEPWLVESGPGTST | |
| YAF2 | EPSEGSAPGSPAMKDKVEKEKSEKETTSKKNSHKKTRPRLKNVDRS | |
| | SAQHLEVTVGDLTVIITDFKEKTKSPPASSAASADQHSQSGSSSDNT | |
| | wherein bold represents KRAB(ZNF10), italics represent YAF2 | |
| | (Yaf2/RYBP C-terminal binding motif), and regular font represents a | |
| | linker | |
| KRAB | MDAKSLTAWSRTLVTFKDVFVDFTREEWKLLDTAQQIVYRN | 3006 |
| (ZNF10)- | VMLENYKNLVSLGYQLTKPDVILRLEKGEEPWLVESGPGTST | |
| MPP8 | EPSEGSAPGSPAMAEAFGDSEEDGEDVFEVEKILDMKTEGGKVLY | |
| | KVRWKGYTSDDDTWEPEIHLEDCKEVLLEFRKKIAENKAKAVRKDI | |
| | QR | |
| | wherein bold represents KRAB(ZNF10), italics represent MPP8 | |
| | chromodomain, and regular font represents a linker | |
| KRAB | MDAKSLTAWSRTLVTFKDVFVDFTREEWKLLDTAQQIVYRN | 3007 |
| (ZNF10)- | VMLENYKNLVSLGYQLTKPDVILRLEKGEEPWLVESGPGTST | |
| RYBP | EPSEGSAPGSPAMPSEANSIQSANATTKTSETNHTSRPRLKNVDRSTA | |
| | QQLAVTVGNVTVIITDFKEKTRSSSTSSSTVTSSAGSEQQNQSSS | |
| | wherein bold represents KRAB(ZNF10), italics represent RYBP | |
| | (Yaf2/RYBP C-terminal binding motif), and regular font represents a | |
| | linker | |
| CBX3VD- | MEEAEPDEFVVEKVLDRRVVNGKVEYFLKWEGFTDADNTW | 3008 |
| KRAB | EPEENLDCPELIEAFLNSQKSRSSGGGSGGSGSMNNSQGRVTFE | |
| (ZIM3) | DVTVNFTQGEWQRLNPEQRNLYRDVMLENYSNLVSVGQGETTKPD | |
| | VILRLEQGKEPWLEEEEVLGSGRAEKNGDIGGQIWKPKDVKESL | |
| | wherein bold represents CBX3 (chromodomain, Q9D, K33E), italics | |
| | represent ZIM3 (KRAB domain), and regular font represents a linker | |
| CBX5VD- | MEDEYVVEKVLDRRVVKGQVEYLLKWEGFSEEHNTWEPEK | 3009 |
| KRAB | NLDCPELISEFMKKYKKMKEGENSRSSGGGSGGSGSMNNSQG | |
| (ZIM3) | RVTFEDVTVNFTQGEWQRLNPEQRNLYRDVMLENYSNLVSVGQGET | |
| | TKPDVILRLEQGKEPWLEEEEVLGSGRAEKNGDIGGQIWKPKDVKE | |
| | SL | |
| | wherein bold represents CBX5 (chromodomain, Q9D, K33E), italics | |
| | represent ZIM3 (KRAB domain), and regular font represents a linker | |

### Example 7. CasPhi.12 Variant Fusions Provide Robust and Durable Epigenetic Silencing

CasPhi.12 repressor fusions, gRNA & controls were delivered to a HEK293T GFP expressing cell line by plasmid transfection. EpiCas fusions tested included CasPhi^{L26R/E567Q}-mDnmt3l-KRAB^{ZNF10} (mDnmt3l is a fragment of mouse DNMT3L), CasPhi^{L26R/E567Q}-mDnmt3l-CBX3VD-KRAB^{ZIM3}, CasPhiL26R/E567Q-mDnmt3lKRAB^{ZNF10}-MPP8, and CRISPRoff (Cas9 positive control, see Nunez et al., Cell (2021) for a detailed description of CRISPRoff). The sequences of all constructs and guides tested are provided in TABLE 18.

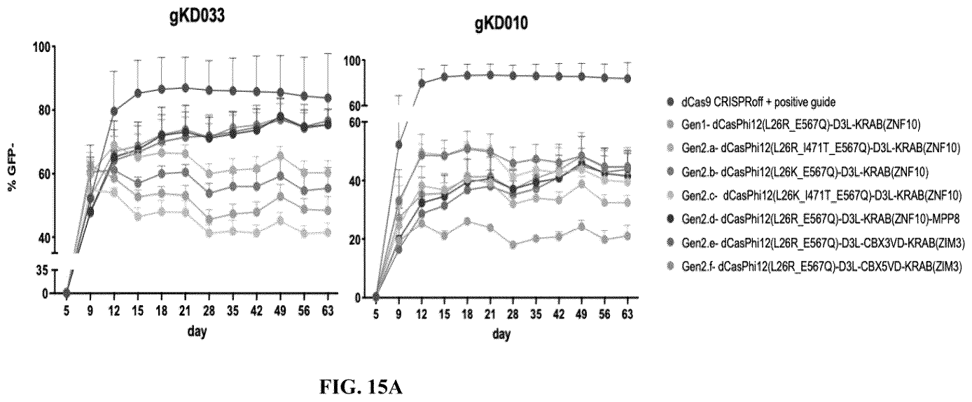

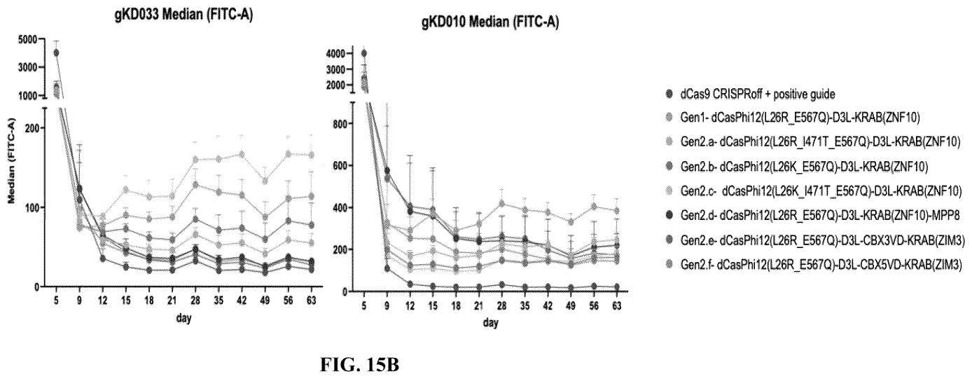

Puromycin selection was administered for 3 days to enrich fortransfected cells. gRNAs for EpiCas and controls were designed to target the promoter regulating GFP. GFP silencing was assayed by flow cytometry at various days post-transfection. CasPhi^{L26R/E567Q}-mDnmt301 CBX3VDtKRAB^{ZM3}, CasPhiL26R/E567QFmDnmt3lKRAB^{ZNF10}-MPP8 showed Cas9-CRISPRoff equivalence out to 63 days. See FIG. 15A (% GFP-cells) and FIG. 15B (FITC positive cells) for results. It is worth noting that, unlike Cas9, these CasPhi.12 fusions and guides are capable of fitting into a single AAV vector.

TABLE 18
Exemplary fusion proteins

| Fusion Protein | | SEQ |
|---|---|---|
| Description | Fusion Protein Sequence | ID |
| dCasPhi12(L26R | MAPKKKRKVGIHGVPAAMIKPTVSQFLTPGFKLIRNHSRTA | 3013 |
| E567Q)-mDnmt3l- | GRKLKNEGEEACKKFVRENEIPKDECPNFQGGPAIANIIAKS | |
| KRAB(ZNF10) | REFTEWEIYQSSLAIQEVIFTLPKDKLPEPILKEEWRAQWLSE | |
| (codon | HGLDTVPYKEAAGLNLIIKNAVNTYKGVQVKVDNKNKNNL | |
| optimization) | AKINRKNEIAKLNGEQEISFEEIKAFDDKGYLLQKPSPNKSIY | |
| variant 1 | CYQSVSPKPFITSKYHNVNLPEEYIGYYRKSNEPIVSPYQFDR | |
| (NLS at positions 1- | LRIPIGEPGYVPKWQYTFLSKKENKRRKLSKRIKNVSPILGII | |
| 17; dCas at | CIKKDWCVFDMRGLLRTNHWKKYHKPTDSINDLFDYFTGD | |
| positions 18-734; | PVIDTKANVVRFRYKMENGIVNYKPVREKKGKELLENICDQ | |
| NLS at positions | NGSCKLATVDVGQNNPVAIGLFELKKVNGELTKTLISRHPTP | |
| 735-752; linker at | IDFCNKITAYRERYDKLESSIKLDAIKQLTSEQKIEVDNYNN | |
| positions 753-806; | NFTPQNTKQIVCSKLNINPNDLPWDKMISGTHFISEKAQVSN | |
| mDNMt31 at | KSEIYFTSTDKGKTKDVMKSDYKWFQDYKPKLSKEVRDAL | |
| positions 807-1021; | SDIEWRLRRESLEFNKLSKSREQDARQLANWISSMCDVIGIQ | |
| KRAB (ZNF10) at | NLVKKNNFFGGSGKREPGWDNFYKPKKENRWWINAIHKAL | |
| positions 1022- | TELSQNKGKRVILLPAMRTSITCPKCKYCDSKNRNGEKFNC | |
| 1096) | LKCGIELNADIDVATENLATVAITAQSMPKPTCERSGDAKKP | |
| | VRARKAKAPEFHDKLAPSYTVVLREAVKRPAATKKAGQAK | |
| | KKKEFGGPSSGAPPPSGGSPAGSPTSTEEGTSESATPESGPGT | |
| | STEPSEGSAPGSPAGSMGPMEIYKTVSAWKRQPVRVLSLF | |
| | RNIDKVLKSLGFLESGSGSGGGTLKYVEDVTNVVRRDVE | |
| | KWGPFDLVYGSTQPLGSSCDRCPGWYMFQFHRILQYAL | |
| | PRQESQRPFFWIFMDNLLLTEDDQETTTRFLQTEAVTLQ | |
| | DVRGRDYQNAMRVWSNIPGLKSKHAPLTPKEEEYLQAQ | |
| | VRSRSKLDAPKVDLLVKNCLLPLREYFKYFSQNSLPLMD | |
| | AKSLTAWSRTLVTFKDVFVDFTREEWKLLDTAQQIVYRNV | |
| | MLENYKNLVSLGYQLTKPDVILRLEKGEEPWLV | |
| dCasPhi12(L26R_I | MAPKKKRKVGIHGVPAAMIKPTVSQFLTPGFKLIRNHSRTA | 3014 |
| 471T_E567Q)- | GRKLKNEGEEACKKFVRENEIPKDECPNFQGGPAIANIIAKS | |
| mDnmt3l- | REFTEWEIYQSSLAIQEVIFTLPKDKLPEPILKEEWRAQWLSE | |
| KRAB(ZNF10) | HGLDTVPYKEAAGLNLIIKNAVNTYKGVQVKVDNKNKNNL | |
| (NLS at positions 1- | AKINRKNEIAKLNGEQEISFEEIKAFDDKGYLLQKPSPNKSIY | |
| 17; dCas at | CYQSVSPKPFITSKYHNVNLPEEYIGYYRKSNEPIVSPYQFDR | |
| positions 18-734; | LRIPIGEPGYVPKWQYTFLSKKENKRRKLSKRIKNVSPILGII | |
| NLS at positions | CIKKDWCVFDMRGLLRTNHWKKYHKPTDSINDLFDYFTGD | |
| 735-752; linker at | PVIDTKANVVRFRYKMENGIVNYKPVREKKGKELLENICDQ | |
| positions 753-806; | NGSCKLATVDVGQNNPVAIGLFELKKVNGELTKTLISRHPTP | |
| mDNMt31 at | IDFCNKITAYRERYDKLESSIKLDAIKQLTSEQKIEVDNYNN | |
| positions 807-1021; | NFTPQNTKQIVCSKLNINPNDLPWDKMTSGTHFISEKAQVSN | |
| linker at positions | KSEIYFTSTDKGKTKDVMKSDYKWFQDYKPKLSKEVRDAL | |
| 1022-1041; KRAB | SDIEWRLRRESLEFNKLSKSREQDARQLANWISSMCDVIGIQ | |
| (ZNF10) at | NLVKKNNFFGGSGKREPGWDNFYKPKKENRWWINAIHKAL | |
| positions 1042- | TELSQNKGKRVILLPAMRTSITCPKCKYCDSKNRNGEKFNC | |
| 1116) | LKCGIELNADIDVATENLATVAITAQSMPKPTCERSGDAKKP | |
| | VRARKAKAPEFHDKLAPSYTVVLREAVKRPAATKKAGQAK | |
| | KKKEFGGPSSGAPPPSGGSPAGSPTSTEEGTSESATPESGPGT | |
| | STEPSEGSAPGSPAGSMGPMEIYKTVSAWKRQPVRVLSLFR | |
| | NIDKVLKSLGFLESGSGSGGGTLKYVEDVTNVVRRDVEKW | |
| | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMDAKSLTAWSRTLVTFKDVFVDFTREEWKLLDTAQ | |
| | QIVYRNVMLENYKNLVSLGYQLTKPDVILRLEKGEEPWLV | |
| dCasPhi12(L26K_ | MAPKKKRKVGIHGVPAAMIKPTVSQFLTPGFKLIRNHSRTA | 3015 |
| E567Q)-mDnmt3l- | GKKLKNEGEEACKKFVRENEIPKDECPNFQGGPAIANIIAKS | |
| KRAB(ZNF10) | REFTEWEIYQSSLAIQEVIFTLPKDKLPEPILKEEWRAQWLSE | |
| (NLS at positions 1- | HGLDTVPYKEAAGLNLIIKNAVNTYKGVQVKVDNKNKNNL | |
| 17; dCas at | AKINRKNEIAKLNGEQEISFEEIKAFDDKGYLLQKPSPNKSIY | |
| positions 18-734; | CYQSVSPKPFITSKYHNVNLPEEYIGYYRKSNEPIVSPYQFDR | |
| NLS at positions | LRIPIGEPGYVPKWQYTFLSKKENKRRKLSKRIKNVSPILGII | |
| 735-752; linker at | CIKKDWCVFDMRGLLRTNHWKKYHKPTDSINDLFDYFTGD | |
| positions 753-806; | PVIDTKANVVRFRYKMENGIVNYKPVREKKGKELLENICDQ | |
| mDNMt31 at | NGSCKLATVDVGQNNPVAIGLFELKKVNGELTKTLISRHPTP | |
| positions 807-1021; | IDFCNKITAYRERYDKLESSIKLDAIKQLTSEQKIEVDNYNN | |
| linker at positions | NFTPQNTKQIVCSKLNINPNDLPWDKMISGTHFISEKAQVSN | |
| 1022-1041; KRAB | KSEIYFTSTDKGKTKDVMKSDYKWFQDYKPKLSKEVRDAL | |
| (ZNF10) at | SDIEWRLRRESLEFNKLSKSREQDARQLANWISSMCDVIGIQ | |
| positions 1042- | NLVKKNNFFGGSGKREPGWDNFYKPKKENRWWINAIHKAL | |
| 1116) | TELSQNKGKRVILLPAMRTSITCPKCKYCDSKNRNGEKFNC | |
| | LKCGIELNADIDVATENLATVAITAQSMPKPTCERSGDAKKP | |
| | VRARKAKAPEFHDKLAPSYTVVLREAVKRPAATKKAGQAK | |
| | KKKEFGGPSSGAPPPSGGSPAGSPTSTEEGTSESATPESGPGT | |
| | STEPSEGSAPGSPAGSMGPMEIYKTVSAWKRQPVRVLSLFR | |
| | NIDKVLKSLGFLESGSGSGGGTLKYVEDVTNVVRRDVEKW | |
| | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMDAKSLTAWSRTLVTFKDVFVDFTREEWKLLDTAQ | |
| | QIVYRNVMLENYKNLVSLGYQLTKPDVILRLEKGEEPWLV | |
| dCasPhi12(L26K_I | MAPKKKRKVGIHGVPAAMIKPTVSQFLTPGFKLIRNHSRTA | 3016 |
| 471T_E567Q)- | GKKLKNEGEEACKKFVRENEIPKDECPNFQGGPAIANIIAKS | |
| mDnmt3l- | REFTEWEIYQSSLAIQEVIFTLPKDKLPEPILKEEWRAQWLSE | |
| KRAB(ZNF10) | HGLDTVPYKEAAGLNLIIKNAVNTYKGVQVKVDNKNKNNL | |
| (NLS at positions 1- | AKINRKNEIAKLNGEQEISFEEIKAFDDKGYLLQKPSPNKSIY | |
| 17; dCas at | CYQSVSPKPFITSKYHNVNLPEEYIGYYRKSNEPIVSPYQFDR | |
| positions 18-734; | LRIPIGEPGYVPKWQYTFLSKKENKRRKLSKRIKNVSPILGII | |
| NLS at positions | CIKKDWCVFDMRGLLRTNHWKKYHKPTDSINDLFDYFTGD | |
| 735-752; linker at | PVIDTKANVVRFRYKMENGIVNYKPVREKKGKELLENICDQ | |
| positions 753-806; | NGSCKLATVDVGQNNPVAIGLFELKKVNGELTKTLISRHPTP | |
| mDNMt31 at | IDFCNKITAYRERYDKLESSIKLDAIKQLTSEQKIEVDNYNN | |
| positions 807-1021; | NFTPQNTKQIVCSKLNINPNDLPWDKMTSGTHFISEKAQVSN | |
| linker at positions | KSEIYFTSTDKGKTKDVMKSDYKWFQDYKPKLSKEVRDAL | |
| 1022-1041; KRAB | SDIEWRLRRESLEFNKLSKSREQDARQLANWISSMCDVIGIQ | |
| (ZNF10) at | NLVKKNNFFGGSGKREPGWDNFYKPKKENRWWINAIHKAL | |
| positions 1042- | TELSQNKGKRVILLPAMRTSITCPKCKYCDSKNRNGEKFNC | |
| 1116) | LKCGIELNADIDVATENLATVAITAQSMPKPTCERSGDAKKP | |
| | VRARKAKAPEFHDKLAPSYTVVLREAVKRPAATKKAGQAK | |
| | KKKEFGGPSSGAPPPSGGSPAGSPTSTEEGTSESATPESGPGT | |
| | STEPSEGSAPGSPAGSMGPMEIYKTVSAWKRQPVRVLSLFR | |
| | NIDKVLKSLGFLESGSGSGGGTLKYVEDVTNVVRRDVEKW | |
| | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMDAKSLTAWSRTLVTFKDVFVDFTREEWKLLDTAQ | |
| | QIVYRNVMLENYKNLVSLGYQLTKPDVILRLEKGEEPWLV | |
| dCasPhi12(L26R | MAPKKKRKVGIHGVPAAMIKPTVSQFLTPGFKLIRNHSRTA | 3017 |
| E567Q)-mDnmt31- | GRKLKNEGEEACKKFVRENEIPKDECPNFQGGPAIANIIAKS | |
| KRAB(ZNF10)- | REFTEWEIYQSSLAIQEVIFTLPKDKLPEPILKEEWRAQWLSE | |
| MPP8 | HGLDTVPYKEAAGLNLIIKNAVNTYKGVQVKVDNKNKNNL | |
| (NLS at positions 1- | AKINRKNEIAKLNGEQEISFEEIKAFDDKGYLLQKPSPNKSIY | |
| 17; dCas at | CYQSVSPKPFITSKYHNVNLPEEYIGYYRKSNEPIVSPYQFDR | |
| positions 18-734; | LRIPIGEPGYVPKWQYTFLSKKENKRRKLSKRIKNVSPILGII | |
| NLS at positions | CIKKDWCVFDMRGLLRTNHWKKYHKPTDSINDLFDYFTGD | |
| 735-752; linker at | PVIDTKANVVRFRYKMENGIVNYKPVREKKGKELLNICDQN | |
| positions 753-806; | GSCKLATVDVGQNNPVAIGLFELKKVNGELTKTLISRHPTPI | |
| mDNMt31 at | DFCNKITAYRERYDKLESSIKLDAIKQLTSEQKIEVDNYNNN | |
| positions 807-1021; | FTPQNTKQIVCSKLNINPNDLPWDKMISGTHFISEKAQVSNK | |
| linker at positions | SEIYFTSTDKGKTKDVMKSDYKWFQDYKPKLSKEVRDALS | |
| 1022-1041; KRAB | DIEWRLRRESLEFNKLSKSREQDARQLANWISSMCDVIGIQN | |
| (ZNF10) at | LVKKNNFGGSGKREPGWDNFYKPKKENRWWINAIHKALTE | |
| positions 1042- | LSQNKGKRVILLPAMRTSITCPKCKYCDSKNRNGEKFNCLK | |
| 1116; linker at | CGIELNADIDVATENLATVAITAQSMPKPTCERSGDAKKPV | |
| positions 1117- | RARKAKAPEFHDKLAPSYTVVLREAVKRPAATKKAGQAKK | |
| 1136; MPP8 at | KKEFGGPSSGAPPPSGGSPAGSPTSTEEGTSESATPESGPGTS | |
| positions 1137- | TEPSEGSAPGSPAGSMGPMEIYKTVSAWKRQPVRVLSLFRNI | |
| 1217) | DKVLKSLGFLESGSGSGGGTLKYVEDVTNVVRRDVEKWGP | |
| | FDLVYGSTQPLGSSCDRCPGWYMFQFHRILQYALPRQESQR | |
| | PFFWIFMDNLLLTEDDQETTTRFLQTEAVTLQDVRGRDYQN | |
| | AMRVWSNIPGLKSKHAPLTPKEEEYLQAQVRSRSKLDAPKV | |
| | DLLVKNCLLPLREYFKYFSQNSLPLGSPAGSPTSTEEGTSES | |
| | ATPMDAKSLTAWSRTLVTFKDVFVDFTREEWKLLDTAQQI | |
| | VYRNVMLENYKNLVSLGYQLTKPDVILRLEKGEEPWLVES | |
| | GPGTSTEPSEGSAPGSPAMAEAFGDSEEDGEDVFEVEKILD | |
| | MKTEGGKVLYKVRWKGYTSDDDTWEPEIHLEDCKEVLLEF | |
| | RKKIAENKAKAVRKDIQR | |
| dCasPhi12(L26R_ | MAPKKKRKVGIHGVPAAMIKPTVSQFLTPGFKLIRNHSRTA | 3018 |
| E567Q)-mDnmt3l- | GRKLKNEGEEACKKFVRENEIPKDECPNFQGGPAIANIIAKS | |
| CBX3VD- | REFTEWEIYQSSLAIQEVIFTLPKDKLPEPILKEEWRAQWLSE | |
| KRAB(ZIM3) | HGLDTVPYKEAAGLNLIIKNAVNTYKGVQVKVDNKNKNNL | |
| (NLS at positions 1- | AKINRKNEIAKLNGEQEISFEEIKAFDDKGYLLQKPSPNKSIY | |
| 17; dCas at | CYQSVSPKPFITSKYHNVNLPEEYIGYYRKSNEPIVSPYQFDR | |
| positions 18-734; | LRIPIGEPGYVPKWQYTFLSKKENKRRKLSKRIKNVSPILGII | |
| NLS at positions | CIKKDWCVFDMRGLLRTNHWKKYHKPTDSINDLFDYFTGD | |
| 735-752; linker at | PVIDTKANVVRFRYKMENGIVNYKPVREKKGKELLNICDQN | |
| positions 753-806; | GSCKLATVDVGQNNPVAIGLFELKKVNGELTKTLISRHPTPI | |
| mDNMt31 at | DFCNKITAYRERYDKLESSIKLDAIKQLTSEQKIEVDNYNNN | |
| positions 807-1021; | FTPQNTKQIVCSKLNINPNDLPWDKMISGTHFISEKAQVSNK | |
| linker at positions | SEIYFTSTDKGKTKDVMKSDYKWFQDYKPKLSKEVRDALS | |
| 1022-1041; | DIEWRLRRESLEFNKLSKSREQDARQLANWISSMCDVIGIQN | |
| CBX3VD at | LVKKNNFFGGSGKREPGWDNFYKPKKENRWWINAIHKALT | |
| positions 1042- | ELSQNKGKRVILLPAMRTSITCPKCKYCDSKNRNGEKFNCL | |
| 1101; linker at | KCGIELNADIDVATENLATVAITAQSMPKPTCERSGDAKKP | |
| positions 1102- | VRARKAKAPEFHDKLAPSYTVVLREAVKRPAATKKAGQAK | |
| 1114; KRAB | KKKEFGGPSSGAPPPSGGSPAGSPTSTEEGTSESATPESGPGT | |
| (ZIM3) at positions | STEPSEGSAPGSPAGSMGPMEIYKTVSAWKRQPVRVLSLFR | |
| 1115-1214) | NIDKVLKSLGFLESGSGSGGGTLKYVEDVTNVVRRDVEKW | |
| | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMEEAEPDEFVVEKVLDRRVVNGKVEYFLKWEGFTD | |
| | ADNTWEPEENLDCPELIEAFLNSQKSRSSGGGSGGSGSMNN | |
| | SQGRVTFEDVTVNFTQGEWQRLNPEQRNLYRDVMLENYSN | |
| | LVSVGQGETTKPDVILRLEQGKEPWLEEEEVLGSGRAEKNG | |
| | DIGGQIWKPKDVKESL | |
| dCasPhi12(L26R | MAPKKKRKVGIHGVPAAMIKPTVSQFLTPGFKLIRNHSRTA | 3019 |
| E567Q)-mDnmt3l- | GRKLKNEGEEACKKFVRENEIPKDECPNFQGGPAIANIIAKS | |
| CBX5VD- | REFTEWEIYQSSLAIQEVIFTLPKDKLPEPILKEEWRAQWLSE | |
| KRAB(ZIM3) | HGLDTVPYKEAAGLNLIIKNAVNTYKGVQVKVDNKNKNNL | |
| (NLS at positions 1- | AKINRKNEIAKLNGEQEISFEEIKAFDDKGYLLQKPSPNKSIY | |
| 17; dCas at | CYQSVSPKPFITSKYHNVNLPEEYIGYYRKSNEPIVSPYQFDR | |
| positions 18-734; | LRIPIGEPGYVPKWQYTFLSKKENKRRKLSKRIKNVSPILGII | |
| NLS at positions | CIKKDWCVFDMRGLLRTNHWKKYHKPTDSINDLFDYFTGD | |
| 735-752; linker at | PVIDTKANVVRFRYKMENGIVNYKPVREKKGKELLENICDQ | |
| positions 753-806; | NGSCKLATVDVGQNNPVAIGLFELKKVNGELTKTLISRHPTP | |
| mDNMt31 at | IDFCNKITAYRERYDKLESSIKLDAIKQLTSEQKIEVDNYNN | |
| positions 807-1021; | NFTPQNTKQIVCSKLNINPNDLPWDKMISGTHFISEKAQVSN | |
| linker at positions | KSEIYFTSTDKGKTKDVMKSDYKWFQDYKPKLSKEVRDAL | |
| 1022-1041; | SDIEWRLRRESLEFNKLSKSREQDARQLANWISSMCDVIGIQ | |
| CBX5VD at | NLVKKNNFFGGSGKREPGWDNFYKPKKENRWWINAIHKAL | |
| positions 1042- | TELSQNKGKRVILLPAMRTSITCPKCKYCDSKNRNGEKFNC | |
| 1104; linker at | LKCGIELNADIDVATENLATVAITAQSMPKPTCERSGDAKKP | |
| positions 1105- | VRARKAKAPEFHDKLAPSYTVVLREAVKRPAATKKAGQAK | |
| 1117; KRAB | KKKEFGGPSSGAPPPSGGSPAGSPTSTEEGTSESATPESGPGT | |
| (ZIM3) at positions | STEPSEGSAPGSPAGSMGPMEIYKTVSAWKRQPVRVLSLFR | |
| 1118-1217) | NIDKVLKSLGFLESGSGSGGGTLKYVEDVTNVVRRDVEKW | |
| | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMEDEYVVEKVLDRRVVKGQVEYLLKWEGFSEEHNT | |
| | WEPEKNLDCPELISEFMKKYKKMKEGENSRSSGGGSGGSGS | |
| | MNNSQGRVTFEDVTVNFTQGEWQRLNPEQRNLYRDVMLE | |
| | NYSNLVSVGQGETTKPDVILRLEQGKEPWLEEEEVLGSGRA | |
| | EKNGDIGGQIWKPKDVKESL | |
| dCasPhi12(E567Q) | MAPKKKRKVGIHGVPAAMIKPTVSQFLTPGFKLIRNHSRTA | 3020 |
| -mDnmt3l- | GLKLKNEGEEACKKFVRENEIPKDECPNFQGGPAIANIIAKS | |
| KRAB(ZNF10) | REFTEWEIYQSSLAIQEVIFTLPKDKLPEPILKEEWRAQWLSE | |
| (NLS at positions 1- | HGLDTVPYKEAAGLNLIIKNAVNTYKGVQVKVDNKNKNNL | |
| 17; dCas at | AKINRKNEIAKLNGEQEISFEEIKAFDDKGYLLQKPSPNKSIY | |
| positions 18-734; | CYQSVSPKPFITSKYHNVNLPEEYIGYYRKSNEPIVSPYQFDR | |
| NLS at positions | LRIPIGEPGYVPKWQYTFLSKKENKRRKLSKRIKNVSPILGII | |
| 735-752; linker at | CIKKDWCVFDMRGLLRTNHWKKYHKPTDSINDLFDYFTGD | |
| positions 753-806; | PVIDTKANVVRFRYKMENGIVNYKPVREKKGKELLENICDQ | |
| mDNMt31 at | NGSCKLATVDVGQNNPVAIGLFELKKVNGELTKTLISRHPTP | |
| positions 807-1021; | IDFCNKITAYRERYDKLESSIKLDAIKQLTSEQKIEVDNYNN | |
| linker at positions | NFTPQNTKQIVCSKLNINPNDLPWDKMISGTHFISEKAQVSN | |
| 1022-1041; KRAB | KSEIYFTSTDKGKTKDVMKSDYKWFQDYKPKLSKEVRDAL | |
| (ZNF10) at | SDIEWRLRRESLEFNKLSKSREQDARQLANWISSMCDVIGIQ | |
| positions 1042- | NLVKKNNFFGGSGKREPGWDNFYKPKKENRWWINAIHKAL | |
| 1116) | TELSQNKGKRVILLPAMRTSITCPKCKYCDSKNRNGEKNCL | |
| | KCGIELNADIDVATENLATVAITAQSMPKPTCERSGDAKKP | |
| | VRARKAKAPEFHDKLAPSYTVVLREAVKRPAATKKAGQAK | |
| | KKKEFGGPSSGAPPPSGGSPAGSPTSTEEGTSESATPESGPGT | |
| | STEPSEGSAPGSPAGSMGPMEIYKTVSAWKRQPVRVLSLFR | |
| | NIDKVLKSLGFLESGSGSGGGTLKYVEDVTNVVRRDVEKW | |
| | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMDAKSLTAWSRTLVTFKDVFVDFTREEWKLLDTAQ | |
| | QIVYRNVMLENYKNLVSLGYQLTKPDVILRLEKGEEPWLV | |
| dCasPhi12(L26R_ | MAPKKKRKVGIHGVPAAMIKPTVSQFLTPGFKLIRNHSRTA | 3021 |
| E567Q)-mDnmt3l- | GRKLKNEGEEACKKFVRENEIPKDECPNFQGGPAIANIIAKS | |
| KRAB(ZNF10) | REFTEWEIYQSSLAIQEVIFTLPKDKLPEPILKEEWRAQWLSE | |
| (codon | HGLDTVPYKEAAGLNLIIKNAVNTYKGVQVKVDNKNKNNL | |
| optimization) | AKINRKNEIAKLNGEQEISFEEIKAFDDKGYLLQKPSPNKSIY | |
| variant 2 | CYQSVSPKPFITSKYHNVNLPEEYIGYYRKSNEPIVSPYQFDR | |
| (NLS at positions 1- | LRIPIGEPGYVPKWQYTFLSKKENKRRKLSKRIKNVSPILGII | |
| 17; dCas at | CIKKDWCVFDMRGLLRTNHWKKYHKPTDSINDLFDYFTGD | |
| positions 18-734; | PVIDTKANVVRFRYKMENGIVNYKPVREKKGKELLENICDQ | |
| NLS at positions | NGSCKLATVDVGQNNPVAIGLFELKKVNGELTKTLISRHPTP | |
| 735-752; linker at | IDFCNKITAYRERYDKLESSIKLDAIKQLTSEQKIEVDNYNN | |
| positions 753-806; | NFTPQNTKQIVCSKLNINPNDLPWDKMISGTHFISEKAQVSN | |
| mDNMt31 at | KSEIYFTSTDKGKTKDVMKSDYKWFQDYKPKLSKEVRDAL | |
| positions 807-1021; | SDIEWRLRRESLEFNKLSKSREQDARQLANWISSMCDVIGIQ | |
| KRAB (ZNF10) at | NLVKKNNFFGGSGKREPGWDNFYKPKKENRWWINAIHKAL | |
| positions 1022- | TELSQNKGKRVILLPAMRTSITCPKCKYCDSKNRNGEKFNC | |
| 1096) | LKCGIELNADIDVATENLATVAITAQSMPKPTCERSGDAKKP | |
| | VRARKAKAPEFHDKLAPSYTVVLREAVKRPAATKKAGQAK | |
| | KKKEFGGPSSGAPPPSGGSPAGSPTSTEEGTSESATPESGPGT | |
| | STEPSEGSAPGSPAGSMGPMEIYKTVSAWKRQPVRVLSLFR | |
| | NIDKVLKSLGFLESGSGSGGGTLKYVEDVTNVVRRDVEKW | |
| | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLMDAKSLTAWSRTLV | |
| | TFKDVFVDFTREEWKLLDTAQQIVYRNVMLENYKNLVSLG | |
| | YQLTKPDVILRLEKGEEPWLV | |
| dCasPhi12(L26K_I | MAPKKKRKVGIHGVPAAMIKPTVSQFLTPGFKLIRNHSRTA | 3022 |
| 471T_E567Q)- | GKKLKNEGEEACKKFVRENEIPKDECPNFQGGPAIANIIAKS | |
| mDnmt3l- | REFTEWEIYQSSLAIQEVIFTLPKDKLPEPILKEEWRAQWLSE | |
| KRAB(ZIM3) | HGLDTVPYKEAAGLNLIIKNAVNTYKGVQVKVDNKNKNNL | |
| (NLS at positions 1- | AKINRKNEIAKLNGEQEISFEEIKAFDDKGYLLQKPSPNKSIY | |
| 17; dCas at | CYQSVSPKPFITSKYHNVNLPEEYIGYYRKSNEPIVSPYQFDR | |
| positions 18-734; | LRIPIGEPGYVPKWQYTFLSKKENKRRKLSKRIKNVSPILGII | |
| NLS at positions | CIKKDWCVFDMRGLLRTNHWKKYHKPTDSINDLFDYFTGD | |
| 735-752; linker at | PVIDTKANVVRFRYKMENGIVNYKPVREKKGKELLENICDQ | |
| positions 753-806; | NGSCKLATVDVGQNNPVAIGLFELKKVNGELTKTLISRHPTP | |
| mDNMt31 at | IDFCNKITAYRERYDKLESSIKLDAIKQLTSEQKIEVDNYNN | |
| positions 807-1021; | NFTPQNTKQIVCSKLNINPNDLPWDKMTSGTHFISEKAQVSN | |
| linker at positions | KSEIYFTSTDKGKTKDVMKSDYKWFQDYKPKLSKEVRDAL | |
| 1022-1041; KRAB | SDIEWRLRRESLEFNKLSKSREQDARQLANWISSMCDVIGIQ | |
| (ZIM3) at positions | NLVKKNNFFGGSGKREPGWDNFYKPKKENRWWINAIHKAL | |
| 1042-1141) | TELSQNKGKRVILLPAMRTSITCPKCKYCDSKNRNGEKFNC | |
| | LKCGIELNADIDVATENLATVAITAQSMPKPTCERSGDAKKP | |
| | VRARKAKAPEFHDKLAPSYTVVLREAVKRPAATKKAGQAK | |
| | KKKEFGGPSSGAPPPSGGSPAGSPTSTEEGTSESATPESGPGT | |
| | STEPSEGSAPGSPAGSMGPMEIYKTVSAWKRQPVRVLSLFR | |
| | NIDKVLKSLGFLESGSGSGGGTLKYVEDVTNVVRRDVEKW | |
| | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMNNSQGRVTFEDVTVNFTQGEWQRLNPEQRNLYRD | |
| | VMLENYSNLVSVGQGETTKPDVILRLEQGKEPWLEEEEVLG | |
| | SGRAEKNGDIGGQIWKPKDVKESL | |
| dCasPhi12(L26K_I | MAPKKKRKVGIHGVPAAMIKPTVSQFLTPGFKLIRNHSRTA | 3023 |
| 471T_E567Q)- | GKKLKNEGEEACKKFVRENEIPKDECPNFQGGPAIANIIAKS | |
| mDnmt31- | REFTEWEIYQSSLAIQEVIFTLPKDKLPEPILKEEWRAQWLSE | |
| KRAB(ZNF10)- | HGLDTVPYKEAAGLNLIIKNAVNTYKGVQVKVDNKNKNNL | |
| MPP8 | AKINRKNEIAKLNGEQEISFEEIKAFDDKGYLLQKPSPNKSIY | |
| (NLS at positions 1- | CYQSVSPKPFITSKYHNVNLPEEYIGYYRKSNEPIVSPYQFDR | |
| 17; dCas at | LRIPIGEPGYVPKWQYTFLSKKENKRRKLSKRIKNVSPILGII | |
| positions 18-734; | CIKKDWCVFDMRGLLRTNHWKKYHKPTDSINDLFDYFTGD | |
| NLS at positions | PVIDTKANVVRFRYKMENGIVNYKPVREKKGKELLENICDQ | |
| 735-752; linker at | NGSCKLATVDVGQNNPVAIGLFELKKVNGELTKTLISRHPTP | |
| positions 753-806; | IDFCNKITAYRERYDKLESSIKLDAIKQLTSEQKIEVDNYNN | |
| mDNMt31 at | NFTPQNTKQIVCSKLNINPNDLPWDKMTSGTHFISEKAQVSN | |
| positions 807-1021; | KSEIYFTSTDKGKTKDVMKSDYKWFQDYKPKLSKEVRDAL | |
| linker at positions | SDIEWRLRRESLEFNKLSKSREQDARQLANWISSMCDVIGIQ | |
| 1022-1041; KRAB | NLVKKNNFFGGSGKREPGWDNFYKPKKENRWWINAIHKAL | |
| (ZNF10) at | TELSQNKGKRVILLPAMRTSITCPKCKYCDSKNRNGEKFNC | |
| positions 1042- | LKCGIELNADIDVATENLATVAITAQSMPKPTCERSGDAKKP | |
| 1116; linker at | VRARKAKAPEFHDKLAPSYTVVLREAVKRPAATKKAGQAK | |
| positions 1117- | KKKEFGGPSSGAPPPSGGSPAGSPTSTEEGTSESATPESGPGT | |
| 1136; MPP8 at | STEPSEGSAPGSPAGMGPMEIYKTVSAWKRQPVRVLSLFRNI | |
| positions 1137- | DKVLKSLGFLESGSGSGGGTLKYVEDVTNVVRRDVEKWGP | |
| 1217) | FDLVYGSTQPLGSSCDRCPGWYMFQFHRILQYALPRQESQR | |
| | PFFWIFMDNLLLTEDDQETTTRFLQTEAVTLQDVRGRDYQN | |
| | AMRVWSNIPGLKSKHAPLTPKEEEYLQAQVRSRSKLDAPKV | |
| | DLLVKNCLLPLREYFKYFSQNSLPLGSPAGSPTSTEEGTSES | |
| | ATPMDAKSLTAWSRTLVTFKDVFVDFTREEWKLLDTAQQI | |
| | VYRNVMLENYKNLVSLGYQLTKPDVILRLEKGEEPWLVES | |
| | GPGTSTEPSEGSAPGSPAMAEAFGDSEEDGEDVFEVEKILD | |
| | MKTEGGKVLYKVRWKGYTSDDDTWEPEIHLEDCKEVLLEF | |
| | RKKIAENKAKAVRKDIQR | |
| dCasPhi12(L26K_I | MAPKKKRKVGIHGVPAAMIKPTVSQFLTPGFKLIRNHSRTA | 3024 |
| 471T_E567Q)- | GKKLKNEGEEACKKFVRENEIPKDECPNFQGGPAIANIIAKS | |
| mDnmt3l- | REFTEWEIYQSSLAIQEVIFTLPKDKLPEPILKEEWRAQWLSE | |
| CBX3VD- | HGLDTVPYKEAAGLNLIIKNAVNTYKGVQVKVDNKNKNNL | |
| KRAB(ZIM3) | AKINRKNEIAKLNGEQEISFEEIKAFDDKGYLLQKPSPNKSIY | |
| (NLS at positions 1- | CYQSVSPKPFITSKYHNVNLPEEYIGYYRKSNEPIVSPYQFDR | |
| 17; dCas at | LRIPIGEPGYVPKWQYTFLSKKENKRRKLSKRIKNVSPILGII | |
| positions 18-734; | CIKKDWCVFDMRGLLRTNHWKKYHKPTDSINDLFDYFTGD | |
| NLS at positions | PVIDTKANVVRFRYKMENGIVNYKPVREKKGKELLENICDQ | |
| 735-752; linker at | NGSCKLATVDVGQNNPVAIGLFELKKVNGELTKTLISRHPTP | |
| positions 753-806; | IDFCNKITAYRERYDKLESSIKLDAIKQLTSEQKIEVDNYNN | |
| mDNMt31 at | NFTPQNTKQIVCSKLNINPNDLPWDKMTSGTHFISEKAQVSN | |
| positions 807-1021; | KSEIYFTSTDKGKTKDVMKSDYKWFQDYKPKLSKEVRDAL | |
| linker at positions | SDIEWRLRRESLEFNKLSKSREQDARQLANWISSMCDVIGIQ | |
| 1022-1041; | NLVKKNNFFGGSGKREPGWDNFYKPKKENRWWINAIHKAL | |
| CBX3VD at | TELSQNKGKRVILLPAMRTSITCPKCKYCDSKNRNGEKFNC | |
| positions 1042- | LKCGIELNADIDVATENLATVAITAQSMPKPTCERSGDAKKP | |
| 1101; linker at | VRARKAKAPEFHDKLAPSYTVVLREAVKRPAATKKAGQAK | |
| positions 1102- | KKKEFGGPSSGAPPPSGGSPAGSPTSTEEGTSESATPESGPGT | |
| 1114; KRAB | STEPSEGSAPGSPAGSMGPMEIYKTVSAWKRQPVRVLSLFR | |
| (ZIM3) at positions | NIDKVLKSLGFLESGSGSGGGTLKYVEDVTNVVRRDVEKW | |
| 1115-1214) | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMEEAEPDEFVVEKVLDRRVVNGKVEYFLKWEGFTD | |
| | ADNTWEPEENLDCPELIEAFLNSQKSRSSGGGSGGSGSMNN | |
| | SQGRVTFEDVTVNFTQGEWQRLNPEQRNLYRDVMLENYSN | |
| | LVSVGQGETTKPDVILRLEQGKEPWLEEEEVLGSGRAEKNG | |
| | DIGGQIWKPKDVKESL | |
| dCasPhi12(L26K_I | MAPKKKRKVGIHGVPAAMIKPTVSQFLTPGFKLIRNHSRTA | 3025 |
| 471T_E567Q)- | GKKLKNEGEEACKKFVRENEIPKDECPNFQGGPAIANIIAKS | |
| mDnmt3l- | REFTEWEIYQSSLAIQEVIFTLPKDKLPEPILKEEWRAQWLSE | |
| CBX5VD- | HGLDTVPYKEAAGLNLIIKNAVNTYKGVQVKVDNKNKNNL | |
| KRAB(ZIM3) | AKINRKNEIAKLNGEQEISFEEIKAFDDKGYLLQKPSPNKSIY | |
| (NLS at positions 1- | CYQSVSPKPFITSKYHNVNLPEEYIGYYRKSNEPIVSPYQFDR | |
| 17; dCas at | LRIPIGEPGYVPKWQYTFLSKKENKRRKLSKRIKNVSPILGII | |
| positions 18-734; | CIKKDWCVFDMRGLLRTNHWKKYHKPTDSINDLFDYFTGD | |
| NLS at positions | PVIDTKANVVRFRYKMENGIVNYKPVREKKGKELLENICDQ | |
| 735-752; linker at | NGSCKLATVDVGQNNPVAIGLFELKKVNGELTKTLISRHPTP | |
| positions 753-806; | IDFCNKITAYRERYDKLESSIKLDAIKQLTSEQKIEVDNYNN | |
| mDNMt31 at | NFTPQNTKQIVCSKLNINPNDLPWDKMTSGTHFISEKAQVSN | |
| positions 807-1021; | KSEIYFTSTDKGKTKDVMKSDYKWFQDYKPKLSKEVRDAL | |
| linker at positions | SDIEWRLRRESLEFNKLSKSREQDARQLANWISSMCDVIGIQ | |
| 1022-1041; | NLVKKNNFFGGSGKREPGWDNFYKPKKENRWWINAIHKAL | |
| CBX5VD at | TELSQNKGKRVILLPAMRTSITCPKCKYCDSKNRNGEKFNC | |
| positions 1042- | LKCGIELNADIDVATENLATVAITAQSMPKPTCERSGDAKKP | |
| 1104; linker at | VRARKAKAPEFHDKLAPSYTVVLREAVKRPAATKKAGQAK | |
| positions 1105- | KKKEFGGPSSGAPPPSGGSPAGSPTSTEEGTSESATPESGPGT | |
| 1117; KRAB | STEPSEGSAPGSPAGSMGPMEIYKTVSAWKRQPVRVLSLFR | |
| (ZIM3) at positions | NIDKVLKSLGFLESGSGSGGGTLKYVEDVTNVVRRDVEKW | |
| 1118-1217) | GPFDLVYGSTQPLGSSCDRCPGWYMFQFHRILQYALPRQES | |
| | QRPFFWIFMDNLLLTEDDQETTTRFLQTEAVTLQDVRGRDY | |
| | QNAMRVWSNIPGLKSKHAPLTPKEEEYLQAQVRSRSKLDAP | |
| | KVDLLVKNCLLPLREYFKYFSQNSLPLGSPAGSPTSTEEGTS | |
| | ESATPMEDEYVVEKVLDRRVVKGQVEYLLKWEGFSEEHNT | |
| | WEPEKNLDCPELISEFMKKYKKMKEGENSRSSGGGSGGSGS | |
| | MNNSQGRVTFEDVTVNFTQGEWQRLNPEQRNLYRDVMLE | |
| | NYSNLVSVGQGETTKPDVILRLEQGKEPWLEEEEVLGSGRA | |
| | EKNGDIGGQIWKPKDVKESL | |

Exemplary Guide Nucleic Acids

| | | |
|---|---|---|
| gKD010 | AUUGCUCCUUACGAGGAGACCGGUCAGAGGGACAGAGA | 2937 |
| | CC | |
| gKD033 | AUUGCUCCUUACGAGGAGACGAGAGAGGCCCAGCUACU | 2940 |
| | CG | |

### Example 8. CasPhi.12 Variant Fusion Silences Native Genes in HEK293T by Targeting Endogenous Promoters

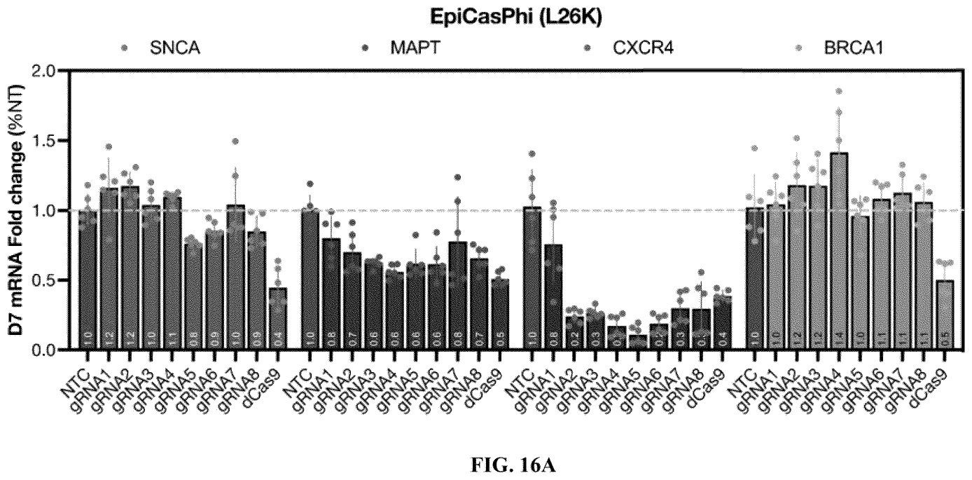

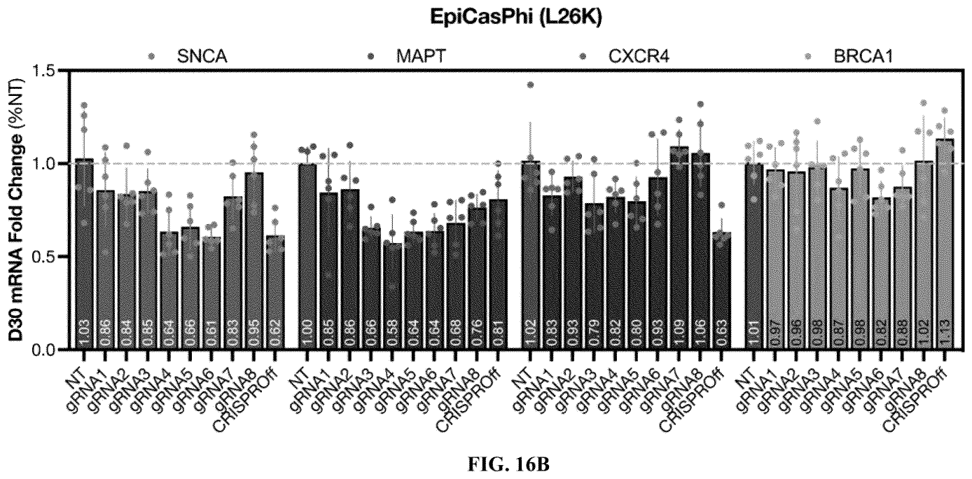

CasPhi.12 repressor fusion CasPhi^{L26K/E567Q}-mDnmt3l-KRAB^{ZNF10}, gRNA & controls were delivered to HEK293T cells by plasmid transfection without selection. gRNAs for CasPhi^{L26K/E567Q}-mDnmt3l-KRAB^{ZNF10} and controls were designed to target the promoter regions for human genes, MAPT, SNCA, BRCA1, and CXCR4. Guide RNA sequences are shown in TABLE 20 (repeat sequence italicized, spacer sequence not italicized). CasPhi^{L26K/E567Q}-mDnmt3l-KRAB^{ZNF10} activity was measured 7 days and 30 days post-transfection using RT-qPCR to quantify relative mRNA levels of each gene and normalized with GAPDH to the non-target control. FIG. 16A shows results at 7 days and FIG. 16B shows results at 30 days. CasPhi^{L26K/E567Q}-mDnmt3l-KRAB^{ZNF10} demonstrates Cas9 equivalence for some of the gRNAs tested. It is worth noting that, unlike Cas9, these CasPhi^{L26K/E567Q}-mDnmt3l-KRABZ^{NF10} and guides are capable of fitting into a single AAV vector. TABLE 19 provides exemplary guide RNAs sequences paired with CasPhi.12 fusions and variants thereof for targeting endogenous promoters.

TABLE 19
Exemplary guide RNAs for CasPhi.12 fusions and variants thereof

| | | SEQ |
|---|---|---|
| gRNA ID | Guide RNA Sequences | ID NO: |
| gSNCA-1 | CUUUCAAGACUAAUAGAUUGCUCCUUACGAGGAGACGUCAGCG | 3026 |
| | CUUCUGCCCGCCC | |
| gSNCA-2 | CUUUCAAGACUAAUAGAUUGCUCCUUACGAGGAGACUCCUCGG | 3027 |
| | CUCUCCGCCCCCA | |
| gSNCA-3 | CUUUCAAGACUAAUAGAUUGCUCCUUACGAGGAGACGGCCCCU | 3028 |
| | UCUGGUCGUCGCC | |
| gSNCA-4 | CUUUCAAGACUAAUAGAUUGCUCCUUACGAGGAGACCGCGUCG | 3029 |
| | CGGCGCUCGGUCG | |
| gSNCA-5 | CUUUCAAGACUAAUAGAUUGCUCCUUACGAGGAGACAAGGCAA | 3030 |
| | GGCGUGAGGGAGC | |
| gSNCA-6 | CUUUCAAGACUAAUAGAUUGCUCCUUACGAGGAGACCCACCCU | 3031 |
| | CGUGAGCGGAGAA | |
| gSNCA-7 | CUUUCAAGACUAAUAGAUUGCUCCUUACGAGGAGACUCCGCUC | 3032 |
| | ACGAGGGUGGAAA | |
| gSNCA-8 | CUUUCAAGACUAAUAGAUUGCUCCUUACGAGGAGACGCGGGUU | 3033 |
| | UGCCUCCCACUCC | |
| gSNCA-dCas9 | GCUCCUCCUUCUCCUUCUCCUGUUUAAGAGCUAUGCUGGAA | 3034 |
| | ACAGCAUAGCAAGUUUAAAUAAGGCUAGUCCGUUAUCAACU | |
| | UGAAAAAGUGGCACCGAGUCGGUGC | |
| gMAPT-1 | CUUUCAAGACUAAUAGAUUGCUCCUUACGAGGAGACCUGAGGC | 3035 |
| | CGGCGGGCGGCGC | |
| gMAPT-2 | CUUUCAAGACUAAUAGAUUGCUCCUUACGAGGAGACGCCGGCG | 3036 |
| | CGCGCCCUCGCAG | |
| gMAPT-3 | CUUUCAAGACUAAUAGAUUGCUCCUUACGAGGAGACGUGCCGG | 3037 |
| | AGCUGGUGGGUGG | |
| gMAPT-4 | CUUUCAAGACUAAUAGAUUGCUCCUUACGAGGAGACUGCCGCC | 3038 |
| | GCCACCACAGCCA | |
| gMAPT-5 | CUUUCAAGACUAAUAGAUUGCUCCUUACGAGGAGACUCCUCCU | 3039 |
| | CCGCUGUCCUCUC | |
| gMAPT-6 | CUUUCAAGACUAAUAGAUUGCUCCUUACGAGGAGACCCUGAUA | 3040 |
| | GUCGACAGAGGCG | |
| gMAPT-7 | CUUUCAAGACUAAUAGAUUGCUCCUUACGAGGAGACGGAGCCG | 3041 |
| | CGGCGCUUACCUG | |
| gMAPT-8 | CUUUCAAGACUAAUAGAUUGCUCCUUACGAGGAGACGUGCGCC | 3042 |
| | GGGCAUCGGUCGG | |
| gMAPT- | GCGAAGAGGGCGCGUUCCUGGUUUAAGAGCUAUGCUGGAAA | 3043 |
| dCas9 | CAGCAUAGCAAGUUUAAAUAAGGCUAGUCCGUUAUCAACUU | |
| | GAAAAAGUGGCACCGAGUCGGUGC | |
| gCXCR4-1 | CUUUCAAGACUAAUAGAUUGCUCCUUACGAGGAGACAUAAAAA | 3044 |
| | CACGCUCCGAGCG | |
| gCXCR4-2 | CUUUCAAGACUAAUAGAUUGCUCCUUACGAGGAGACUAUAAAA | 3045 |
| | GUCCGGCCGCGGC | |
| gCXCR4-3 | CUUUCAAGACUAAUAGAUUGCUCCUUACGAGGAGACCUGGCCG | 3046 |
| | CGGCCGGACUUUU | |
| gCXCR4-4 | CUUUCAAGACUAAUAGAUUGCUCCUUACGAGGAGACUUGGCUG | 3047 |
| | CGGCAGCAGGUAG | |
| gCXCR4-5 | CUUUCAAGACUAAUAGAUUGCUCCUUACGAGGAGACCUACCUG | 3048 |
| | CUGCCGCAGCCAA | |
| gCXCR4-6 | CUUUCAAGACUAAUAGAUUGCUCCUUACGAGGAGACCCAUGGA | 3049 |
| | GGGGAUCAGUGUA | |
| gCXCR4-7 | CUUUCAAGACUAAUAGAUUGCUCCUUACGAGGAGACAACUUAG | 3050 |
| | AGCGCAGCCCCUC | |
| gCXCR4-8 | CUUUCAAGACUAAUAGAUUGCUCCUUACGAGGAGACGGAGUAC | 3051 |
| | GGGUACCUCCAAU | |
| gCXCR4- | GCAGGUAGCAAAGUGACGCCGAGUUUAAGAGCUAUGCUGGA | 3052 |
| dCas9 | AACAGCAUAGCAAGUUUAAAUAAGGCUAGUCCGUUAUCAAC | |
| | UUGAAAAAGUGGCACCGAGUCGGUGC | |
| gBRCA1-1 | CUUUCAAGACUAAUAGAUUGCUCCUUACGAGGAGACCGUGGCA | 3053 |
| | ACGGAAAAGCGCG | |
| gBRCA1-2 | CUUUCAAGACUAAUAGAUUGCUCCUUACGAGGAGACAUUUAUC | 3054 |
| | UGUAAUUCCCGCG | |
| gBRCA1-3 | CUUUCAAGACUAAUAGAUUGCUCCUUACGAGGAGACAAACUGC | 3055 |
| | GACUGCGCGGCGU | |
| gBRCA1-4 | CUUUCAAGACUAAUAGAUUGCUCCUUACGAGGAGACCUGGACG | 3056 |
| | GGGGACAGGCUGU | |
| gBRCA1-5 | CUUUCAAGACUAAUAGAUUGCUCCUUACGAGGAGACUCAGAUA | 3057 |
| | ACUGGGCCCCUGC | |
| gBRCA1-6 | CUUUCAAGACUAAUAGAUUGCUCCUUACGAGGAGACCCCAGAG | 3058 |
| | CAGAGGGUGAAGG | |
| gBRCA1-7 | CUUUCAAGACUAAUAGAUUGCUCCUUACGAGGAGACCCGGGAC | 3059 |
| | UCUACUACCUUUA | |
| gBRCA1-8 | CUUUCAAGACUAAUAGAUUGCUCCUUACGAGGAGACCGAAGCU | 3060 |
| | GACAGAUGGGUAU | |
| | GGCUCGCUGAGACUUCCUGGAGUUUAAGAGCUAUGCUGGAA | 3061 |
| gBRCA1- | ACAGCAUAGCAAGUUUAAAUAAGGCUAGUCCGUUAUCAACU | |
| dCas9 | UGAAAAAGUGGCACCGAGUCGGUGC | |

### Example 9. Pooling Guides can have Additive Effect on dCasPhi.12 Mediated Gene Repression

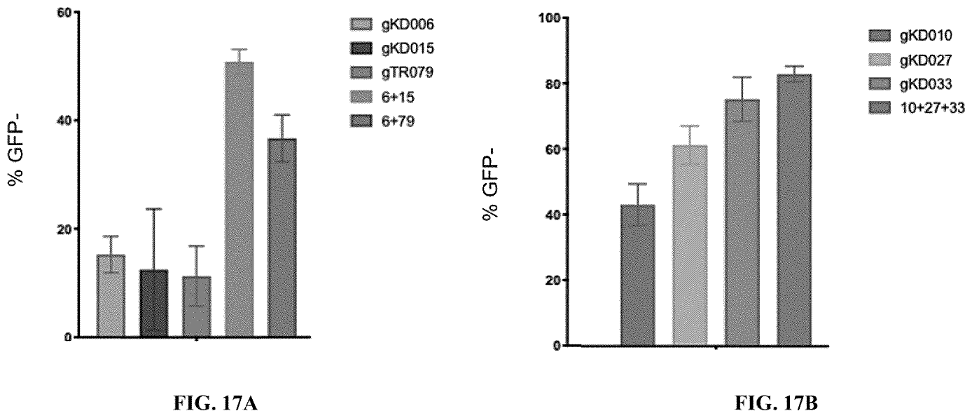

A HEK293T GFP reporter line was seeded at 30K cells/well in 96 well plates. 24 hours later, cells were transfected with 150 ng of effector protein (dCasPhi12(L26R_E567Q)-D3L-CBX3VD-KRAB(ZIM3) (SEQ ID NO: 3024)) encoding plasmid and 150 ng of plasmid encoding gRNA(s). Puromycin selection was conducted 48 hrs post transfection. Cells were passaged every 3-4 days, and flow cytometry readout obtained every 3-7 days. Flow cytometry readout gating was performed for live, single cells, and % repression gated against early day5/day9 dcas9+non targeting guide. Measurements at day 63 are shown in FIGS. 17A and 17B for two different guide poolings. Guides in the legend from top to bottom correspond to data points shown in the graph from left to right (gKD010 (SEQ ID NO: 2937), gTR079 (SEQ ID NO: 2942), gKD015 (SEQ ID NO: 2938), gKD027 (SEQ ID NO: 2939) and gKD033 (SEQ ID NO: 2940)).

### Example 10. DCasPhi.12 Fusion Constructs for Gene Silencing

A HEK293T GFP reporter line was seeded at 30K cells/well in 96 well plates. 24 hours later, cells were transfected with 150 ng of effector protein fusion protein encoding plasmid and 150 ng of plasmid encoding gRNA(s). Sequences of these fusion proteins are described in Example 7:dCasPhi12(E567Q)-D3L-KRAB(ZNF10) (SEQ ID NO: 3020), dCasPhi12(L26R_E567Q)-D3L-KRAB(ZNF10) (SEQ ID NO: 3021), dCasPhi12(L26K_I471T_E567Q)-D3L-KRAB(ZIM3) (SEQ ID NO: 3022), dCasPhi12(L26K_I471T_E567Q)-D3L-KRAB(ZNF10)-MPP8 (SEQ ID NO: 3023), dCasPhi12(L26K_I471T_E567Q)-D3L-CBX3VD-KRAB(ZIM3) (SEQ ID NO: 3024), and dCasPhi12(L26K_I471T_E567Q)-D3L-CBX5VD-KRAB(ZIM3) (SEQ ID NO: 3025). Puromycin selection was conducted 48 hrs post transfection. Cells were passaged every 3-4 days, and flow cytometry readout obtained every 3-7 days.

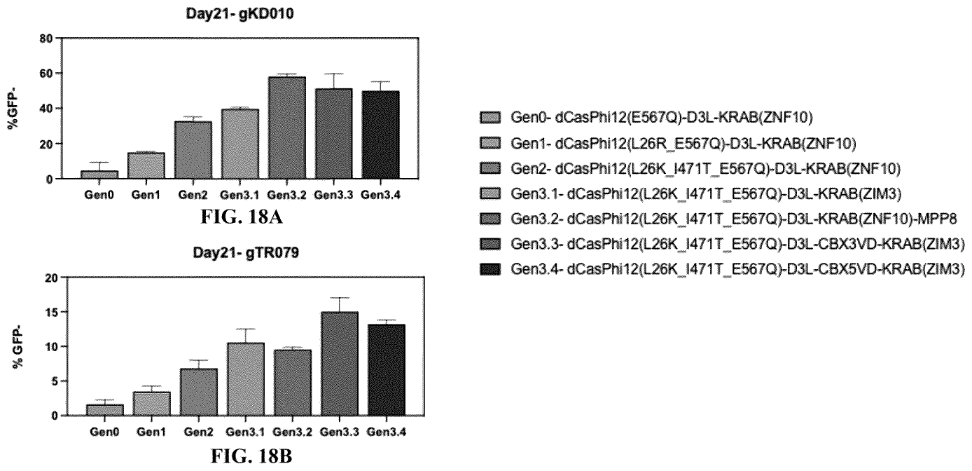

Flow cytometry readout gating was performed for live, single cells, and % repression gated against early day5/day9 dcas9+non targeting guide. Measurements at day 21 are shown in FIG. 18A-18B for two different guide nucleic acids (FIG. 18A for gKD010 (SEQ ID NO: 2937) and FIG. 18B for gTRO79 (SEQ ID NO: 2942)). Fusion proteins in the legend from top to bottom correspond to data points shown in the graphs from left to right.

1. A composition comprising a fusion protein or a nucleic acid encoding the fusion protein, wherein the fusion protein comprises:
a) an effector protein comprising an amino acid sequence that is at least 90% identical to SEQ ID NO: 1, wherein the effector protein comprises at least one amino acid substitution relative to SEQ ID NO: 1, wherein the amino acid substitution is selected from: L26R, L26K, K38R, Q54R, Q79R, K92E, S108R, E109R, T114R, E119S, A121Q, L182R, Q183R, K184R, K189P, S198R, H208R, Y220S, S223P, E258K, D369A, D369N, N406K, K435Q, I471T, V521T, E567A, E567Q, N568D, S638K, D658A, and D658N, and a combination thereof;
b) a methyltransferase; and
c) a repressor domain.

2. (canceled)

3. The composition of claim 1, wherein the effector protein comprises a first amino acid substitution selected from: L26R, L26K, K38R, Q54R, Q79R, K92E, S108R, E109R, T114R, E119S, A121Q, L182R, Q183R, K184R, K189P, S198R, H208R, Y220S, S223P, E258K, N406K, K435Q, I471T, V521T, N568D, and S638K, and a second amino acid substitution selected from: D369A, D369N, D658A, D658N, E567A, and E567Q.

4. The composition of claim 3, wherein the effector protein comprises the amino acid substitutions L26K, K189P, S223, E258K, K435Q, I471T, and E567Q.

5. (canceled)

6. (canceled)

7. The composition of claim 1, wherein the length of the effector protein is 400 to 800 amino acids.

8. (canceled)

9. The composition of claim 1, wherein the at least one methyltransferase is selected from DNMT3A, and DNMT3L, and a combination thereof.

10. The composition of claim 1, wherein the methyltransferase comprises an amino acid sequence that is at least 90% identical to a sequence selected from SEQ ID NO: 10 and SEQ ID NO: 11.

11. The composition of claim 1, wherein the repressor domain is selected from KRAB(ZNF10), KRAB (ZIM3), YAF2, MPP8, RYBP, CBX3VD, and CBX5VD, and a combination thereof.

12. The composition of claim 1, wherein the repressor domain comprises an amino acid sequence that is at least 90% identical to a sequence selected from SEQ ID NOs: 12 and 3005-3009.

13. The composition of claim 1, comprising a guide nucleic acid or a nucleic acid encoding the guide nucleic acid, wherein the guide nucleic acid comprises a protein binding sequence and a spacer sequence, and wherein the spacer sequence is at least 90% complementary to a target sequence in a human genome.

14. (canceled)

15. (canceled)

16. The composition of claim 13, wherein the protein binding sequence comprises a sequence that is at least 80% identical to a nucleotide sequence selected from SEQ ID NOs: 5-9.

17. The composition of claim 13, wherein the target sequence is located in a human gene selected from TABLE 11.

18. (canceled)

19. (canceled)

20. The composition of claim 13, wherein the target sequence is adjacent to a protospacer adjacent motif of 5′-NTTN-3′.

21. (canceled)

22. (canceled)

23. (canceled)

24. (canceled)

25. (canceled)

26. The composition of claim 1, wherein amongst the effector protein, methyltransferase, and repressor domain, the effector protein is nearest to the N terminus, the repressor domain is located nearest to the C terminus, and the methyltransferase is located between the effector protein and the repressor domain.

27. The composition of claim 1, wherein the nucleic acid encoding the fusion protein is an adeno associated viral (AAV) vector.

28. (canceled)

29. The composition of claim 1, further comprising a lipid nanoparticle (LNP).

30. The composition of claim 29, wherein the nucleic acid encoding the fusion protein is a messenger RNA.

31. (canceled)

32. A composition comprising a fusion protein or a nucleic acid encoding the fusion protein, wherein the fusion protein comprises:
a) an effector protein; and
b) a methyltransferase, wherein the methyltransferase is DNMT3L,
wherein the composition does not comprise DNMT3A.

33. (canceled)

34. (canceled)

35. (canceled)

36. (canceled)

37. (canceled)

38. (canceled)

39. (canceled)

40. (canceled)

41. A method of modifying a target nucleic acid or the expression thereof, the method comprising contacting the target nucleic acid with the composition of claim 1.

42. A cell modified by the composition of claim 1.

43. (canceled)

44. A method of reducing expression of a gene in a cell, the method comprising contacting the cell with the composition of claim 1.

45. (canceled)
